(19) United States
(12) Patent Application Publication
(10) Pub. No.: US 2009/0029346 A1
(43) Pub. Date: Jan. 29, 2009

(54) Detection of human papilloma virus

(75) Inventors: Douglas Spencer Millar, New South Wales (AU); George Gabor L. Miklos, New South Wales (AU); John Robert Melki, New South Wales (AU)

Correspondence Address: KNOBBE MARTENS OLSON & BEAR LLP, 2040 MAIN STREET, FOURTEENTH FLOOR, IRVINE, CA, 92614, US

(73) Assignee: Human Genetic Signatures Pty., Ltd., Macquarie Park, NSW (AU)

(21) Appl. No.: 11/660,586

(22) PCT Filed: Dec. 22, 2005
(86) PCT No.: PCT/AU2005/001963

§ 371 Date: Aug. 7, 2008

Related U.S. Application Data
(60) Provisional application No. 60/638,625, filed on Dec. 23, 2004.

(51) Int. Cl. C12Q 1/70

(52) U.S. Cl. 435/5

(57) ABSTRACT

An assay for detecting HPV comprising treating the viral nucleic acid with an agent that modifies cytosine to form derivative viral nucleic acid, amplifying at least a part of the derivative viral nucleic acid to form an HPV-specific nucleic acid molecule, and looking for the presence of an HPV-specific nucleic acid molecule, wherein detection of the HPV-specific nucleic acid molecule is indicative HPV.

### TECHNICAL FIELD

The invention relates to assays for detection of human papilloma virus.

### BACKGROUND ART

### Human Papilloma Virus

It has been challenging to implement reliable and robust DNA-based detection systems that recognise all the different HPV types in a single assay, since not only are there cross hybridization problems between different HPV genomic types, but the exact classification of what constitutes an HPV type is dependent upon genomic sequence similarities which have significant bioinformatic limitations. Thus, while new HPV types have been defined as ones where there is less than 90% sequence similarity with previous HPV types, finer taxonomic subdivisions are more problematic to deal with. Thus, a new HPV ‘subtype’ is defined when the DNA sequence similarity is in the 90-98% range relative to previous subtypes. A new ‘variant’ is defined when the sequence similarity is between 98-100% of previous variants (1993, Van Rast, M. A., et al., Papillomavirus Rep, 4, 61-65; 1998, Southern, S. A. and Herrington, C. S. Sex. Transm. Inf. 74, 101-109). This spectrum can broaden further to the point where variation could be measured based on comparing single genomes from single isolated viral particles. In such a case, a ‘genotype’ would be any fully sequenced HPV genome that minimally differs by one base from any other fully sequenced HPV genome. This includes all cases where a single base at a defined position can exist in one of four states, G, A, T or C, as well as cases where the base at that given position has been altered by deletion, addition, amplification or transposition to another site.

The difficulties faced by existing HPV detection systems in the context of disease risk assessment are largely threefold. First limitations of the technology systems themselves. Secondly, limitations of the pathological interpretations of diseased cell populations. Thirdly, limitations at the clinical level of assessing disease progression in different human populations that are subject to differences in genetic background as well as contributing cofactors.

### Clinical Detection of Cervical Abnormalities

HPVs of certain types are implicated in cancers of the cervix and contribute to a more poorly defined fraction of cancers of the vagina, vulvae, penis and anus. The ring of tissue that is the cervical transformation zone is an area of high susceptibility to HPV carcinogenicity, and assessment of its state from complete cellular normalcy to invasive carcinoma has been routinely evaluated using visual or microscopic criteria via histological, cytological and molecular biological methodologies. The early detection of virally-induced abnormalities at both the viral level and that of the compromised human cell, would be of enormous clinical relevance if it could help in determining where along a molecular trajectory, from normal to abnormal tissue, a population of cells has reached. However, despite the use of the Pap smear for half a century, a solid early risk assessment between abnormal cervical cytological diagnoses and normalcy is currently still problematical. Major problems revolve around the elusive criteria on which to define ‘precancer’, such as the various grades of Cervical Intraepithelial Neoplasia, (CIN1, CIN2 and CIN3) and hence on the clinical decisions that relate to treatment options. Precancer definitions are considered by some clinicians to be a pseudo-precise way in which to avoid using CIN2, CIN3 and carcinoma in situ. There is great heterogeneity in microscopic diagnoses and even in the clinical meaning of CIN2, (2003, Schiffman, M., J. Nat. Cancer Instit. Monog. 31, 14-19). Some CIN2 lesions have a bad microscopic appearance but will nevertheless be overcome by the immune system and disappear, whereas other lesions will progress to invasive carcinoma. Thus CIN2 is considered by some as a buffer zone of equivocal diagnosis although the boundary conditions of such a zone remain controversial. Some clinicians consider it to be poor practice to combine CIN2 and CIN3, whereas others will treat all lesions of CIN2 or worse. Finally, the literature indicates that between a third and two thirds of CIN3 assigned women will develop invasive carcinoma, but even this occurs in an unpredictable time-dependent fashion, (2003, Schiffman, M., J. Nat. Cancer. Instit. Monog. 31, 14-19; 1978, Kinlen, L. J., et al., Lancet 2, 463-465; 1956, Peterson, O. Am. J. Obstet. Gynec. 72, 1063-1071).

The central problem still confronting physicians today is that defining low grade cytological abnormalities such as atypical squamous cells of undetermined significance, (ASCUS), or squamous intraepithelial lesions (SILs) is difficult. ‘In fact, ASCUS is not a proper diagnosis but rather is a “wastebasket” category of poorly understood changes’, (1996, Lorincz, A. T., 1996, J. Obstet. Gyncol. Res. 22, 629-636). The whole spectrum of precancerous lesions is difficult to interpret owing to cofactor effects from oral contraceptive use, smoking, pathogens other than HPV such as Chlamydia trachomatis and Herpes Simplex Virus type 2, antioxidant nutrients and cervical inflammation, all of which are claimed to modulate the risk of progression from high grade squamous intraepithelial lesions (HSILs) to cervical cancer (2003, Castellsague, X. J. Nat. Cancer Inst. Monog. 31, 20-28). The introduction of the Bethesda system of classification and its revision in 2001 has done little to reduce the confusion among clinicians, since it was initially found unhelpful to include koilocytotic atypia with CIN1 into the newer category of low-grade squamous intraepithelial lesions, (LSILs). The result of the introduction of the Bethesda system was that many clinicians would not carry out colposcopy on koilocytotic atypia, ‘but felt compelled do so on patients with CIN1’, (1995, Hatch, K. D., Am. J. Obstet. Gyn. 172, 1150-1157). It was clear that although colposcopic expertise required many years of training, subjective cytological criteria still lead to inconsistencies and non-reproducibilities, (1994, Sherman, M. E., Am. J. Clin. Pathology, 102, 182-187; 1988, Giles, J. A., Br. Med. J., 296, 1099-1102).

The continuing diagnostic hurdle is that vague diagnoses such as ‘atypia’ can account for 20% or more of diagnoses in some settings, (1993, Schiffman, M. Contemporary OB/GYN, 27-40). This is illustrated by a test designed specifically to evaluate the level of independent diagnostic agreement of pathologists on smears that were ‘atypical’. It was found that exact agreement between five professional pathologists on an identical set of samples occurred in only 29% of cases, (1994, Sherman, M. E., et al., Am. J. Clin. Pathology, 102, 182-187). The net result is that cervical cytology continues to have high false negative rates (termed low sensitivity) and high false positive rates, (termed low specificity). The cytological interpretations of various pathologists yield a false negative rate of up to 20% or so and a false positive rate of up to 15% (1993, Koss, L. G., Cancer, 71, 1406-1412). False positive results lead to unnecessary colposcopic examinations, biopsies and treatments, all of which add to the health care cost burden. False negative results lead to potential malpractice law suits with their associated costs. It was into this arena that molecular diagnoses of early stages of cervical abnormalities using tests for HPV offer a less subjective test than cytological ones.

### Limitations of Assays for HPV Detection.

The presence of HPV DNA was originally assayed by low stringency Southern Blot technology applied to DNA from samples from exophytic condylomata acuminata, (1975, Southern, E. M., J. Mol. Biol. 98, 503-527; 1993, Brown, D. R., et al., J. Clinical Microbiology, 31, 2667-2673). However, in a clinical setting, the technique was found to be ‘tedious, time consuming and requires fresh tissue samples’ and there was extensive between-laboratory variation. The technology was deemed ‘unsuitable for clinical use’ (1995, Ferenozy, A, Int. J. Gynecol. Cancer, 5, 321-328).

The introduction of a modification of the Southern Blot, namely the Dot Blot, was US Food and Drug Administration (FDA) approved and marketed as Virapap™ and Viratype™ (Life Technologies Inc, Gaithersburg, Md.). The detection limits were 3 picograms of HPV DNA per millilitre of sample, which is approximately 375,000 viral genomes per ml. However, the sensitivity of the Virapap™ kit turned out to be less than that of cytological methods, (1991, Bauer, H. M., JAMA, 265, 472-477). In addition such kits used radioactive nucleic acids for detection, were labour intensive, expensive in a clinical setting, and there was widespread confusion about their clinical applicability. Finally, the molecular hybridization conditions for Viratype™ gave cross hybridization between different HPV types. Hence precisely determining which HPV types were present in a sample meant that the Viratype™ test had to be run a second time at higher stringencies of hybridization than those stipulated by the manufacturer.

At the in situ cytological level, matters were little better. Much of the early data on HPV detection using Fluorescent In Situ Hybridization (FISH) were erroneous and there was misclassification of HPV types; (1996, Schiffman, M.; in Richart, Contemporary OB/GYN, July 1996, pp 80). Currently, hybridization to paraffin-embedded sections using Omniprobe™ (Digene Diagnostics Inc, Silver Spring, Md.) to detect HPV sequences yields a sensitivity that is claimed to be 20 to 50 viruses per cell, and the Enzo PathoGene HPV In Situ Typing Assay (Enzo Life Sciences 60 Executive Boulevard, Farmingdale, N.Y.) is in use for determining the presence of HPV DNA beginning with formalin fixed, paraffin embedded tissue sections.

In situ hybridization tests are exacting, labour intensive and time consuming. Even with the most advanced Fluorescent In Situ Hybridization technology (FISH), it is currently not possible to routinely assay for a single full length viral genome, or a small segment of a viral genome that may be integrated into a single chromosomal site in the human genome. Routine FISH is best achieved using probes which are the size of Bacterial Artificial Chromosomes (of the order of 100 kilobases). These are over ten times the size of the full HPV genome and 100 times the size of an HPV gene such as E6 or E7.

Immunohistochemistry, using an antibody directed against an epitope of the L1 capsid protein of all relevant HPV types is another detection method (2004, Griesser, H., et al., Analyt. Quant. Cytol. Histol. 26, 241-245), but again it is labour intensive and time consuming.

The first generation HPV Hybrid Capture kit developed by Digene Diagnostics utilized non radioactive RNA probes to detect lesional HPV DNA and its non-radioactive nature made for easier and more economical use. Hybrid Capture used signal amplification rather than amplification of the target DNA to obtain sensitivity. However, as pointed out by Richart (Contemporary OB/GYN, July 1996), Hybrid Capture was prone to false positive results, owing to cross hybridization between novel HPV types and other HPV probes, and particularly when chemiluminescent values suddenly spiked. In addition, first generation Hybrid Capture detected only one third to one half of the infections detected by PCR. Hybrid Capture has since been upgraded, so that the Hybrid Capture 2™ (Digene Corporation, Gaithersburg, Md.) test now contains a mixture of thirteen HPV probes for types, 16, 18, 31, 33, 35, 39, 45, 51, 52, 56, 58, 59 and 68 and the US FDA approved threshold has been set at 1 picogram of HPV DNA per ml of test solution, equivalent to 125,000 viral genomes per ml, (2001, Salomon, D., J. Nat. Cancer Instit. 93, 293-299). Hybrid Capture 3™ (Digene Corporation, Gaithersburg, Md.) utilizes an even more complex mixture of biotinylated capture oligonucleotides, and unlabelled ‘blocker’ oligonucleotides, that together are claimed to eliminate the issue of probe cross-reactivity seen with Hybrid Capture 2™. However, Hybrid Capture 2™, with its known problems of probe cross hybridization, is still the only FDA approved product, (2001, Lorincz, A. & Anthony, J. Papillomavirus Report, 12, 145-154).

Hybrid Capture has also been adapted to measuring the RNA expression that derives from the genes comprising the HPV genome (U.S. Pat. No. 6,355,424). Specifically, the ratio of E6 and/or E7 RNA levels relative to E2 and/or L1 RNA levels is assessed. This is done by hybridization of biotinylated DNA probes to viral RNA from cells lysed in a microtiter plate. The RNA:DNA hybrids are captured by antibody binding as in the previous embodiment of the Hybrid Capture technology and assayed as previously using a chemiluminescent reagent.

The most sensitive HPV detection methodology is polymerase chain reaction (PCR) which readily detects a single viral copy in a human genome. The first HPV PCR detection kit was the L1 consensus primer polymerase chain reaction method from Roche Molecular Systems with a practical lower detection limit of about 100 viral genomes. This test was evaluated by direct comparisons between Southern Blot and PCR technologies (1991, Schiffman, M. H., J. Clin. Microbiol, 29, 573-577) and was found to be very labour intensive, (see 1995, Schiffman, M. H., J. Clin. Microbiol, 33, 545-550).

Given all the problems and shortcomings outlined above, there is still controversy as regards the clinical impact of DNA methodologies in screening for preneoplastic lesions. Sensitive early molecular prognostic indicators of cellular abnormalities would be extremely valuable.

The present inventors have developed new methods, kits and integrated bioinformatic platforms for detecting HPV and differentiating between different types of HPV.

### DISCLOSURE OF INVENTION

In a first aspect, the present invention provides an assay for detecting human papilloma virus (HPV) comprising:

treating the viral nucleic acid with an agent that modifies cytosine to form derivative viral nucleic acid;

amplifying at least a part of the derivative viral nucleic acid to form an HPV-specific nucleic acid molecule; and

looking for the presence of an HPV-specific nucleic acid molecule, wherein detection of the HPV-specific nucleic acid molecule is indicative of HPV.

preferably, the assay further comprises:

providing HPV primers capable of allowing amplification of an HPV-specific nucleic acid molecule.

Preferably, the virus is in a sample. The sample can be any suitable clinical, clinical product or environmental sample. Typically, the sample will be swab, biopsy, smear, Pap smear, blood, plasma, serum, blood product, surface scrape, spatula, liquid suspension, frozen material, paraffin blocks, glass slides, forensic collection systems or archival material. Preferably, the sample is a smear, Pap smear or liquid suspension of cells.

Preferably, the agent modifies cytosine to form uracil in the derivative nucleic acid. Preferably, the agent is selected from bisulfite, acetate or citrate. More preferably, the agent is sodium bisulfite.

Preferably, the agent modifies an cytosine to a uracil in each strand of complementary double stranded viral nucleic acid forming two derivative but non-complementary viral nucleic acid molecules.

Preferably, the agent modifies cytosine to uracil which is then replaced as a thymine during amplification of the derivative nucleic acid. Preferably, the agent used for modifying cytosine is sodium bisulfite. Other agents that similarly modify cytosine, but not methylated cytosine can also be used in the method of the invention. Examples include, but not limited to bisulfite, acetate or citrate. Preferably, the agent is sodium bisulfite, a reagent, which in the presence of acidic aqueous conditions, modifies cytosine into uracil.

Sodium bisulfite (NaHSO₃) reacts readily with the 5,6-double bond of cytosine to form a sulfonated cytosine reaction intermediate which is susceptible to deamination, and in the presence of water gives rise to a uracil sulfite. If necessary, the sulfite group can be removed under mild alkaline conditions, resulting in the formation of uracil. Thus, potentially all cytosines will be converted to uracils. Any methylated cytosines, however, cannot be converted by the modifying reagent due to protection by methylation.

Preferably, the derivative viral nucleic acid has a reduced total number of cytosines compared with the corresponding untreated viral nucleic acid.

Preferably, the amplification is carried out by polymerase chain reaction (PCR), ligase chain reaction (LCR), isothermal amplification, signal amplification or combination of the above. More preferably, the amplification is carried out by PCR.

Usually, amplification forms an HPV-specific nucleic acid molecule that does not form part of a natural HPV genome.

In a preferred form, the HPV-specific nucleic acid molecule is specific for an HPV species, a type of HPV or sub-type of HPV. The HPV type can confer a high, medium or low level oncogenic status on a given tissue in a particular human ethnic lineage. High risk HPV types are HPV16, 18, 45 and 56, medium risk HPV types are HPV31, 33, 35, 39, 51, 52, 56, 58, 59 and 68, and low risk strains are HPV6, 11, 30, 42, 43, 44, 53, 54, and 55. Preferably, high-risk HPV16, 18, 45 or 56 and medium risk HPV 31, 33, 35, 39, 51, 52, 58, 59 and 68 are detected.

It will be appreciated that the HPV-specific nucleic acid is detected by any suitable means. Examples include, but not limited to, gel electrophoresis, hybridisation with labelled probes, use of tagged primers that allow subsequent identification, an enzyme linked assay, or use of fluorescently-tagged primers that give rise to a signal upon hybridisation with the target DNA.

In a second aspect, the present invention provides an HPV primer or probe comprising one or more of SEQ ID NO: 1 to SEQ ID NO: 516.

Preferably, the HPV primer or probe for detecting high-medium risk HPV strains includes one or more of SEQ ID NO: 333 to SEQ ID NO: 350.

Preferably, the HPV primer or probe for detecting HPV includes SEQ ID NO: 462, SEQ ID NO: 479, SEQ ID NO: 463, SEQ ID NO: 478, SEQ ID NO: 470, SEQ ID NO: 485, or SEQ ID NO: 486.

In a third aspect, the present invention provides a kit for the detection of HPV comprising two or more HPV primers or probes according to the second aspect of the present invention together with suitable reagent or diluent.

In a fourth aspect, the present invention provides a derivative HPV nucleic acid.

Preferably, the derivative HPV nucleic acid is from high-risk HPV16, 18, 45 or 56 and medium risk HPV 31, 33, 35, 39, 51, 52, 58, 59 and 68.

More preferably, the derivative HPV nucleic acid comprises any one or more of SEQ ID NO: 614, SEQ ID NO: 617, SEQ ID NO: 620, SEQ ID NO: 623, SEQ ID NO: 626, SEQ ID NO: 629, SEQ ID NO: 632, SEQ ID NO: 635, SEQ ID NO: 638, SEQ ID NO: 641, SEQ ID NO: 644, SEQ ID NO: 647, SEQ ID NO: 650, SEQ ID NO: 653, SEQ ID NO: 656, SEQ ID NO: 659, SEQ ID NO: 662, SEQ ID NO: 665, SEQ ID NO: 668, SEQ. ID NO: 671, SEQ ID NO: 674, SEQ ID NO: 677, SEQ ID NO: 680, SEQ ID NO: 683, SEQ ID NO: 686, or SEQ ID NO: 689, parts thereof comprising at least 15 nucleotides, and nucleic acid molecules capable of hybridizing under stringent conditions to SEQ ID NO: 614, SEQ ID NO: 617, SEQ ID NO: 620, SEQ ID NO: 623, SEQ ID NO: 626, SEQ ID NO: 629, SEQ ID NO: 632, SEQ ID NO: 635, SEQ ID NO: 638, SEQ ID NO: 641, SEQ ID NO: 644, SEQ ID NO: 647, SEQ ID NO: 650, SEQ ID NO: 653, SEQ ID NO: 656, SEQ ID NO: 659, SEQ ID NO: 662, SEQ ID NO: 665, SEQ ID NO: 668, SEQ ID NO: 671, SEQ ID NO: 674, SEQ ID NO: 677, SEQ ID NO: 680, SEQ ID NO: 683, SEQ ID NO: 686, or SEQ ID NO: 689.

The parts of the derivative HPV nucleic acid can be at least 15, 16, 17, 18, 19, 20, 21, 22, 23, 24, 25, 26, 27, 28, 29, 30 etc or more nucleotides.

In a fifth aspect, the present invention provides a simplified HPV nucleic acid.

Preferably, the simplified HPV nucleic acid is from high-risk HPV16, 18, 45 or 56 and medium risk HPV 31, 33, 35, 39, 51, 52, 58, 59 and 68.

More preferably, the simplified HPV nucleic acid comprises any one or more of SEQ ID NO: 615, SEQ ID NO: 618, SEQ ID NO: 621, SEQ ID NO: 624, SEQ ID NO: 627, SEQ ID NO: 630, SEQ ID NO: 633, SEQ ID NO: 636, SEQ ID NO: 639, SEQ ID NO: 642, SEQ ID NO: 645, SEQ ID NO: 648, SEQ ID NO: 651, SEQ ID NO: 654, SEQ ID NO: 657, SEQ ID NO: 660, SEQ ID NO: 663, SEQ ID NO: 666, SEQ ID NO: 669, SEQ ID NO: 672, SEQ ID NO: 675, SEQ ID NO: 678, SEQ ID NO: 681, SEQ ID NO: 684, SEQ ID NO: 687, or SEQ ID NO: 690; parts thereof comprising at least 15 nucleotides, and nucleic acid molecules capable of hybridizing under stringent conditions to SEQ ID NO: 615, SEQ ID NO: 618, SEQ ID NO: 621, SEQ ID NO: 624, SEQ ID NO: 627, SEQ ID NO: 630, SEQ ID NO: 633, SEQ ID NO: 636, SEQ ID NO: 639, SEQ ID NO: 642, SEQ ID NO: 645, SEQ ID NO: 648, SEQ ID NO: 651, SEQ ID NO: 654, SEQ ID NO: 657, SEQ ID NO: 660, SEQ ID NO: 663, SEQ ID NO: 666, SEQ ID NO: 669, SEQ ID NO: 672, SEQ ID NO: 675, SEQ ID NO: 678, SEQ ID NO: 681, SEQ ID NO: 684, SEQ ID NO: 687, or SEQ ID NO: 690.

The parts of the simplified HPV nucleic acid can be at least 15, 16, 17, 18, 19, 20, 21, 22, 23, 24, 25, 26, 27, 28, 29, 30 etc nucleotides.

In a sixth aspect, the present invention provides use of the derivative or simplified HPV nucleic acid according to the fourth or fifth aspects of the present invention to obtain probes or primers for HPV detection.

In a seventh aspect, the present invention provides an assay for detecting the presence of HPV in a sample comprising:

obtaining viral nucleic acid from a sample;

treating the viral nucleic acid with bisulphite under conditions that cause cytosines in the viral nucleic acid to be converted to uracil to form derivative viral nucleic acid;

providing primers capable of binding to regions of derivative viral nucleic acid, the primers being capable of allowing amplification of a desired HPV-specific nucleic acid molecule to the derivative viral nucleic acid;

carrying out an amplification reaction on the derivative viral nucleic acid; and

looking for the presence of a desired amplified nucleic acid product, wherein detection of the amplified product is indicative of the presence of HPV in the sample.

In one preferred form, the assay further comprises:

treating a sample having HPV present with an additional test which can determine the type, subtype, variant or genotype of HPV in the sample.

The additional test is preferably an amplification reaction using primers specific for a given HPV type or group of types, wherein the presence of an amplified product is indicative of the HPV type or group of types.

In an eighth aspect, the present invention provides a method for producing an HPV-specific nucleic acid comprising:

treating a sample containing HPV nucleic acid with an agent that modifies cytosine to form derivative HPV nucleic acid; and

amplifying at least part of the derivative HPV nucleic acid to form a simplified HPV nucleic acid having a reduced total number of cytosines compared with the corresponding untreated HPV nucleic acid, wherein the simplified nucleic acid molecule includes a nucleic acid sequence specific for HPV.

For double stranded DNA which contains no methylated cytosines, the treating step results in two derivative nucleic acids, each containing the bases adenine, guanine, thymine and uracil. The two derivative nucleic acids are produced from the two single strands of the double stranded DNA. The two derivative nucleic acids have substantially no cytosines but still have the same total number of bases and sequence length as the original untreated DNA molecule. Importantly, the two derivatives are not complimentary to each other and form a top and a bottom strand. One or more of the strands can be used as the target for amplification to produce the simplified nucleic acid molecule.

Typically, the simplified nucleic acid sequence specific for HPV does not occur naturally in an untreated HPV genome.

In a preferred form, the method further comprises:

detecting the HPV-specific nucleic acid having a nucleic acid sequence indicative of a particular HPV type.

The HPV-specific nucleic acid can be detected by any suitable means. Examples include, but not limited to, gel electrophoresis, hybridisation with labelled probes, use of tagged primers that allow subsequent identification (eg by an enzyme linked assay), and use of fluorescently-tagged primers that give rise to a signal upon hybridisation with the target DNA (eg Beacon and TaqMan systems).

Preferably, the HPV-specific nucleic acid molecule is detected by:

providing a detector ligand capable of binding to a target region of the nucleic acid molecule and allowing sufficient time for the detector ligand to bind to the target region; and

measuring binding of the detector ligand to the nucleic acid molecule to detect the presence of the target nucleic acid molecule. It will be appreciated that the nucleic acid molecule can be detected by any suitable means known to the art.

In a ninth aspect, the present invention provides a method for obtaining an HPV-specific nucleic acid molecule comprising:

treating HPV nucleic acid from representative types of HPV with an agent that modifies cytosine to form a derivative HPV nucleic acid molecule for each type;

amplifying at least part of the derivative HPV nucleic acid molecule from each type to form simplified nucleic acid molecules having a reduced total number of cytosines compared with the corresponding untreated HPV nucleic acid molecules; and

obtaining an HPV-specific nucleic acid molecule for a type or types by identifying common or unique sequence or sequences in the simplified nucleic acid molecules.

It will be appreciated that the method can be carried out bioinformatically (in silico) from known nucleic acid sequences of HPV types where each cytosine in the original sequences is changed to thymine to obtain the simplified HPV nucleic acid molecules directly. Sequence identity can be determined from the simplified nucleic acid sequences.

For example, treating step can be carried out bioinformatically by replacing all cytosines in the representative HPV genomes with uracil to form derivative HPV nucleic acid molecules for each type. Each derivative HPV nucleic acid molecule will have the same total number of bases as the corresponding untreated HPV genome. It will be appreciated that each uracil in the derivative HPV nucleic acid molecule will be copied to a thymine during the amplification process. Accordingly, the amplified sequences forming the simplified nucleic acid molecules will not correspond to sequences in the original HPV genome. Each strand (‘top’ and ‘bottom’) of the derivative nucleic acid will not be complimentary so therefore they form two possible templates for amplification.

When an HPV-specific nucleic acid molecule has been obtained for any given HPV type by this method, probes or primers can be designed to ensure amplification of the region of interest in PCR or other suitable amplification reaction. It is important to note that both strands of a treated and thus converted genome, (hereafter termed “derivative’) can be analyzed for primer design, since treatment or conversion leads to asymmetries of sequence, (see below), and hence different primer sequences are required for the detection of the ‘top’ and ‘bottom’ strands of the same locus. Thus, there are two populations of molecules, the converted genome as it exists immediately after conversion, and the population of molecules that results after the derivative is replicated by conventional enzymological means (PCR). Primers are typically designed for the converted top strand for convenience but primers can also be generated for the bottom strand. Thus, it will be possible to carry out clinical or scientific assays on samples to detect a given type of HPV.

The present invention also allows the generation of probes or primers that are indicative of all representative types of HPV which can be used to determine whether any HPV genome is present in a given sample. Further HPV type-specific probes can be used to actually detect or identify a given, type, subtype, variant and genotype examples of HPV.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred embodiments will be described with reference to the following drawings and examples.

### BRIEF DESCRIPTION OF THE DRAWINGS

FIG. 1 shows DNA alignment of the ‘top’ strand of the same 8 base pair genomic region of individual viral types, HPV 33, 35, 39, 52, 58, 16, 18, 45 and 56, before bisulphite treatment and the corresponding sequence of the derivative after bisulphite conversion. The cytosines have been converted to uracils and the uracils are represented as thymines. Nucleotide positions that vary between the types are shown as bold. (SEQ ID NO is listed after each sequence).

FIG. 2 shows DNA alignment of the ‘top’ strand of a 17 base pair genomic region of individual viral types HPV 6, 11, 43, 44, 53, 55, 30, 31, 39, 51, 52, 16, 18 and 45, and the ‘complexity-reduction’ following bisulphite treatment of the DNA sample that gives rise to the derivative sequence. The consensus primers for the derivatives of the ‘top’ and ‘bottom’ strands will differ after bisulphite treatment; only primers for one strand are illustrated. The cytosines have been converted to uracils and the uracils are represented as thymines. Nucleotide positions that vary between the HPV types are shown as bold. (SEQ ID NO: is listed after each sequence).

FIG. 3 shows DNA alignment of the ‘top’ strand of a 20 base pair region of individual viral types (HPV 6, 43, 44, 54, 55, 30, 33, 58, 18 and 45) and identification of regions of >90% sequence similarity in the derivative sequences using the HGS complexity-reduction method. The consensus primers for the ‘top’ and ‘bottom’ strands will differ after bisulphite treatment; only primers for one strand are illustrated. The cytosines have been converted to uracils and the uracils are represented as thymines. Nucleotide positions that vary between the HPV types are shown as bold. (SEQ ID NO: is listed after each sequence).

FIG. 4 shows DNA alignment of the ‘top’ strand of a 20 base pair region of individual viral types (HPV 6, 43, 44, 54, 55, 30, 33, 58, 18 and 45) and the sequence of shorter high affinity INA primers or probes that can be used more effectively in hybridization reactions than standard oligonucleotides. The consensus primers for the ‘top’ and ‘bottom’ strands will differ after bisulphite treatment; only primers for one strand are illustrated. The cytosines have been converted to uracils and the uracils are represented as thymines. (SEQ ID NO: is listed after each sequence).

FIG. 5 shows the results of a PCR amplification using universal HGS complexity-reduced primers for the ‘top’ strand of the L1 region of bisulphite-treated HPV DNA extracted from liquid-based cytology (LBC) specimens from sixteen patients #s 1 to 16.

FIG. 6 shows multiplex PCR amplification using HGS complexity-reduced primers for the ‘top’ strand of the E7 region of the high-risk bisulphite-treated complexity-reduced derivative from HPV16, 18, 45 and 56. The DNA was extracted from liquid-based cytology specimens from the same patients #s 1 to 16. The arrow indicates the expected size of the amplified nucleic acid products.

FIG. 7 shows a PCR amplification using HGS complexity-reduced primers for the ‘top’ strand of the E7 region of the high risk bisulphite-treated complexity-reduced derivative from HPV16. The DNA was extracted from liquid based cytology specimens from the same patient samples #s 1 to 16.

FIG. 8 shows a PCR amplification using HGS complexity-reduced primers for the ‘top’ strand of the E7 region of the high risk bisulphite-treated complexity-reduced derivative from HPV18: The DNA was extracted from liquid based cytology specimens from the same patient samples #s 1 to 16.

FIG. 9 shows a PCR amplification using HGS complexity-reduced primers for the ‘top’ strand of the E4, E6 and E7 regions of the high risk bisulphite-treated complexity-reduced derivative from HPV16. The DNA was extracted from liquid based cytology specimens from the same patient samples #s 1 to 16. The arrows indicate the expected size of the amplified nucleic acid products.

FIG. 10 shows a PCR amplification using HGS complexity-reduced primers for the ‘top’ strand of the E4, E6 and E7 regions of the high risk bisulphite-treated complexity-reduced derivative from HPV18. The DNA was extracted from liquid based cytology specimens from the same patient samples #s 1 to 16. The arrows indicate the expected size of the amplified nucleic acid products.

FIG. 11 summarizes the three different derivative regions, (E4, E6 and E7) that have been PCR amplifiable from HPV derivatives of various risk types, using complexity-reduced primers for the ‘top’ strand on samples from normal or abnormal cervical tissues from liquid-based cytology samples from patients #s A to T. The results of 580 PCR tests generated from Liquid Based Cytology samples from 20 patients [denoted #s A-T] and examined for size by gel electrophoresis, and in some cases by direct sequence analysis to verify the identity of the product. Primers were made to determine the presence [denoted positive, and shaded], or absence [negative] of regions of the E4, E6 and E7 regions of various HPV types. A universal nested primer set to a part of the L1 region of all HPV types, irrespective of risk status, [denoted Uni], is shown for column 2. For the purposes of this figure high risk HPV strains are defined as HPV 16, 18, 45 and 56, medium risk strains as HPV 30, 31, 33, 35, 39, 51, 52, 56, 58, 59 and 66, while low risk strains are defined as HPV6, 11, 42, 43, 44, 53, 54, and 55. A multiplex nested primer set to a part of the E7 region of all high-risk HPV types [denoted High] is shown for column 3. A multiplex nested primer set to a part of the E7 region of all medium-risk HPV types [denoted Medium] is shown for column 4. A multiplex nested primer set to a part of the E7 region of all low-risk HPV types [denoted Low] is shown for column 5. The presence of a band on a gel is indicative of the designated viral fragment in the clinical sample.

FIG. 12 illustrates the effects of primer degeneracy on the probability of obtaining a PCR product on bisulphite-treated samples from patients #s 21 to 42. Primers were made to the ‘top’ strand only. The effect of the degeneracy level of a single member of a 23-mer primer pair on the efficiency of PCR amplification reactions. In PCR reaction HPV-HM, the number of possible primer combinations for primer #1 is 72. In PCR reaction HPV-HML, the number of possible primer combinations for primer #1 is greatly increased to 2304. Amplified nucleic acid products are visible in PCR reaction HPV-HM but not in PCR reaction HPV-HML. The symbols G, A, T and C denote the form normal bases, while D, K, W, and H are the standard symbols for mixtures of different bases at that position. (D=A, G or T; K=G or T; W=A or T; H=A, T or C). (SEQ ID NO: is listed after each sequence).

FIG. 13 shows the top strand of the HPV16 viral nucleic acid molecule in its three possible sequences; A. the normal viral sequence (SEQ ID NO: 613); B. the derivative sequence with uracils replacing cytosines (SEQ ID NO: 614); and C. the genomically simplified sequence where uracils have been replaced by thymines (SEQ ID NO: 615).

FIG. 14 shows the bottom strand of the HPV16 viral nucleic acid molecule in its three possible sequences; A. the normal viral sequence (SEQ ID NO: 616), B. the derivative sequence with uracils replacing cytosines (SEQ ID NO: 617); and C. the genomically simplified sequence where uracils have been replaced by thymines (SEQ ID NO: 618).

FIG. 15 is a schematic of the genomic landscape of the top strand of HPV 16 from nucleotide position #1 to nucleotide position #7904 with the boxes indicating the positions of various nested primer sets used for amplification purposes. The positions of primer sets for primers that are useful for amplifying DNA from a combinations of HPV types, such as high and medium risk, (HM) and high, medium and low risk, (HML); high, (H) and high and medium, (HM) combinations are as indicated.

FIG. 16 is a schematic of the genomic landscape of the bottom strand of HPV 16 from nucleotide position #1 to nucleotide position #7904 with the boxes indicating the positions of various nested primer sets used for amplification purposes. The positions of primer sets for primers that are useful for amplifying DNA from a combinations of HPV types, such as high and medium risk, (HM) and high, medium and low risk, (HML); high, (H) and high and medium, (HM) combinations are as indicated.

FIG. 17 shows a tissue section from an individual with cervical carcinoma. Arrow 1 reveals a darkened area of cancerous cells with large nuclei. Arrow 2 shows normal connective tissue.

FIG. 18 shows the results of a PCR amplification using the high-medium risk HGS complexity-reduced primers (for the detection of thirteen HPV types, namely HPV 16, 18, 31, 33, 35, 39, 45, 51, 52, 56, 58, 59 and 68) for the ‘top’ strand of the E7 region of bisulphite-treated HPV DNA extracted from liquid-based cytology (LBC) specimens from twelve patient samples in which cytological analyses had been completed, (denoted #s 1 to 12).

FIG. 19 shows the results of a PCR amplification using material from clinical samples #2, #4, #7 and #11 from the patients that were positive for a high-medium risk HPV in FIG. 18 and a determination of exactly which of the HPV types (HPV 16, 18, 31, 33, 35, 39, 45, 51, 52, 56, 58, 59 and 68), was responsible for each of the amplicons visible in FIG. 18.

FIG. 20 shows the results of PCR amplification from archival paraffin sections from material from 16 patients with High grade Squamous Intraepithelial Lesions (HSILs), using high-medium risk primer sets (HPV 16, 18, 31, 33, 35, 39, 45, 51, 52, 56, 58, 59 and 68), made to the genomically simplified top strand of HPV.

FIG. 21 A shows the results of PCR amplification from Liquid Based Cytology samples using primers made to the bottom strand of bisulphite converted, genomically simplified DNA. The primers target HPV types (High-medium risk types HPV 16, 18, 31, 33, 35, 39, 45, 51, 52, 56, 58, 59, 68 and low risk types HPV 6, 11, 42, 43, 44, 53, 54 and 55).

FIG. 21 B shows the results of PCR amplification from Liquid Based Cytology samples using primers made to the top strand of bisulphite converted, genomically simplified DNA. The primers target the thirteen high-medium risk HPV types, (HPV 16, 18, 31, 33, 35, 39, 45, 51, 52, 56, 58, 59 and 68).

FIG. 22 shows results of DNA sequencing of an HPV amplicon genotyped as HPV 16 from portion of an automated gel read. The peaks correspond to the DNA bases as indicated.

FIG. 23 shows the top strand of the HPV18 viral nucleic acid molecule in its three possible sequences; A. the normal viral sequence (SEQ ID NO: 619); B. the derivative sequence with uracils replacing cytosines (SEQ ID NO: 620); and C. the genomically simplified sequence where uracils have been replaced by thymines (SEQ ID NO: 621).

FIG. 24 shows the bottom strand of the HPV18 viral nucleic acid molecule in its three possible sequences; A. the normal viral sequence (SEQ ID NO: 622), B. the derivative sequence with uracils replacing cytosines (SEQ ID NO: 623); and C. the genomically simplified sequence where uracils have been replaced by thymines (SEQ ID NO: 624).

FIG. 25 shows the top strand of the HPV31 viral nucleic acid molecule in its three possible sequences; A. the normal viral sequence (SEQ ID NO: 625); B. the derivative sequence with uracils replacing cytosines (SEQ ID NO: 626); and C. the genomically simplified sequence where uracils have been replaced by thymines (SEQ ID NO: 627).

FIG. 26 shows the bottom strand of the HPV31 viral nucleic acid molecule in its three possible sequences; A. the normal viral sequence (SEQ ID NO: 628), B. the derivative sequence with uracils replacing cytosines (SEQ ID NO: 629); and C. the genomically simplified sequence where uracils have been replaced by thymines (SEQ ID NO: 630).

FIG. 27 shows the top strand of the HPV33 viral nucleic acid molecule in its three possible sequences; A. the normal viral sequence (SEQ ID NO: 631); B. the derivative sequence with uracils replacing cytosines (SEQ ID NO: 632); and C. the genomically simplified sequence where uracils have been replaced by thymines (SEQ ID NO: 633).

FIG. 28 shows the bottom strand of the HPV33 viral nucleic acid molecule in its three possible sequences; A. the normal viral sequence (SEQ ID NO: 634), B. the derivative sequence with uracils replacing cytosines (SEQ ID NO: 635); and C. the genomically simplified sequence where uracils have been replaced by thymines (SEQ ID NO: 636).

FIG. 29 shows the top strand of the HPV35 viral nucleic acid molecule in its three possible sequences; A. the normal viral sequence (SEQ ID NO: 637); B. the derivative sequence with uracils replacing cytosines (SEQ ID NO: 638); and C. the genomically simplified sequence where uracils have been replaced by thymines (SEQ ID NO: 639).

FIG. 30 shows the bottom strand of the HPV35 viral nucleic acid molecule in its three possible sequences; A. the normal viral sequence (SEQ ID NO: 640), B. the derivative sequence with uracils replacing cytosines (SEQ ID NO: 641); and C. the genomically simplified sequence where uracils have been replaced by thymines (SEQ ID NO: 642).

FIG. 31 shows the top strand of the HPV39 viral nucleic acid molecule in its three possible sequences; A. the normal viral sequence (SEQ ID NO: 643); B. the derivative sequence with uracils replacing cytosines (SEQ ID NO: 644); and C. the genomically simplified sequence where uracils have been replaced by thymines (SEQ ID NO: 645).

FIG. 32 shows the bottom strand of the HPV39 viral nucleic acid molecule in its three possible sequences; A. the normal viral sequence (SEQ ID NO: 646), B. the derivative sequence with uracils replacing cytosines (SEQ ID NO: 647); and C. the genomically simplified sequence where uracils have been replaced by thymines (SEQ ID NO: 648).

FIG. 33 shows the top strand of the HPV45 viral nucleic acid molecule in its three possible sequences; A. the normal viral sequence (SEQ ID NO: 649); B. the derivative sequence with uracils replacing cytosines (SEQ ID NO: 650); and C. the genomically simplified sequence where uracils have been replaced by thymines (SEQ ID NO: 651).

FIG. 34 shows the bottom strand of the HPV45 viral nucleic acid molecule in its three possible sequences; A. the normal viral sequence (SEQ ID NO: 652), B. the derivative sequence with uracils replacing cytosines (SEQ ID NO: 653); and C. the genomically simplified sequence where uracils have been replaced by thymines (SEQ ID NO: 654).

FIG. 35 shows the top strand of the HPV51 viral nucleic acid molecule in its three possible sequences; A. the normal viral sequence (SEQ ID NO: 655); B. the derivative sequence with uracils replacing cytosines (SEQ ID NO: 656); and C. the genomically simplified sequence where uracils have been replaced by thymines (SEQ ID NO: 657).

FIG. 36 shows the bottom strand of the HPV51 viral nucleic acid molecule in its three possible sequences; A. the normal viral sequence (SEQ ID NO: 658), B. the derivative sequence with uracils replacing cytosines (SEQ ID NO: 659); and C. the genomically simplified sequence where uracils have been replaced by thymines (SEQ ID NO: 660).

FIG. 37 shows the top strand of the HPV52 viral nucleic acid molecule in its three possible sequences; A. the normal viral sequence (SEQ ID NO: 661); B. the derivative sequence with uracils replacing cytosines (SEQ ID NO: 662); and C. the genomically simplified sequence where uracils have been replaced by thymines (SEQ ID NO: 663).

FIG. 38 shows the bottom strand of the HPV52 viral nucleic acid molecule in its three possible sequences; A. the normal viral sequence (SEQ ID NO: 664), B. the derivative sequence with uracils replacing cytosines (SEQ ID NO: 665); and C. the genomically simplified sequence where uracils have been replaced by thymines (SEQ ID NO: 666).

FIG. 39 shows the top strand of the HPV56 viral nucleic acid molecule in its three possible sequences; A. the normal viral sequence (SEQ ID NO: 667); B. the derivative sequence with uracils replacing cytosines (SEQ ID NO: 668); and C. the genomically simplified sequence where uracils have been replaced by thymines (SEQ ID NO: 669).

FIG. 40 shows the bottom strand of the HPV56 viral nucleic acid molecule in its three possible sequences; A. the normal viral sequence (SEQ ID NO: 670), B. the derivative sequence with uracils replacing cytosines (SEQ ID NO: 671); and C. the genomically simplified sequence where uracils have been replaced by thymines (SEQ ID NO: 672).

FIG. 41 shows the top strand of the HPV58 viral nucleic acid molecule in its three possible sequences; A. the normal viral sequence (SEQ ID NO: 673); B. the derivative sequence with uracils replacing cytosines (SEQ ID NO: 674); and C. the genomically simplified sequence where uracils have been replaced by thymines (SEQ ID NO: 675).

FIG. 42 shows the bottom strand of the HPV58 viral nucleic acid molecule in its three possible sequences; A. the normal viral sequence (SEQ ID NO: 676), B. the derivative sequence with uracils replacing cytosines (SEQ ID NO: 677); and C. the genomically simplified sequence where uracils have been replaced by thymines (SEQ ID NO: 678).

FIG. 43 shows the top strand of the HPV59 viral nucleic acid molecule in its three possible sequences; A. the normal viral sequence (SEQ ID NO: 679); B. the derivative sequence with uracils replacing cytosines (SEQ ID NO: 680); and C. the genomically simplified sequence where uracils have been replaced by thymines (SEQ ID NO: 681).

FIG. 44 shows the bottom strand of the HPV59 viral nucleic acid molecule in its three possible sequences; A. the normal viral sequence (SEQ ID NO: 682), B. the derivative sequence with uracils replacing cytosines (SEQ ID NO: 683); and C. the genomically simplified sequence where uracils have been replaced by thymines (SEQ ID NO: 684).

FIG. 45 shows the top strand of the HPV68a viral nucleic acid molecule in its three possible sequences; A. the normal viral sequence (SEQ ID NO: 685); B. the derivative sequence with uracils replacing cytosines (SEQ ID NO: 686); and C. the genomically simplified sequence where uracils have been replaced by thymines (SEQ ID NO: 687).

FIG. 46 shows the bottom strand of the HPV68a viral nucleic acid molecule in its three possible sequences; A. the normal viral sequence (SEQ ID NO: 688), B. the derivative sequence with uracils replacing cytosines (SEQ ID NO: 689); and C. the genomically simplified sequence where uracils have been replaced by thymines (SEQ ID NO: 690).

### MODE(S) FOR CARRYING OUT THE INVENTION

### Definitions

The term “genomic simplification” as used herein means the genomic (or other) nucleic acid is modified from being comprised of four bases adenine (A), guanine (G), thymine (T) and cytosine (C) to substantially containing the bases adenine (A), guanine (G), thymine (T) but still having substantially the same total number of bases.

The term “derivative nucleic acid” as used herein means a nucleic acid that substantially contains the bases A, G, T and U (or some other non-A, G or T base or base-like entity) and has substantially the same total number of bases as the corresponding unmodified nucleic acid. Substantially all cytosines in the untreated nucleic acid will have been converted to uracil (or some other non-A, G or T base or base-like entity) during treatment with the agent. It will be appreciated that altered cytosines, such as by methylation, may not necessarily be converted to uracil (or some other non-A, G or T base or base-like entity). Preferably, cytosine is modified to uracil.

The term “derivative HPV nucleic acid” as used herein means an HPV nucleic acid that substantially contains the bases A, G, T and U (or some other non-A, G or T base or base-like entity) and has substantially the same total number of bases as the corresponding unmodified HPV nucleic acid. Substantially all cytosines in the HPV DNA will have been converted to uracil (or some other non-A, G or T base or base-like entity) during treatment with the agent. It will be appreciated that altered cytosines, such as by methylation, may not necessarily be converted to uracil (or some other non-A, G or T base or base-like entity). As HPV nucleic acid typically does not contain methylated cytosine (or other cytosine alterations) the treated step preferably converts all cytosines. Preferably, cytosine is modified to uracil.

The term “converted genome” as used herein means an HPV genome that substantially contains the bases A, G, T and U (or some other non-A, G or T base or base-like entity) and has substantially the same total number of bases as the corresponding unconverted HPV genome. Substantially all cytosines in the HPV genome will have been converted to uracil (or some other non-A, G or T base or base-like entity).

The term “simplified nucleic acid” as used herein means the resulting nucleic acid product obtained after amplifying derivative nucleic acid. Uracil in the derivative nucleic acid is then replaced as a thymine (T) during amplification of the derivative nucleic acid to form the simplified nucleic acid molecule. The resulting product has substantially the same number of total bases as the corresponding unmodified nucleic acid but is substantially made up of a combination of three bases (A, G and T).

The term “simplified HPV nucleic acid” as used herein means the resulting HPV nucleic acid product obtained after amplifying derivative HPV nucleic acid. Uracil in the derivative nucleic acid is then replaced as a thymine (T) during amplification of the derivative nucleic acid to form the simplified HPV nucleic acid molecule. The resulting product has substantially the same number of total bases as the corresponding unmodified HPV nucleic acid but is substantially made up of a combination of three bases (A, G and T).

The term “simplified sequence” as used herein means the resulting nucleic acid sequence obtained after amplifying derivative nucleic acid to form a simplified nucleic acid. The resulting simplified sequence has substantially the same number of total bases as the corresponding unmodified nucleic acid sequence but is substantially made up of a combination of three bases (A, G and T).

The term “simplified HPV sequence” as used herein means the resulting nucleic acid sequence obtained after amplifying derivative HPV nucleic acid to form a simplified HPV nucleic acid. The resulting simplified sequence has substantially the same number of total bases as the corresponding unmodified HPV nucleic acid sequence but is substantially made up of a combination of three bases (A, G and T).

The term “non-converted sequence” as used herein means the nucleic acid sequence prior to treatment and amplification. A non-converted sequence typically is the sequence of the naturally occurring nucleic acid.

The term “non-converted HPV sequence” as used herein means the HPV nucleic acid sequence prior to treatment and amplification. A non-converted sequence typically is the sequence of the naturally occurring HPV nucleic acid.

The term “modifies” as used herein means the conversion of an cytosine to another nucleotide. Preferably, the agent modifies unmethylated cytosine to uracil to form a derivative nucleic acid.

The term “agent that modifies cytosine” as used herein means an agent that is capable of converting cytosine to another chemical entity. Preferably, the agent modifies cytosine to uracil which is then replaced as a thymine during amplification of the derivative nucleic acid. Preferably, the agent used for modifying cytosine is sodium bisulfite. Other agents that similarly modify cytosine, but not methylated cytosine can also be used in the method of the invention. Examples include, but not limited to bisulfite, acetate or citrate. Preferably, the agent is sodium bisulfite, a reagent, which in the presence of acidic aqueous conditions, modifies cytosine into uracil. Sodium bisulfite (NaHSO₃) reacts readily with the 5,6-double bond of cytosine to form a sulfonated cytosine reaction intermediate which is susceptible to deamination, and in the presence of water gives rise to a uracil sulfite. If necessary; the sulfite group can be removed under mild alkaline conditions, resulting in the formation of uracil. Thus, potentially all cytosines will be converted to uracils. Any methylated cytosines, however, cannot be converted by the modifying reagent due to protection by methylation. It will be appreciated that cytosine (or any other base) could be modified by enzymatic means to achieve a derivative nucleic acid as taught by the present invention.

There are two broad generic methods by which bases in nucleic acids may be modified: chemical and enzymatic. Thus, modification for the present invention can also be carried out by naturally occurring enzymes, or by yet to be reported artificially constructed or selected enzymes. Chemical treatment, such as bisulphite methodologies, can convert cytosine to uracil via appropriate chemical steps. Similarly, cytosine deaminases, for example, may carry out a conversion to form a derivative nucleic acid. The first report on cytosine deaminases to our knowledge is 1932, Schmidt, G., Z. physiol. Chem., 208, 185; (see also 1950, Wang, T. P., Sable, H. Z., Lampen, J. O., J. Biol. Chem, 184, 17-28, Enzymatic deamination of cytosines nucleosides). In this early work, cytosine deaminase was not obtained free of other nucleo-deaminases, however, Wang et al. were able to purify such an activity from yeast and E. coli. Thus any enzymatic conversion of cytosine to form a derivative nucleic acid which ultimately results in the insertion of a base during the next replication at that position, that is different to a cytosine, will yield a simplified genome. The chemical and enzymatic conversion to yield a derivative followed by a simplified genome are applicable to any nucleo-base, be it purines or pyrimidines in naturally occurring nucleic acids of microorganisms.

The term “simplified form of the HPV genome or nucleic acid” as used herein means that an HPV genome or nucleic acid, which usually contains the four common bases G, A, T and C, now consists largely of only three bases, G, A and T since most or all of the Cs in the genome have been converted to Ts by appropriate chemical modification and subsequent amplification procedures. The simplified form of the genome means that relative genomic complexity is reduced from a four base foundation towards a three base composition.

The term “base-like entity” as used herein means an entity that is formed by modification of cytosine. A base-like entity can be recognised by a DNA polymerase during amplification of a derivative nucleic acid and the polymerase causes A, G or T to be placed on a newly formed complementary DNA strand at the position opposite the base-like entity in the derivate nucleic acid. Typically, the base-like entity is uracil that has been modified from cytosine in the corresponding untreated nucleic acid. Examples of a base-like entity includes any nucleo-base, be it purine or pyrimidine.

The term “natural HPV genome” as used herein means the genome of a virus as it exists in nature. A natural HPV genome comprises a sequence of nucleotide bases forming an HPV nucleic acid molecule.

The term “relative complexity reduction” as used herein relates to probe length, namely the increase in average probe length that is required to achieve the same specificity and level of hybridization of a probe to a specific locus, under a given set of molecular conditions in two genomes of the same size, where the first genome is “as is” and consists of the four bases, G, A T and C, whereas the second genome is of exactly the same length but some cytosines, (ideally all cytosines), have been converted to thymines. The locus under test is in the same location in the original unconverted as well as the converted genome. On average, an 11-mer probe will have a unique location to which it will hybridize perfectly in a regular genome of 4,194,304 bases consisting of the four bases G, A, T and C, (4¹¹ equals 4,194,304). However, once such a regular genome of 4,194,304 bases has been converted by bisulfite or other suitable means, this converted genome is now composed of only three bases and is clearly less complex. However the consequence of this decrease in genomic complexity is that our previously unique 11-mer probe no longer has a unique site to which it can hybridize within the simplified genome. There are now many other possible equivalent locations of 11 base sequences that have arisen de novo as a consequence of the bisulfite conversion. It will now require a 14-mer probe to find and hybridize to the original locus. Although it may initially appear counter intuitive, one thus requires an increased probe length to detect the original location in what is now a simplified three base genome, because more of the genome looks the same, (it has more similar sequences). Thus the reduced relative genomic complexity, (or simplicity of the three base genome), means that one has to design longer probes to find the original unique site.

The term “relative genomic complexity reduction” as used herein can be measured by increased probe lengths capable of being HPV-specific as compared with unmodified DNA. This term also incorporates the type of probe sequences that are used in determining the presence of HPV. These probes may have non-conventional backbones, such as those of PNA or LNA or modified additions to a backbone such as those described in INA. Thus, a genome is considered to have reduced relative complexity, irrespective of whether the probe has additional components such as Intercalating pseudonucleotides, such as in INA. Examples include, but not limited to, DNA, RNA, locked nucleic acid (LNA), peptide nucleic acid (PNA), MNA, altritol nucleic acid (ANA), hexitol nucleic acid (HNA), intercalating nucleic acid (INA), cyclohexanyl nucleic acid (CNA) and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. Non-naturally occurring nucleotides include, but not limited to the nucleotides comprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides (IPN). The presence of IPN is not part of the complexity description for nucleic acid molecules, nor is the backbone part of that complexity, such as in PNA.

By “INA” is meant an intercalating nucleic acid in accordance with the teaching of WO 03/051901, WO 03/052132, WO 03/052133 and WO 03/052134 (Unest A/S) incorporated herein by reference. An INA is an oligonucleotide or oligonucleotide analogue comprising one or more intercalator pseudonucleotide (IPN) molecules.

By “HNA” is meant nucleic acids as for example described by Van Aetschot et al., 1995.

By “MNA” is meant nucleic acids as described by Hossain et al, 1998.

“ANA” refers to nucleic acids described by Allert et al, 1999.

“LNA” may be any LNA molecule as described in WO 99/14226 (Exiqon), preferably, LNA is selected from the molecules depicted in the abstract of WO 99/14226. More preferably, LNA is a nucleic acid as described in Singh et al, 1998, Koshkin et al, 1998 or Obika et al., 1997.

“PNA” refers to peptide nucleic acids as for example described by Nielsen et al, 1991.

“Relative complexity reduction” as used herein, does not refer to the order in which bases occur, such as any mathematical complexity difference between a sequence that is ATATATATATATAT (SEQ ID NO: 691) versus one of the same length that is AAAAAAATTTTTTT (SEQ ID NO: 692), nor does it refer to the original re-association data of relative genome sizes, (and inferentially, genomic complexities), introduced into the soientific literature by Waring, M. & Britten R. J. 1966, Science, 154, 791-794; and Britten, R. J and Kohne D E., 1968, Science, 161, 529-540, and earlier references therein that stem from the Carnegie Institution of Washington Yearbook reports.

An example clarifies the consequences of such a conversion process when applied to individual viral genomes, or to a mixture of viral genomes that occurs in a clinical sample containing both human cells and viral genomes, or parts thereof.

A normal 10 base genomic sequence which is 5′ GGGGAAATTC 3′ (SEQ ID NO: 693) (the ‘top’ strand) will have a complementary ‘bottom’ strand that is 5′ GAATTTCCCC 3′ (SEQ ID NO: 694). Following denaturation and bisulphite treatment, the ‘top’ strand becomes 5′ GGGGAAATTU 3′ (SEQ ID NO: 695) and the ‘bottom’ strand becomes 5′ GAATTTUUUU 3′ (SEQ ID NO: 696). Since cytosines have been converted to uracils, and uracils are equivalent to thymines in terms of recognition by DNA polymerase machinery ex vivo, the top strand derivative is essentially 5′ GGGGAAATTT 3′ (SEQ ID NO: 696) and the bottom strand derivative is 5′ GAATTTTTTT 3′ (SEQ ID NO: 697). Thus an initially normal genome has been converted from one in which the top and bottom strands between them had 5 Cs and 5 Ts, to a derivative population of polymers in which the top and bottom strands between them now have no Cs and 10 Ts. The normal genome has been reduced from a four base entity to a three base derivative. It has been “complexity-reduced”. In addition, a ‘locus’ in a derivative population refers only to positional coordinates within that derivative. After bisulphite conversion for example, a locus is stripped of all functional biological characteristics at any network level. If it was previously coding, regulatory or structural, it is now biological gibberish in both strands. A derivative population is thus a collection of functionless chemical polymers that now represent two non-complementary ghosts of the previously complementary strands of a genome that is now informationally impotent. Furthermore, the derivatives are unique and do not represent, except by statistical accident, sequences generated by normal evolutionary processes in any cellular, (or viral or viroid), life forms.

### Probes and Complexity-Reduction.

In the formal sense of molecular probes, we define herein ‘complexity-reduction’ in terms of the increase in probe length (IPL) that is required to achieve the same specificity and level of hybridization of a probe to a specific locus, under a given set of molecular conditions in two entities of the same size, the first being the normal genome and the second being the simplified sequence. For the purposes of molecular utility, IPL is an integer equal to or greater than 1. Each locus remains in the same location in the normal genome as well as the simplified nucleic acid.

Although it may appear counter intuitive, an increased oligonucleotide probe length may be required to detect the original locus in what is now a T-enriched simplified HPV nucleic acid. Thus the reduced-complexity of a simplified HPV nucleic acid means longer probes may need to be designed for the ‘top’ and ‘bottom’ strands of a locus to find the original unique site in the simplified HPV nucleic acid. However, as shown below, the use of Intercalating Nucleic Acid (INA) probes allows for much shorter probes than conventional oligonucleotides, and so overcomes this requirement for increased lengths, if required.

The principle of complexity-reduction, defined in terms of probe lengths and different probe sequences for ‘top’ and ‘bottom’ strands at a locus, is a relative term applicable to different structural or modified probes and primers in different molecular milieu. An example for INAs clarifies this relativity. The significant advantages of INAs over the standard oligonucleotide probes are that INAs can be made much shorter than conventional oligonucleotides and still achieve equivalent hybridization results, (INA length<oligonucleotide length). This is due to the high affinity of INA for complementary DNA owing to the Intercalating Pseudo Nucleotides, IPNs, that are a structural component of INAs. Thus if it requires an INA of length X nucleotides, with a given number of IPNs, to achieve successful and specific hybridization to an unconverted genome, it will still require an INA of length >X to hybridize to the same locus in a bisulphite converted genome under the same molecular conditions.

It is also particularly important to note that in the case of host-pathogen interactions, (where both viral and host genomes co-exist in the same clinical sample but in very different concentrations), ‘complexity-reduction’ and the use of INAs or other probes introduce new advantageous conditions into hybridization protocols, particularly since INAs have a preference for hybridizing to nucleic acid sequences that are AT-enriched. For example, in a pure solution of wild type HPV DNA, the approximate length of a viral probe or primer that is required to find and hybridize to a unique locus in the 7904 base HPV16 genome is approximately a 6-mer probe/primer, (46 equals 4096 bases). Following bisulphite treatment to generate a T-enriched simplified HPV nucleic acid, it now requires an approximately 8-mer probe or primer to find this unique location, (3⁸ equals 6561 bases) under the same molecular conditions.

However, when two grossly unequally sized genomes are initially present in a sample, such as the HPV genome of 7904 base pairs and the human genome of approximately 3,000,000,000 base pairs, and both genomes are ‘complexity-reduced’ to their respective derivatives, the probes or primers for a unique viral sequence now hybridize to their derivative targets in a solution that is overwhelmingly dominated by the T-enriched human simplified nucleic acid. If, for example, there was one simplified HPV nucleic acid for each human simplified nucleic acid in the sample, then viral probes or primers are hybridizing to a 3,000,007,904 base pair simplified nucleic acid. Hence assaying for a unique viral sequence now requires approximately 14-mer probes or primers, to avoid hybridization signals emanating from viral decoy loci that have newly arisen in human sequences.

In addition to ‘complexity-reduction’ issues involving probe and primer lengths, there are also important changes to the kinetics of hybridization and the ability to detect PCR products when the number of degenerate primers used in a PCR reaction is modest. Owing to the extensive genomic variation between HPV types, prior art amplifications have required the use of a large number of degenerate primers to produce relevant amplified nucleic acid products or amplicons from multiplex PCR reactions. However, the greater the degeneracy in the probe/primer pool, the lower is the concentration of any individual relevant probe or primer in solution. Such a situation has analogies to the kinetics and fidelity of hybridizations in the driver-tracer reactions carried out on complex eukaryotic genomes, and first introduced into the scientific literature in 1966 by Waring, M. & Britten R. J. Science, 154, 791-794; and in 1968 by Britten, R. J and Kohne D E., Science, 161, 529-540, (and earlier references therein that stem from the Carnegie Institution of Washington Yearbook reports).

In addition, when HPV PCR primers are in high concentration relative to human derivatives, the dominant force in the hybridization reaction is the HPV primer. For example, if the viral load in a sample is high, (say of the order of 100,000 HPV genomes to a single human genome), then the kinetics of hybridization of viral primers would be a 100,000 times faster than if there were only one HPV derivative per human derivative. In the former case the viral component behaves in solution as if it were a highly repetitive component of a genome. However, in order to detect different HPV types of different risk in a clinical sample by means of a single PCR reaction, different primers are typically required from each HPV type necessitating the use of degenerate entities. The net result is that the primer population can be combinatorially staggering in a conventional multiplex PCR reaction on mixed normal genomes. There can literally be thousands of different primers competing for hybridization sites with the net result that PCR amplifications fail, or the amplified nucleic acid product distribution becomes heavily biased in favour of a particular HPV type present in the sample. This presents a major problem for the generation of data from clinical samples in which conventional unconverted genomes are present.

The present invention of ‘complexity-reduction’, combined with the optional use of INA probes and primers overcomes many of the difficulties of these prior art problems.

The term “capable of specifically hybridizing” is used interchangeably with the term “capable of hybridizing under stringent conditions” herein to mean that nucleic acids having the ability to hybridize under stringent conditions with all or parts of an other nucleic acid molecule. Nucleotide sequence that is complementary with at least one helical turn (about 10 to 15 nucleotides) of a + or − strand of a DNA segment. By capable of hybridizing under stringent conditions it is meant that annealing the subject nucleic acid with at least a region of nucleic acid occurs under standard conditions, e.g., high temperature and/or low salt content, which tend to preclude hybridization of noncomplementary nucleotide sequences. An example of a stringent protocol for hybridization of nucleic acid probes to immobilised DNA (involving 0.1×SSC, 68° C. for 2 hours) is described in Maniatis, T., et al., Molecular Cloning: A Laboratory Manual, Cold Springs Harbor Laboratory, 1982, at pages 387-389, although conditions will vary depending on the application.

The term “nucleotide sequence” is used herein to refer to a sequence of nucleosides or nucleotides.

The term “contiguous nucleotide sequences” is used herein to refer to a sequence of nucleotides linked in a serial array, one following the other.

The term “PCR” (polymerase chain reaction) is used herein to refer to the process of amplifying DNA segments through the use of a DNA template molecule, two oligonucleotide primers, and a DNA polymerase enzyme. The DNA template is dissociated at high temperature from primers that may be annealed to the template. The DNA polymerase copies the template starting at the primers. The process is repeated about 30 to 40 times to amplify and enrich the template-specific molecules in the reaction product.

### Primers and Complexity-Reduction

It should be noted that complexity-reduction differs depending upon whether the population of molecules that has been converted, (the derivatives), remains in the converted state, or is subjected to further amplification. In the examples discussed above, the derivative population remained unamplified, as it would exist in a clinical sample. Recall that the top strand (5′ GGGGAATTC 3′) (SEQ ID NO: 693), and the bottom strand (5′ GAATTTCCCC 3′) (SEQ ID NO: 694), were converted to 5′ GGGGAAATTU 3′ (SEQ ID NO: 695) and 5′ GAATTTUUUU 3′ (SEQ ID NO: 696) respectively. Since cytosines have been converted to uracils, and uracils are equivalent to thymines in terms of recognition by DNA polymerase machinery ex vivo, the top strand derivative is essentially 5′ GGGGAAATTT 3′ (SEQ ID NO: 697) and the bottom strand derivative is 5′ GAATTTTTTT 3′ (SEQ ID NO: 698). However, if the derivative population is now replicated ex vivo by enzymological means, four distinct derivative populations ensue, these being [5′ GGGGAAATTT 3′] (SEQ ID NO: 697), [5′ AAATTTCCCC 3′] (SEQ ID NO: 699), [5′ AAAAAAATTC 3′] (SEQ ID NO: 700) and [GAATTTTTTT 3′] (SEQ ID NO: 698). These derivatives are indeed complexity reduced, but not to the same extent as the original unreplicated derivatives that exist immediately after conversion. Hence when PCR primers are made to the original non-replicated derivative strands, it is necessary to judiciously decide which amplified nucleic acid products one wishes to examine, as the choice of primers to either the top or bottom strands will influence the output. The differences between dealing with two non-complementary derivative populations that constitute the output of a converted genome, versus the four derivative populations that exist after replication are not intuitively clears, but can have important implications for primer design.

Finally, the issue of longer probes or primers that was introduced earlier to formalize and quantitated ‘complexity-reduction’ only assumes relevance when searching for a unique sequence within a derivative population of molecules. An important foundation of the present invention, however, can be the choice of derivative loci that are maximally similar between HPV types, allowing all HPV types to be assayed in one initial test, if required. These chosen loci will vary depending upon whether the top or bottom strand derivatives are chosen and such loci will be in different regions in the top strand as compared to the bottom strand.

The practical-importance of the requirement for longer probes and primers in derivative populations is overshadowed by the practical advantages that are gained for HPV detection owing to the generation of loci that are rendered more sequence similar by conversion using the HGS bisulphite treatment in the present invention. They are also overshadowed by the optional use of INAs that allow for shorter probe and primer molecules than is the case for conventional oligonucleotides. In addition, application of the nested PCR approach to derivative populations requires two primers to bind in the same neighbourhood in order to allow for amplified nucleic acid product production. If one of the PCR primers has sequence similarity to a decoy locus that is outside the targeted neighbourhood, it is unlikely that the other member of its primer pair would also have a decoy locus nearby in the same non-targeted region. It is even more unlikely that the inner primers of such a nested PCR approach would again have decoy loci in the same non-targeted region as the first round primers. The probability of spurious amplification is extremely unlikely.

### Human Papilloma Virus

The term “viral-specific nucleic acid molecule” as used herein means a molecule which has been determined or obtained using the method according to the present invention which has one or more sequences specific to a virus or virus type.

The term ‘taxonomic level of the virus’ as used herein includes type, subtype, variant and genotype. The fluidity of viral genomes is recognized. Different viral populations may furthermore be polymorphic for single nucleotide changes or be subject to hyper- or hypo-mutability if they reside within certain cancerous cells where normal DNA repair processes are no longer functioning.

The term “HPV-specific nucleic acid molecule” as used herein means a specific nucleic acid molecule present in treated or converted viral DNA which can be indicative of the virus or virus type.

The term “HPV type” as used herein refers to any existing or new HPV population where there is less than 90% sequence similarity with previously isolated and characterized HPV types, (1993, Van Rast, M. A., et al., Papillomavirus Rep, 4, 61-65; 1998, Southern, S. A. and Herrington, C. S., Sex. Transm. Inf. 74, 101-109).

The term “HPV subtype” as used herein refers to any existing or new HPV population where the sequence similarity is in the 90-98% range relative to previous subtypes, (1993, Van Rast, M. A., et al., Papillomavirus Rep, 4, 61-65; 1998, Southern, S. A. and Herrington, C. S., Sex. Transm. Inf. 74, 101-109).

The term “HPV variant” as used herein refers to any existing or new HPV population where the sequence similarity is between 98-100% of previous variants, (1993, Van Rast, M. A., et al., Papillomavirus Rep, 4, 61-65; 1998, Southern, A. and Herrington, C. S. Sex. Transm. Inf. 74, 101-109).

The term “HPV genotype” as used herein is as follows; a genotype is any fully sequenced HPV genome that minimally differs by one base from any other fully sequenced HPV genome including whether that single base exists as either a G, A, T or C, or whether the base at a given position in the standard comparator, (namely HPV16 from position 1 to position 7904) has been altered by deletion, addition, amplification or transposition to another site. We compare all other HPV genotypes relative to the HPV16 standard using prior art BLAST methodologies.

All the bioinformatic HPV comparisons used in the present patent specification were made relative to the HPV16 genome (using positions 1 to 7904 of HPV16 as the standard comparator), and using prior art BLAST methodologies, (1996, Morgenstern, B., et al., Proc. Natl. Acad. Sci. USA. 93, 12098-12103). The standard HPV ‘type’ utilized herein for reference purposes is HPV16 of the Papillomaviridae, a papillomavirus of 7904 base pairs (National Center for Biotechnology Information, NCBI locus NC_{—}001526; version NC_{—}001526.1; GI:9627100; references, Medline, 91162763 and 85246220; PubMed 1848319 and 2990099).

### Primers for Amplification Via PCR.

The amplification methodology according to the present invention consists of an oligonucleotide primer set directed to the genomically simplified top and/or bottom strands of HPV. The list of such primers that have produced HPV-specific products from both liquid based cytology and archival paraffin samples from human patients is summarized in Table 1. Most primers are directed to the top strand derivatives of the different HPV types, but a smaller number have been directed towards the bottom strand derivative (HPVB).

TABLE 1

Examples of 516 forward and reverse primers
suitable for detection of various types of
HPV, using either the top or bottom
derivative strands of HPV.

| | | |
|---|---|---|
| | | SEQ |
| | | ID |
| Primer | Sequence | NO |

| | | |
|---|---|---|
| HPV11-E4-1 | ATTATTGGGAAGTATGTTATGGTAGT | 1 |
| HPV11-E4-2 | GTTTTTTTGTATTTGTATTTAGT | 2 |
| HPV11-E4-3 | TACTTATTATAATTATCAATAAC | 3 |
| HPV11-E4-4 | AAATCACCTTACAATTACACTATAAAC | 4 |
| HPV11-E7-1 | GTTATGAGTAATTAGAAGATAGT | 5 |
| HPV11-E7-2 | ATTATTAAATATTGATTTGTTGT | 6 |
| HPV11-E7-3 | ATACCTATAATATACTCTACTATAAC | 7 |
| HPV11-E7-4 | CAAAATTTTATATAATATACCTATC | 8 |
| HPV16-E4-1 | GAATATATTTTGTGTAGTTTAAAGATGATGT | 9 |
| HPV16-E4-2 | GTTTTATATTTGTGTTTAGTAGT | 10 |
| HPV16-E4-3 | CCTTTTAAATATACTATAAATATAATATTAC | 11 |
| HPV16-E4-4 | CACACAATATACAATATACAATAC | 12 |
| HPV16E5-1 | GTTTATATGATAAATTTTGATATTGT | 13 |
| HPV16E5-2 | TTGTGTGTTTTTGTGTGTTTGTT | 14 |
| HPV16E5-3 | ATATTAAAAATAATAATATATAAAC | 15 |
| HPV16E5-E | ATATATAACAATTACATTATATAC | 16 |
| HPV16-E6-1 | GAAAGTTATTATAGTTATGTATAGAGT | 17 |
| HPV16-E6-2 | ATTAGAATGTGTGTATTGTAAGTAAT | 18 |
| HPV16-E6-3 | ACTACAATATAAATATATCTCCATAC | 19 |
| HPV16-E6-4 | AAACTATCATTTAATTACTCATAAC | 20 |
| HPV16-E7-1 | TATGTATGGAGATATATTTATATTGT | 21 |
| HPV16-E7-2 | GTTATGAGTAATTAAATGATAGTTT | 22 |
| HPV16-E7-3 | TAAAACACACAATTCCTAATATAC | 23 |
| HPV16-E7-4 | CCCATTAATACCTACAAAATCAAC | 24 |
| HPV18-E4-1 | GGGAATATAGGTAAGTGGGAAGTAT | 25 |
| HPV18-E4-2 | GATTGTAATGATTTTATGTGTAGTATT | 26 |
| HPV18-E4-3 | AAATAATATATCTCTATAATAATC | 27 |
| HPV18-E4-4 | TTCATTACCTACACCTATCCAATACC | 28 |
| HPV18E5-1 | ATATGATAATGTAATATATATGT | 29 |
| HPV18E5-2 | GTGTATGTATGTATGTGTGTTGTT | 30 |
| HPV18E5-3 | CATATATATACAATAATAACATAAAC | 31 |
| HPV18E5-4 | CAACCTATACAATTACTATAAAAAC | 32 |
| HPV18-E6-1 | GATAGTATATAGTATGTTGTATGTT | 33 |
| HPV18-E6-2 | ATTTAGATTTTGTGTATGGAGATAT | 34 |
| HPV18-E6-3 | ATCTTACAATATTACCTTAAATCCATAC | 35 |
| HPV18-E6-4 | AAATTTCATTTTAAAACTCTAAATAC | 36 |
| HPV18-E7-1 | GTATGGATTTAAGGTAATATTGTAAGAT | 37 |
| HPV18-E7-2 | GTATTTAGAGTTTTAAAATGAAATTT | 38 |
| HPV18-E7-3 | AACACACAAAAAACAAAATATTC | 39 |
| HPV18-E7-4 | ACCATTATTACTTACTACTAAAATAC | 40 |
| HPV26E4-1 | GTATTTAGTATTTGTAGTAGT | 41 |
| HPV26E4-2 | TTATTGTTAAAATTGTTGAGTT | 42 |
| HPV26E4-3 | AATAATAACCTCCACTTATAC | 43 |
| HPV26E4-4 | AAATATACTATAAACACAATTTAATC | 44 |
| HPV26E6-1 | GTTTGAATATTATTTTGTAAAATTTGT | 45 |
| HPV26E6-2 | TATTGTAAGGAAATTTTATAATGGGT | 46 |
| HPV26E6-3 | CTTTATTTTTCTTCTAACCCCAATAAC | 47 |
| HPV26E6-4 | ATACACAACCCTTTCCACTACCCTAC | 48 |
| HPV26E7-1 | GAAATATAAGTGTAAAGAATAATGT | 49 |
| HPV26E7-2 | GAATAATTGGATTATGAATAATTTGAT | 50 |
| HPV26E7-3 | TCTTCCATTAACATCTACTCCAAC | 51 |
| HPV26E7-4 | TTACTATACAACACACTAATAAC | 52 |
| HPV30-E4-1 | GTATAAAGGTATATGGGAAGTGT | 53 |
| HPV30-E4-2 | GATTTTGTGTTTAGTATTTTTAGATT | 54 |
| HPV30-E4-3 | CATATAACTCCACCAAAACACTATC | 55 |
| HPV30-E4-4 | TCTATTTAATTCACCTTTTAAATAC | 56 |
| HPV30E6-1 | GTATAGTTTATAGAAAGGGAGTGAT | 57 |
| HPV30E6-2 | GTATTAAAYGGATAGTGTATTTATGGT | 58 |
| HPV30E6-3 | TACACACTACATATAAACTA | 59 |
| HPV30E6-4 | CCCATACAATAAATAATTATAATATC | 60 |
| HPV30-E7-1 | GATAATTTATAGAAGTAGTTATAGT | 61 |
| HPV30-E7-2 | TTTTGTTATTTAATTAATATATAG | 62 |
| HPV30-E7-3 | CCCATCTAAATCTAATACTATAC | 63 |
| HPV30-E7-4 | CTATATTATTATTACATTACTATTATC | 64 |
| HPV31-E4-1 | TTTTTGAATTTGTATTTAGT | 65 |
| HPV31E4-1A | GAATTAAATATTTTTATAGTAAGT | 66 |
| HPV31-E4-2 | ATTTTTTGTTGGGATTGTTATAAAGT | 67 |
| HPV31E4-2A | GTGTTATTATTTGTGTGTTTTGTT | 68 |
| HPV31-E4-3 | TCAATAACCCCACAATTAACACTATC | 69 |
| HPV31E4-3A | ATAATAAAATATATATAAACAC | 70 |
| HPV31-E4-4 | CTTATTTAATTTATACATACAACTAC | 71 |
| HPV31E4-4A | AAAAAATACATATATATAAATTAC | 72 |
| HPV31E6-1 | TTTAGTATAAAAAAGTAGGGAGTGAT | 73 |
| HPV31E6-2 | GGTATATAAAGTATATAGTATTTTGTGT | 74 |
| HPV31E6-3 | AATCTTAAACATTTTATACACACTC | 75 |
| HPV31E6-4 | CACACTATATCTATACCATCTAAATTC | 76 |
| HPV31-E7-1 | GTAATTGATTTTTATTGTTATGAGT | 77 |
| HPV31-E7-2 | GTTATAGATAGTTTAGTTGGATAAGT | 78 |
| HPV31-E7-3 | CTAAATCAACCATTATAATTACAATC | 79 |
| HPV31-E7-4 | CCTATCTATCTATCAATTACTAC | 80 |
| HPV33-E4-1 | GTGGGTGGTTAGGTAATTGTTTGTT | 81 |
| HPV33E4-1A | GTAAAAATATTATTTATTGTGT | 82 |
| HPV33-E4-2 | TTAAATATTTATTATTGAAATTGT | 83 |
| HPV33E4-2A | GTGTATATTATAAGTTAATATGTGT | 84 |
| HPV33-E4-3 | CCTTTTAAATACACTATAAATAC | 85 |
| HPV33E4-3A | AAAAATCCCACAAACACCCAAAACAAC | 86 |
| HPV33-E4-4 | CTAATCCAATACCAAATAAATAAC | 87 |
| HPV33E4-4A | TACTCTTATTATATCATATACTATAC | 88 |
| HPV33E6-1 | GTATATATAAAGTAAATATTTTGT | 89 |
| HPV33E6-2 | GGTATTGTAYGATTATGTTTTAAGAT | 90 |
| HPV33E6-3 | CTCTATATACAACTATTAAATCTAC | 91 |
| HPV33E6-4 | CCATATACAAAATAATTATAATATC | 92 |
| HPV33-E7-1 | TTTTGTATATGGAAATATATTAGAAT | 93 |
| HPV33-E7-2 | TAGGTGTATTATATGTTAAAGATT | 94 |
| HPV33-E7-3 | CCTCATCTAAACTATCACTTAATTAC | 95 |
| HPV33-E7-4 | TAACTAATTATACTTATCCATCTAAC | 96 |
| HPV35-E4-1 | GGGTGGTTAGGTAATTGTTTGTTT | 97 |
| HPV35E4-1A | GGATATATGTTTATATGATAGATT | 98 |
| HPV35-E4-2 | ATTTATTGTTGAAATTGTTATATAGT | 99 |
| HPV35E4-2A | ATAGTTTTTAGTATTGTGTTGT | 100 |
| HPV35-E4-3 | CAAATATAAAATAAACCCCTCTATC | 101 |
| HPV35E4-3A | CAATTACTATACTACCAAATATTATAC | 102 |
| HPV35-E4-4 | ATATACTATAAATATAATTATAC | 103 |
| HPV35E4-4A | CCACCATACACACATATTACACAATAC | 104 |
| HPV35E6-1 | GGTTGTTATAAAAGTAGAAGTGT | 105 |
| HPV35E6-2 | AAAAGTAGAAGTGGATAGATATTG | 106 |
| HPV35E6-3 | ACACAAATCATAACATACAAAATC | 107 |
| HPV35E6-4 | ATACATACTCCATATAACTAACC | 108 |
| HPV35-E7-1 | AAATAATGTAATAAATAGTTATGTT | 109 |
| HPV35-E7-2 | GTTGTGTTTAGTTGAAAAGTAAAGAT | 110 |
| HPV35-E7-3 | CCATATATATACTCTATACACACAAAC | 111 |
| HPV35-E7-4 | AAACACACTATTCCAAATATAC | 112 |
| HPV39-E4-1 | GTAAATGGGAAGTGTATTATAATGGT | 113 |
| HPV39-E4-2 | AATTTATTGTTTTGATTTTATGTGT | 114 |
| HPV39-E4-3 | AATTATTAAAATAATCCAAAAAC | 115 |
| HPV39-E4-4 | TAATATTACCACAACTAAAATAC | 116 |
| HPV39E5-1 | GTATATGTTTTATTGGGTTATATGAT | 117 |
| HPV39E5-2 | GTATATATATATGTTGTAATGTT | 118 |
| HPV39E5-3 | ATCTATACAACAACCACATAAAC | 119 |
| HPV39E5-4 | CAATACTATATCATATCCATTAC | 120 |
| HPV39E6-1 | TTTATAATATTTTATAAGTATT | 121 |
| HPV39E6-2 | GTTTAAAAAAAGGGAGTAAT | 122 |
| HPV39E6-3 | CATAATTAACATACAACTAATAATTC | 123 |
| HPV39E6-4 | ATTATATTTTCTAATATAATTAC | 124 |
| HPV39-E7-1 | TTAAAGTTTATTTTGTAGGAAATTG | 125 |
| HPV39-E7-2 | GATTTATGTTTTTATAATGAAATATAGT | 126 |
| HPV39-E7-3 | CTAATAAATCCATAAACAACTAC | 127 |
| HPV39-E7-4 | CATAACAAATTACTAATTTACATTTAC | 128 |
| HPV40E4-1 | ATGTAGATTTTGTAAAGGAAGTAT | 129 |
| HPV40E4-2 | GGATTATTTATTGTTGAGATTGT | 130 |
| HPV40E4-3 | CCTATACCCRTTATTACTTTCAAAATC | 131 |
| HPV40E4-4 | TCTAAAACACTTTAAACAATTAAC | 132 |
| HPV40E6-1 | GATTTTGTATGAATTGTGTGATTAGTGT | 133 |
| HPV40E6-2 | GTTTTAAAAATAGTTGAGGTATTGGTT | 134 |
| HPV40E6-3 | CAAAAACTTATAACACTTACAAC | 135 |
| HPV40E6-4 | TCCAACAATATAAACAATACCCTATC | 136 |
| HPV40E7-1 | GTATTTTGAATTTGTATGTTTAAATTGT | 137 |
| HPV40E7-2 | GATAGTTTAGATTTAGAAGATGAT | 138 |
| HPV40E7-3 | TATATAATATACCCATCAACAACTAC | 139 |
| HPV40E7-4 | CACTCTATAACTACACAATTAAAAC | 140 |
| HPV42-E4-1 | GTAGAGATATTTTTTATTGGATT | 141 |
| HPV42-E4-2 | GTTGGTATAATAAGTGTGTAT | 142 |
| HPV42-E4-3 | ATTCTAACCCCACACAATCCAAAATC | 143 |
| HPV42-E4-4 | TAACCTAACTTCCACAATAATTC | 144 |
| HPV42-E7-1 | GTATATAGTGGAGAAAGAAATTGGAT | 145 |
| HPV42-E7-2 | GAATAATAAATTAGATGTGTTTTGTGTT | 146 |
| HPV42-E7-3 | CCAATTATTCATAACAATACAAATC | 147 |
| HPV42-E7-4 | ACTTAATCATCTTCATCTAAAC | 148 |
| HPV43-E4-1 | TTATATATAGTATGTGGGTAAAAGT | 149 |
| HPV43-E4-2 | TTTTTGTATTTGTATTTAGTAT | 150 |
| HPV43-E4-3 | ATTATACCCTCTAAAATAATAATC | 151 |
| HPV43-E4-4 | ACTTCACCTTATAACTATATTATAAAC | 152 |
| HPV43E6-1 | TTATACTTGTAGTTTAAGGTGGGAT | 153 |
| HPV43E6-2 | TTATAGTTTGTGGGGTATAATGAT | 154 |
| HPV43E6-3 | TTTTCCATAAAACTATAAACAAAC | 155 |
| HPV43E6-4 | TATAACACTTACAACATCTAATAC | 156 |
| HPV43-E7-1 | GTATAGTATATTGTGTAAAAGGT | 157 |
| HPV43-E7-2 | TTGTTTATATTGTTGGAAATTATGT | 158 |
| HPV43-E7-3 | CTTAATATCACTATCAAAACACTAC | 159 |
| HPV43-E7-4 | TTTTAATATACCCAACAACAAATC | 160 |
| HPV44-E4-1 | GAYGTATTTATTGTTGGGTTTGT | 161 |
| HPV44-E4-2 | GGTTTTATTTATATTGTTTATTGGT | 162 |
| HPV44-E4-3 | TACCTATACAATAATTATTATC | 163 |
| HPV44-E4-4 | TCACCTTATAATTAAACTACAAAC | 164 |
| HPV44-E7-1 | GGAAATTTTTTATTTGTAGTTTGTGTT | 165 |
| HPV44-E7-2 | ATAAGGTAAGGTTAATTAATTTAGGT | 166 |
| HPV44-E7-3 | CCTTTAAAATAATATAATTTCCATAC | 167 |
| HPV44-E7-4 | CCTACAAAATCAAAAAATTCCAAC | 168 |
| HPV45E4-1 | GTGGGAAGTATAATATGGGGGT | 169 |
| HPV45E4-2 | GTAATGATTTTATGTGTAGTATT | 170 |
| HPV45E4-3 | TATTACTTATACTTAAACACAAAAAC | 171 |
| HPV45E4-4 | TATCACCTTTTAAATATATTATAAAC | 172 |
| HPV45E6-1 | ATATTATATAAAAAAGGGTGTAAT | 173 |
| HPV45E6-2 | GTATATAAAAGTTTTGTGGAAAAGTGT | 174 |
| HPV45E6-3 | TACACTATACATAAATCTTTAAAAAC | 175 |
| HPV45E6-4 | TACATTTATAACATACAACATATAC | 176 |
| HPV45-E7-1 | GTAAGAAATTGTATTGTATTTGGAATT | 177 |
| HPV45-E7-1A | GTAAGAAATTGTATTGTATTTGGAATT | 178 |
| HPV45-E7-2 | GAATGAATTAGATTTTGTTGATTTG | 179 |
| HPV45-E7-2A | GAATGAATTAGATTTTGTTGATTTG | 180 |
| HPV45-E7-3 | CAACTACTATAATATTCTAAAATC | 181 |
| HPV45-E7-3A | CAACTACTATAATATTCTAAAATC | 182 |
| HPV45-E7-4 | AACACACAAAAAACAAAATACTC | 183 |
| HPV45-E7-4A | AACACACAAAAAACAAAATACTC | 184 |
| HPV51-E4-1 | TATATGGGGTATAATAGTGGGAGGTT | 185 |
| HPV51-E4-2 | GTTTTGAATATGTATTTAGTATTTGT | 186 |
| HPV51-E4-3 | CTTTAATACCCTCCAATATTAATAC | 187 |
| HPV51-E4-4 | CAATTTATATCACCTTTTAAATAC | 188 |
| HPV51-E7-1 | TTGTGTATGGTATTATATTAGAGGT | 189 |
| HPV51-E7-2 | GATGTTAAAGATTATTTGGGTT | 190 |
| HPV51-E7-3 | TCCTCTAAACTATCAAATTAC | 191 |
| HPV51-E7-4 | CAACCCRTCTTTCTAATAACTAATC | 192 |
| HPV52E4-1 | GGTGGTTAGGTAATTGTTTGTTTTGT | 193 |
| HPV52E4-2 | ATTGAAATTGTTGTTTATTTATGT | 194 |
| HPV52E4-3 | CTTTATTTATACACTCAATTACAATAAC | 195 |
| HPV52E4-4 | TAAATACAATACAAATTATATATAC | 196 |
| HPV52-E7-1 | TTTGAAATAATTGATTTATATTGT | 197 |
| HPV52-E7-2 | ATGAGTAATTAGGTGATAGT | 198 |
| HPV52-E7-3 | CTATACCTTCAAAATCCTCCATTAC | 199 |
| HPV52-E7-4 | TTATTACCTCTACTTCAAACCAAC | 200 |
| HPV53E4-1 | TATGGGAAAATAAAGTATTTATTGTTT | 201 |
| HPV53E4-2 | GATTTTGTGTTTAGTATTTTTAGATT | 202 |
| HPV53E4-3 | ATTTATATTATCTATATTCCTTAC | 203 |
| HPV53E4-4 | CAAATACAATCTTATAACCACTATC | 204 |
| HPV53-E7-1 | TAGTGTAYGGGGTTAGTTTGGAAGT | 205 |
| HPV53-E7-2 | ATTTGATTTATTAATAAGGTGT | 206 |
| HPV53-E7-3 | CTATAATATATTATAAAAATATTAATAC | 207 |
| HPV53-E7-4 | CAATTACTCATAACATTACAAATC | 208 |
| HPV54-E4-1 | GGGAGGTGYGTATGGGTAGTAGT | 209 |
| HPV54-E4-2 | GGTATTGTTGAATATATTAGATTAGTT | 210 |
| HPV54-E4-3 | ATAATATCACCACTACTTATATAC | 211 |
| HPV54-E4-4 | CCTAAAACATTTTAATATATTAAATTC | 212 |
| HPV54-E7-1 | AGGATTTATTTGTGGTGTGGAGAT | 213 |
| HPV54-E7-2 | GTATGTGTATTGTGTTTAGAATTGT | 214 |
| HPV54-E7-3 | ACATTATAACTTCCAACAATATAAAC | 215 |
| HPV54-E7-4 | CAAAAACTATATCCTCAATTATAAC | 216 |
| HPV55-E4-1 | GGAGGTTTGTATTGGTAGTAGTGTT | 217 |
| HPV55-E4-2 | GTATTTATATTTAGTATTGTGT | 218 |
| HPV55-E4-3 | AATAAATATTATTATTTATACTATC | 219 |
| HPV55-E4-4 | AAATCACCTTATAATTAAACTACAAAC | 220 |
| HPV55-E7-1 | GGTTAATTAATTTAGGTATTTTGAT | 221 |
| HPV55-E7-2 | GTATTAATAGTGGAAGAAGAGAT | 222 |
| HPV55-E7-3 | CCTACAAAATCAAAAAAATCCAAC | 223 |
| HPV55-E7-4 | AACTAATTCATCCACCTCATCCTCTAAAC | 224 |
| HPV56E4-1 | ATTATATAGATTTTGAATAAGAGGTT | 225 |
| HPV56E4-2 | GAAAATGAGAGTATTTATTGTTTTGAT | 226 |
| HPV56E4-3 | ATTATTAATACTTCTACTTCTACTATC | 227 |
| HPV56E4-4 | AATTCACCTTTTAAATATACTACAAAC | 228 |
| HPV56E6-1 | TATTTTTATATATTGGGAGTGAT | 229 |
| HPV56E6-2 | TTGTGTGGATATATTTATGGAGTT | 230 |
| HPV56E6-3 | CACTAATTTTAATTCAATACATAC | 231 |
| HPV56E6-4 | CAATAAACATACTCTACACACTAC | 232 |
| HPV56-E7-1 | GATTTATAGTGTAATGAGTAATTGGATAGT | 233 |
| HPV56-E7-1A | GATTTATAGTGTAATGAGTAATTGGATAGT | 234 |
| HPV56-E7-2 | GGTTATAGTAAGTTAGATAAGT | 235 |
| HPV56-E7-2A | GGTTATAGTAAGTTAGATAAGT | 236 |
| HPV56-E7-3 | TCCCCATCTATACCTTCAAATAAC | 237 |
| HPV56-E7-3A | TCCCCATCTATACCTTCAAATAAC | 238 |
| HPV56-E7-4 | CCTATTTTTTTTTCTACAATTAC | 239 |
| HPV56-E7-4A | CCTATTTTTTTTTCTACAATTAC | 240 |
| HPV57E4-1 | GGATTTTGGAATAGAGGTTTTGATT | 241 |
| HPV57E4-2 | GTATTTGTGTTTAGTATTTAGGT | 242 |
| HPV57E4-3 | TAAAATCRAACTATTACCACTACTATC | 243 |
| HPV57E4-4 | TTTAACACTCCACCTACCCTTCTCTAAC | 244 |
| HPV57E6-1 | GTTTTAGGAATATTTTTTTGT | 245 |
| HPV57E6-2 | GTGTAGAGAGTATGGTTTGGAGT | 246 |
| HPV57E6-3 | CCAATACCTATATTATCTAAATTTTAC | 247 |
| HPV57E6-4 | CAACTAAAATATAAATAATCCTATC | 248 |
| HPV57E7-1 | GATAATTTAGAAGAAGATAT | 249 |
| HPV57E7-2 | AATTGATAGAATTAGTTGTGTAGGTT | 250 |
| HPV57E7-3 | CATAAATTATTATAACTTCCACATAAAAC | 251 |
| HPV57E7-4 | CCTCATCCTCRTCACTAAATACCTAAC | 252 |
| HPV58-E4-1 | GTTTTATATTTATATTTAGTGATT | 253 |
| HPV58E4-1A | GTAAATTATAAGTTAATATGTGTTGT | 254 |
| HPV58-E4-2 | ATTGAAATTGTTGATTTAAAGATT | 255 |
| HPV58E4-2A | GTTTTATATTGTTTTTATGTTTGTGT | 256 |
| HPV58-E4-3 | TACACRATAAATAAAACTTTAAAAC | 257 |
| HPV58E4-3A | TAACTTTATTAAATTAAATATTATAC | 258 |
| HPV58-E4-4 | TAAACATTTTAAACTATTTAAATC | 259 |
| HPV58E4-4A | TACCATACCACCATATACAAAAC | 260 |
| HPV58E6-1 | TTAAATTATAATGTTAAATTTTG | 261 |
| HPV58E6-2 | GTAGATATTTTTTGGTAGGTTATTGT | 262 |
| HPV58E6-3 | TTACATACTACAAATAAATTTC | 263 |
| HPV58E6-4 | TATCTATACTCACTTATTTTAAATAAC | 264 |
| HPV58-E7-1 | TATTTTGAATTAATTGATTTATTTTGT | 265 |
| HPV58-E7-2 | ATGAGTAATTATGTGATAGTTT | 266 |
| HPV58-E7-3 | ATACATATACCCATAAACAACTAC | 267 |
| HPV58-E7-4 | TTATTACTATACACAACTAAAAC | 268 |
| HPV66-E4-1 | TATATAGATTTTGAATAGGAGGTT | 269 |
| HPV66-E4-2 | GAGTATTTATTGTTTTGATTTTGTGTT | 270 |
| HPV66-E4-3 | TAATTTTATCACCACAATAAC | 271 |
| HPV66-E4-4 | CACCTTTTAAATAAATTACAAAC | 272 |
| HPV66-E7-1 | GTATTATAAATATTTAGTGTATGGGGT | 273 |
| HPV66-E7-2 | GTTATTTGATTTATTAATAAGGTGT | 274 |
| HPV66-E7-3 | TCCAATTACTCATTACATTATAAATC | 275 |
| HPV66-E7-4 | TATATTATTCAACTTATCTAAC | 276 |
| HPV6-E4-1 | AATAATGGGAAGTATGTTATGGTAGT | 277 |
| HPV6-E4-2 | TATATAAGAAGTATTTATTTTTG | 278 |
| HPV6-E4-3 | TACTATCACATCCACAACAACAAATC | 279 |
| HPV6-E4-4 | CTCTAATATCTATTTCTATACACTAC | 280 |
| HPV6-E7-1 | GATATTTTGATTATGTTGGATATGT | 281 |
| HPV6-E7-2 | GTTGAAGAAGAAATTAAATAAGAT | 282 |
| HPV6-E7-3 | TACTATCACATCCACAACAACAAATC | 283 |
| HPV6-E7-4 | CTCTAATATCTATTTCTATACACTAC | 284 |
| HPV73E4-1 | GGGTGGTTAGGTAATATGTTGTGT | 285 |
| HPV73E4-2 | TTTGAAATTGTTAATTTATTGT | 286 |
| HPV73E4-3 | CATTATATATAATACACTAAATAC | 287 |
| HPV73E4-4 | ACTATTTTTATCACCTTTTAAATAC | 288 |
| HPV73E6-1 | AAATTTGGATTGTGTGTTTTGTT | 289 |
| HPV73E6-2 | GAAAGGATAAATTATATGGTGTATGT | 290 |
| HPV73E6-3 | CATACTTTTACTTTTCCAATAAAC | 291 |
| HPV73E6-4 | CCACAATTACAAATAATCTCCAAC | 292 |
| HPV73E7-1 | GTATGGAAAAAAAATAATTTTGT | 293 |
| HPV73E7-2 | GATTTTATATGTTAYGAGTTATTGGAT | 294 |
| HPV73E7-3 | CACAATACCTAATATACCCATAAAC | 295 |
| HPV73E7-4 | TAAATTTCTAAAACAATTAAAAC | 296 |
| HPV82E4-1 | GTGTGGTAATGTAATAATATGTTT | 297 |
| HPV82E4-2 | TTTTATTATAATTGTTGAATAGT | 298 |
| HPV82E4-3 | CAATTTTAATTACACTAAAATACC | 299 |
| HPV82E4-4 | CTTAAACATTTTAAACAATTTATTAC | 300 |
| HPV82E6-1 | GAGTAGATGTGTATAATGTAGT | 301 |
| HPV82E6-2 | GTTATATGTAGTATGTAAAAAATGTT | 302 |
| HPV82E6-3 | CACCACCTTTTACTTTTCTTCAAAC | 303 |
| HPV82E6-4 | TTATCTTAATAATTTTCTACAATTTAC | 304 |
| HPV82E7-1 | AATTTGAAATTGATTTGTAATGT | 305 |
| HPV82E7-2 | GTGATTAGTTAGTTAGATAAGT | 306 |
| HPV82E7-3 | CACACCACRAACACACCAAAC | 307 |
| HPV82E7-4 | CTCTATACCTTCACTATCCATTAC | 308 |
| HPV83E4-1 | GATTTTGTATTTAGTATTTAGGAT | 309 |
| HPV83E4-2 | TTTGTTGTAATTAGTATTAGGT | 310 |
| HPV83E4-3 | TTATACAAACACTATCACTACTATATC | 311 |
| HPV83E4-4 | ATTCACTATATCCCTTATAAC | 312 |
| HPV83E6-1 | GAATTAATAATAGTAGAAGTGTTGTT | 313 |
| HPV83E6-2 | GGAGTTGTGTATTAGTGGGATT | 314 |
| HPV83E6-3 | CTCAACRACTTCAAACACATATAAC | 315 |
| HPV83E6-4 | TACATAATACCCTACAATAACAAC | 316 |
| HPV83E7-1 | GGTTATATAGTAATAATAGT | 317 |
| HPV83E7-2 | GTAATGAATAAGGTATAGATAGT | 318 |
| HPV83E7-3 | CTATATCCACTACATTCACCAAAAAATC | 319 |
| HPV83E7-4 | TAAATTCCCCAATCCCAATATCTATAC | 320 |
| HPV84E4-1 | ATGTATGYGATTTTGTATTTAGT | 321 |
| HPV84E4-2 | ATTGTTGAAATTGTTGTATAGTTGT | 322 |
| HPV84E4-3 | CAATTATTATTTATCCTTATACTAC | 323 |
| HPV84E4-4 | TAAATATAAAACAAATACACTATC | 324 |
| HPV84E6-1 | GGAAGGYGAAGTGTTGGTTTTTGT | 325 |
| HPV84E6-2 | GGTATAATTTTTTTTATGGGGTGTGT | 326 |
| HPV84E6-3 | TCCTTTTCCTAATAACACAATAACTTAC | 327 |
| HPV84E6-4 | CTATCCAACTATTTTATAAATTAAC | 328 |
| HPV84E7-1 | GTTGTTATTTTATAAAATAGTTGGAT | 329 |
| HPV84E7-2 | GGAAGTGTTGTAATTGTAGGGTAAT | 330 |
| HPV84E7-3 | CTTTCTAAAATCTTCCACTCCACAAAAC | 331 |
| HPV84E7-4 | TAAACACTACTTCCACTATAAACTACTAC | 332 |
| HPV-HM-1 | GATTTDKWDTGWATGAGTAATT | 333 |
| HPV-HM-1A | GATTTDTWVDTGTWATGAGTAATT | 334 |
| HPV-HM-2 | RRYRRKTTAGABGADGA | 335 |
| HPV-HM-2A | RRTRRKTTAGABGADGA | 336 |
| HPV-HM-2B | RRYRRKTTAGAKGADGA | 337 |
| HPV-HM-2C | RRTRRKTTAGAKGADGA | 338 |
| HPV-HM-3 | YDATACCTWCWMAWWHVDCCAT | 339 |
| HPV-HM-3A | YWATACCTWCWMAWWHRDCCAT | 340 |
| HPV-HM-3B | YWATACCTWCWAAWWHRDCCAT | 341 |
| HPV-HM-3C | YWATACCTWCWMAWWMRDCCAT | 342 |
| HPV-HM-3D | YWATACCTWCWMAWWHVDCCAT | 343 |
| HPV-HM-4A | ACHWMAAACCAHCCWHWACAHCC | 344 |
| HPV-HM-4B | ACHWMAAACCAWCCWHWACAHCC | 345 |
| HPV-HM-4C | ACHWHAAACCAHCCWHWACAHCC | 346 |
| HPV-HML-1 | GRKTTDKWDTGTWRKGARTAATT | 347 |
| HPV-HML-2 | RRHRRKTTWGANKWDGA | 348 |
| HPV-HML-3 | YDATACCTWHWHHDWHNDCCAT | 349 |
| HPV-HML-4 | ACHHHAAACCAHCCHHWACAHCC | 350 |
| HPV-Uni-1 | GATGGKGATATGRTDSATRTWGGDTWTGG | 351 |
| HPV-Uni-2 | TAARTATTTWGATTATWTDDRAATG | 352 |
| HPV-Uni-3 | TATTWTAWCCYTAHRCHYWHTAHAACCA | 353 |
| HPV-Uni-4 | AMAAAHAMHTAATTHYHMMAACAWAYACC | 354 |
| HPV-Uni-5 | TAAAAHAYAAAYTAYAMWTCAWAYTCYTC | 355 |
| HR1F | TRTATGGARWDATATTRGAA | 356 |
| HR2F | TRATTTRTTAATWAGGTGT | 357 |
| HR3R | AAYAYAWHWTCWTACAAYAT | 358 |
| HR4R | AATTACTCATWACAHWAHAAATCA | 359 |
| HR5F | GAGGGDAWGGGDTGTWRTGGWTGGTTT | 360 |
| HR6F | GATRWWATATTAGATGATGA | 361 |
| HR7R | TWWACTATYTCTWHHTCTACCTA | 362 |
| HR8R | CYAHAWTCTTTCATTTTAA | 363 |
| LR1F | TATDDWTATATDTARAGKKTDAT | 364 |
| LR2F | GGGWRTGGTDWTRTTDDTRTTA | 365 |
| LR3R | HAYWATWMWWCWAYTYTT | 366 |
| LR4R | TAWWHHHYWAAYAYATTTAA | 367 |
| R1HML-1F | RGGWGKRATTGAAWDDGGTK | 368 |
| R1HML-2F | ATTRAAADTGGWDDDTATA | 369 |
| R1HML-3R | HHHYYTACAHMMHAYACA | 370 |
| R1HML-4R | AWWMWWMHWHWWAHAYMTC | 371 |
| R1HM-1F | GGGWGTRATTGAAADDGGTK | 372 |
| R1HM-2F | ATTRAAAWTGGTDDDTATA | 373 |
| R1HM-3R | HHHYYTACAHTMHACACA | 374 |
| R1HM-4R | AWAMWAMHTHWWATACMTC | 375 |
| R2HML-1F | TTDDWDTGTWRKGARTAATT | 376 |
| R2HML-2F | ATGGWDDWWDDWDWAGGTAT | 377 |
| R2HML-3R | YHAHWWACTTTCATTTTAMH | 378 |
| R2HML-4R | MWAYWACCATWHMYACTAWM | 379 |
| R2HM-1F | ATGGWKDWWTKWGWAGGTAT | 380 |
| R2HM-2F | GGGDTGTWDDGGDTGGTTT | 381 |
| R2HM-3R | ACTAYYTCHHHHTCYACCTA | 382 |
| R2HM-4R | YHAHAWACTTTCATTTTAMH | 383 |
| R2HM-5R | MWAYWACCATAHCCACTATC | 384 |
| R3HML-10R | WMWHMHWWMATWHCCATC | 385 |
| R3HML-11R | AYHWMYMHHWWWHHYYWATAYTT | 386 |
| R3HML-1F | RTTTAARGADDKDTWTGGDDT | 387 |
| R3HML-2F | RRAGTRATARDWKWDKDTGT | 388 |
| R3HML-3F | TRTDDWTATWTDTARWGKTT | 389 |
| R3HML-3R | AAMCWYTAHAWATAWHHAYA | 390 |
| R3HML-4F | TTWTTWWRRWTRTWDAK | 391 |
| R3HML-4R | MTHWAYAWYYWWAAWAA | 392 |
| R3HML-5F | ATGRTDTARTGGGTWTWTGATWAT | 393 |
| R3HML-5R | ATWATCAWAWACCCAYTAHAYCAT | 394 |
| R3HML-6F | GADGADWRTDWDATDGTDT | 395 |
| R3HML-6R | AHACHATHWHAYWHTCHTC | 396 |
| R3HML-7F | GATTGTGKDDKWATGKKWWRRT | 397 |
| R3HML-7R | AYYWMMCATWMHHMCACAATC | 398 |
| R3HML-8F | DRTDTTWAARWADARTTGT | 399 |
| R3HML-8R | ACAAYTHTWYTTWAAHAYH | 400 |
| R3HML-9F | AATKTWDDDAGTTATTTTTGGTT | 401 |
| R3HML-9R | AACCAAAAATAACTHHHWAMATT | 402 |
| R3HM-10F | TTDGTWGAWDKWRATAGTAATGT | 403 |
| R3HM-10R | ACATTACTAYTWMHWTCWACHAA | 404 |
| R3HM-11F | GATTGTGKDRTWATGKKWWRRT | 405 |
| R3HM-11R | AYYWWMMCATWAYHMCACAATC | 406 |
| R3HM-12F | RRKGADGRDGGDRATTGGA | 407 |
| R3HM-12R | TCCAATYHCCHYCHTCMYY | 408 |
| R3HM-13F | GGWRTDTTWAARWAWARTTGT | 409 |
| R3HM-13R | ACAAYTWTWYTTWAAHAYWCC | 410 |
| R3HM-14F | AATTTWDDDAGTTATTTTTGGTT | 411 |
| R3HM-14R | AACCAAAAATAACTHHHWAAATT | 412 |
| R3HM-15F | GATGGDWATKWWDKWWKW | 413 |
| R3HM-15R | WMWWMHWWMATWHCCATC | 414 |
| R3HM-16F | AARTATWRRDDWTWRDKRTARWTRDW | 415 |
| R3HM-16R | WHYAWYTAYMHYWAWHHYYWATAYTT | 416 |
| R3HM-17R | ATAYWYWAMATTHCYATTWWHATC | 417 |
| R3HM-18R | AAAYYTAATYTAMACCAHATM | 418 |
| R3HM-1F | ATTTAAAGADDTDTWTGGDDT | 419 |
| R3HM-2F | ARAGTRATARWWKWWKDTGT | 420 |
| R3HM-3F | TRTDDWTATWTDTARWGTTTA | 421 |
| R3HM-3R | TAAACWYTAHAWATAWHHAYA | 422 |
| R3HM-4F | GTDKWAARADKAGRDWAAT | 423 |
| R3HM-4R | ATTWHYCTMHTYTTWMHAC | 424 |
| R3HM-5F | TTWTTWAAAWTRTGDAGT | 425 |
| R3HM-5R | ACTHCAYAWTTTWAAWAA | 426 |
| R3HM-6F | TTRTATTKKTWTWRAATWGKWWTRTT | 427 |
| R3HM-6R | AAYAWWMCWATTYWAWAMMAATAYAA | 428 |
| R3HM-7F | TARTATRGWWTWDAKKAT | 429 |
| R3HM-7R | ATMHTMWAWWCYATAYTA | 430 |
| R3HM-8F | ATGRTRTARTGGGTWTWTGATWATGA | 431 |
| R3HM-8R | TCATWATCAWAWACCCAYATYAYCAT | 432 |
| R3HM-9F | GATGAWAGTKAWATDGTDTWT | 433 |
| R3HM-9R | AWAHACHATWTMACTWTCATC | 434 |
| R4HM-1F | TWKWRKAWAATTTDKTWTWTGA | 435 |
| R4HM-2F | TTDGATTTDGATTTTWTRRATAT | 436 |
| R4HM-3R | ACTHAHATCHTAATAAWAATA | 437 |
| R4HM-4R | HHHTYYWAWTYAYAWTTC | 438 |
| R4HM-5R | ACATAHAYATCAWAHMWW | 439 |
| R5HM-1F | TTARTGADRDTAWDGTDTATTT | 440 |
| R5HM-2F | ATWRRTATWTWTTATTATGT | 441 |
| R5HM-3R | CYAAAYTTATTWAAATCHAAYAA | 442 |
| R5HM-4R | ACCHAYYTCHAHHCCHAYACAWMCCCA | 443 |
| R5HM-5R | WMHAHTTTCWATATCATCHWA | 444 |
| R6HM-1F | GGDTWTGGDKKWATGGATTTT | 445 |
| R6HM-2F | WKTRTWTGTAARTATTTWGAT | 446 |
| R6HM-3F | GTWAGRTATTWWTDKAATWR | 447 |
| R6HM-3R | YWATTMHAWWAATAYCTWAC | 448 |
| R6HM-4F | TDTTWAGTGGDTTWATDGT | 449 |
| R6HM-4R | ACHATWAAHCCACTWAAHA | 450 |
| R6HM-5F | TARWTDTTTAATAARTTDTATTGG | 451 |
| R6HM-5R | CCAATAHAAYTTATTAAAHAWYTA | 452 |
| R6HM-6F | GGWTATAATAATGGTRTWTGTTGG | 453 |
| R6HM-6R | CCAACAWAYACCATTATTATAWCC | 454 |
| R6HM-7F | GATATWATWWKDARTATWAAT | 455 |
| R6HM-7R | ATTWATAYTHMWWATWATATC | 456 |
| R6HM-8R | TAAAAHAYAAAYTAYAAWTCAWAYTC | 457 |
| R6HM-9R | ATYCATHHHATAWAWATAWAHCAT | 458 |
| HPVB-1F | TTGWADTAAAAATTTDTKDTTWARDG | 459 |
| HPVB-2F | TTTTKWARRTTWATWKKTTAAAAW | 460 |
| HPVB-3F | KKTTKTTGRTADKTWRTDGT | 461 |
| HPVB-4F | ATWKTRTAWARTTGAAAWATAAATTGTA | 462 |
| HPVB-5F | TTGAAAWATAAATVGTARDTTAWATTTTTT | 463 |
| HPVB-6F | TTGRTTRTKTTARTAWATRTTATTRT | 464 |
| HPVB-7F | TTARTAWGGTTTATTRAAWAWTTGDG | 465 |
| HPVB-8F | TATTTKWADATARTTWGGATATTTRTA | 466 |
| HPVB-9F | TRTKWATTATRTTDTTRTTTTKWA | 467 |
| HPVB-10F | RTATARTTGDGTTTGTTTDKDRTT | 468 |
| HPVB-11F | ATTTTWADDTTWRTATADKTTTA | 469 |
| HPVB-12F | ATTTKDTTTTGDWWKDWATTTAAATG | 470 |
| HPVB-13F | KWWARWGGTTKTARTTAAAARTGRTT | 471 |
| HPVB-14F | TTTATWKWAAADWRTGATTTDTTTGT | 472 |
| HPVB-15F | TTKTTKDGTTTKTTTRTARTGTTKD | 473 |
| HPVB-16F | TATARTTTTTWADDWWTTTDGTTTG | 474 |
| HPVB-3R | ACHAYWAMHTAYCAAMAAMM | 475 |
| HPVB-4R | TACAATTTATWTTTCAAYTWTAYAMWAT | 476 |
| HPVB-5R | AAAAAATWTAAHYTACAATTTATWTTTCAA | 477 |
| HPVB-6R | AYAATAAYATWTAYTAAMAYAAYCAA | 478 |
| HPVB-7R | CHCAAWTWTTYAATAAACCWTAYTAA | 479 |
| HPVB-8R | TAYAAATATCCWAAYTATHTWMAAATA | 480 |
| HPVB-9R | TWMAAAAYAAHAAYATAATWMAYA | 481 |
| HPVB-10R | AAYHMHAAACAAACHCAAYTATAY | 482 |
| HPVB-11AR | TAAAMHTATAYWAAHHTWAAAAT | 483 |
| HPVB-11BR | ATHMMHTTACCHAAYCCHAAYAAATT | 484 |
| HPVB-11CR | TATTTTYTTWCAAATAWCHHYWTAAC | 485 |
| HPVB-12BR | ACWTAATMCAAATTAAAYTTAMW | 486 |
| HPVB-12CR | AHTHAAYAAYAWAAAYTAAAAA | 487 |
| HPVB-14R | ACAAAHAAATCAYWHTTTWMWATAAA | 488 |
| HPVB-15R | HMAACAYTAYAAAMAAACHMAAMAA | 489 |
| HPVB-16R | CAAACHAAAWWHHTWAAAAAYTATA | 490 |
| HPVB-17R | AMATAATACAATAAACMTWYAAYMAYAA | 491 |
| HPVB-18R | AAWAMHACMCCHAAATAAATWM | 492 |
| HPV59E6-1 | GATTATATAAATTGTTTGATTTGAGT | 493 |
| HPV59E6-2 | CAATATTGAATATTTTTTTGT | 494 |
| HPV59E6-3 | CTTACATAAAATAAAATACATTTCAAAC | 495 |
| HPV59E6-4 | CTCATATAACRATATCTTAATTTCAAC | 496 |
| HPV59E7-1 | AAATTATGAGGAAGTTGATTTTGTGTGT | 497 |
| HPV59E7-2 | GAGTTAATTATTTTTTGTTATTAGT | 498 |
| HPV59E7-3 | TATATCCATAAACAACTACTATAAAAC | 499 |
| HPV59E7-4 | ATCTATACCTTCCRAATCRACCATTAC | 500 |
| HPV59E4-1 | GTTATTGATTGTTATGATTTTATGTGT | 501 |
| HPV59E4-2 | GTATTTATTGTTGGATTTTTTGAGT | 502 |
| HPV59E4-3 | CTAAATTATCACAATAATCCACTAAC | 503 |
| HPV59E4-4 | TAATATTACTACAAAAAATATAC | 504 |
| HPV59E5-1 | GTTTGTGTGTGTGTTGTAATGTTT | 505 |
| HPV59E5-2 | GTTTTTGTAATTTGTTTATATGTGTGT | 506 |
| HPV59E5-3 | TTATTATATAAACAATATTACATAAAC | 507 |
| HPV59E5-4 | AAAATATACTATACAATACAATAC | 508 |
| HPV68E6-1 | TTATTGTAGAAGGTAATTATAAYGGAT | 509 |
| HPV68E6-2 | GGGAYGGGGTATTATTAGTTGTATGT | 510 |
| HPV68E6-3 | CTCAATAATTTCAAACAACACATAC | 511 |
| HPV68E6-4 | AAATCTTCRTTTTAAATTTAAATAC | 512 |
| HPV68E7-1 | AATAGYGTTATATAATTTAGTGTAT | 513 |
| HPV68E7-2 | GTAGTAGAAGYGTYGYGGGAGAATT | 514 |
| HPV68E7-3 | ATCCCCATCTATACCTTCACAATTAAC | 515 |
| HPV68E7-4 | TTATTTATCTACTATTACTTATAC | 516 |

The present inventors have found that optimal primers for the detection of High-risk HPV are primers SEQ ID NO: 333 to SEQ ID NO: 350, these primers are top strand primers. These primers work using 1 and 4 for the 1st round PCR and 2 and 3 for the second round PCR.

The present inventors have found that optimal primers for the detection of all ano-genital HPV are the following bottom strand primers SEQ ID NO: 462 with SEQ ID NO: 479 1st round, SEQ ID NO: 463 and SEQ ID NO: 478 2nd round or SEQ ID NO: 470 and SEQ ID NO: 485 1st round, SEQ ID NO: 470 and SEQ ID NO: 486 2nd round.

Viral primers sequences have been generated using multiple alignments to different HPV types to generate primers for the detection of Universal HPV (denoted Uni), High risk types (denoted HR), medium risk types (denoted HM), Low risk types (denoted LR). And various combinations of such HPV types. The combinations are denoted High and Medium risk HPV types (HM), High, Medium and Low risk HPV types (HML).

What constitutes high, medium and low risk types of HPV varies depending on geographic location and on the ethnic lineage of the individuals under test. The FDA approved Hybrid Capture 2 test utilizes thirteen viral types, HPV16, 18, 31, 33, 35, 39, 45, 51, 52, 56, 58, 59 and 68, which we collectively term high-medium risk. For the purposes of the present invention, a subgroup of these is referred to as high risk, namely HPV 16, 18, 45 and 58, and the other subgroup is referred to as medium risk, 31, 33, 35, 39, 51, 52, 58, 59 and 68. The low risk types are HPV6, 11, 26, 30, 40, 42, 43, 44, 53, 54, 55, 57, 66, 73, 82, 83, and 84. The primers were designed for individual HPV types based on the E6, E7, E4, E5 genes of the above HPV virus types.

An example of the designations used in Table 1, such as HPV11-E4-1 indicates primers targeting the top strand of HPV11 using the E4 gene region. The −1 indicates the specific primer number.

Since more than one base needs to be used at a particular position in order to overcome the degeneracy issue, the following symbols designate the base additions; N=A, G, T or C, D=A, G or T, H=A, T or C, B=G, T or C, V=G, A or C, K=G or T, S=C or G; Y=T or C, R=A or G, M=A or C and W=A or T.

### HPV Assay

The HPV detection method according to the present invention (namely genomic complexity-reduction followed by amplification technologies), can be combined with other assays of quite different types for the evaluation of changed cellular status within a cell population, for risk assessment underpinned by deranged transcriptomic, proteomic, metabolite or methylomic networks within infected cells, for monitoring the progression of an infection and for evaluating a therapeutic regimen such as antiviral therapy.

For example, a molecular assay measuring HPV specific nucleic acid molecules can be combined with:
assays using pattern recognition and high throughput robotic imaging technology such as the Multi-Epitope-Ligand-Kartographie (MELK) system for automated quantitation of fluorescent signals in tissue sections,
assays using light, confocal, transmission or electron microscopic analyses for Fluorescent In Situ Hybridizations (FISH), cytological or histological analyses that detect gross levels of chromosomal disturbance within cells, such as aneuploidy, or abnormal organelles (in terms of number, type or morphological appearance),
assays using nucleic acid or polypeptide aptamers; Spiegelmers, (mirror image high-affinity oligonucleotide ligands); multicoloured nanocrystals (quantum dot bioconjugates), for ultrasensitive non-isotopic detection of molecules, or biomarkers for cell surface or internal components; combinatorial chemistry approaches involving Systematic Evolution of Ligands by Exponential Enrichment (SELEX) and high affinity aptamer ligands targeted to different cellular components,
assays using laser-capture of cells or immunomagnetic cell enrichment technologies, or microsphere-based technologies interfaced with flow cytometry, or optical barcoding of colloidal suspensions containing various nucleic acid or peptide/protein moieties,
assays using single cell comparative genomic hybridization aimed at detecting gross genomic imbalances such as duplications, deficiencies, transpositions, rearrangements and their associated in situ technologies,
assays reporting on transcriptomic modulations, such as robogenomic microarray technologies including Serial Analysis of Gene Expression (SAGE), Total Gene Expression Analyses, (TOGA), randomly ordered addressable high density fiber-optic sensor arrays, Massively Parallel Signature Sequencing (MPSS) on microbeads,
assays reporting on proteomic modulations using various technologies including cellular analyses via protein microarrays, Matrix Assisted Laser Desorption Ionization-Time of Flight (MALDI-TOF) methods, Fourier Transformed Ion Cyclotron Resonance Mass Spectrometry, (FTICR), LC MS-MS and Rapid Evaporative cooling Mass Spectrometry, (RapEvap MS),
assays using Multi Photon Detection (MPD) technologies where the detection levels approach zeptomole (10⁻²¹) sensitivity,
assays using methylomic technologies to interrogate the methylome of cells from clinical samples to determine the position of a the cell population along a given trajectory from normalcy to cervical cancer; preferably to determine the altered methylation signature of genomic loci in cells which are affected by viral infection, or immune cells which have been recruited to the site of infection or inflammation.

Some of the above technologies have been previously evaluated (2001, Miklos and Maleszka, Proteomics, 1, 30-41).

### Data Collection, Integration and Management Systems

The data collection and the data management systems for the material associated with the present invention can be combined with clinical patient data and analysed using specialized algorithmic methods. Robotic platform management and data collection can be automatically stored and the collected data combined with an informatics infrastructure and software tools that interface with gene ontologies, (GO), with disease ontologies as exemplified by the National Library of Medicine's Medical Subject Headings (MeSH) thesaurus, the Online Mendelian Inheritance in Man, (OMIM), or with knowledge databases such the Human Genome Mutation Database (HGMD) or PubMed. Software pipelines that interface with the latest human genome assemblies and provide access to, and downloading of, information from sources such as Genbank and RefSeq, can be combined with assays reporting on the genomic status of cells that are HPV infected, or that have been influenced by cells owing to HPV presence elsewhere in the body.

The database infrastructure integrating HPV data with clinical and relevant bioinformatics data can, for example, utilize a loosely-coupled modular architecture which facilitates better software engineering and database management. A relational database management system (RDBMS), (such as Postgresq1 version 7.3) is open source and robust, and serves as an example of part of an integrated system to evaluate and better predict clinical outcomes in the HPV arena. Additional features involving web based Graphical User Interfaces (GUI) would allow for integrated cytological and histological analysis to be combined with molecular HPV data together with therapeutic and pharmaceutical data available in very diverse formats. The integration of enhanced digital technology for image analysis, remote image sharing by pathologists and automated visualization systems is envisaged as an integrated part of an automated molecular kit platform.

### Cell Sampling

HPV detection protocols can be implemented on samples from any portion of the body, including samples from pre-blastocyst stages, embryonic tissues, perinatal material, cadavers or forensic sources. Preferably they are from cervicovaginal areas such as the cervix and vagina but can also be from cutaneous sources. Preferably they are from the cervical transformation zone. The samples can be collected using the CervexBrush, Therapak Corp, Irwindale, Calif., USA; Digene Cervical sampler cervical brush, Digene Corp. Gaitherburg, Md., USA; a plastic spatula/brush combination, Cooper Instruments, Hollywood, Fla., USA; or using dacron swabs or any suitable material for obtaining samples from the ano-genital area or by any standard biopsy procedure such as a needle biopsy. The samples can be placed in various media, such as PreserveCyte, Cytyc Corp. MA, USA or AutoCyte PREP from TriPath Imaging Burlington, N.C., USA. Preferably, initial tests are conducted on Liquid based Cytology, but planar platforms such as paraffin sections and slides are also suitable.

### Kits

The present invention can be implemented in the form of various kits, or combination of kits and instantiated in terms of manual, semi automated or fully robotic platforms. In a preferred form, the MethyEasy™ or HighThroughput MethylEasy™ kits (Human Genetic Signatures Pty Ltd, Australia) allow conversion of nucleic acids in 96 or 384 plates using a robotic platform such as EpMotion.

### Human Papilloma Virus

Mature human papilloma virus DNA is encapsulated within an icosahedral capsid coat consisting of two virally encoded proteins. The double stranded circular DNA genome is 7904 base pairs in length for HPV16, but among the common medium-risk types varies from 7808 base pairs of HPV51 to 7942 base pairs of HPV52. The regions of the viral genome are presented below in the order in which they occur on the circular molecule. The virus has a non-coding region termed URR followed by number of coding regions denoted, E6, E7, E1, E2, E4, E5, L2 and L1. Some viral types may lack a functional E5 region. The E4 region produces multiple protein products which cause disturbances of the cytoplasmic keratin network, leading to a cytoplasmic “halo effect” termed koilocytosis. The different HPV types are epitheliotopic and after infection can lead to koilocytosis, dyskeratosis, multinucleation, abnormalities such as nuclear enlargement and low grade squamous intraepithelial lesions (LSILs), all of these changes applying only to the cervix. Viral infection and chromosome abnormalities can be correlated in cervical carcinoma, but the multiparametric changes observed in neoplastic lesions, and their association with viral infection, viral gene expression, viral integration, cellular differentiation and genomic abnormalities is very poorly understood (1998, Southern, S. A. et al., Sex Transm Inf., 74, 101-109). It is for this reason that detection of different viral types and their differing effects in different genetic backgrounds is of such critical importance.

Additionally, although the designation of HPV types into cutaneous and mucosal categories and into high-, medium- and low-risk categories is accepted in the prior art, these categories exhibit some fraying and overlap even between the cutaneous and mucosal subcategories of HPV. For example HPV7 has been associated with cutaneous waits as well as oral lesions. HPV26 has been isolated in the context of generalized verrucosis as well as anogenital lesions. Furthermore, although HPV6 and HPV11 have been classified as low-risk types, they have been isolated from Buschke-Lowenstein tumors as well as laryngeal and vulval carcinomas and condylomata acuminata, (1986, Boshart, M. et al., J. Virology, 58, 963-966; 1992, Rubben, A., et al., J Gen Virol., 73, 3147-3153).

Viral integration into the host genome leads to linearization between the E1 and L1 gene regions with retention of the URR, E6 and E7 regions, but with deletion of gene regions such as E1, L1 and L2 and inactivation or deletion of E2. The E6 and E7 regions are generally retained in cervical carcinoma whereas E2 protein expression is absent. E2 damage has been associated with poor prognosis and shortened survival.

### Patient Samples

Cell samples were collected by family physicians from the surface of the uterine cervix using a cervix sampling device supplied by Cytyc Corporation USA. The patients had given consent for the sample to be taken as part of a routine cancer screening program or as a monitoring test for previous cervical disease. The physicians transferred the cells from the collection device to a methanol/water solution for preservation of the cells and transport to the laboratory for testing. The cell sample was assessed for changes due to pre-cancer or viral infections using routine morphological preparations. A separate aliquot of the cell sample was used for DNA testing as outlined in this specification.

### Extraction of DNA

Viral DNA can be obtained from any suitable source. Examples include, but not limited to, cell cultures, broth cultures, environmental samples, clinical samples, bodily fluids, liquid samples, solid samples such as tissue. Viral DNA from samples can be obtained by standard procedures. An example of a suitable extraction is as follows. The sample of interest is placed in 400 μl of 7 M Guanidinium hydrochloride, 5 mM EDTA, 100 mMTris/HCl pH6.4, 1% Triton-X-100, 50 mM Proteinase K (Sigma), 100 μg/ml yeast tRNA. The sample is thoroughly homogenised with disposable 1.5 ml pestle and left for 48 hours at 60° C. After incubation the sample is subjected to five freeze/thaw cycles of dry ice for 5 minutes/95° C. for 5 minutes. The sample is then vortexed and spun in a microfuge for 2 minutes to pellet the cell debris. The supernatant is removed into a clean tube, diluted to reduce the salt concentration then phenol:chloroform extracted, ethanol precipitated and resuspended in 50 μl of 10 mM Tris/0.1 mM EDTA.

Surprisingly, it has been found by the present inventors that there is no need to separate the viral DNA from other sources of nucleic acids. The treatment step can be used for a vast mixture of different DNA types and yet a viral-specific nucleic acid can be still identified by the present invention. It is estimated that the limits of detection in a complex DNA mixtures are that of the limits of standard PCR detection which can be down to a single copy of a target viral nucleic acid molecule.

### Bisulphite Treatment

An exemplary protocol for effective bisulphite treatment of nucleic acid is set out below. The protocol results in retaining substantially all DNA treated. This method is also referred to herein as the Human Genetic Signatures (HGS) method. It will be appreciated that the volumes or amounts of sample or reagents can be varied.

Preferred method for bisulphite treatment can be found in U.S. Ser. No. 10/428,310 or PCT/AU2004/000549 incorporated herein by reference.

To 2 μg of DNA, which can be pre-digested with suitable restriction enzymes if so desired, 2 μl ( 1/10 volume) of 3 M NaOH (6 g in 50 ml water, freshly made) was added in a final volume of 20 μl. This step denatures the double stranded DNA molecules into a single stranded form, since the bisulphite reagent preferably reacts with single stranded molecules. The mixture was incubated at 37° C. for 15 minutes. Incubation at temperatures above room temperature can be used to improve the efficiency of denaturation.

After the incubation, 208 μl 2 M Sodium Metabisulphite (7.6 g in 20 ml water with 416 ml 10 N NaOH; BDH AnalaR #10356.4D; freshly made) and 12 μl of 10 mM Quinol (0.055 g in 50 ml water, BDH AnaIR #103122E; freshly made) were added in succession. Quinol is a reducing agent and helps to reduce oxidation of the reagents. Other reducing agents can also be used, for example, dithiothreitol (DTT), mercaptoethanol, quinone (hydroquinone), or other suitable reducing agents. The sample was overlaid with 200 μl of mineral oil. The overlaying of mineral oil prevents evaporation and oxidation of the reagents but is not essential. The sample was then incubated overnight at 55° C. Alternatively the samples can be cycled in a thermal cycler as follows: incubate for about 4 hours or overnight as follows: Step 1, 55° C./2 hr cycled in PCR machine; Step 2, 95° C./2 min. Step 1 can be performed at any temperature from about 37° C. to about 90° C. and can vary in length from 5 minutes to 8 hours. Step 2 can be performed at any temperature from about 70° C. to about 99° C. and can vary in length from about 1 second to 60 minutes, or longer.

After the treatment with Sodium Metabisulphite, the oil was removed, and 1 μl tRNA (20 mg/ml) or 2 μl glycogen were added if the DNA concentration was low. These additives are optional and can be used to improve the yield of DNA obtained by co-precitpitating with the target DNA especially when the DNA is present at low concentrations. The use of additives as carrier for more efficient precipitation of nucleic acids is generally desired when the amount nucleic acid is <0.5 μg.

An isopropanol cleanup treatment was performed as follows: 800 μl of water were added to the sample, mixed and then 1 ml isopropanol was added. The water or buffer reduces the concentration of the bisulphite salt in the reaction vessel to a level at which the salt will not precipitate along with the target nucleic acid of interest. The dilution is generally about ¼ to 1/1000 so long as the salt concentration is diluted below a desired range, as disclosed herein.

The sample was mixed again and left at 4° C. for a minimum of 5 minutes. The sample was spun in a microfuge for 10-15 minutes and the pellet was washed 2× with 70% ETOH, vortexing each time. This washing treatment removes any residual salts that precipitated with the nucleic acids.

The pellet was allowed to dry and then resuspended in a suitable volume of T/E (10 mM Tris/0.1 mM EDTA) pH 7.0-12.5 such as 50 μl. Buffer at pH 10.5 has been found to be particularly effective. The sample was incubated at 37° C. to 95° C. for 1 min to 96 hr, as needed to suspend the nucleic acids.

Another example of bisulfite treatment can be found in WO 2005021778 (incorporated herein by reference) which provides methods and materials for conversion of cytosine to uracil. In some embodiments, a nucleic acid, such as gDNA, is reacted with bisulfite and a polyamine catalyst, such as a triamine or tetra-amine: Optionally, the bisulfite comprises magnesium bisulfite. In other embodiments, a nucleic acid is reacted with magnesium bisulfite, optionally in the presence of a polyamine catalyst and/or a quaternary amine catalyst. Also provided are kits that can be used to carry out methods of the invention. It will be appreciated that these methods would also be suitable for the present invention in the treating step.

### Amplification

PCR amplifications were performed in 25 μl reaction mixtures containing 2 μl of bisulphite-treated genomic DNA, using the Promega PCR master mix, 6 ng/μl of each of the primers. Strand-specific nested primers are used for amplification. 1st round PCR amplifications were carried out using PCR primers 1 and 4 (see below). Following 1st round amplification, 1 μl of the amplified material was transferred to 2nd round PCR premixes containing PCR primers 2 and 3 and amplified as previously described. Samples of PCR products were amplified in a ThermoHybaid PX2 thermal cycler under the conditions: 1 cycle of 95° C. for 4 minutes, followed by 30 cycles of 95° C. for 1 minute, 50° C. for 2 minutes and 72° C. for 2 minutes; 1 cycle of 72° C. for 10 minutes.

A representation of the fully nested PCR approach is shown below:

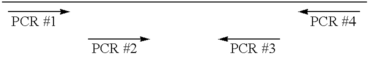

### Multiplex Amplification

One μl of bisulphite treated DNA is added to the following components in a 25 μl 20 reaction volume, ×1 Qiagen multiplex master mix, 5-100 ng of each 1st round INA or oligonucleotide primer 1.5-4.0 mM MgSO₄, 400 μM of each dNTP and 0.5-2 units of the polymerase mixture. The components are then cycled in a hot lid thermal cycler as follows. Typically there can be up to 200 individual primer sequences in each amplification reaction:

Step 1; 94° C. 15 minute 1 cycle

Step 2; 94° C. 1 minute; 50° C. 3 minutes 35 cycles; 68° C. 3 minutes.

Step 3 68° C. 10 minutes 1 cycle

A second round amplification is then performed on a 1 μl aliquot of the first round amplification that is transferred to a second round reaction tube containing the enzyme reaction mix and appropriate second round primers. Cycling is then performed as above.

### HGS ‘Complexity-Reduced’ Primers and Probes

Any suitable PCR primers or probes can be used for the present invention. A primer or probe typically has a complementary sequence to a sequence which will be amplified. Primers or probes are typically oligonucleotides but can be nucleotide analogues such as INAs. Primers to the ‘top’ and ‘bottom’ strands will differ in sequence.

### Probes and Primers

A probe or primer may be any suitable nucleic acid molecule or nucleic acid analogue. Examples include, but not limited to, DNA, RNA, locked nucleic acid (LNA), peptide nucleic acid (PNA), MNA, altritol nucleic acid (ANA), hexitol nucleic acid (HNA), intercalating nucleic acid (INA), cyclohexanyl nucleic acid (CNA) and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. Non-naturally occurring nucleotides include, but not limited to the nucleotides comprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

The probes or primers can be DNA or DNA oligonucleotides containing one or more internal IPNs forming INA.

### Detection Methods

Numerous possible detection systems exist to determine the status of the desired sample. It will be appreciated that any known system or method for detecting nucleic acid molecules could be used for the present invention. Detection systems include, but not limited to:
I. Hybridization of appropriately labelled DNA to a micro-array type device which could select for 10->200,000 individual components. The arrays could be composed of either INAs, PNAs or nucleotide or modified nucleotides arrays onto any suitable solid surface such as glass, plastic, mica, nylon, bead, magnetic bead, fluorescent bead or membrane;
II. Southern blot type detection systems;
III. Standard PCR detection systems such as agarose gel, fluorescent read outs such as Genescan analysis. Sandwich hybridisation assays, DNA staining reagents such as ethidium bromide, Syber green, antibody detection, ELISA plate reader type devices, fluorimeter devices;
IV. Real-Time PCR quantitation of specific or multiple genomic amplified fragments or any variation on that
V. Any of the detection systems outlined in the WO 2004/065625 such as fluorescent beads, enzyme conjugates, radioactive beads and the like;
VI. Any other detection system utilizing an amplification step such as ligase chain reaction or Isothermal DNA amplification technologies such as Strand Displacement Amplification (SDA).
VII. Multi-photon detection systems.
VIII. Electrophoresis and visualisation in gels.
IX. Any detection platform used or could be used to detect nucleic acid.

### Electrophoresis

Electrophoresis of samples was performed according to the E-gel system user guide (www.invitrogen.doc).

### Intercalating Nucleic Acids

Intercalating nucleic acids (INA) are non-naturally occurring polynucleotides which can hybridize to nucleic acids (DNA and RNA) with sequence specificity. INA are candidates as alternatives/substitutes to nucleic acid probes and primers in probe-, or primer-based, hybridization assays because they exhibit several desirable properties. INAs are polymers which hybridize to nucleic acids to form hybrids which are more thermodynamically stable than a corresponding naturally occurring nucleic acid/nucleic acid complex. They are not substrates for the enzymes which are known to degrade peptides or nucleic acids. Therefore, INAs should be more stable in biological samples, as well as having a longer shelf-life than naturally occurring nucleic acid fragments. Unlike nucleic acid hybridization which is very dependent on ionic strength, the hybridization of an INA with a nucleic acid is fairly independent of ionic strength and is favoured at low ionic strength under conditions which strongly disfavour the hybridization of naturally occurring nucleic acid to nucleic acid. The binding strength of INA is dependent on the number of intercalating groups engineered into the molecule as well as the usual interactions from hydrogen bonding between bases stacked in a specific fashion in a double stranded structure. Sequence discrimination is more efficient for INA recognizing DNA than for DNA recognizing DNA.

Preferably, the INA is the phosphoramidite of (S)-1-O-(4,4′-dimethoxytriphenylmethyl)-3-O-(1-pyrenylmethyl)-glycerol.

INAs are synthesized by adaptation of standard oligonucleotide synthesis procedures in a format which is commercially available. Full definition of INAs and their synthesis can be found in WO 03/051901, WO 03/052132, WO 03/052133 and WO 03/052134 (Unest A/S) incorporated herein by reference.

There are indeed many differences between INA probes and primers and standard nucleic acid probes and primers. These differences can be conveniently broken down into biological, structural, and physico-chemical differences. As discussed above and below, these biological, structural, and physico-chemical differences may lead to unpredictable results when attempting to use INA probes and primers in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. INA, however, is a recently developed totally artificial molecule, conceived in the minds of chemists and made using synthetic organic chemistry. It has no known biological function.

Structurally, INAs also differ dramatically from nucleic acids. Although both can employ common nucleobases (A, C, G, T, and U), the composition of these molecules is structurally diverse. The backbones of RNA, DNA and INA are composed of repeating phosphodiester ribose and 2-deoxyribose units. INA differs from DNA or RNA in having one or more large flat molecules attached via a linker molecule(s) to the polymer. The flat molecules intercalate between bases in the complementary DNA stand opposite the INA in a double stranded structure.

The physico/chemical differences between INA and DNA or RNA are also substantial. INA binds to complementary DNA more rapidly than nucleic acid probes or primers bind to the same target sequence. Unlike DNA or RNA fragments, INA bind poorly to RNA unless the intercalating groups are located in terminal positions. Because of the strong interactions between the intercalating groups and bases on the complementary DNA strand, the stability of the INA/DNA complex is higher than that of an analogous DNA/DNA or RNA/DNA complex.

Unlike other nucleic acids such as DNA or RNA fragments or PNA, INAs do not exhibit self aggregation or binding properties.

In summary, as INAs hybridize to nucleic acids with sequence specificity, INAs are useful candidates for developing probe-, or primer-based assays and are particularly adapted for kits and screening assays. INA probes and primers, however, are not the equivalent of nucleic acid probes and primers. Consequently, any method, kits or compositions which could improve the specificity, sensitivity and reliability of probe-, or primer-based assays would be useful in the detection, analysis and quantitation of DNA containing samples. INAs have the necessary properties for this purpose.

### EXAMPLES

To reiterate the foundations on which we have based our bioinformatic analyses in silico, the standard HPV type utilized for reference purposes is HPV16 of the Family Papovaviridae, Genus Papillomavirus, originally designated as such by the International Committee on Taxonomy of Viruses, ICTV, (1993, Van Rast, M. A., et al., Papillomavirus Rep, 4, 61-65; see also, 1998 Southern, S. A. and Herrington, C. S. Sex. Transm. Inf. 74, 101-109), although taxonomic upgrades to the Papillomaviridae are sometimes used interchangeably in the prior art. To avoid ambiguity, we use the fully sequenced 7904 base pair genome of HPV16 as a standard comparator (National Center for Biotechnology Information, NCBI locus NC_{—}001526; version NC_{—}001526.1; GI:9627100; references, Medline, 91162763 and 85246220; PubMed 1848319 and 2990099).

In addition, we used the fully sequenced genomes of the so called high-risk HPV types 16, 18, 45 and 56 with NCBI accession numbers of NC-001526, NC-001357, NC-001590 and NC-001594 respectively.

We used the fully sequenced genomes of the so called medium risk HPV types 31, 33, 35, 39, 51, 52, 58 and 66 with NCBI accession numbers NC-001527, NC-001528, NC-001529, NC-001535, NC-001533, NC-001592, NC-001443 and NC-001695 respectively.

We used the fully sequenced genomes of the so called low risk HPV types 6, 11, 30, 42, 43, 44, 53, 54 and 55 with NCBI accession numbers of NC-000904, NC-001525, NC-001585, NC-001534, NC-005349, NC-001689, NC-001593, NC-001676 and NC-001692 respectively.

As we have demonstrated, the detection of human papilloma viral DNA in various clinical samples via conventional DNA tests is hampered by a number of technical, methodological and clinical problems. The present invention provides a solution to many of the difficulties encountered in the prior art, since the bisulphite conversion of HPV DNA reduces the complexity of the HPV derivative sequence pool. This complexity-reduction allows for a more efficient initial screening of the different HPV types within a sample and hence for a more appropriate and accurate interface with the clinical data.

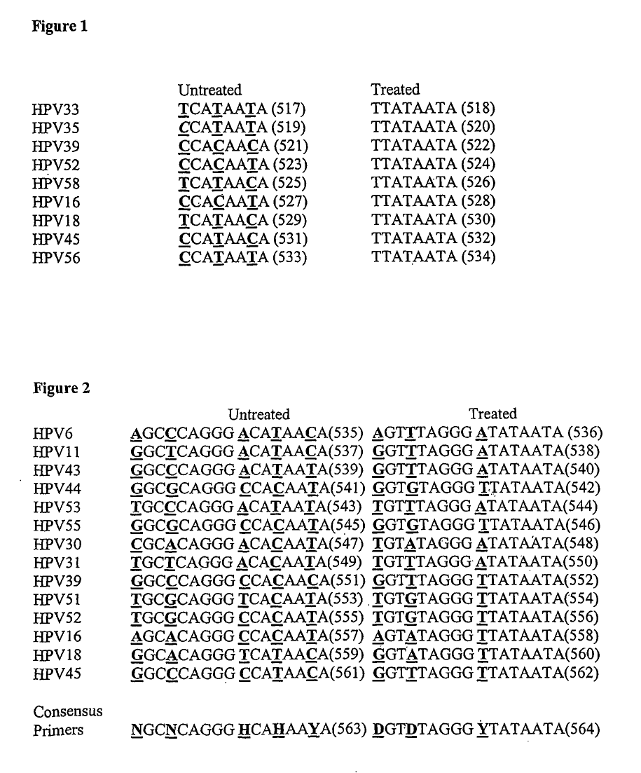

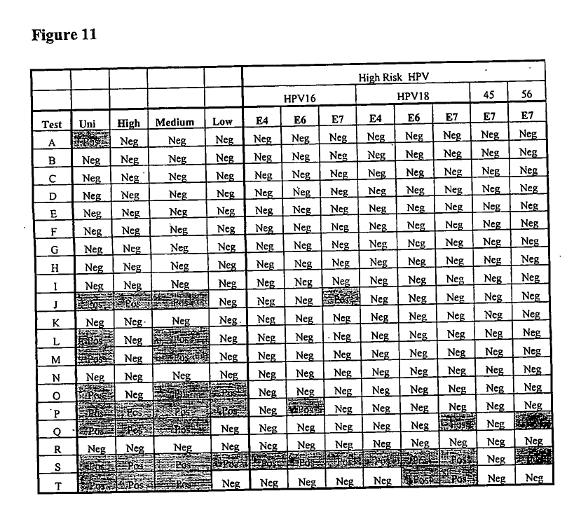

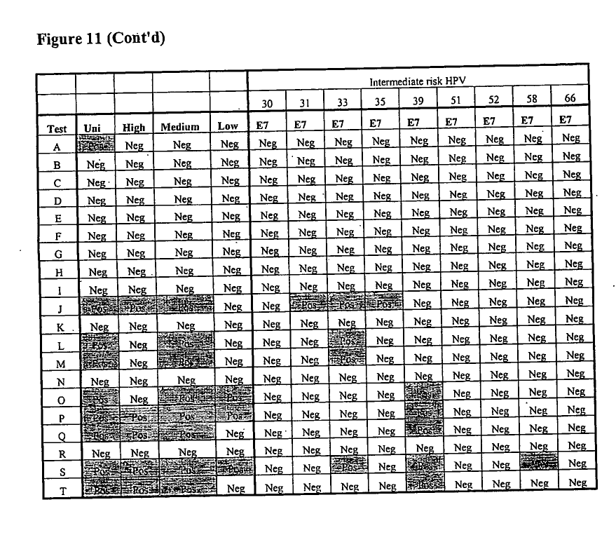

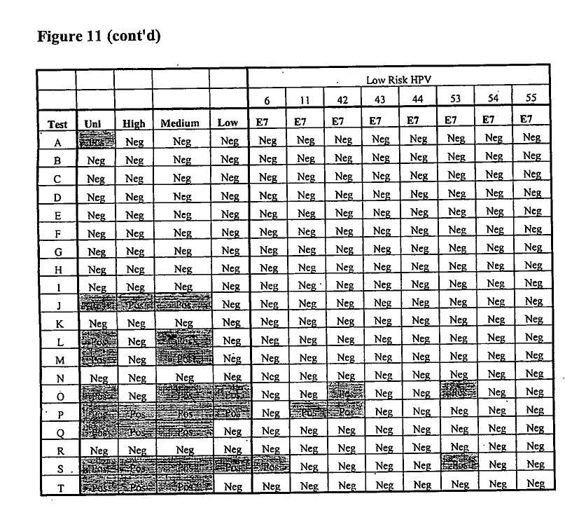

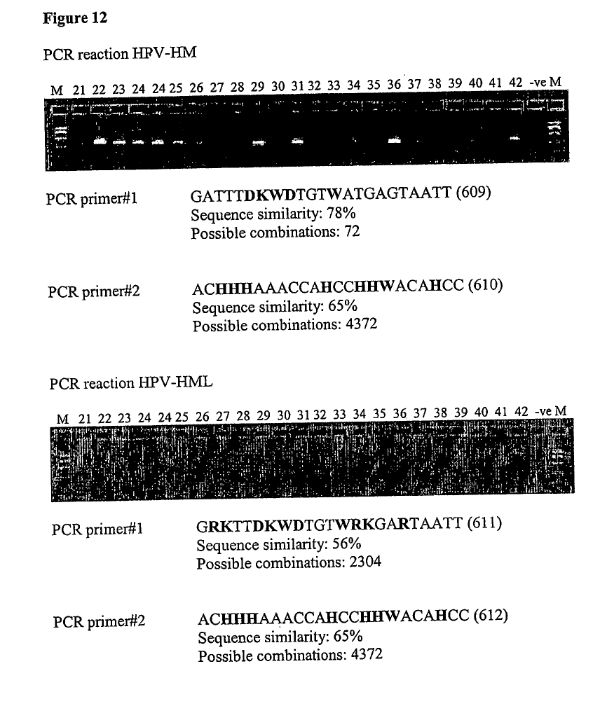

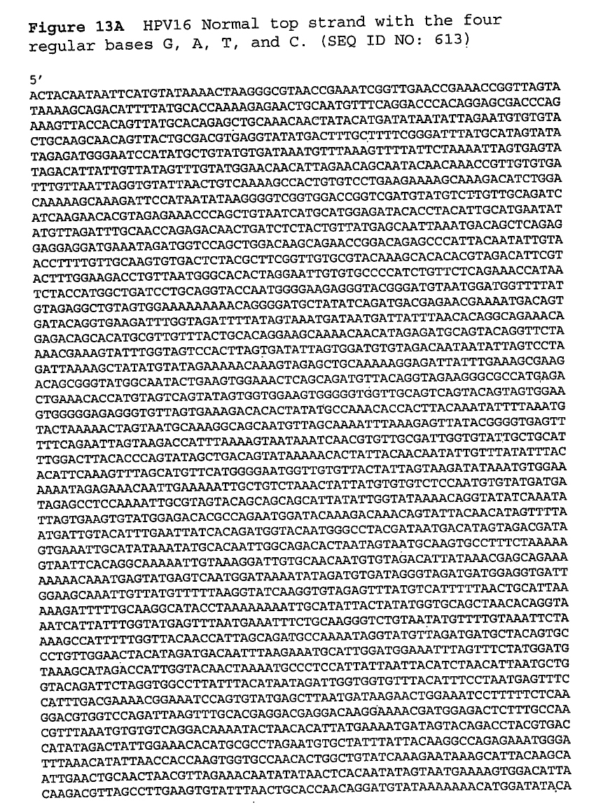

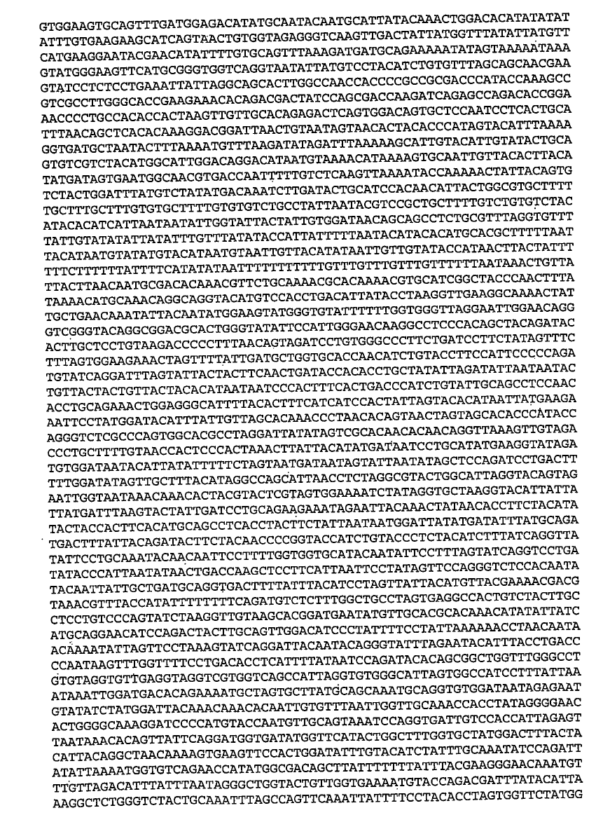

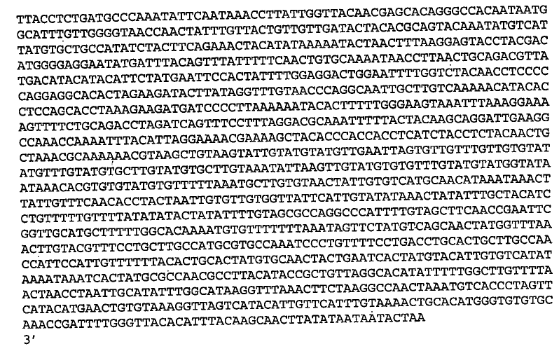

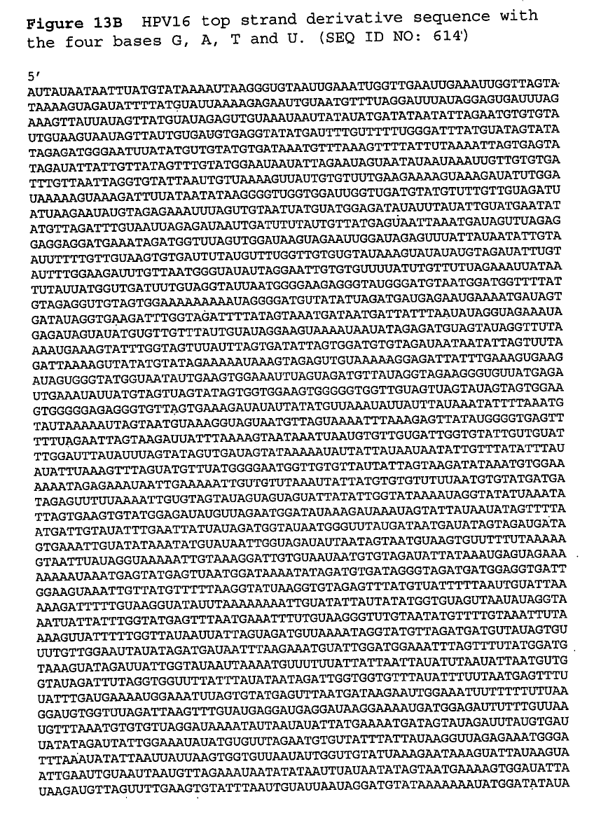

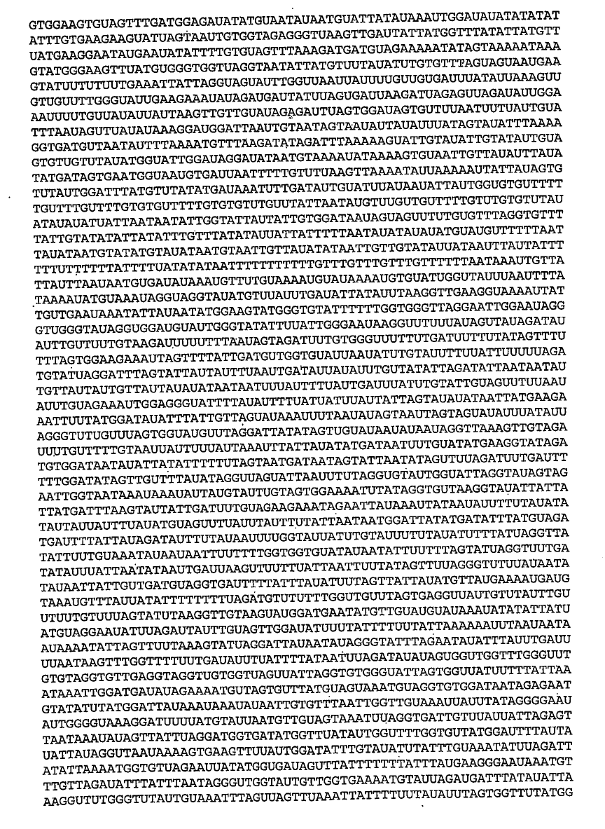

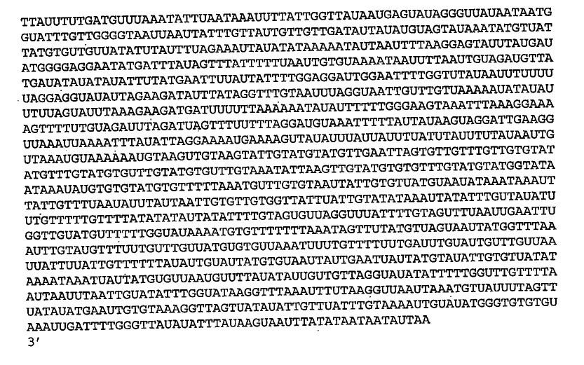

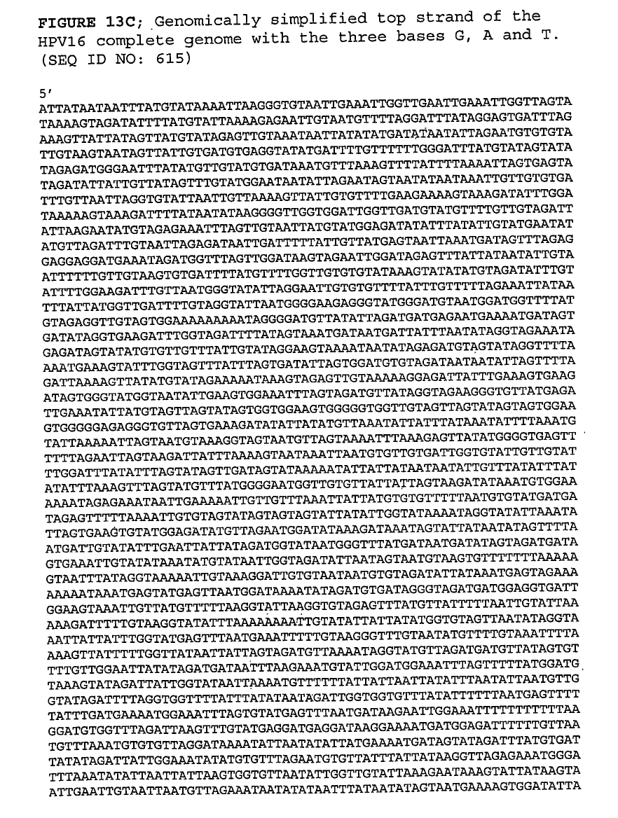

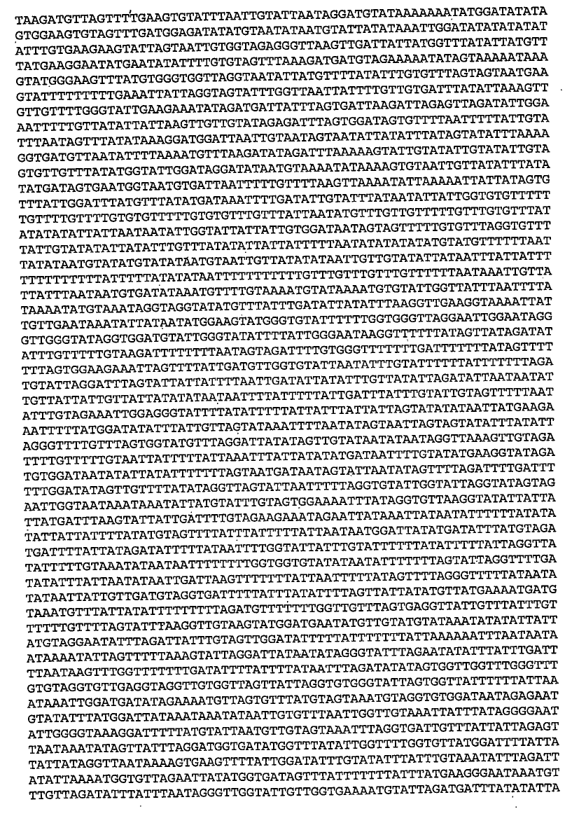

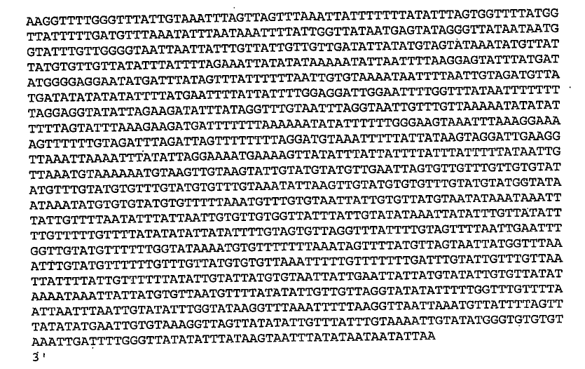

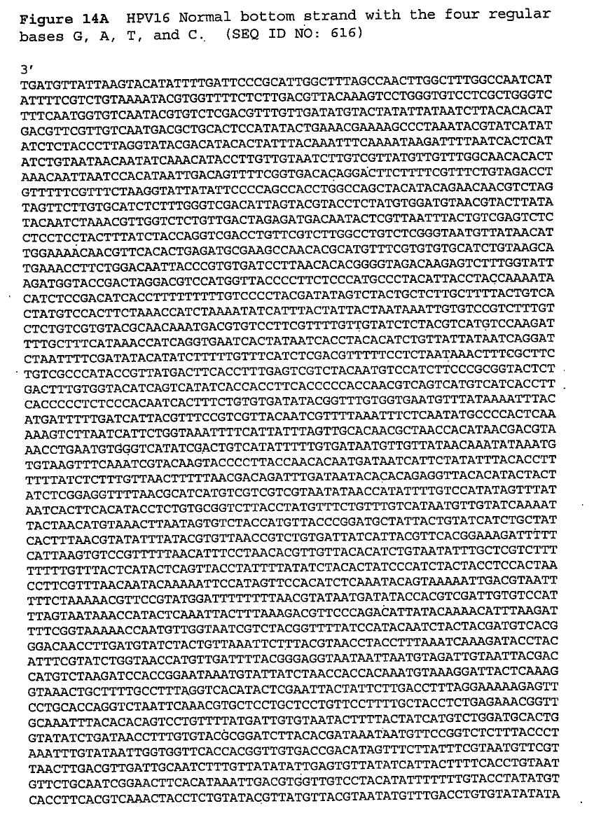

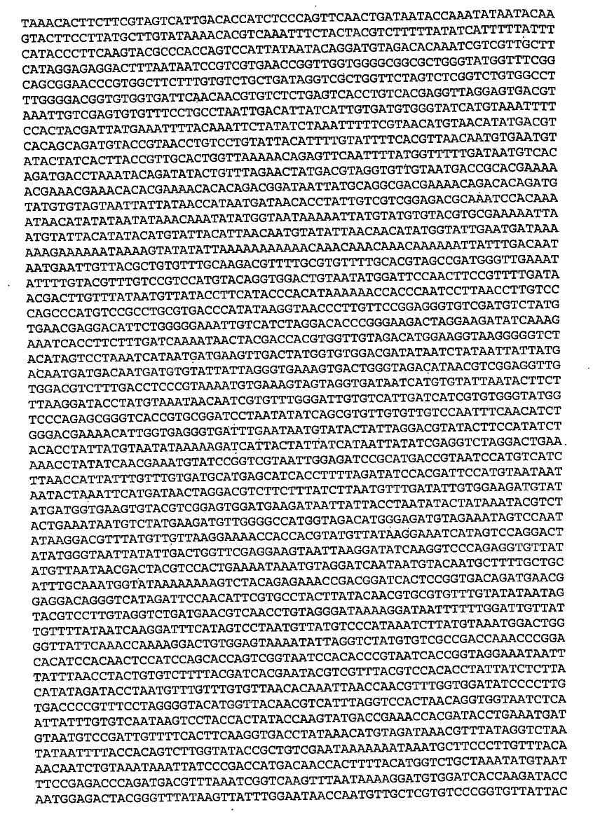

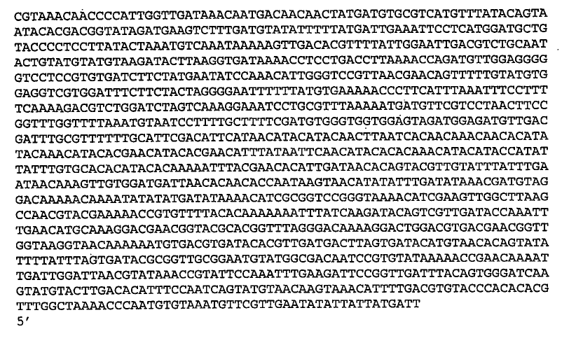

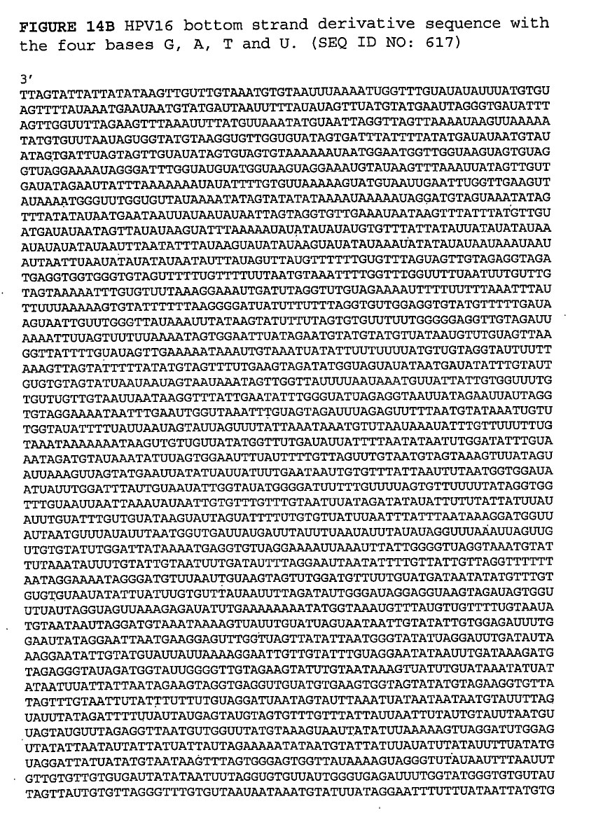

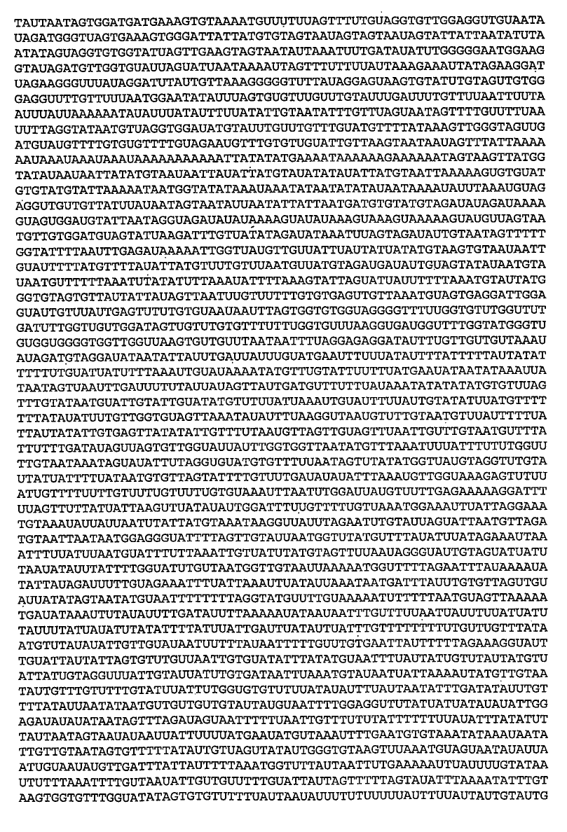

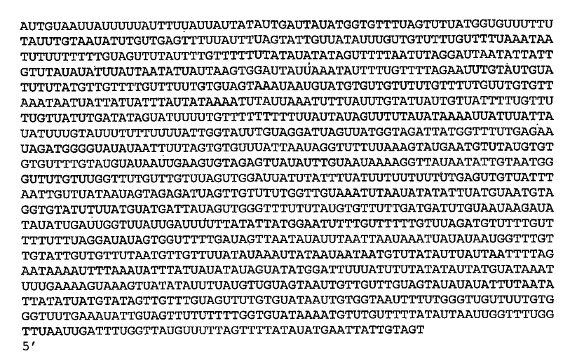

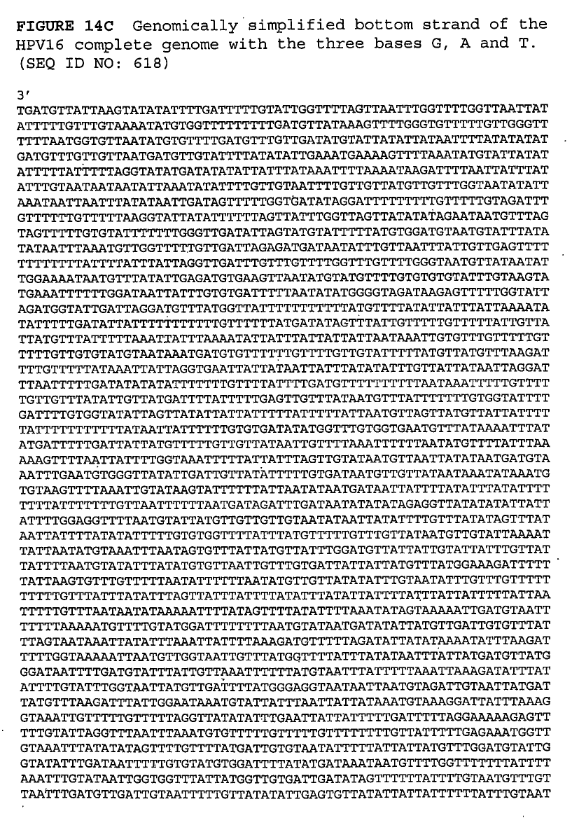

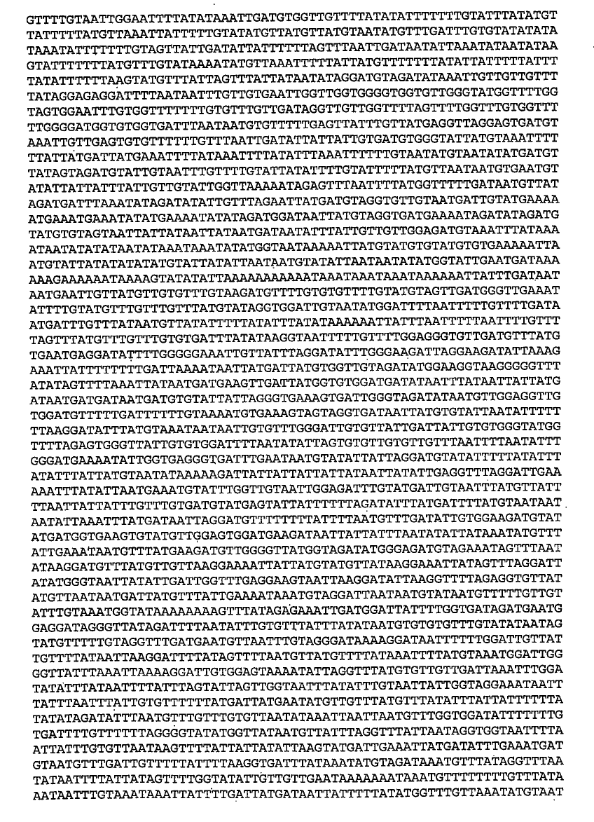

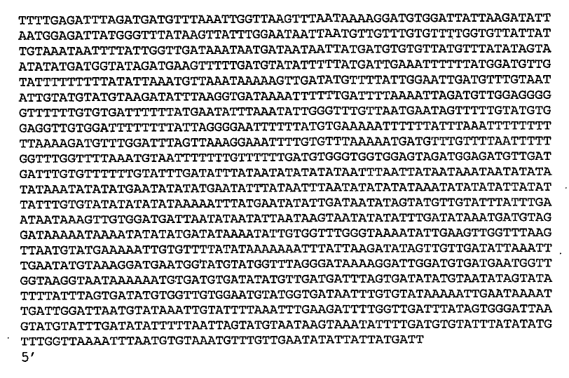

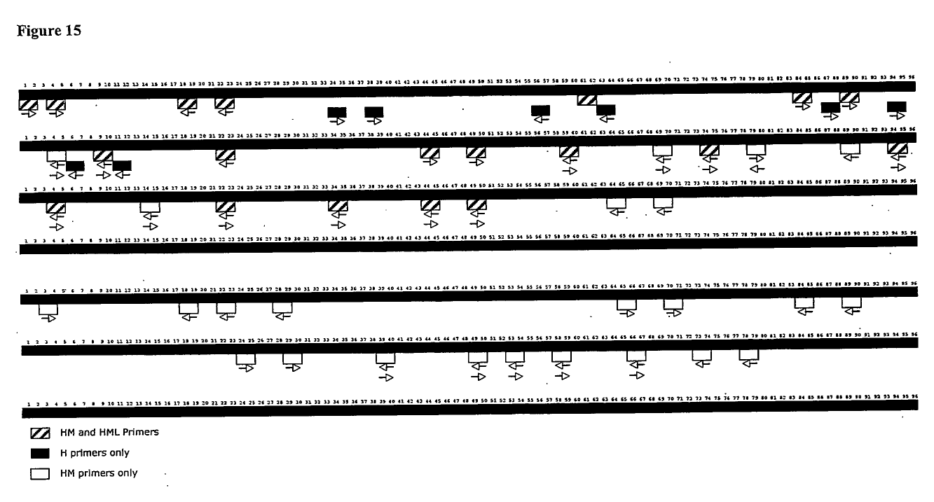

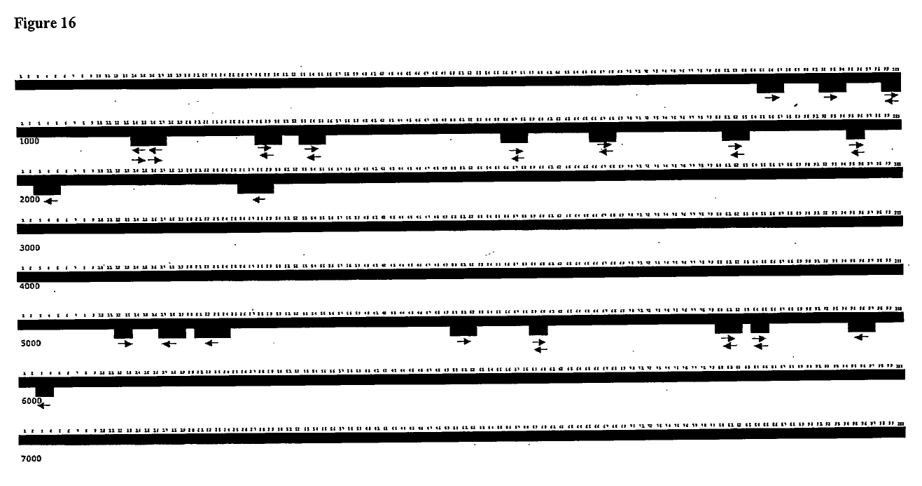

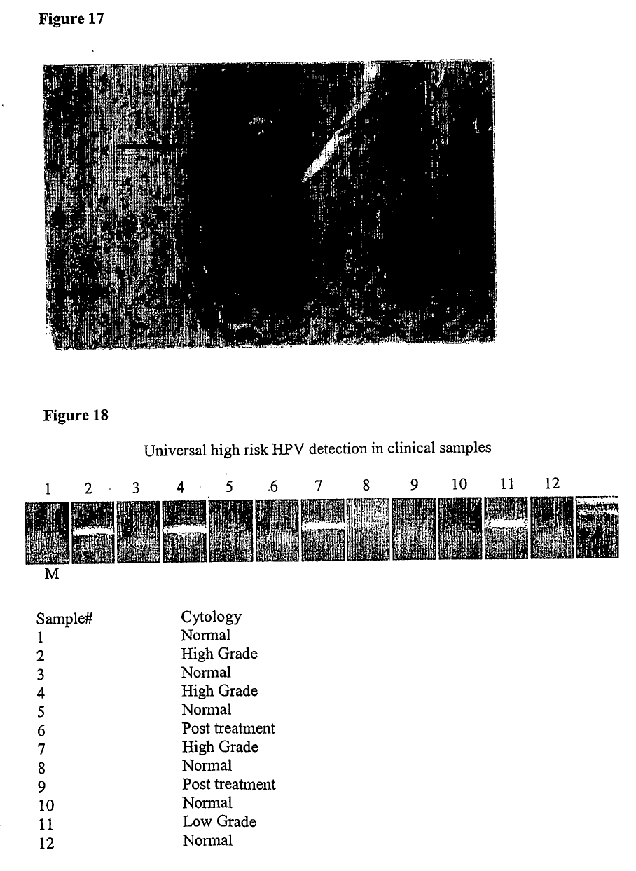

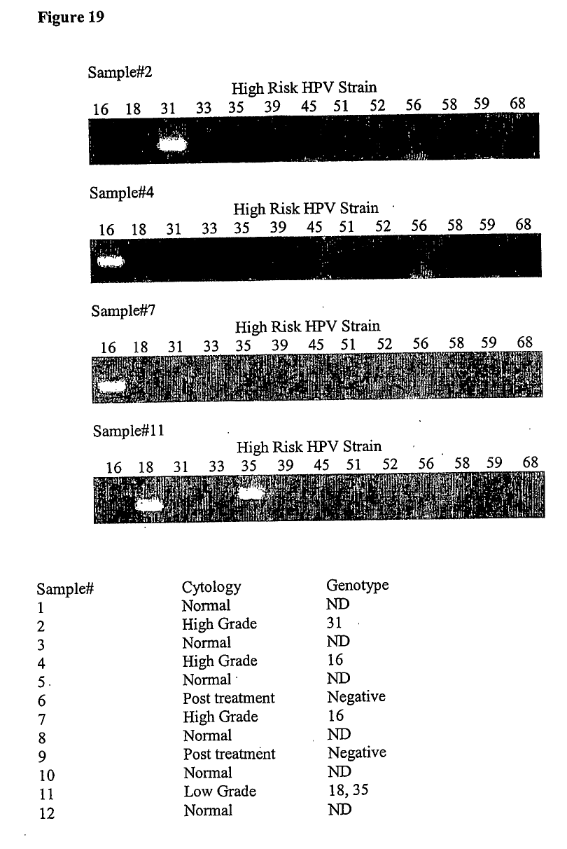

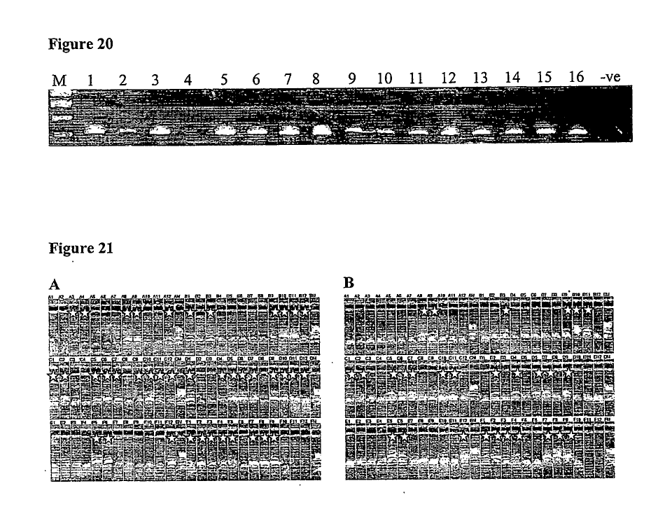

FIGS. 1 to 4 depict the in silico groundwork that allowed for the optimum design of primers and probes for the detection of portions of what was the original HPV genome, but is now its converted derivative. FIGS. 5 to 10 show PCR amplified nucleic acid products generated from different regions of different HPV types, of different oncogenic risk types, using ‘universal’ primers or combinations of primers in multiplex PCR reactions using clinical samples from 16 different patients. FIG. 11 tabulates these results. FIG. 12 illustrates the consequences of primer degeneracy on the outcome of PCR reactions and the advantages of the current invention. FIGS. 13 and 14 illustrate the normal, derivative and genomically simplified sequences of the top and bottom strands of HPV16. FIGS. 15 and 16 illustrate the “helicopter” view of where the preferred primers are to be found on the top and bottom strands of the HPV sequence. FIG. 17 shows a section of a clinical sample revealing cancerous cells of the cervix surrounded by normal stromal cells. FIGS. 18 and 19 illustrate the two stages of typing clinical samples, with the former figure revealing that a high-medium risk HPV is present in a Liquid Based Cytology sample, and the latter revealing the exact viral type in the same sample. FIG. 20 shows the results of identifying high-medium risk HPV types (HPV 16, 18, 31, 33, 35, 39, 45, 51, 52, 56, 58, 59 and 68) from archival paraffin sections, rather than liquid based samples. FIG. 21 demonstrates that HPV typing can be done not just by using primers made to the genomically simplified top strand, but to the genomically simplified bottom strand as well. Furthermore, the invention is also taught in Tables 1 through 4, where the sequences of the primers; some examples of the expected amplicon sizes; and the results of HPV typing in hundreds of clinical samples, are compared to the current FDA approved Hybrid Capture 2 methodology applied to the same samples.

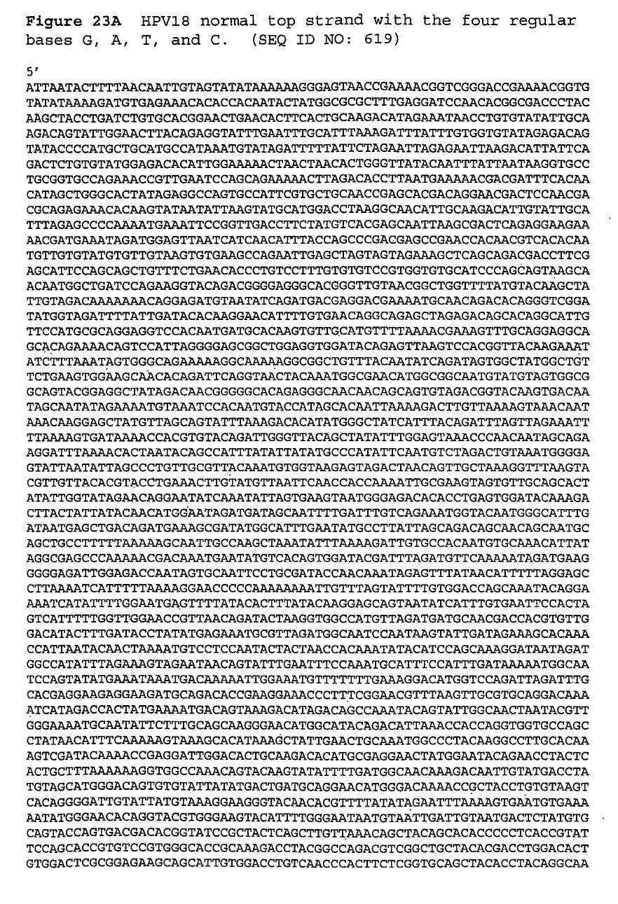

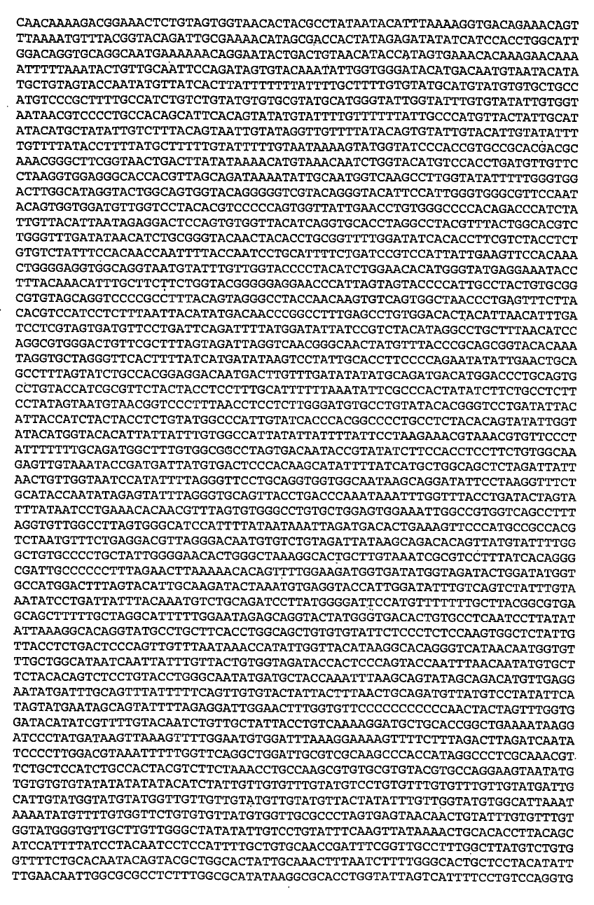

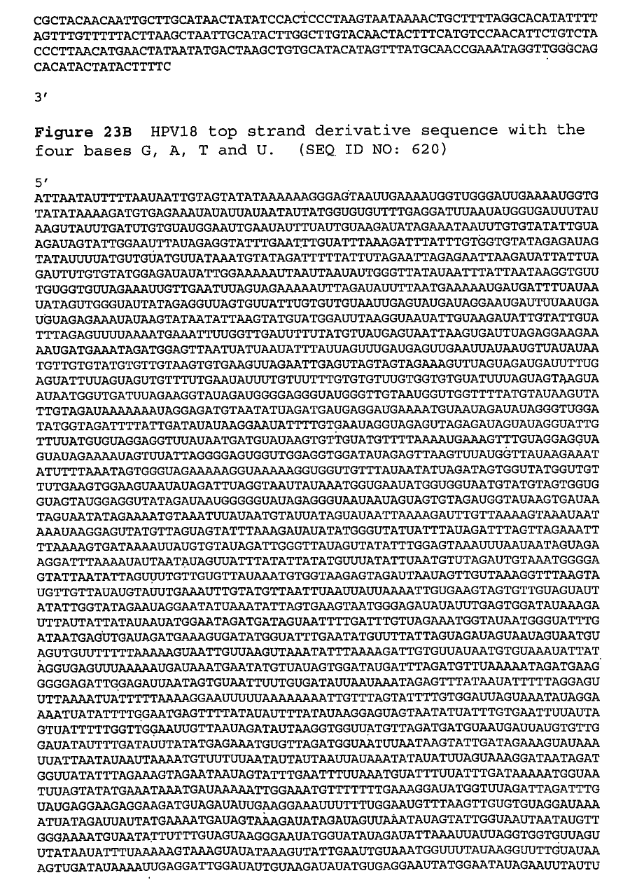

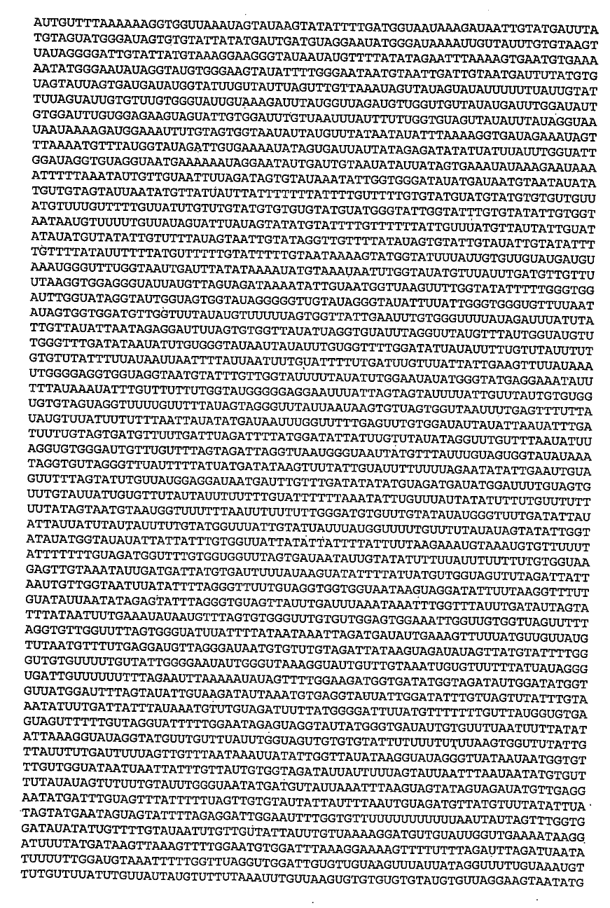

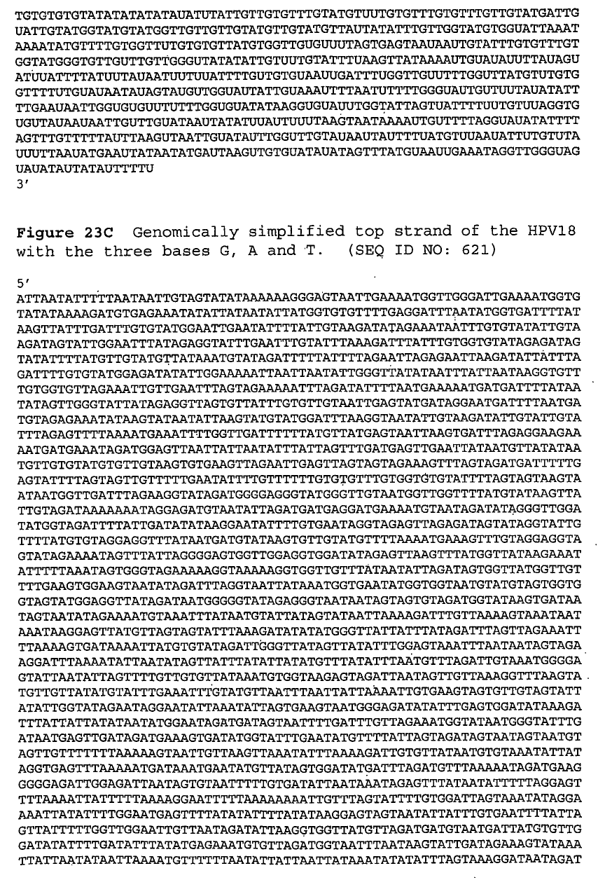

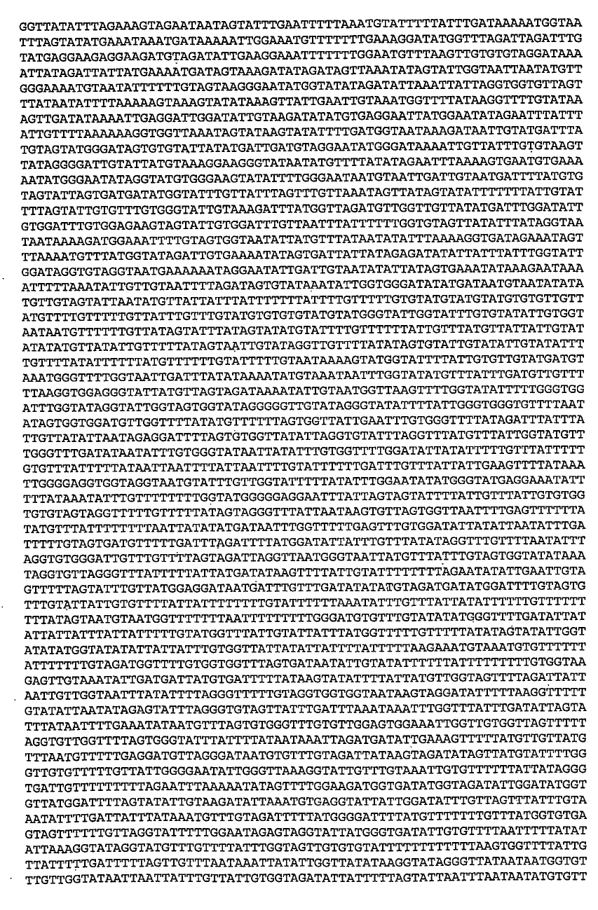

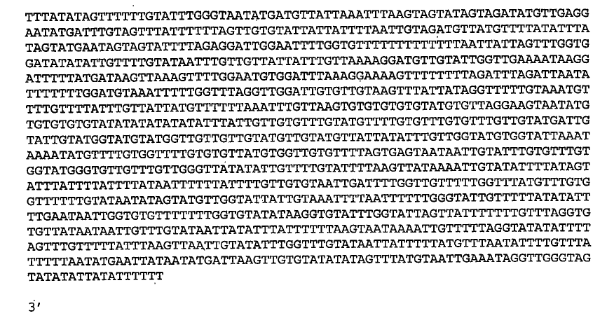

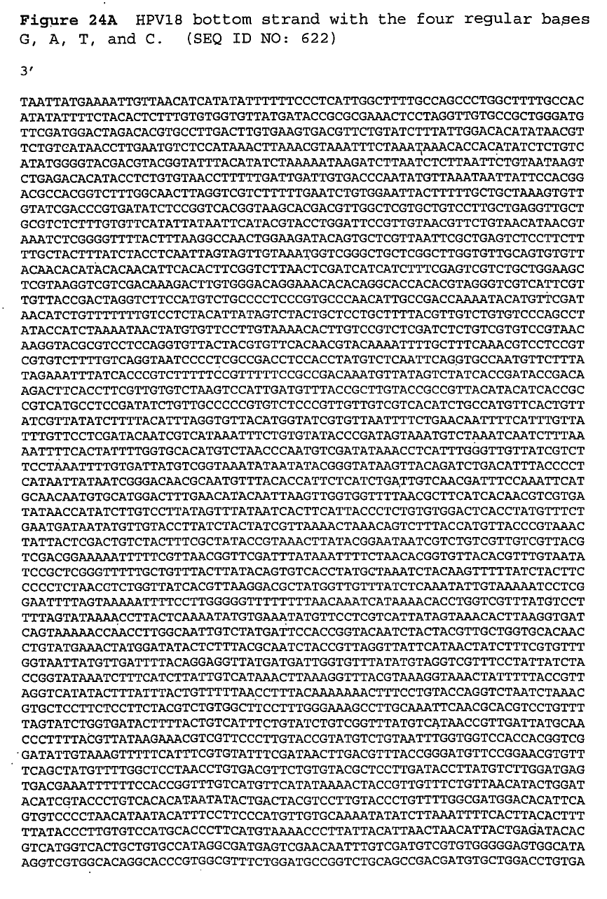

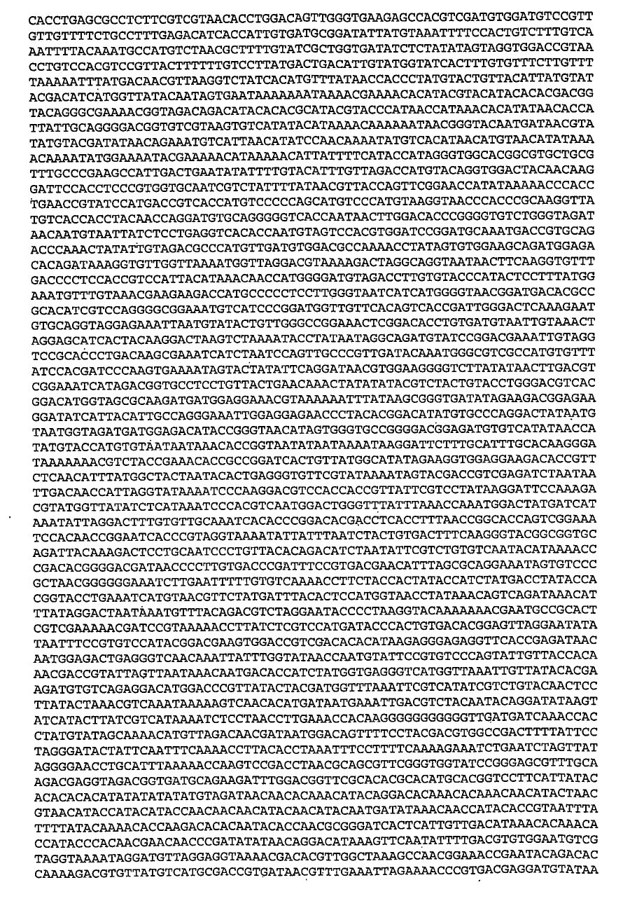

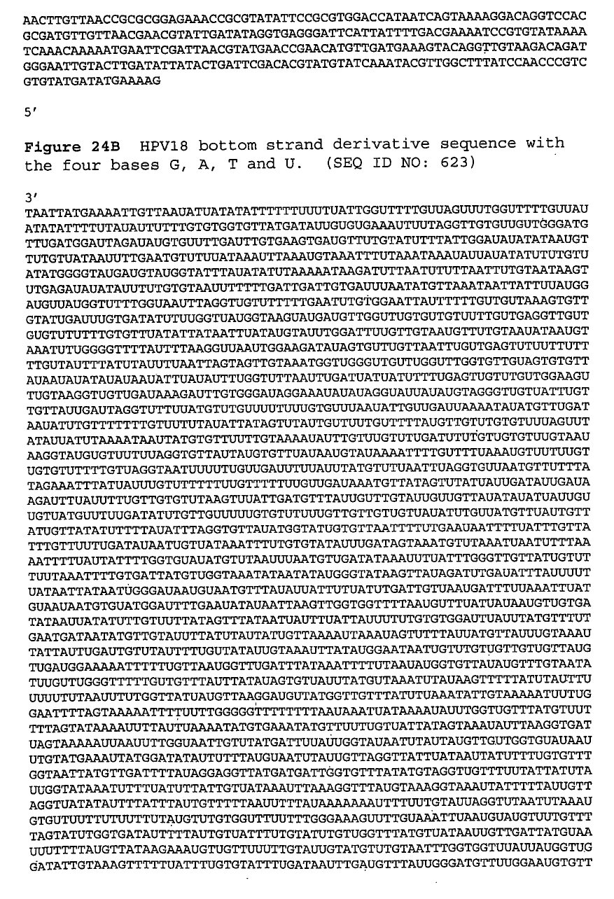

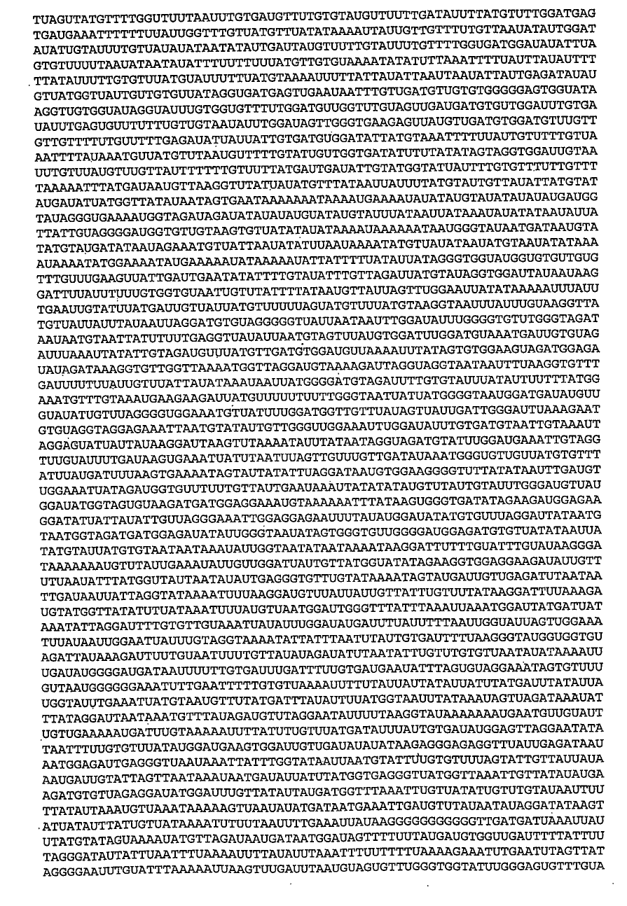

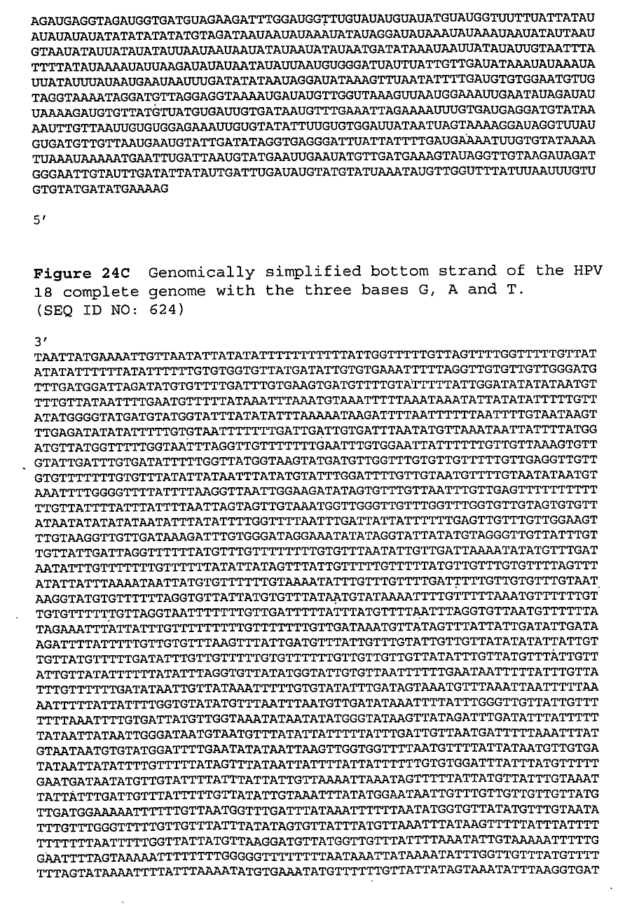

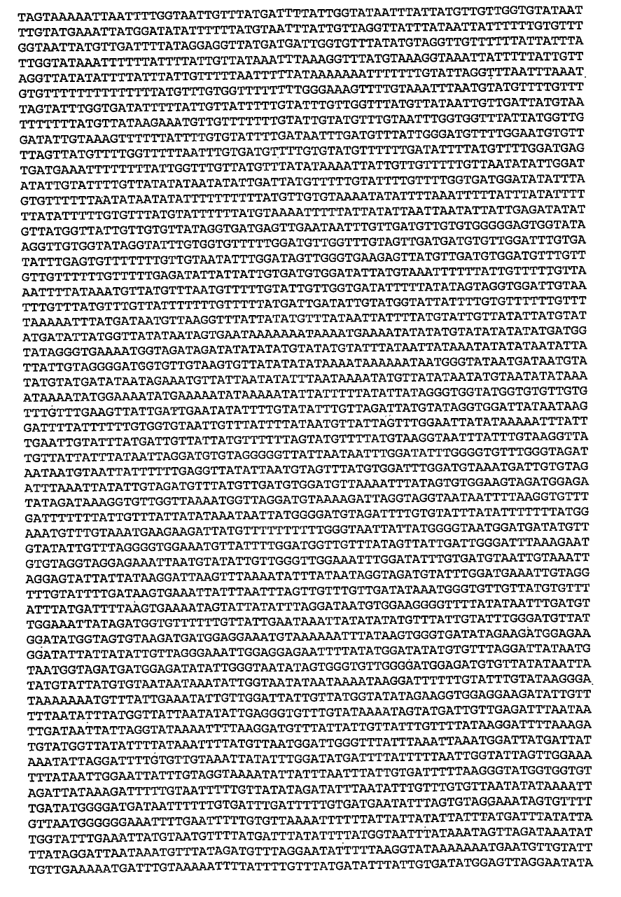

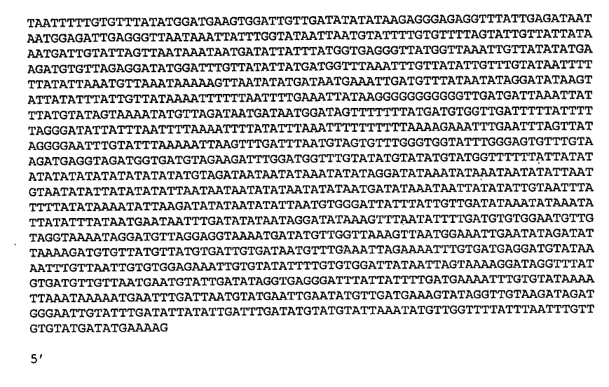

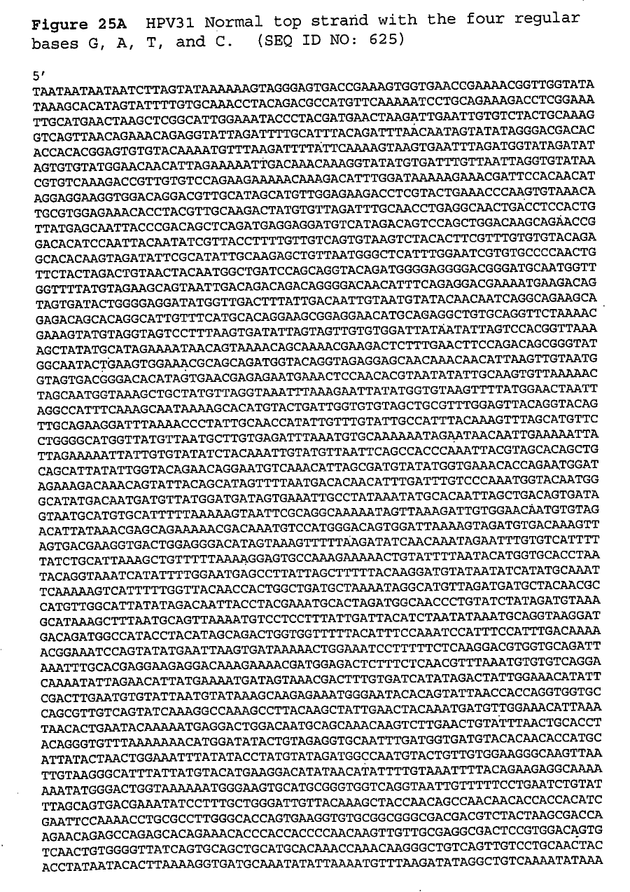

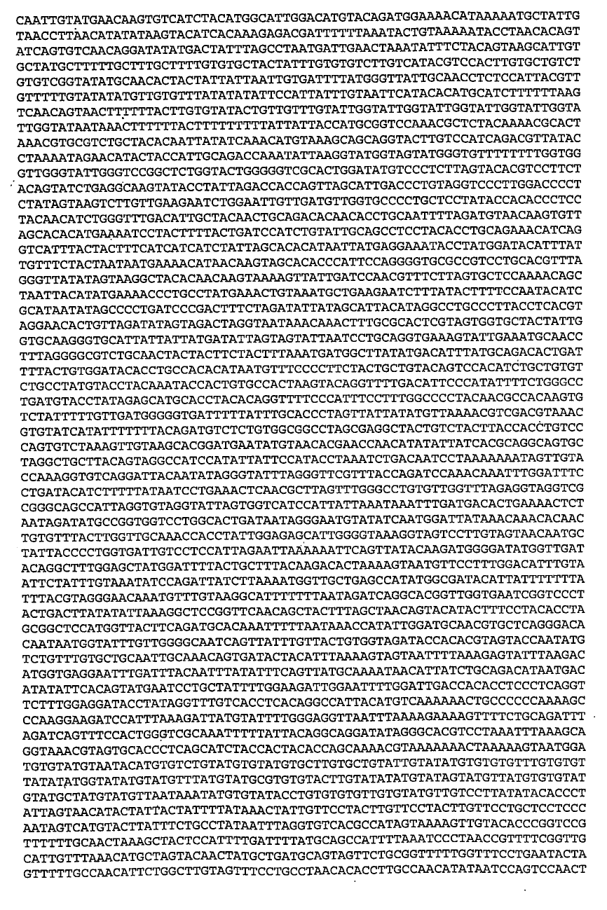

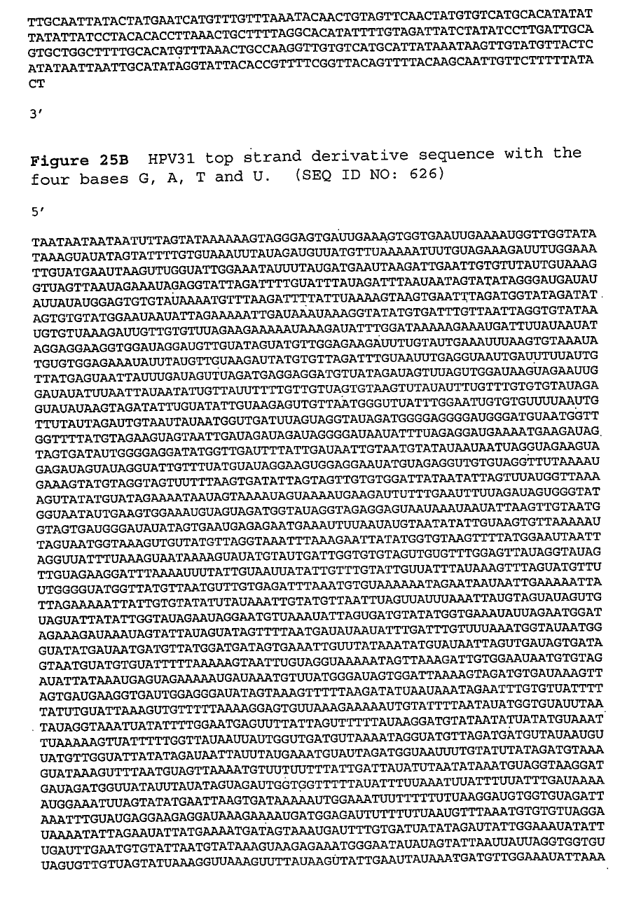

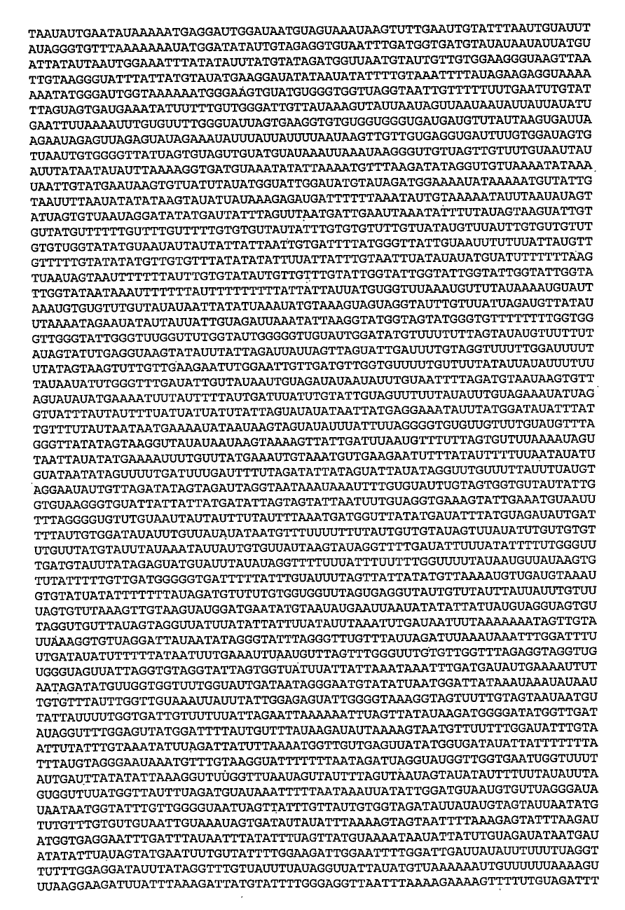

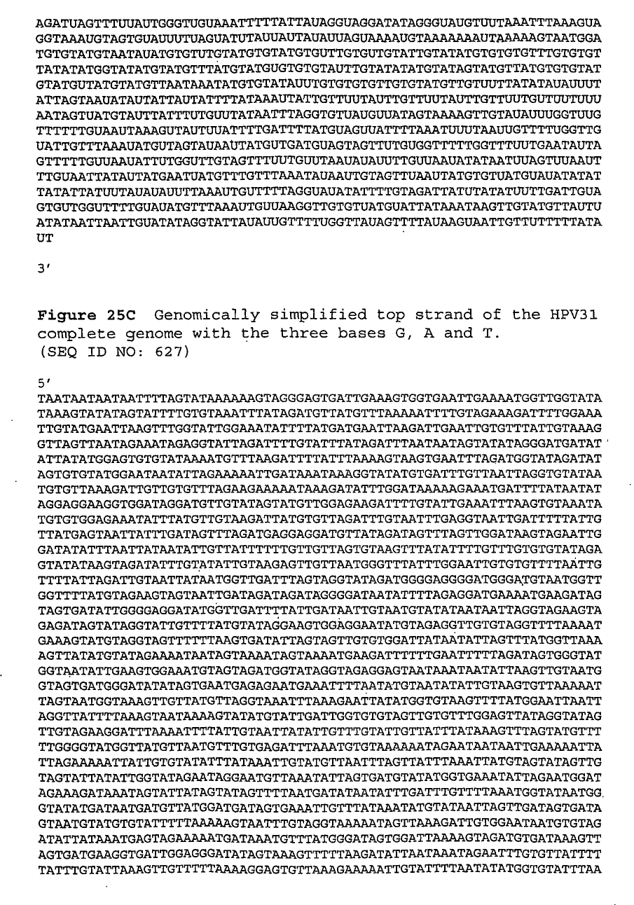

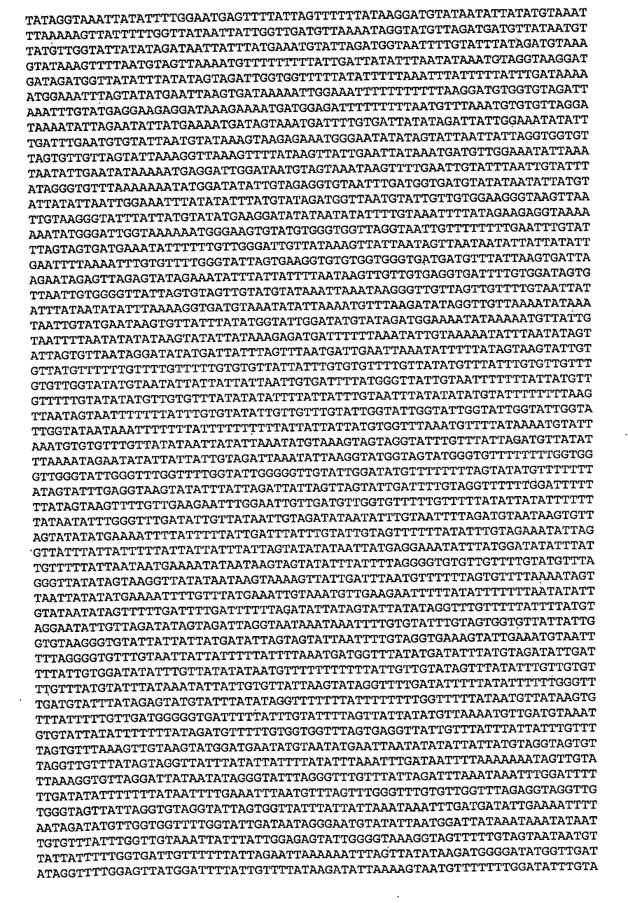

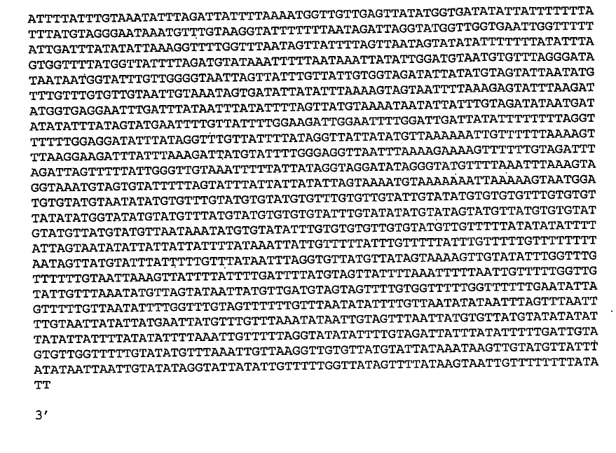

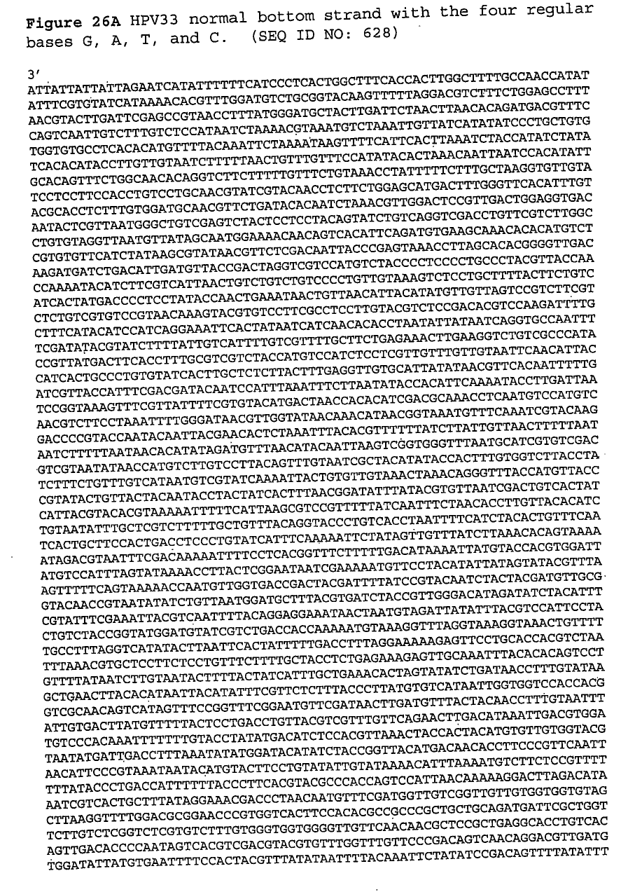

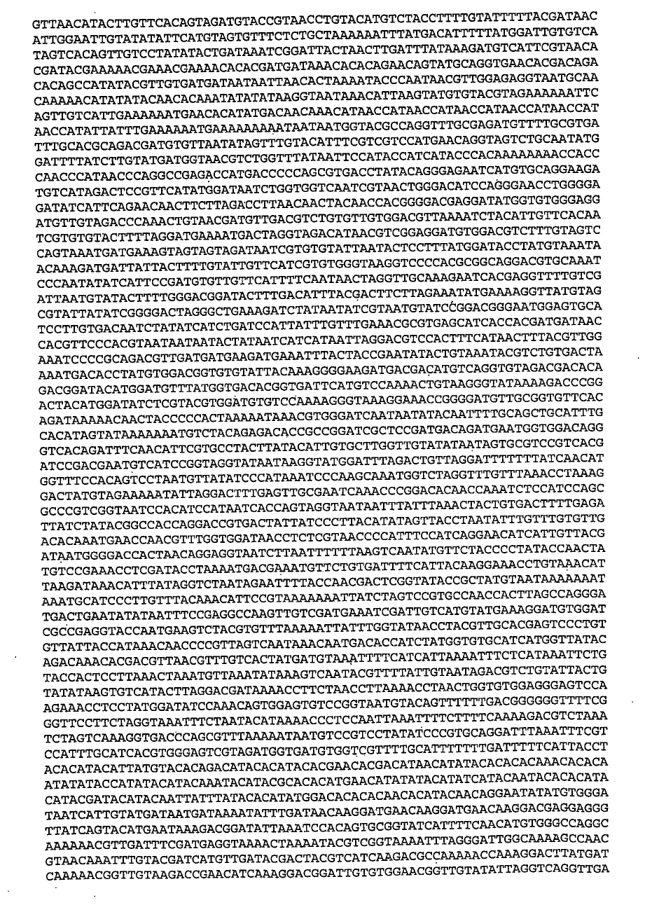

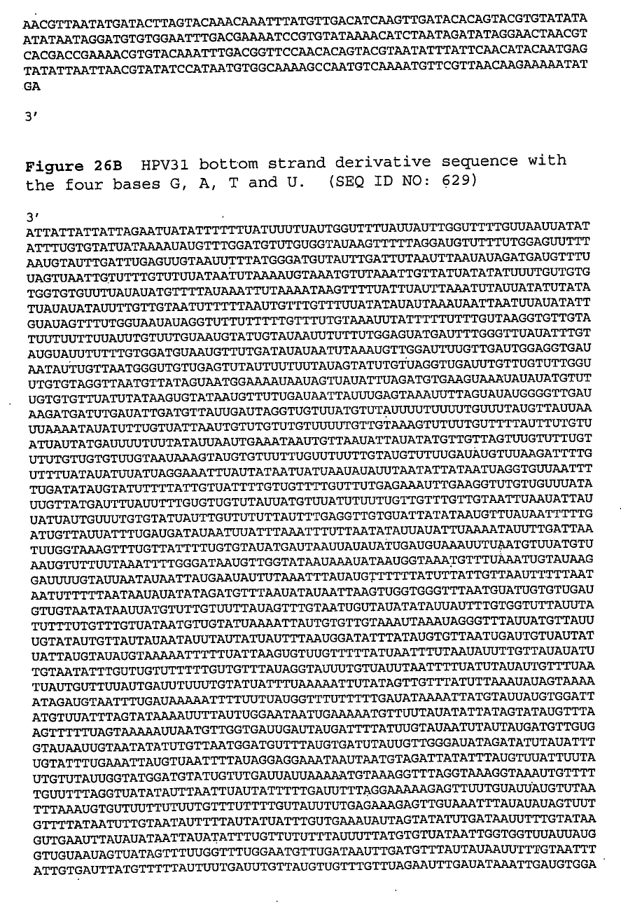

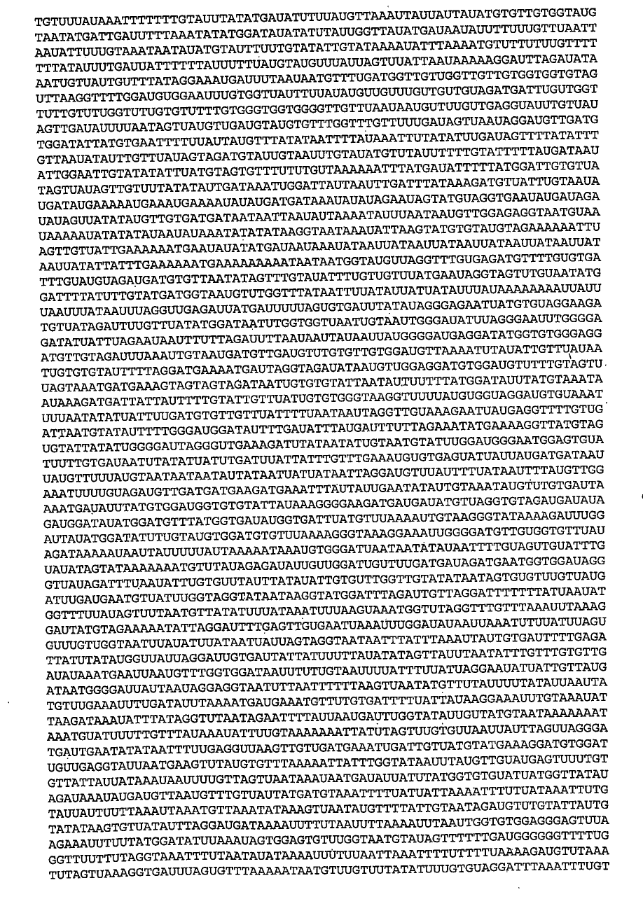

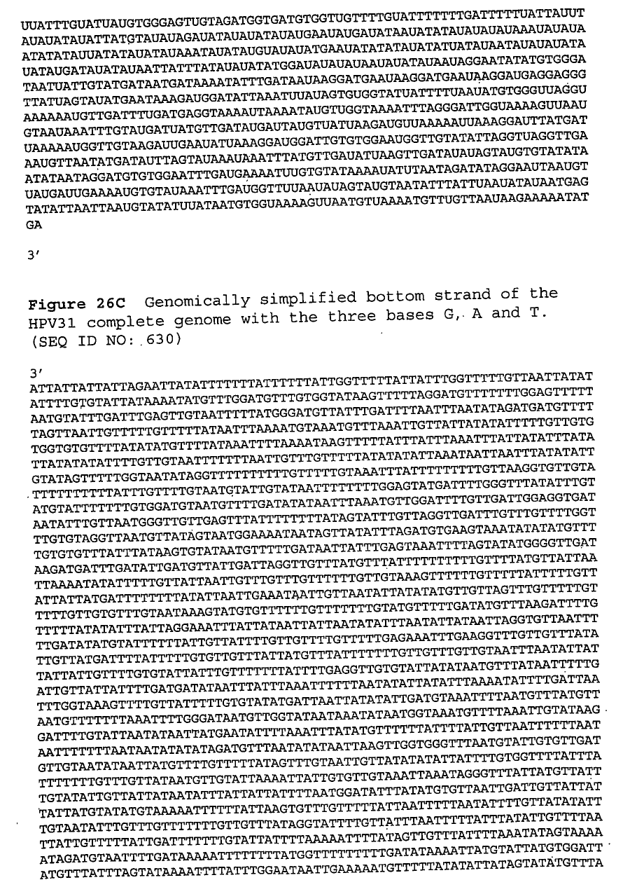

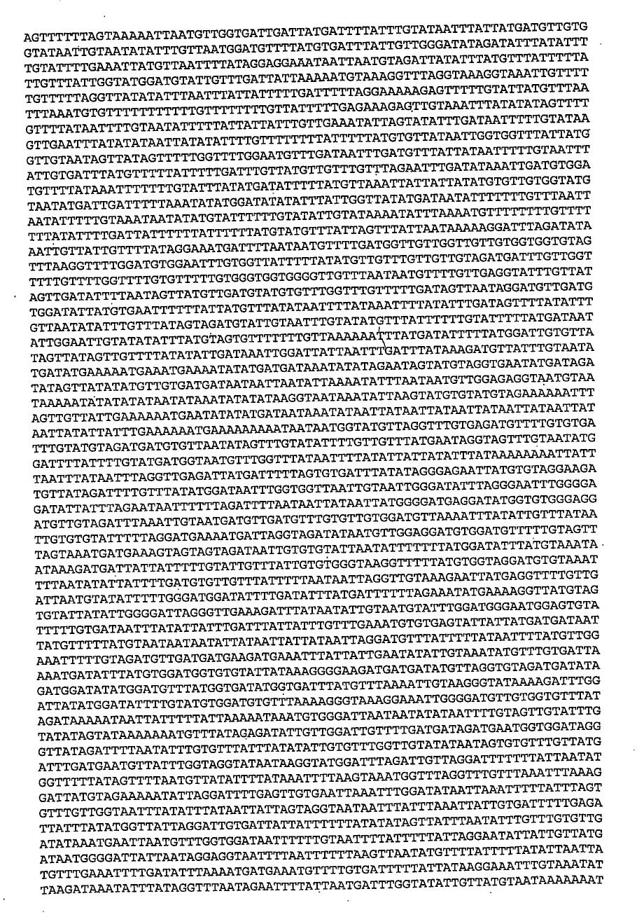

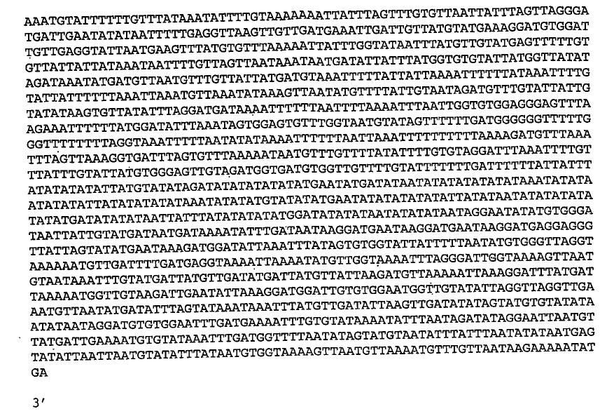

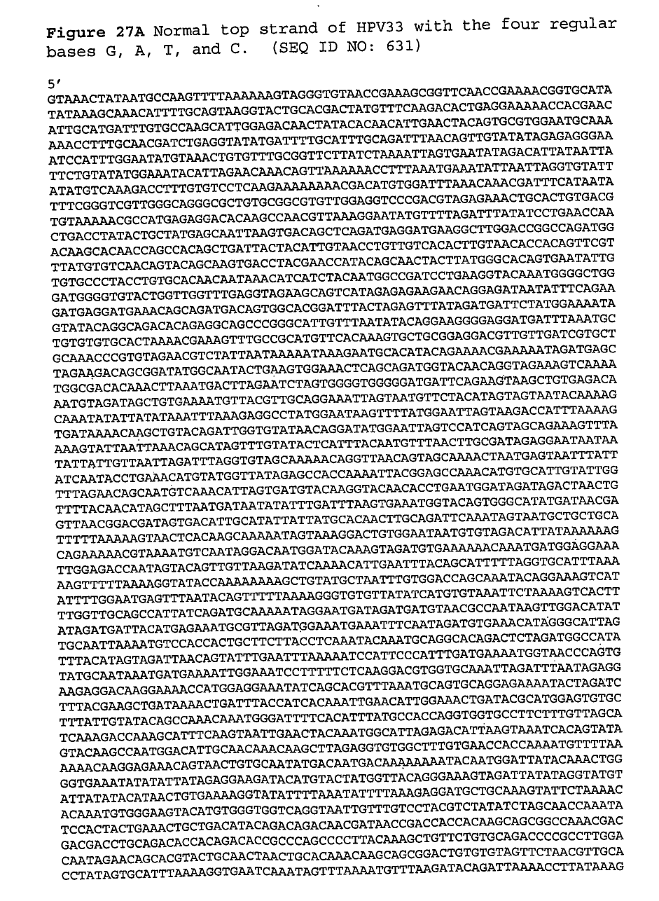

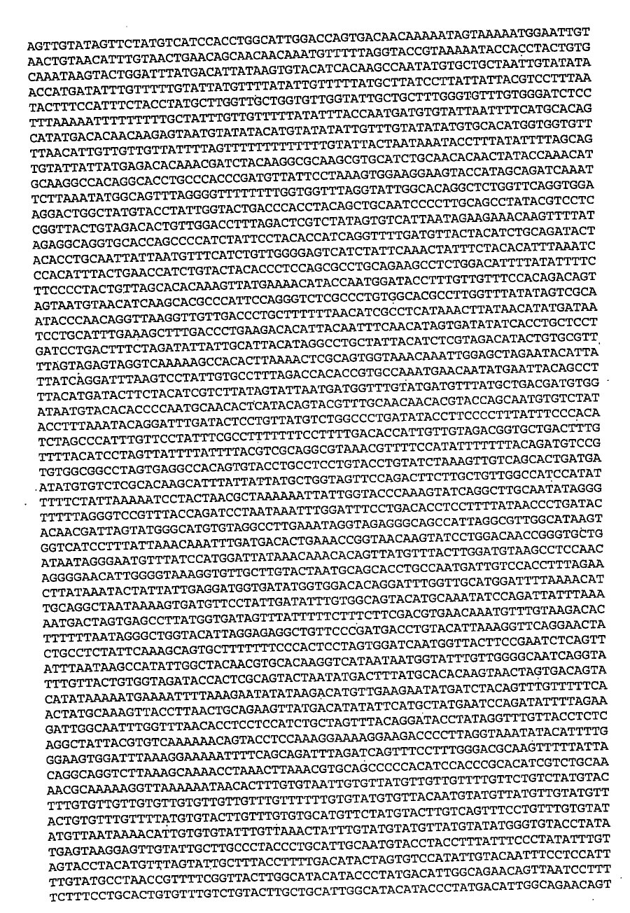

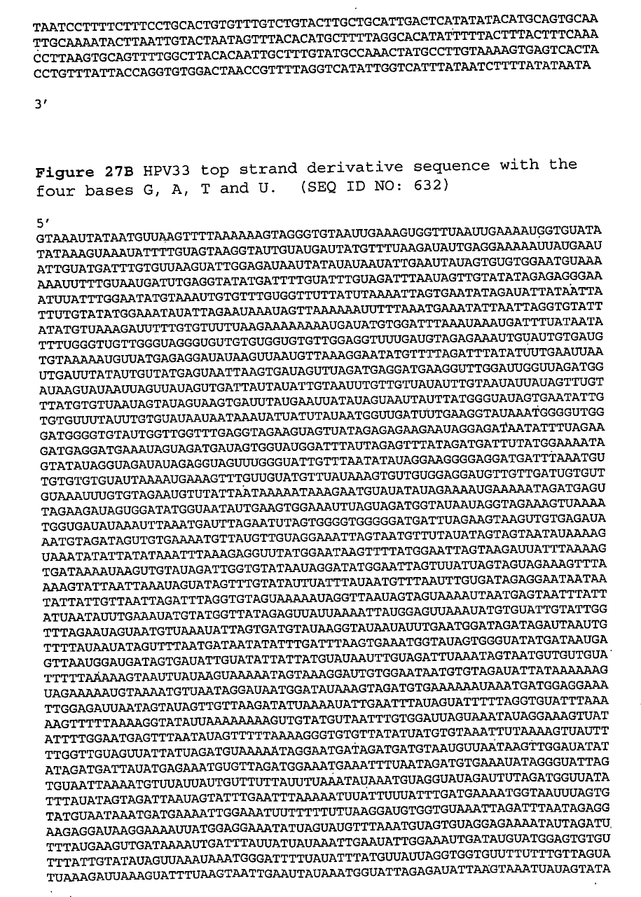

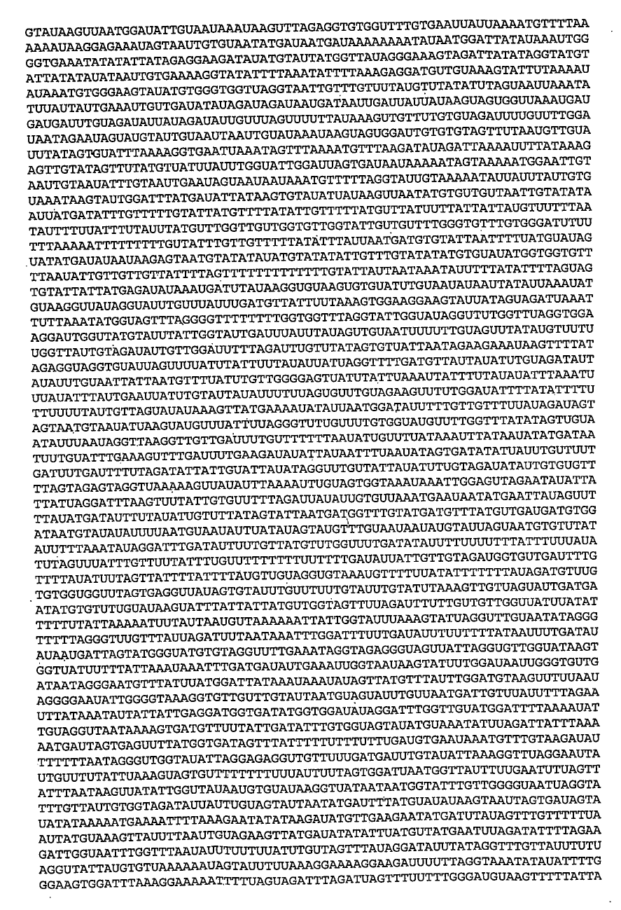

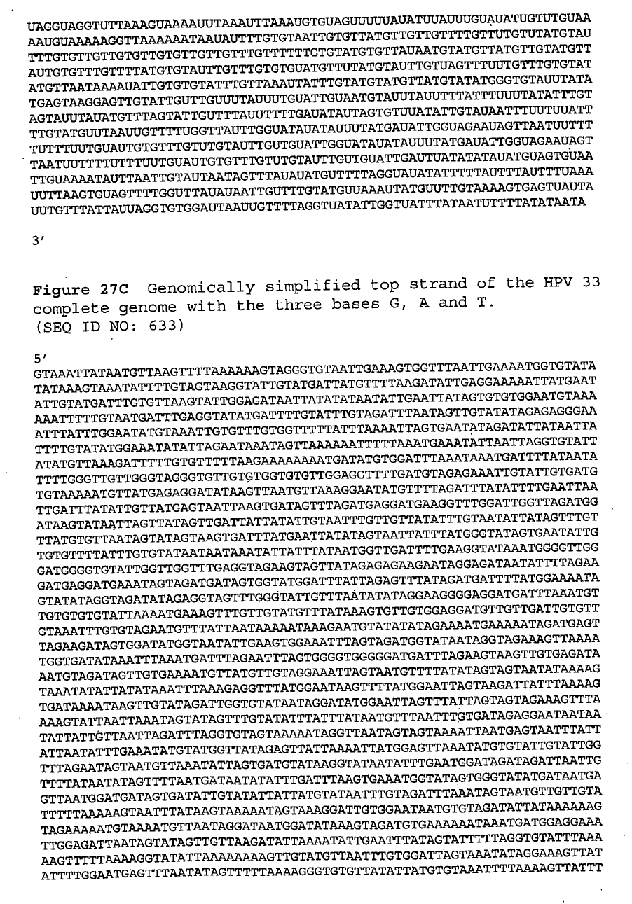

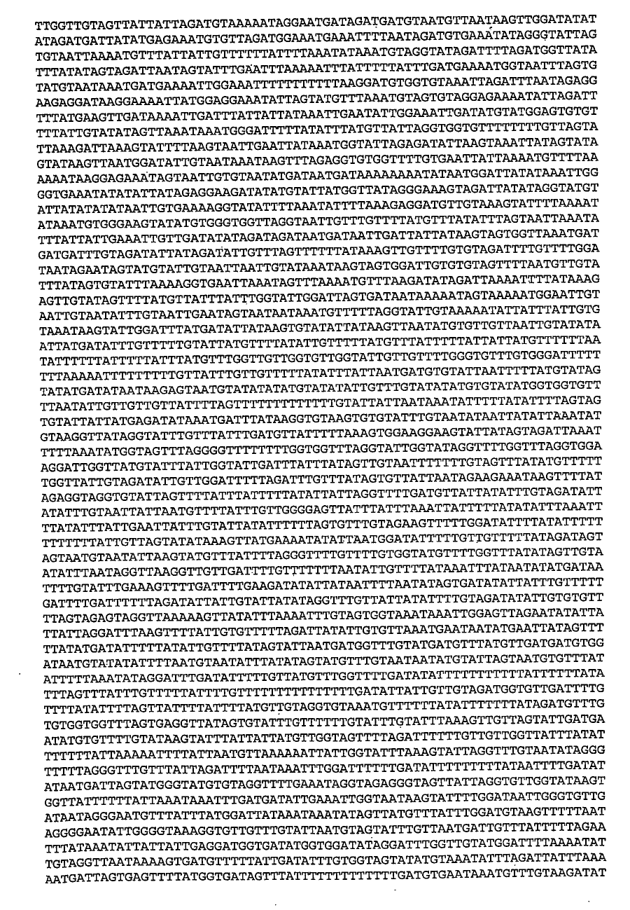

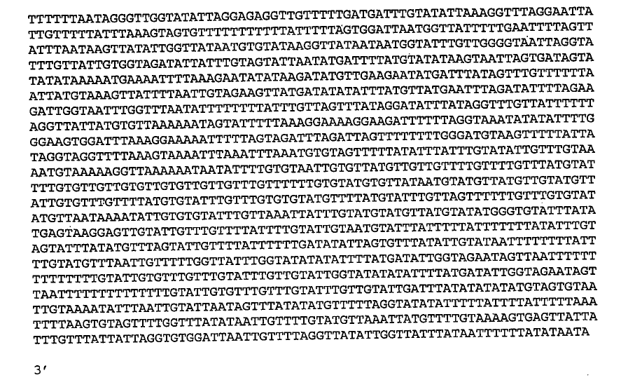

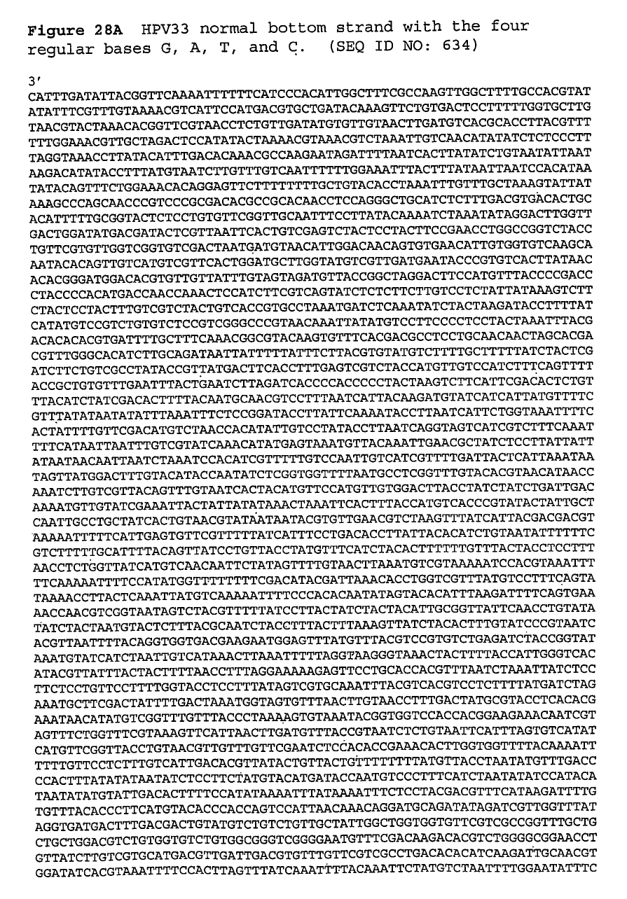

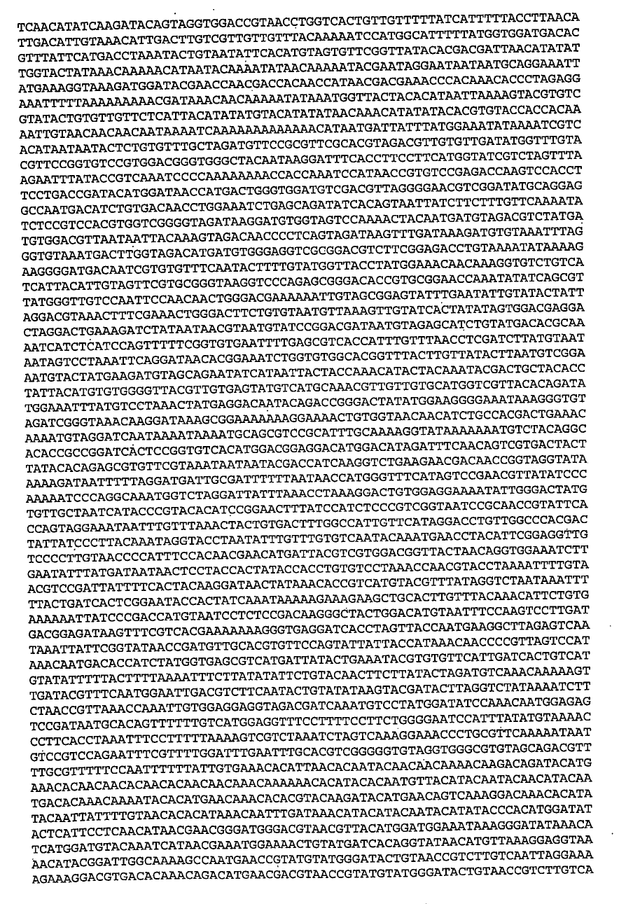

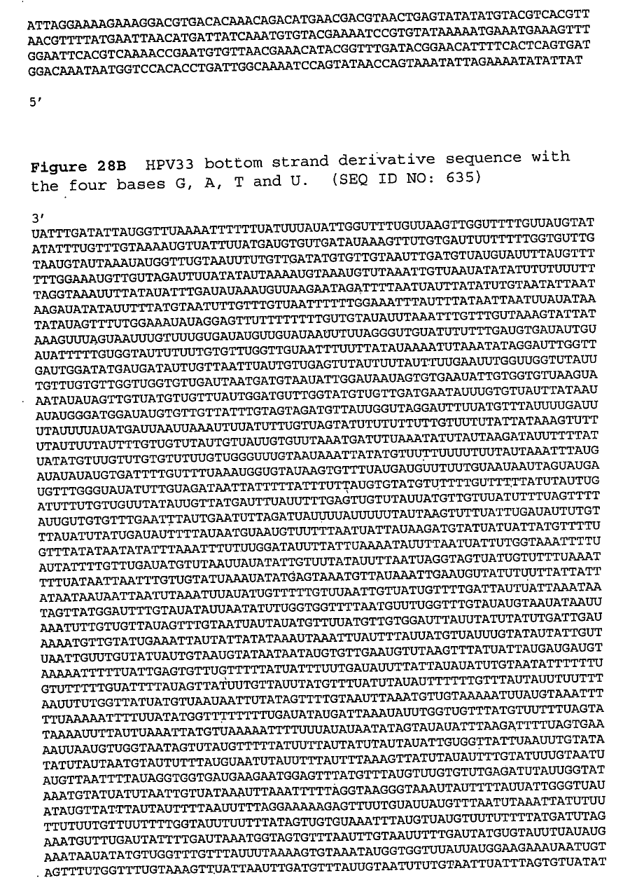

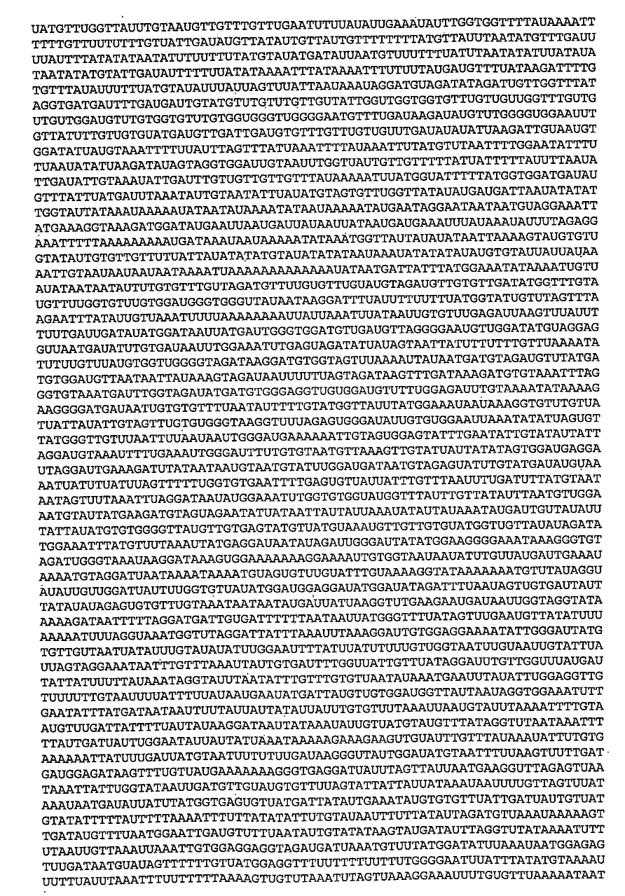

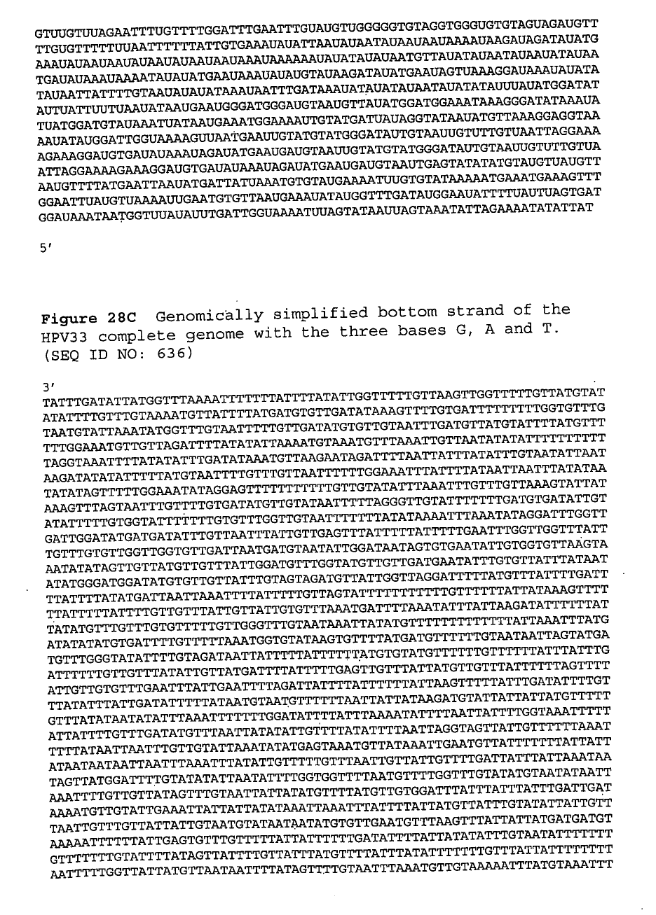

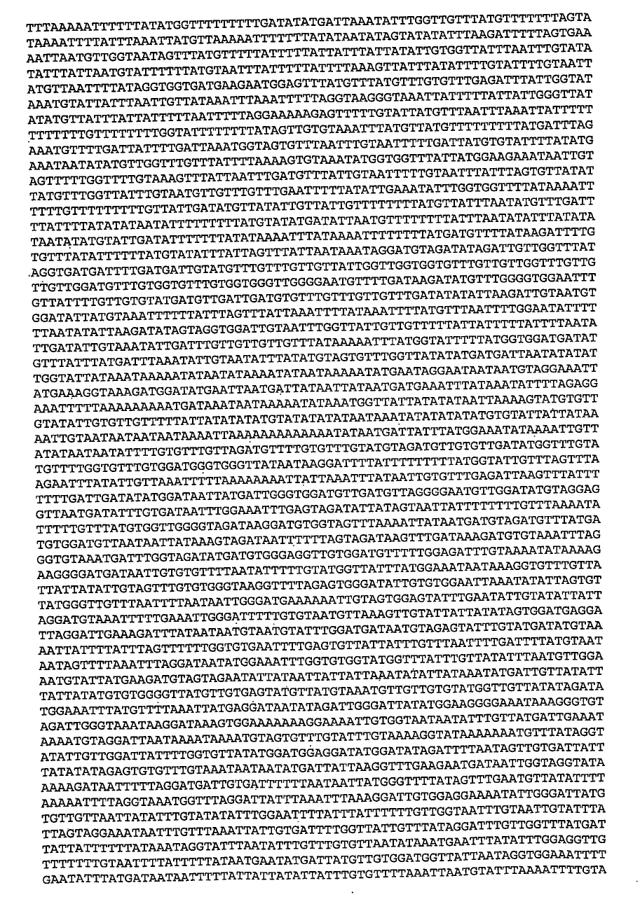

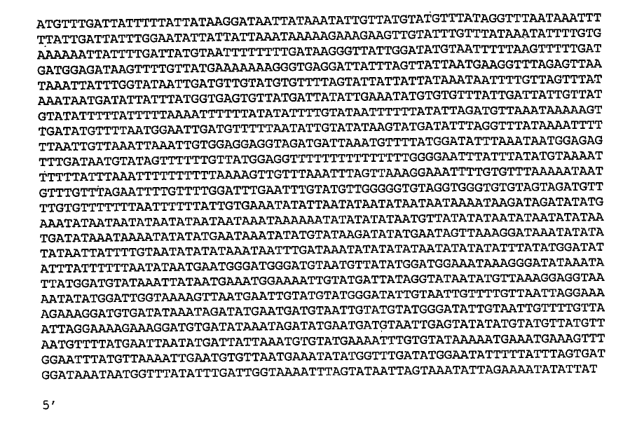

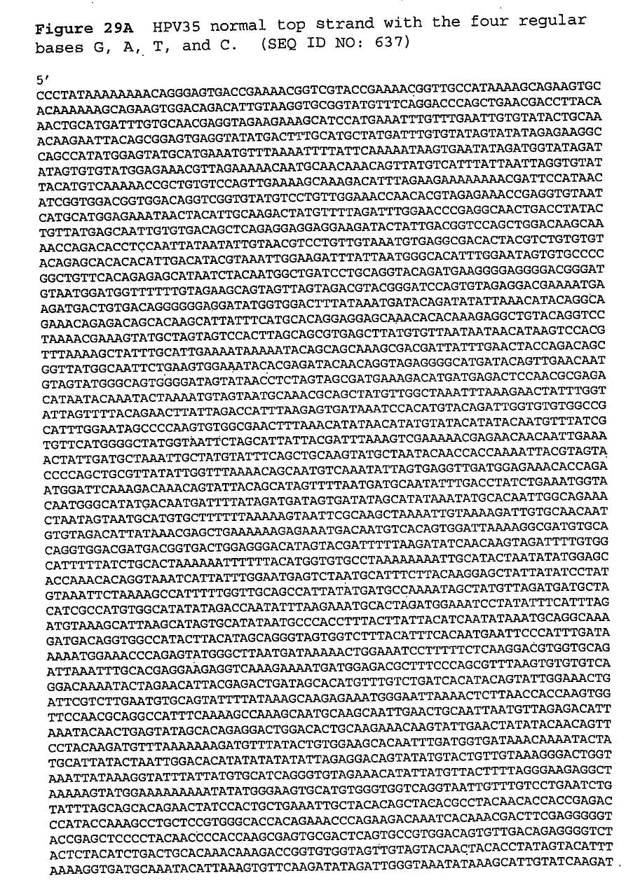

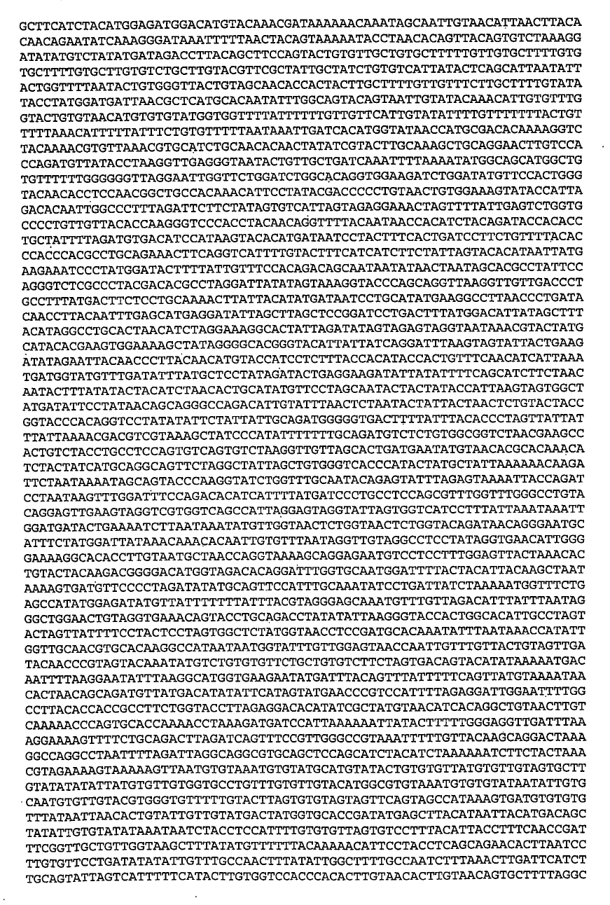

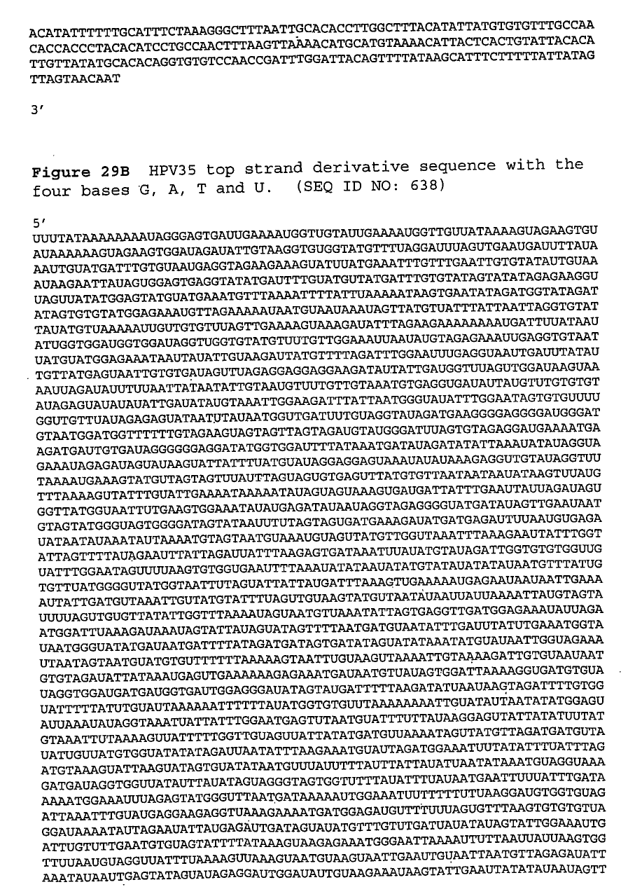

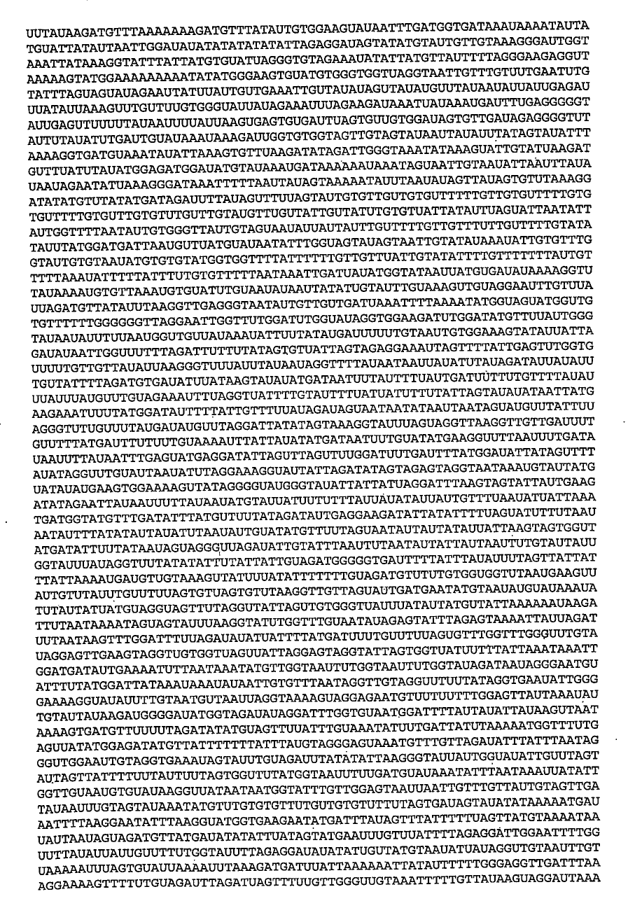

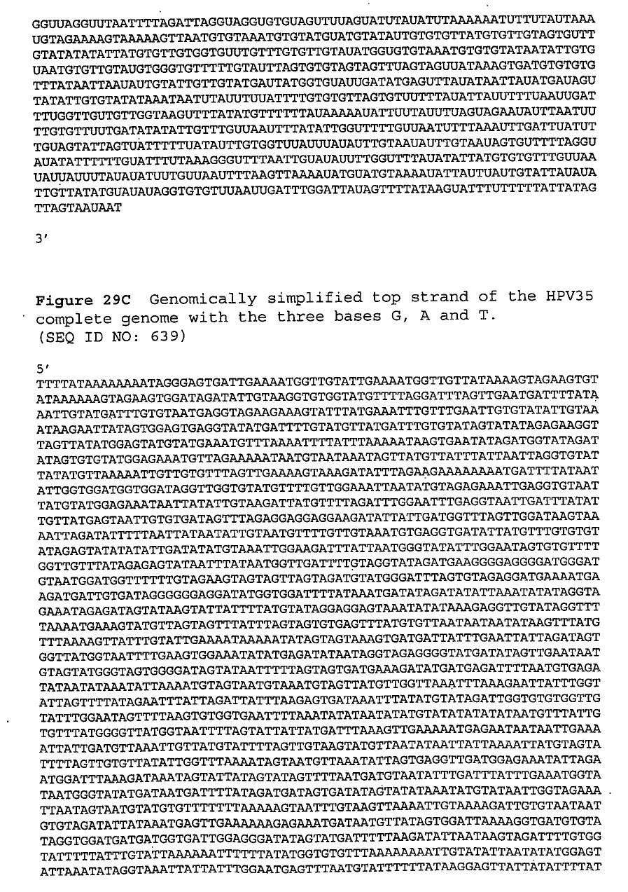

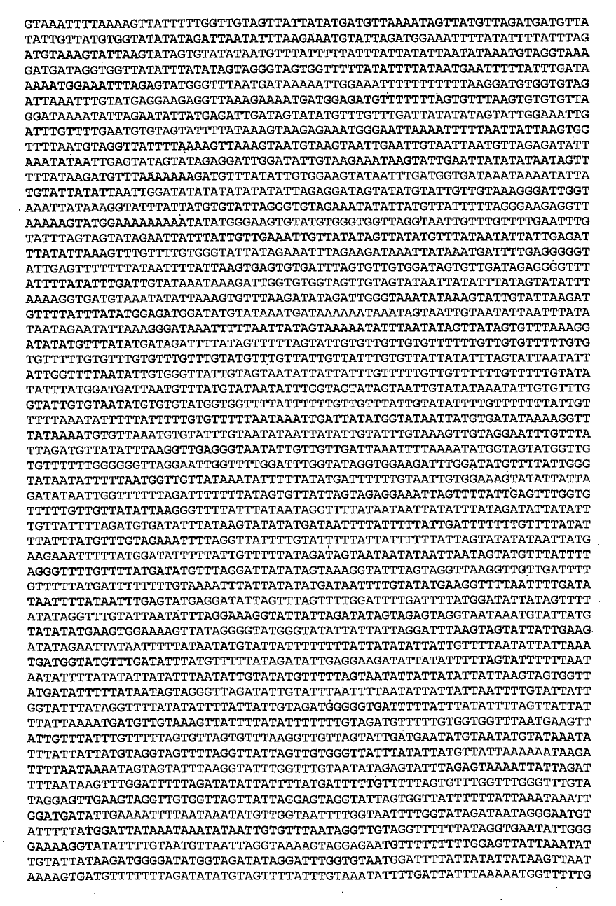

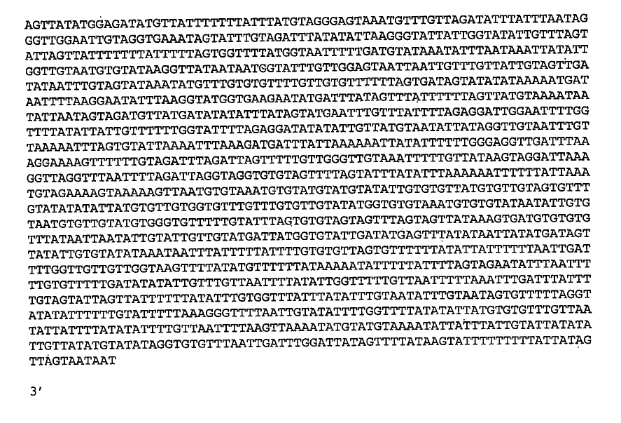

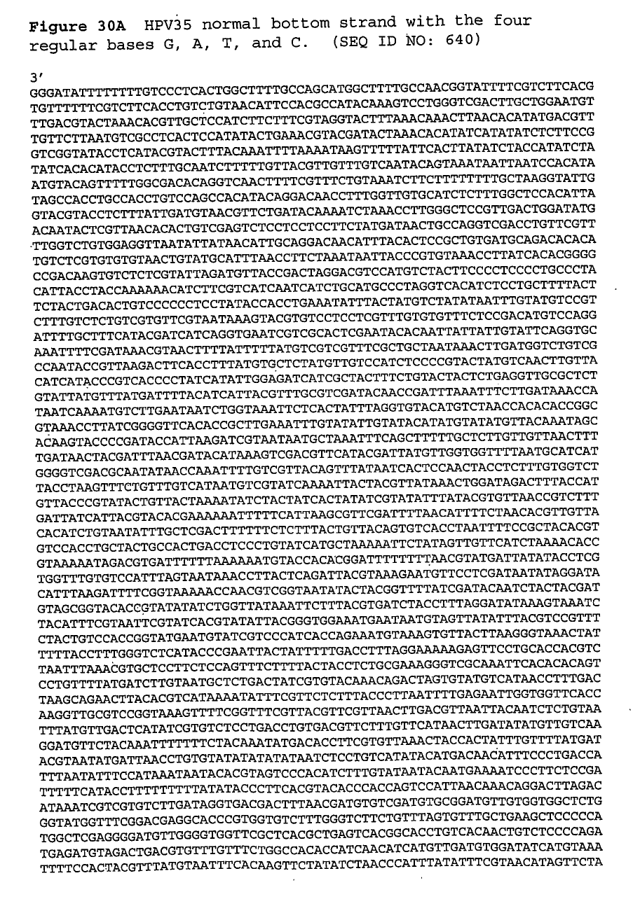

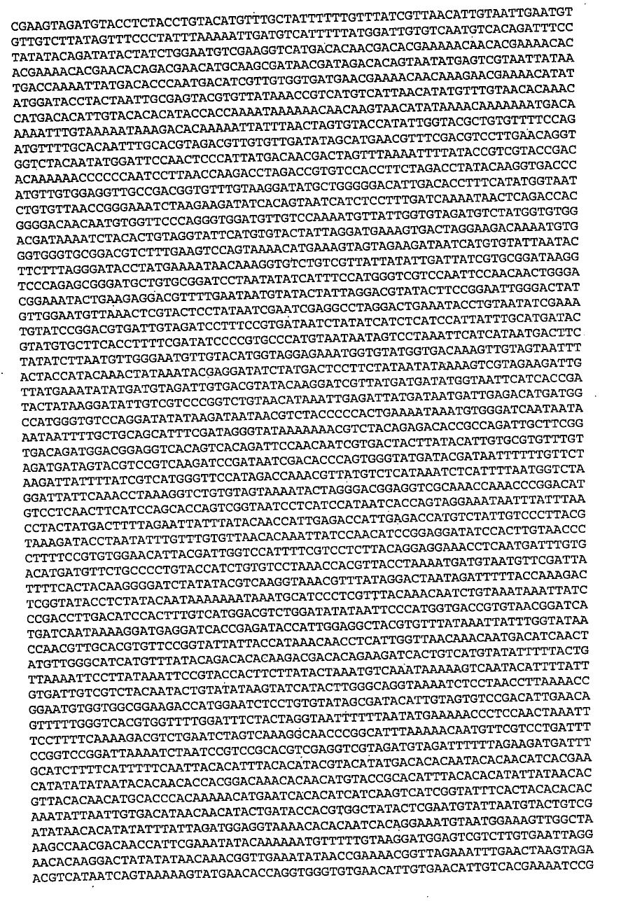

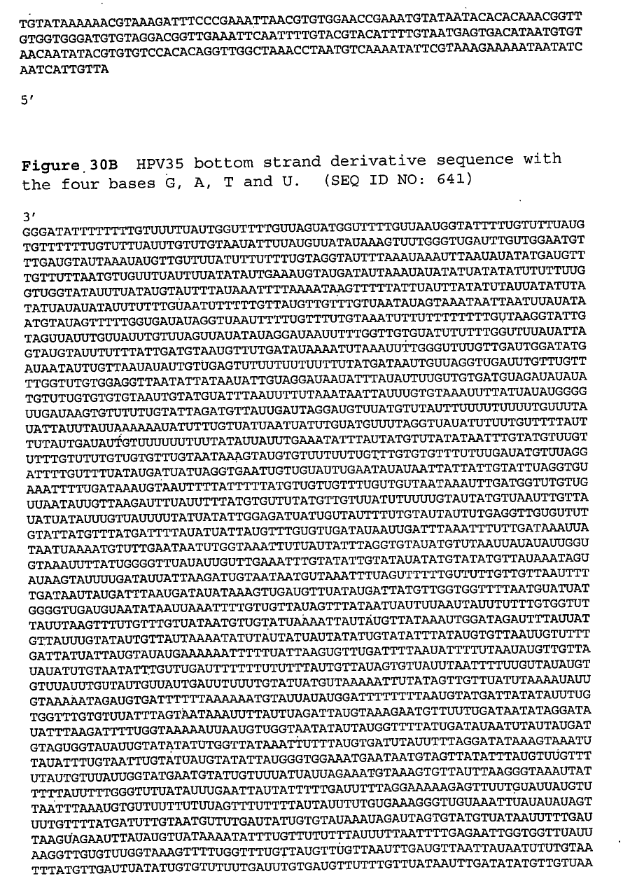

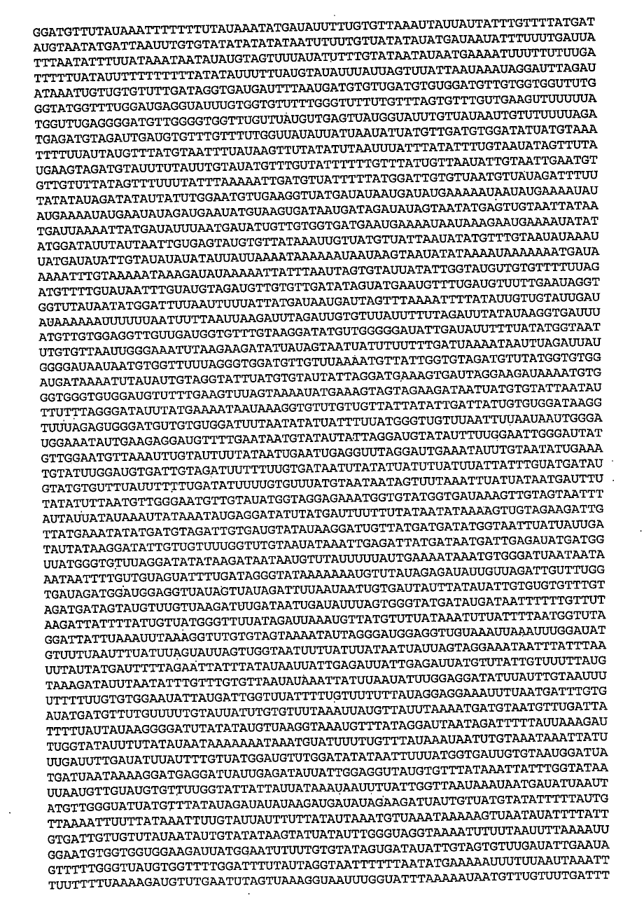

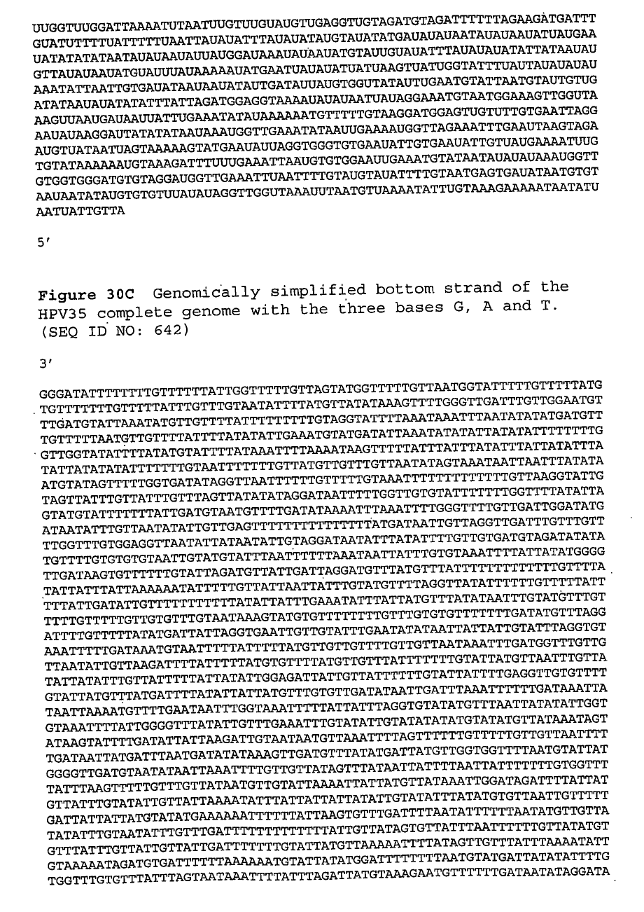

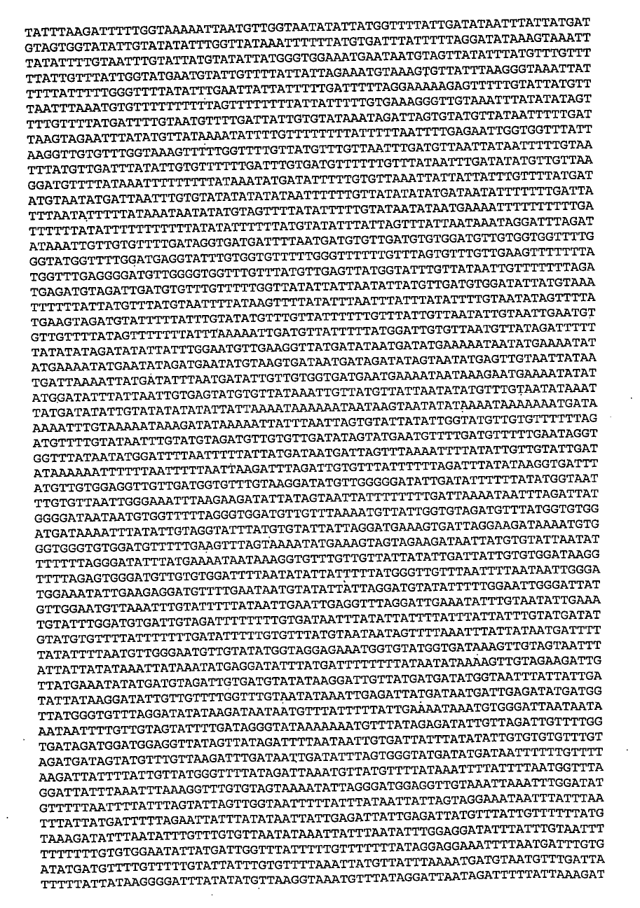

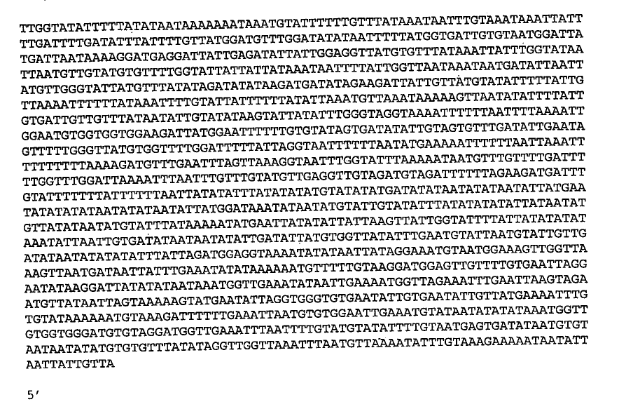

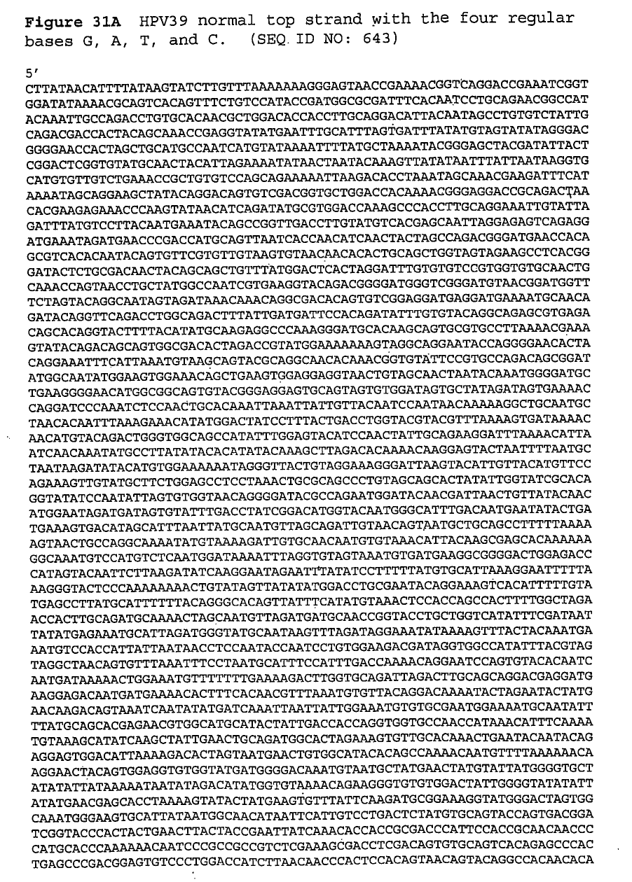

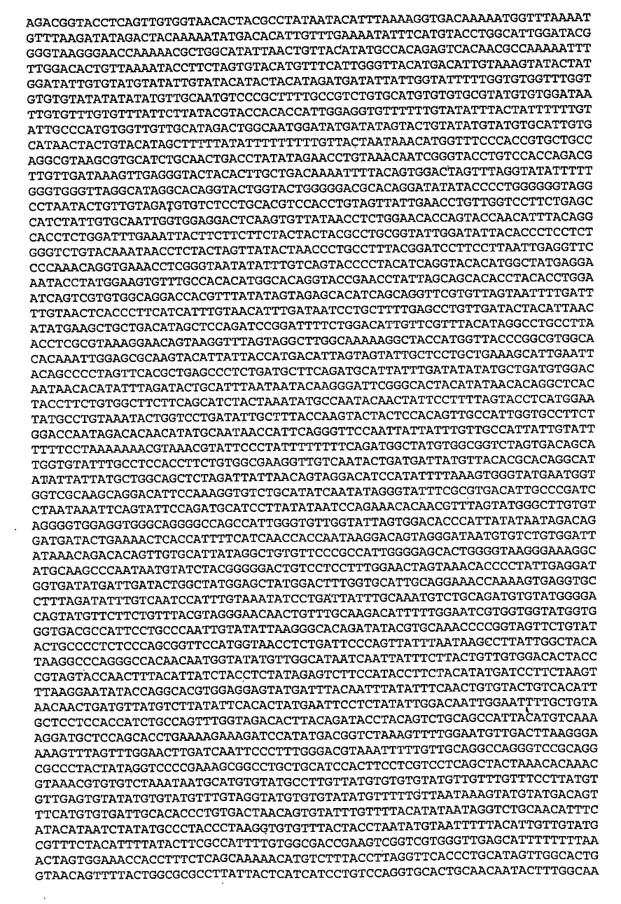

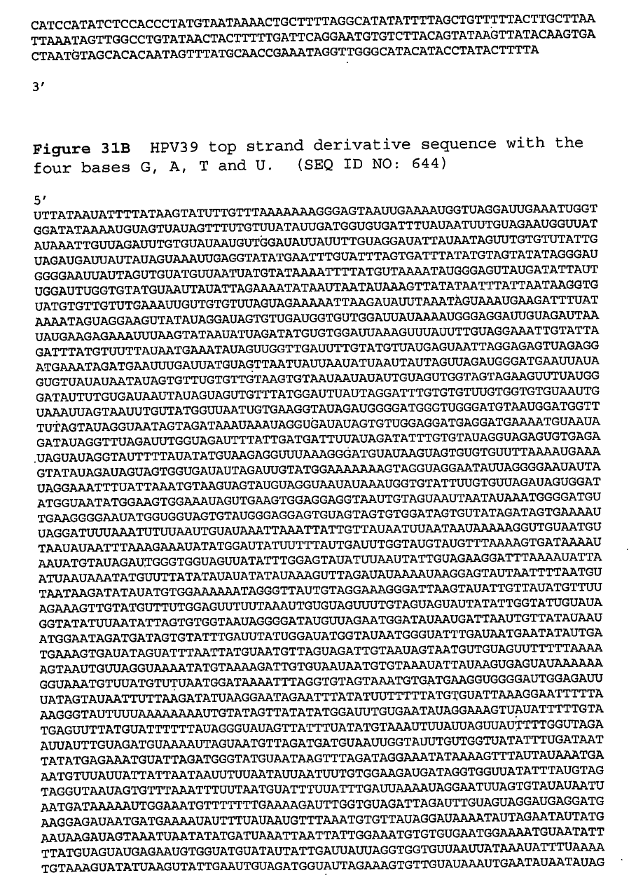

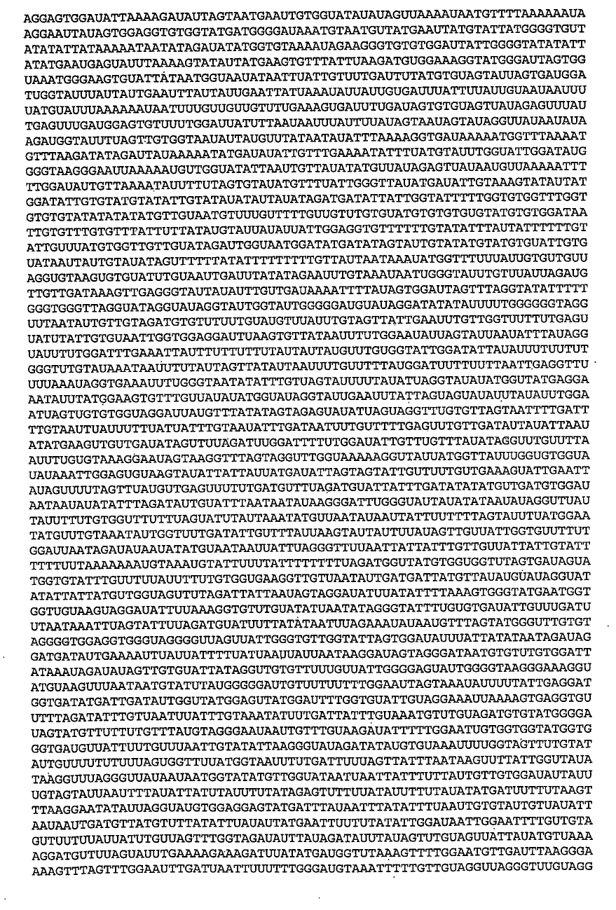

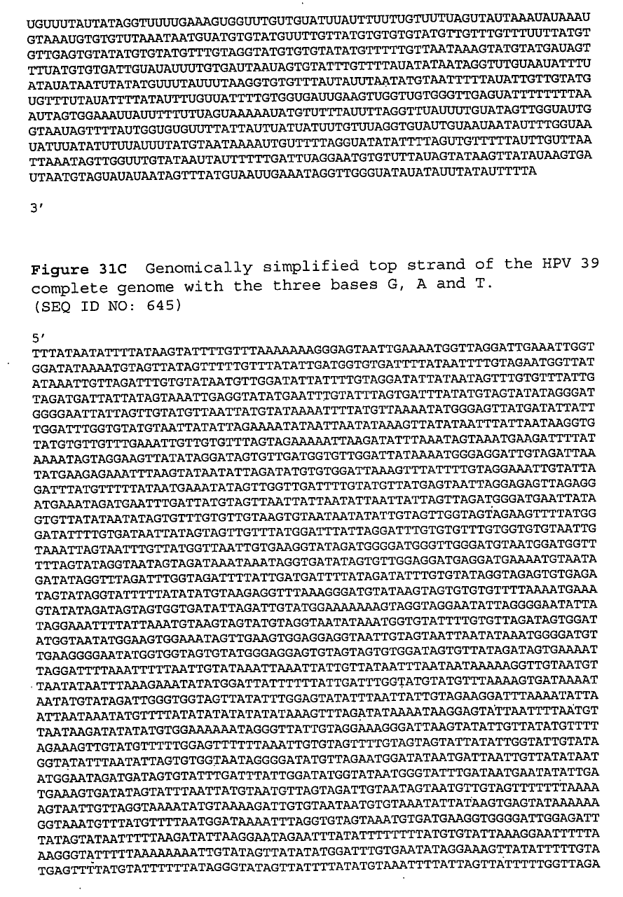

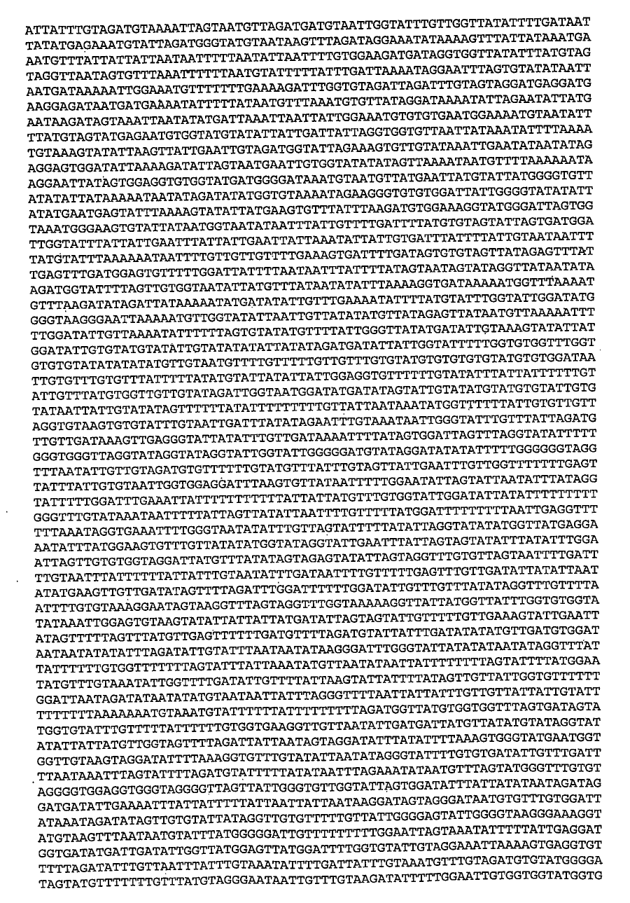

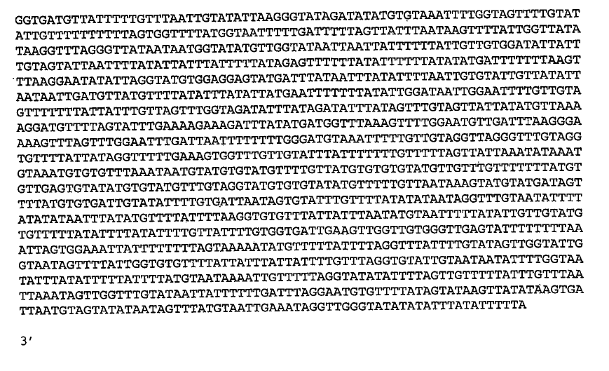

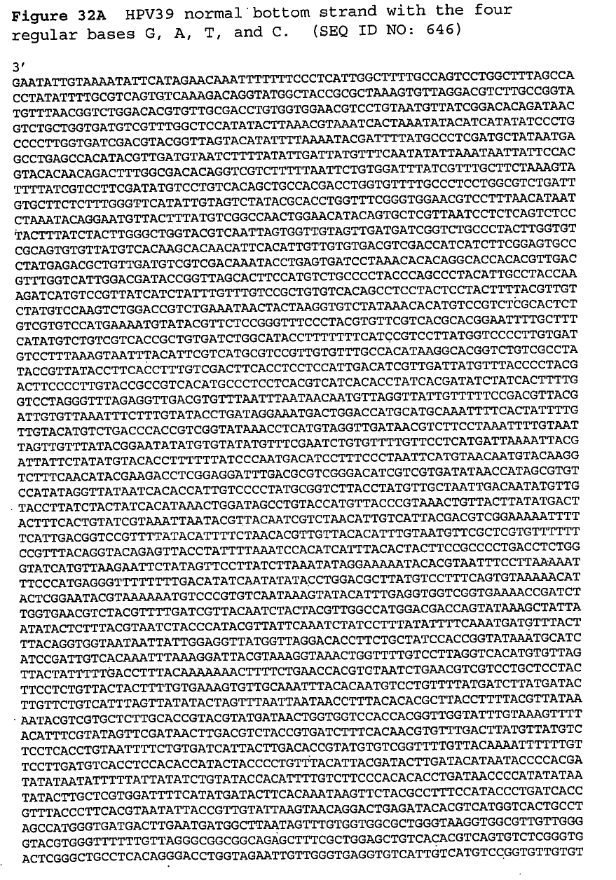

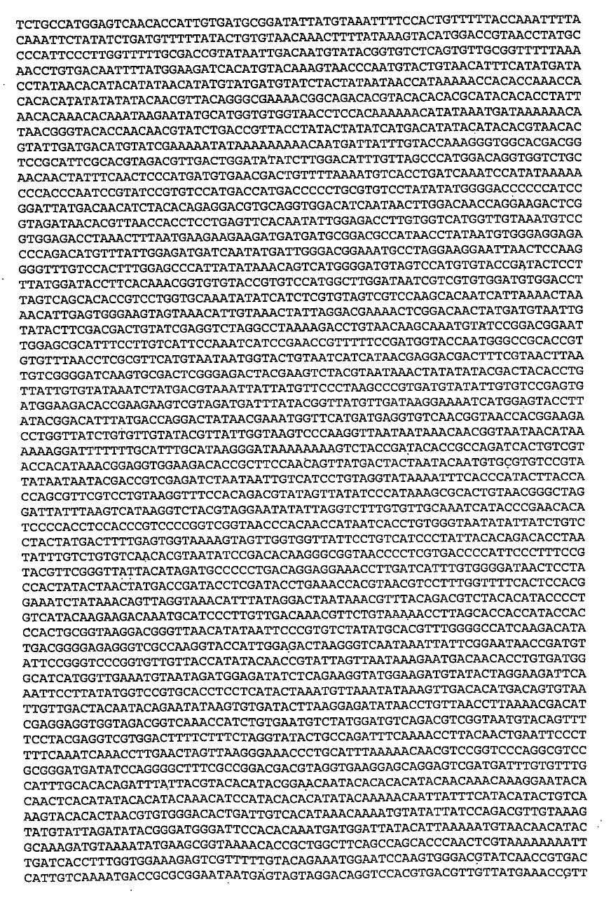

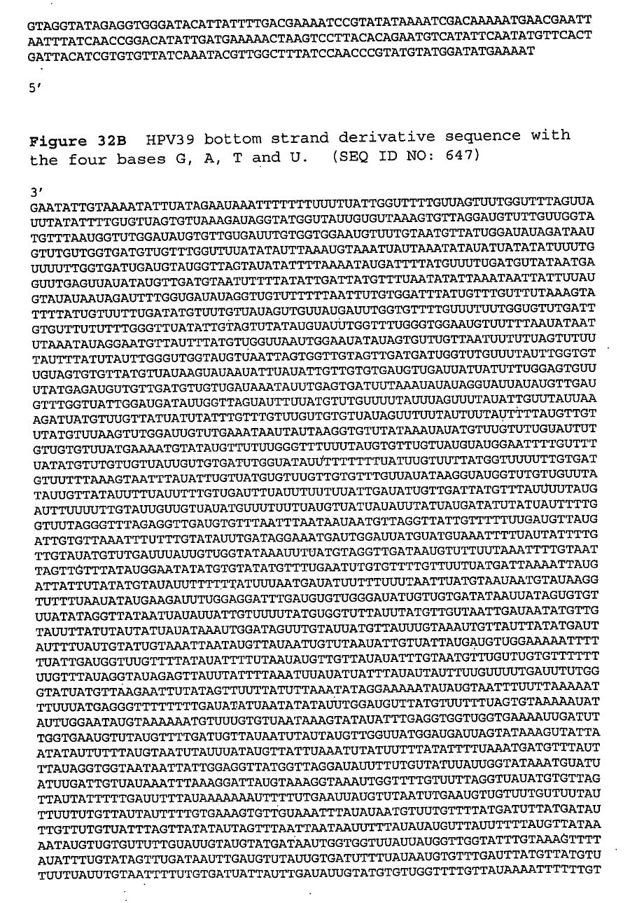

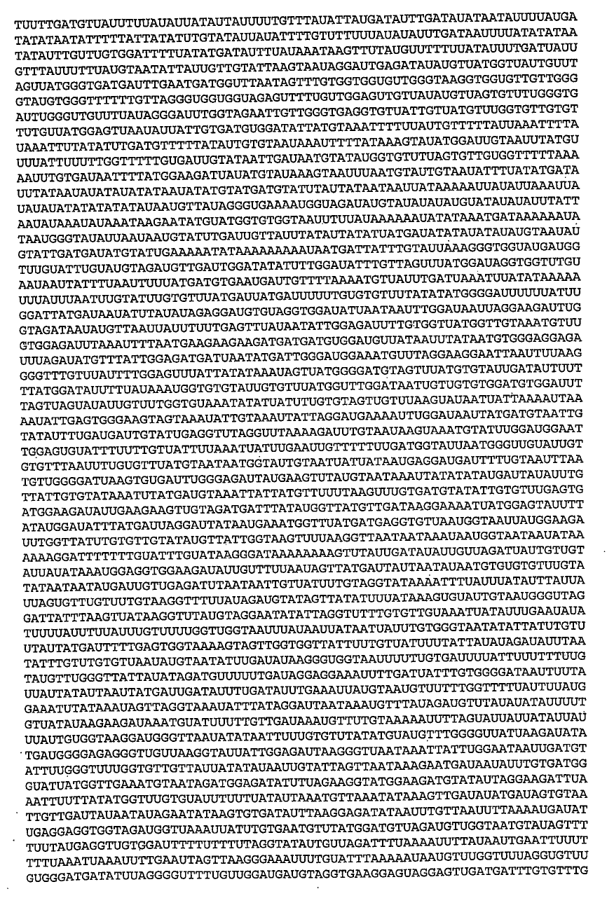

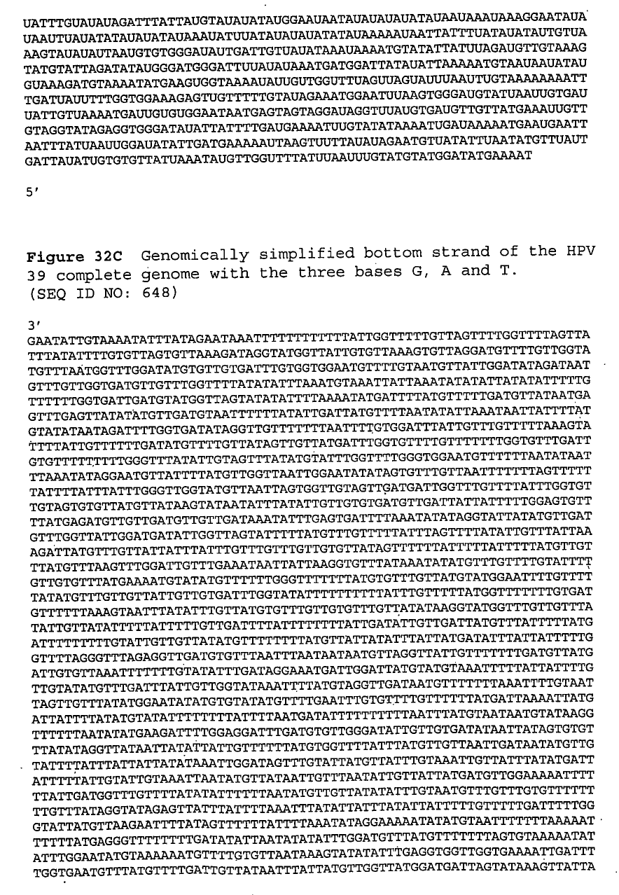

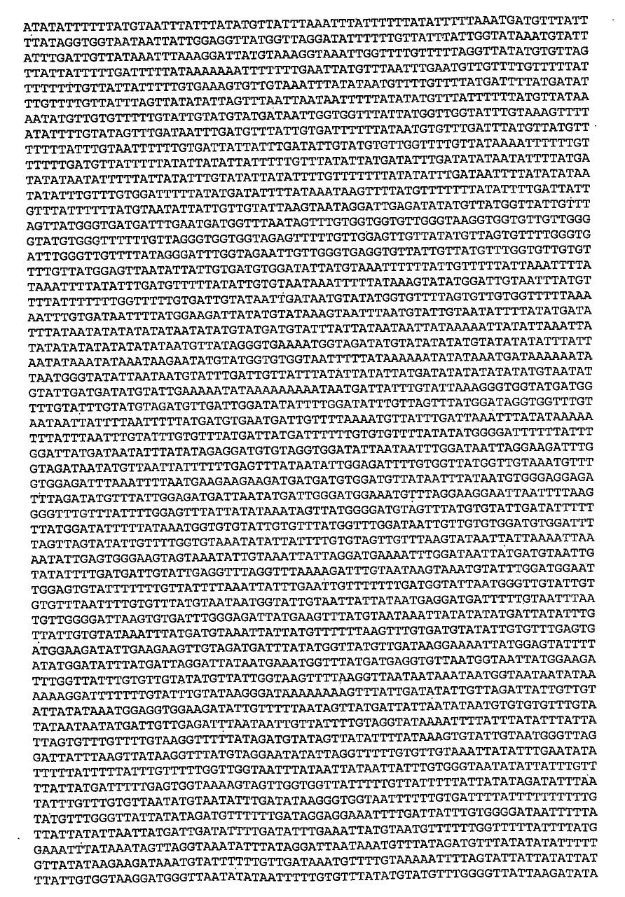

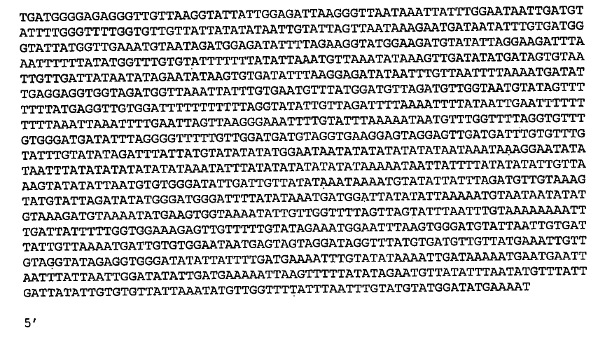

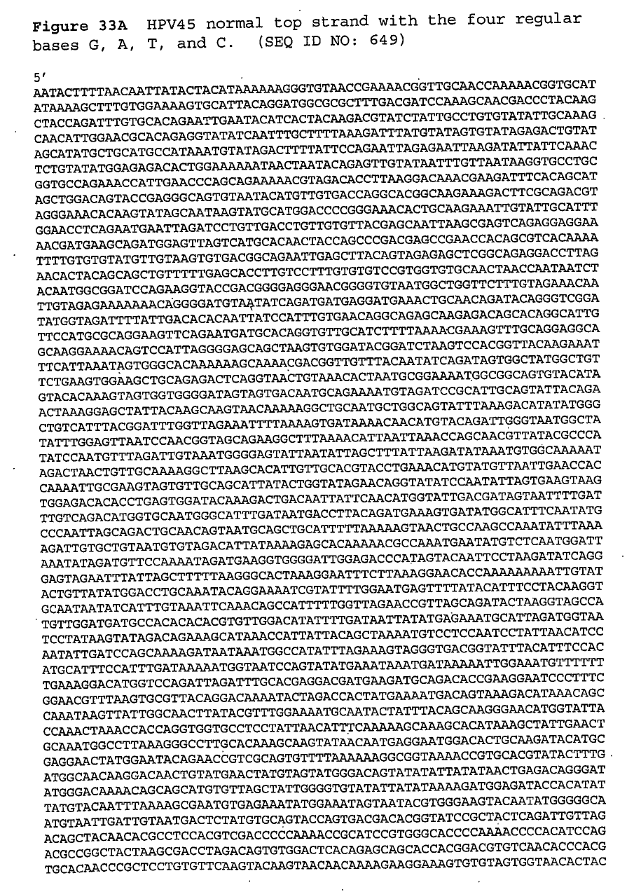

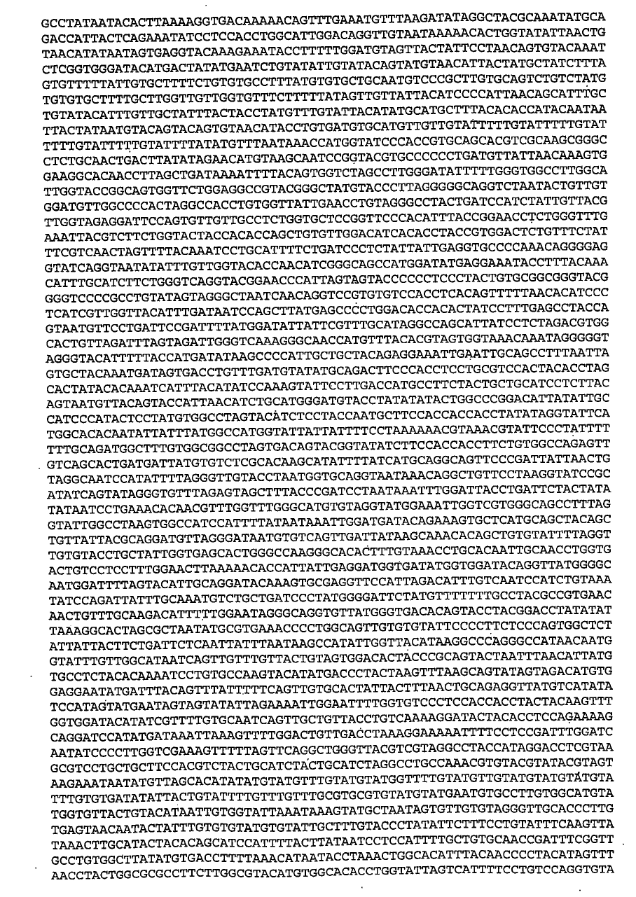

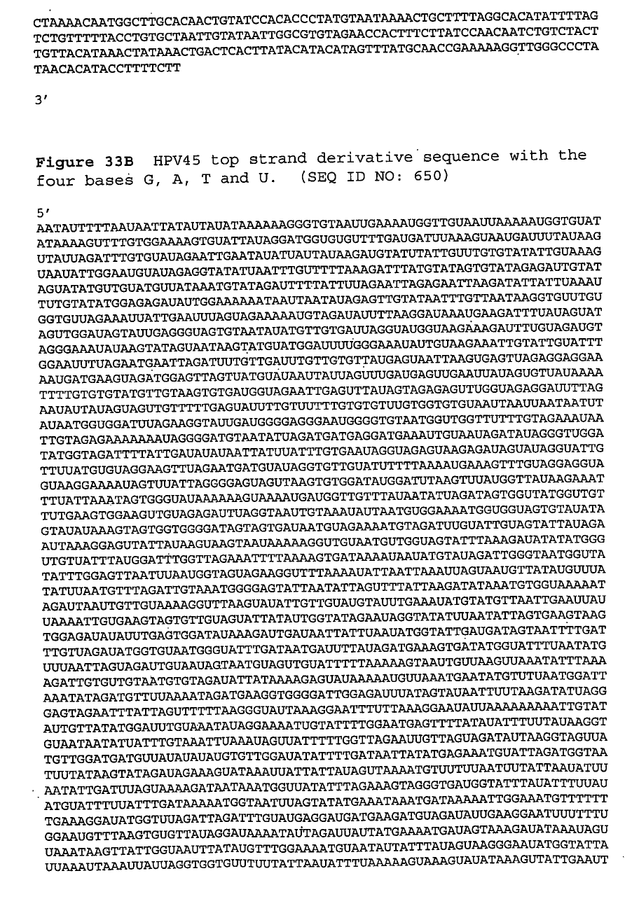

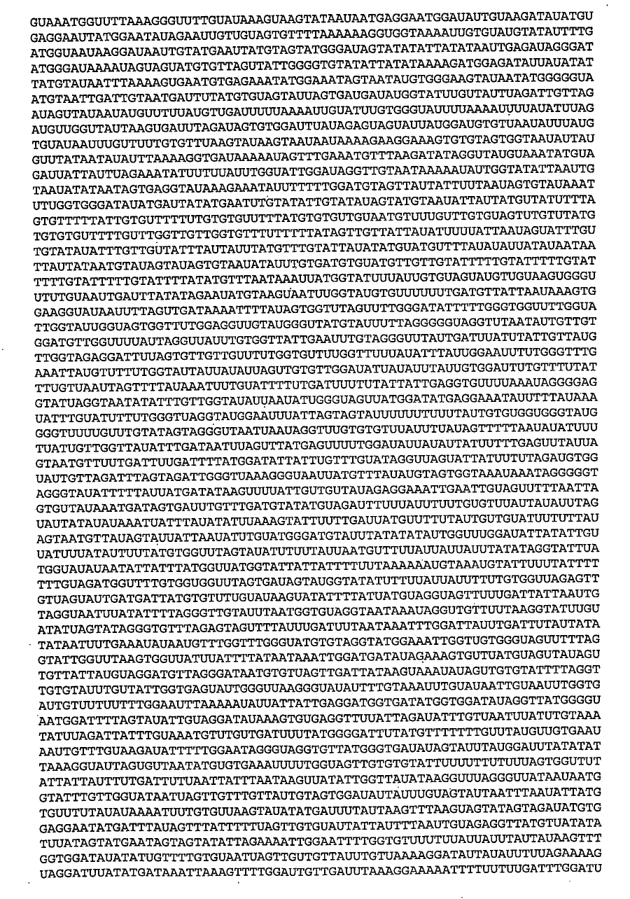

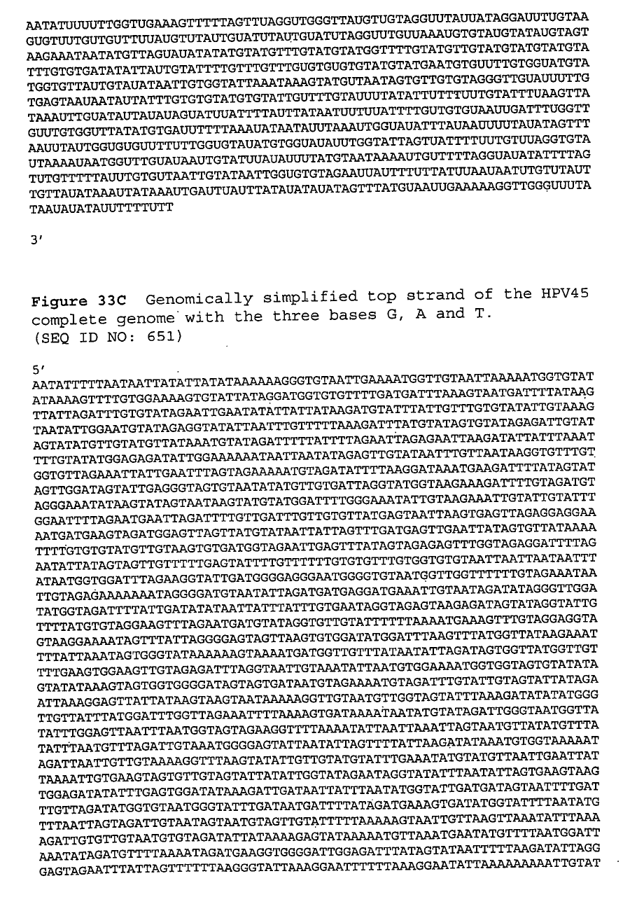

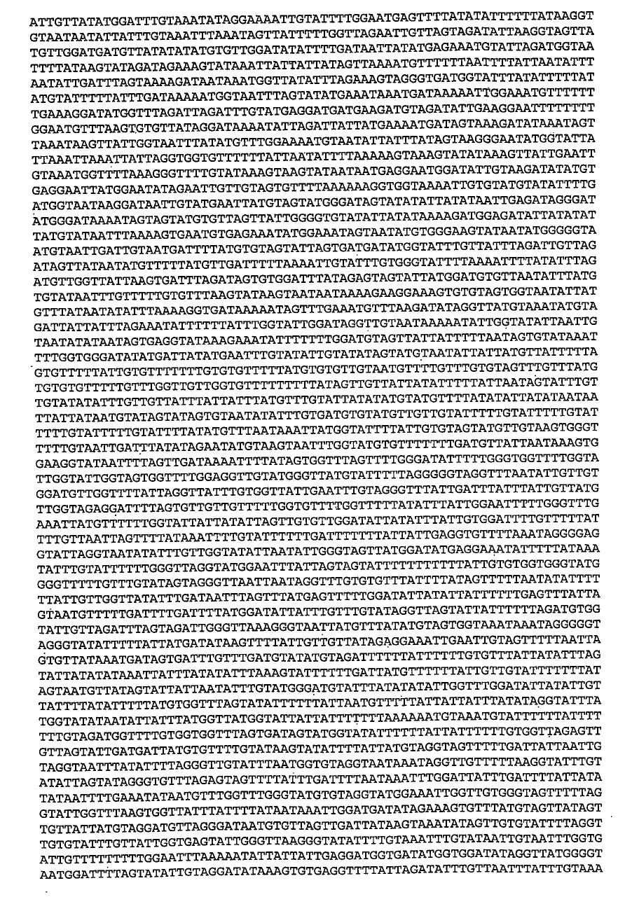

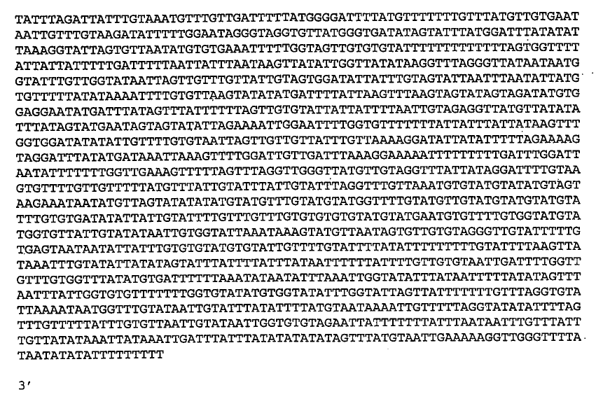

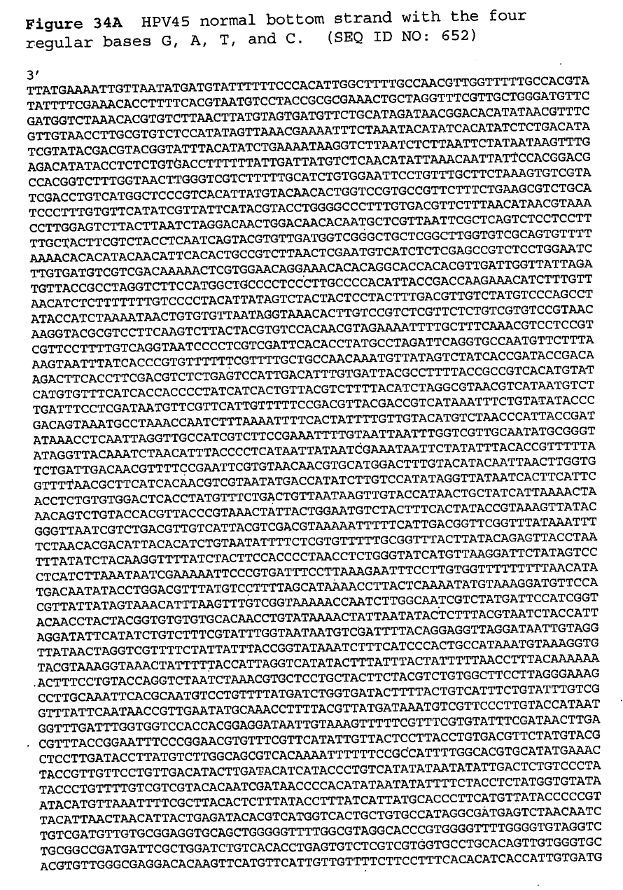

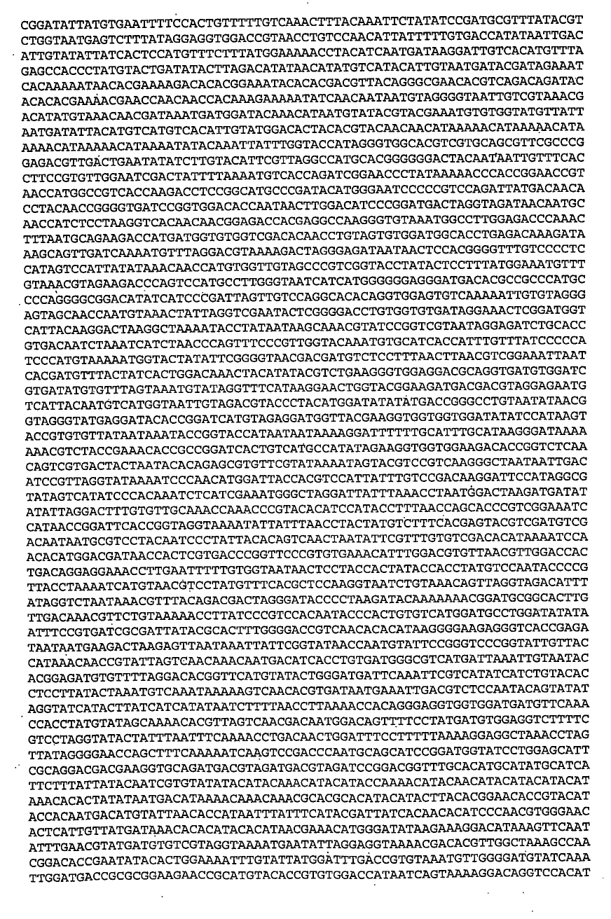

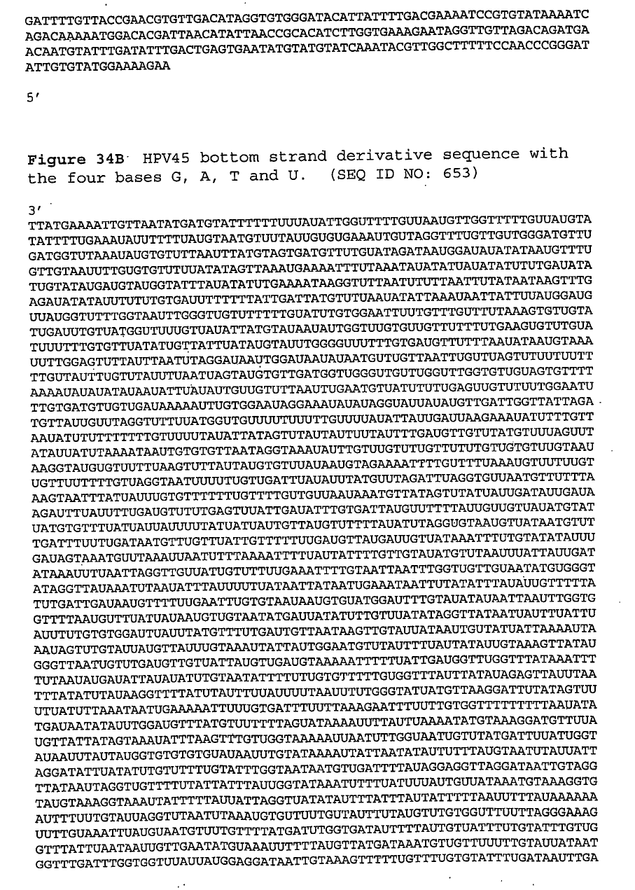

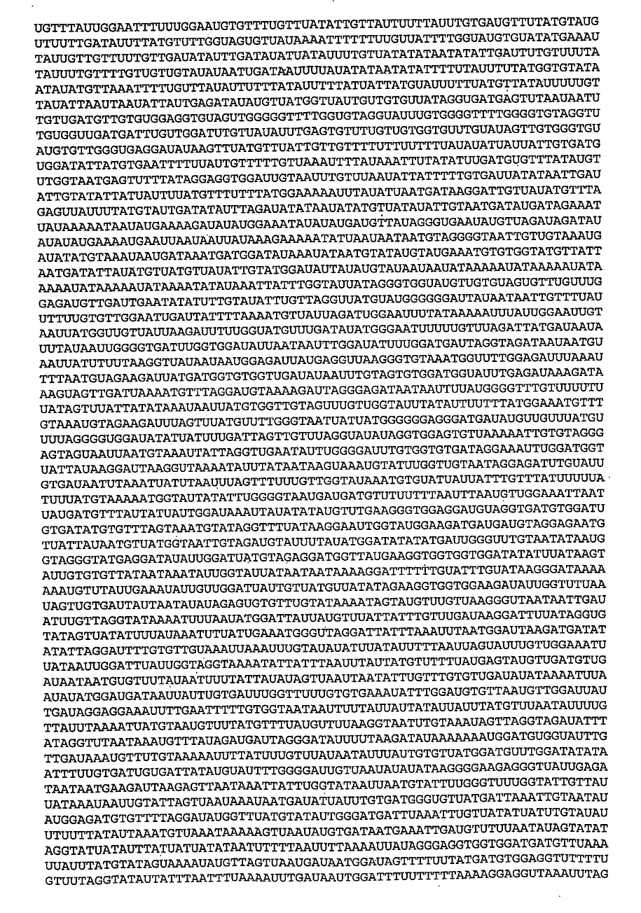

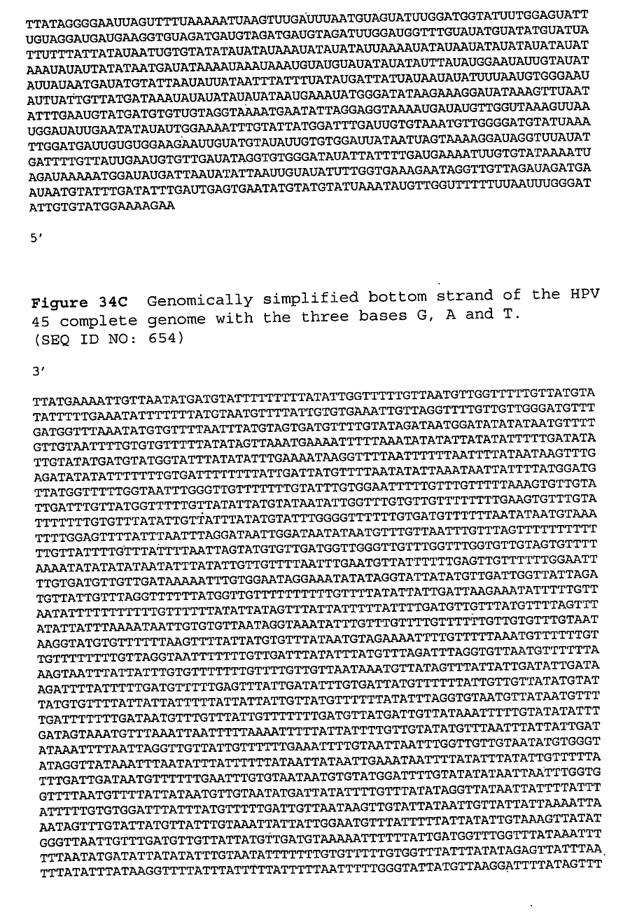

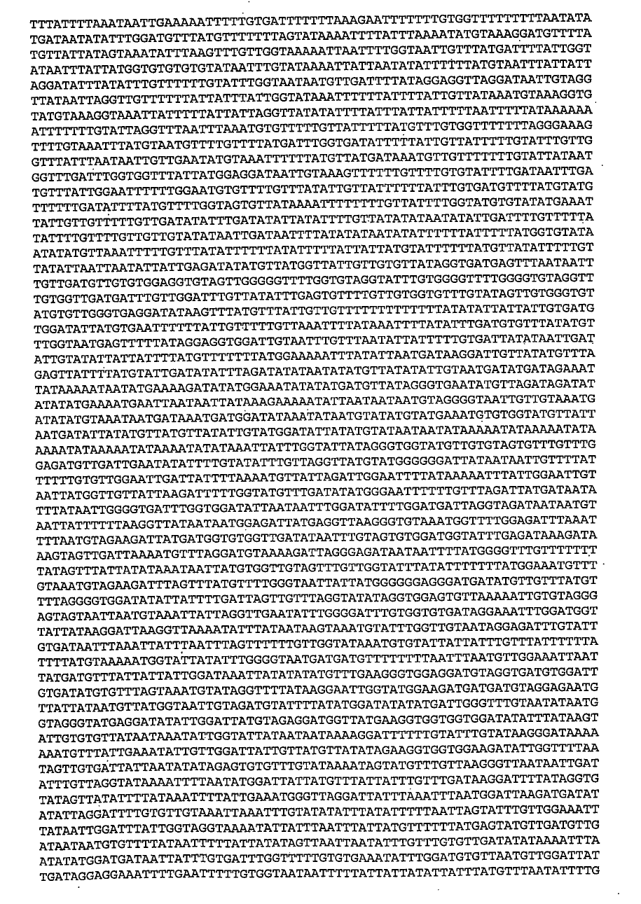

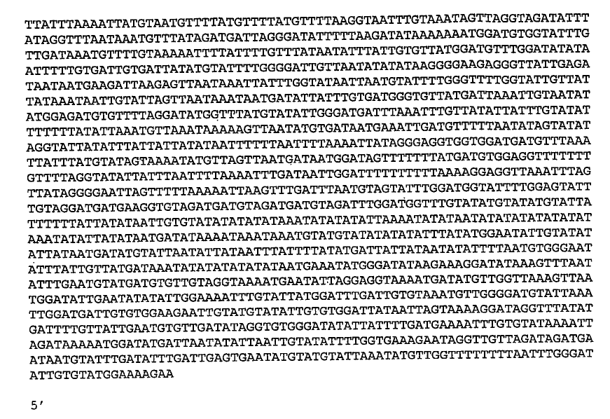

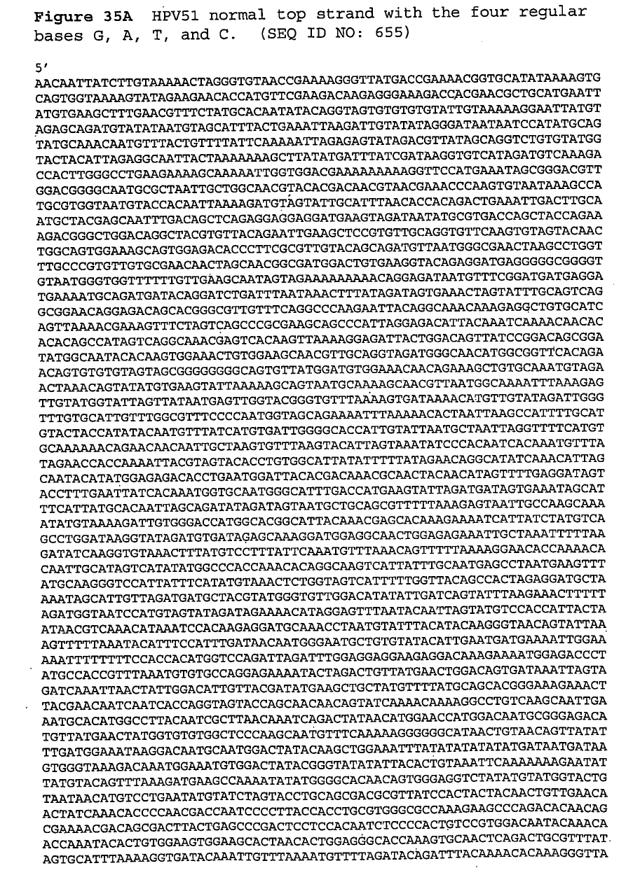

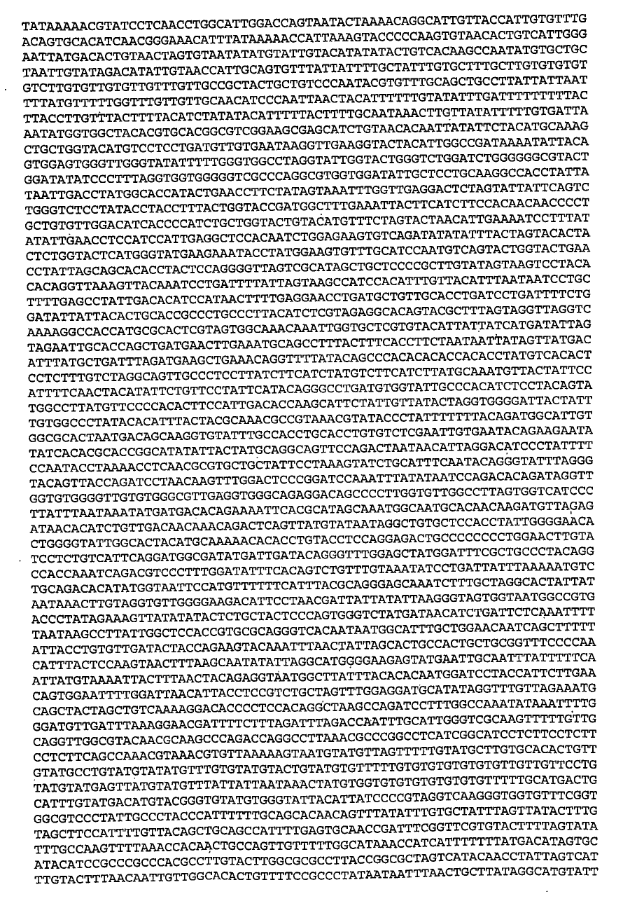

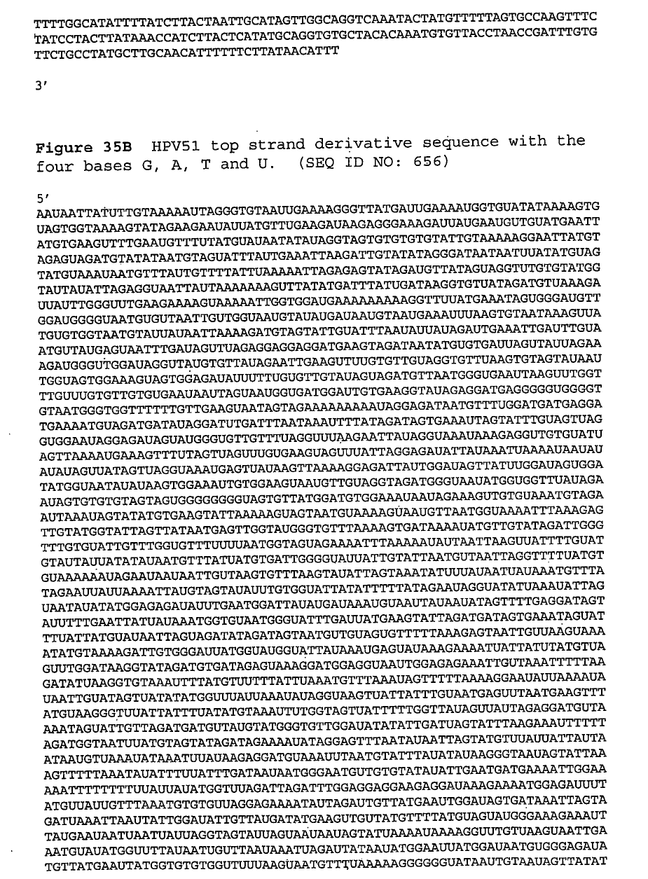

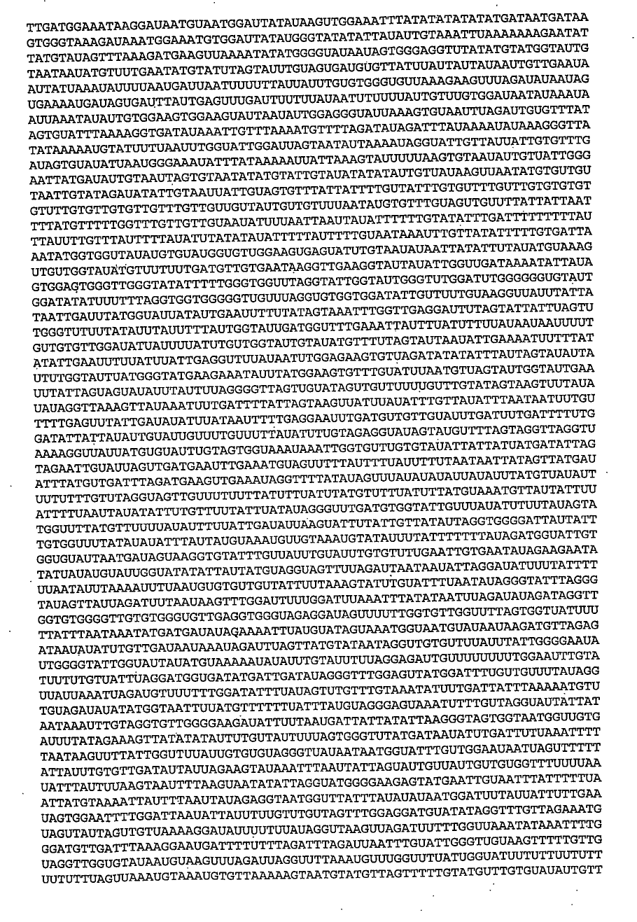

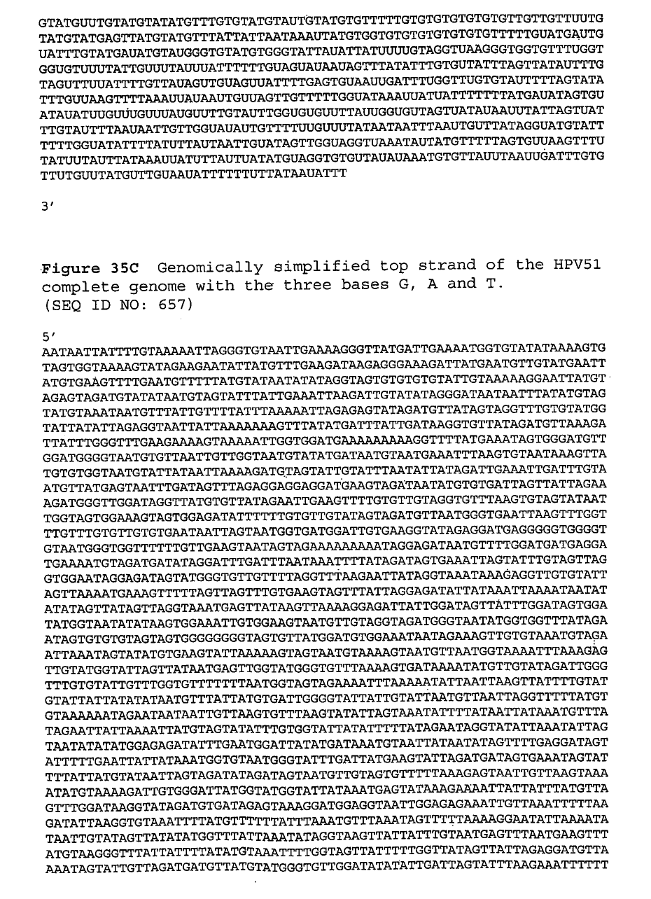

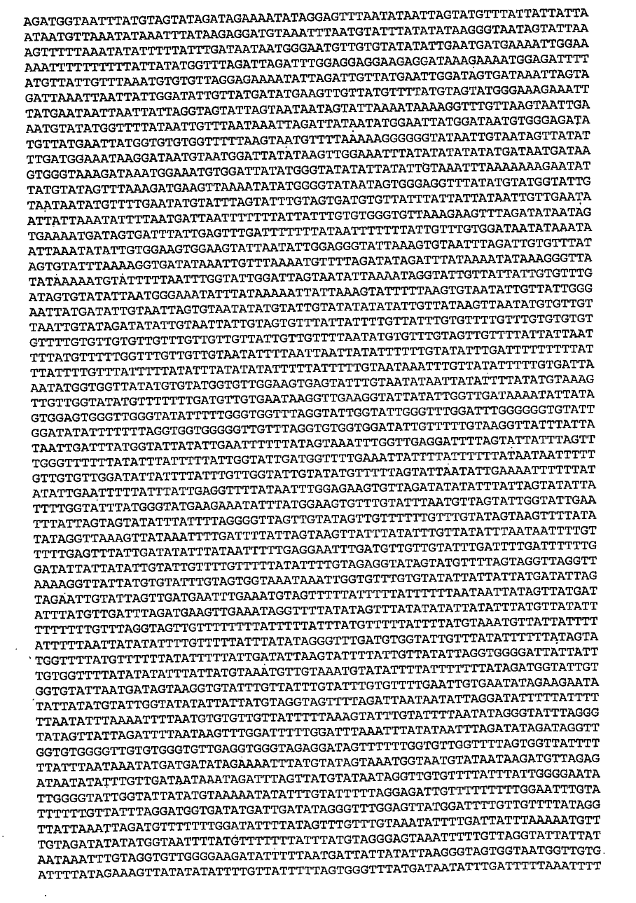

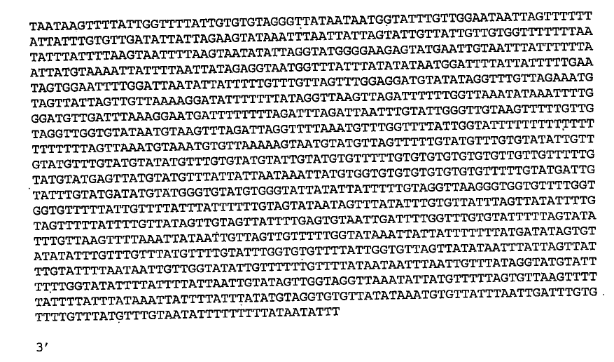

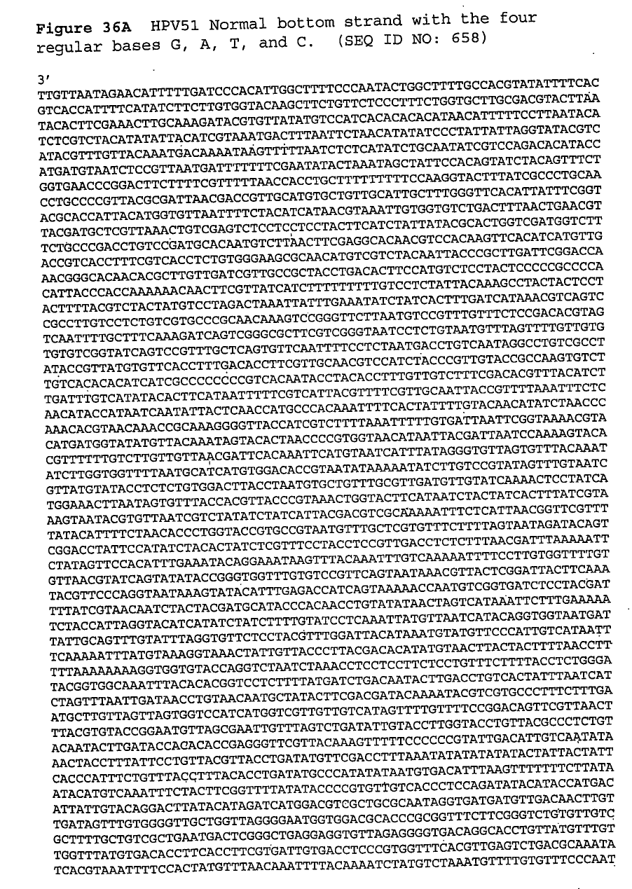

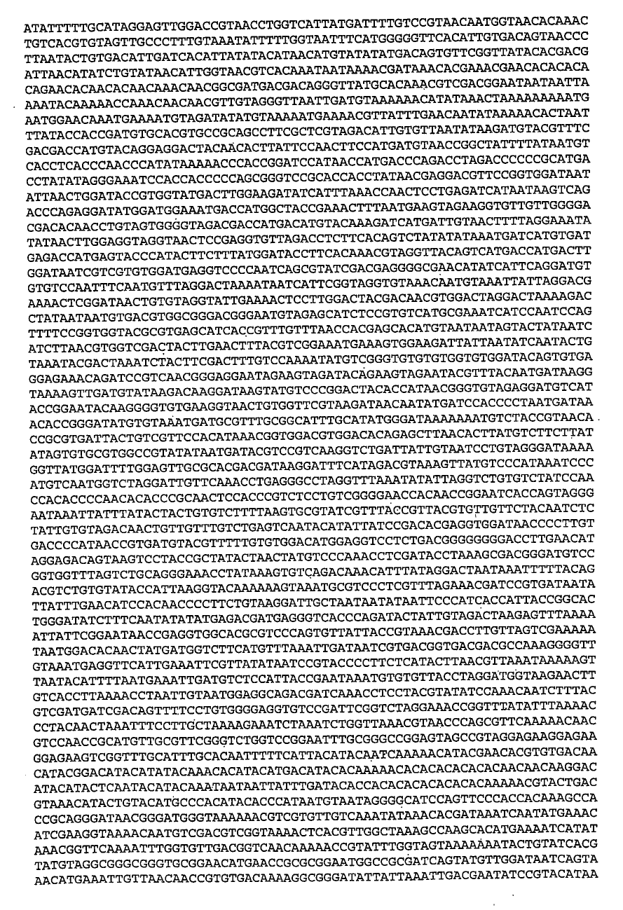

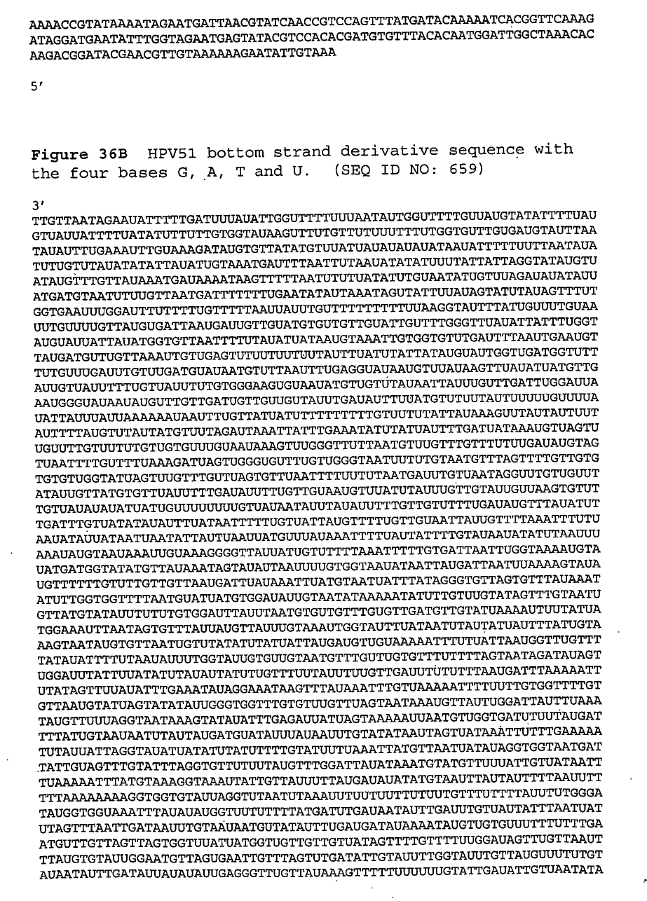

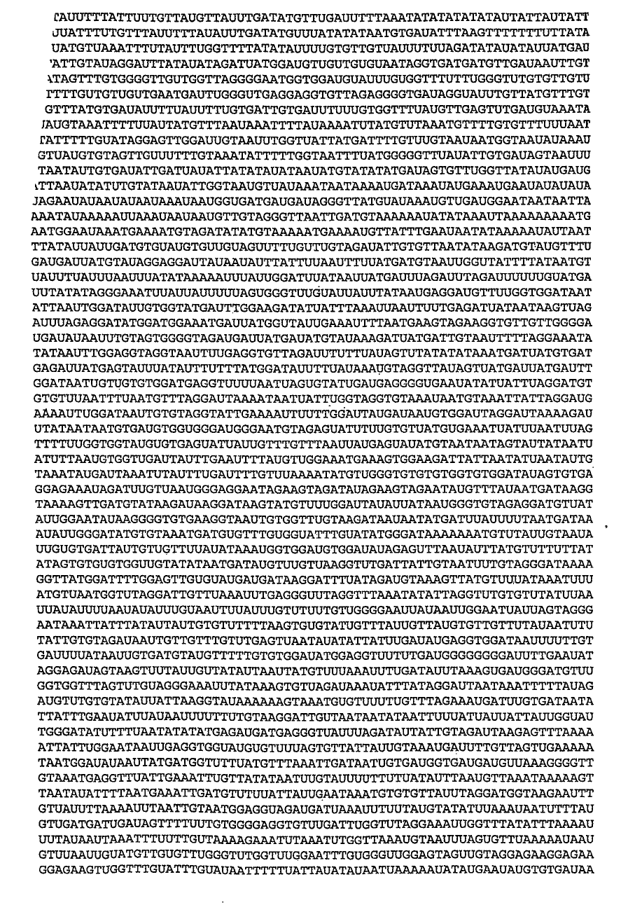

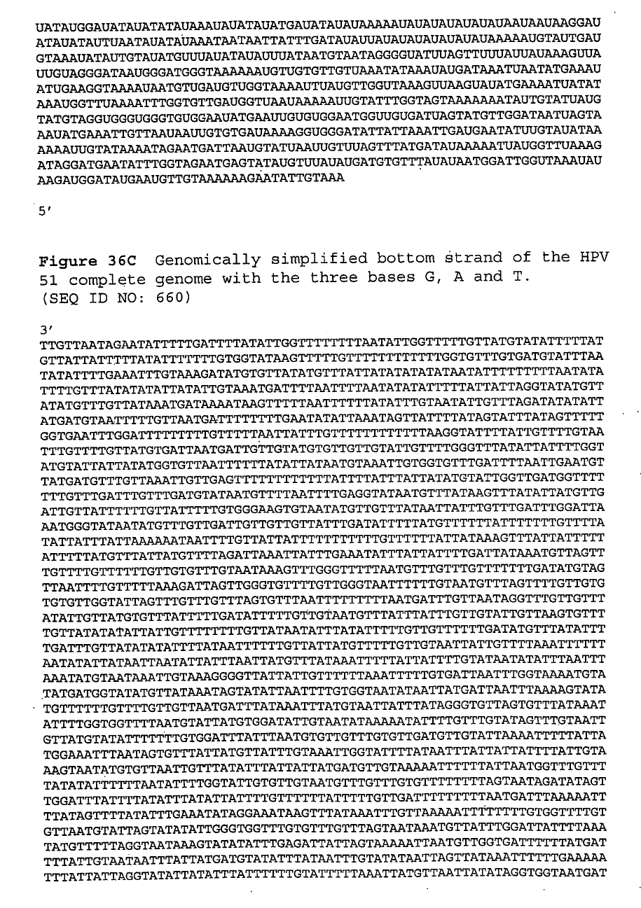

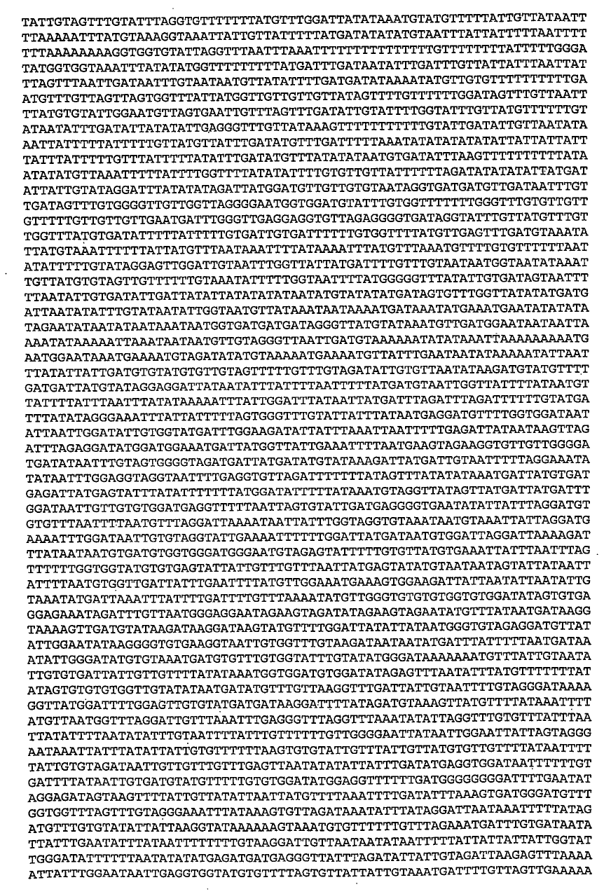

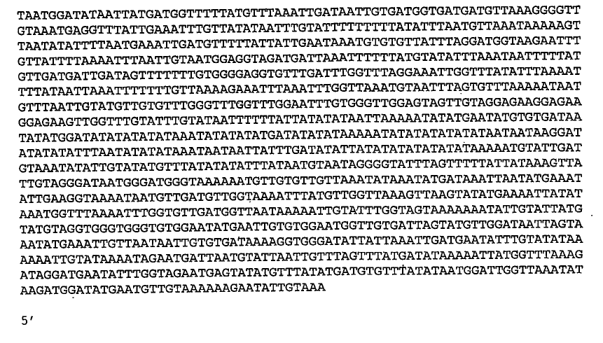

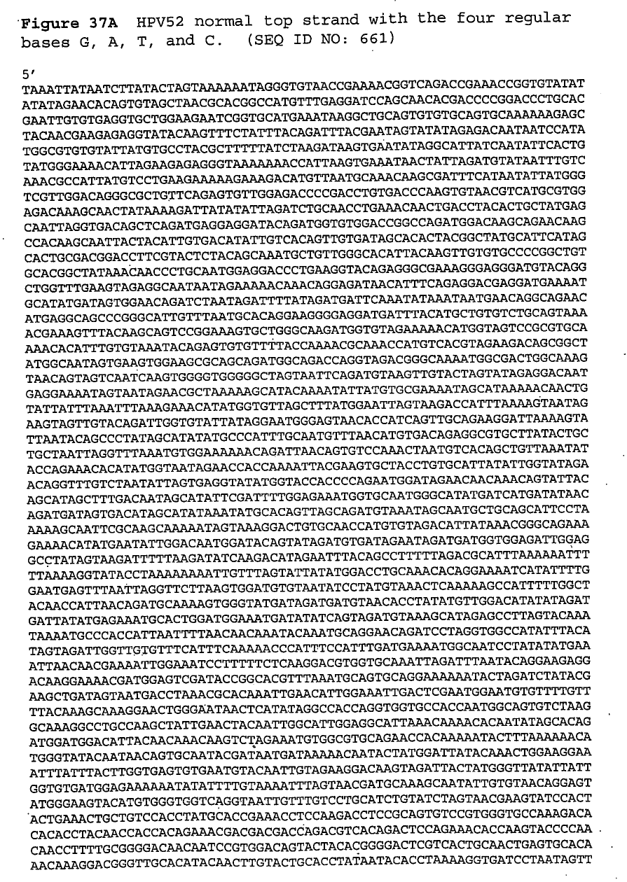

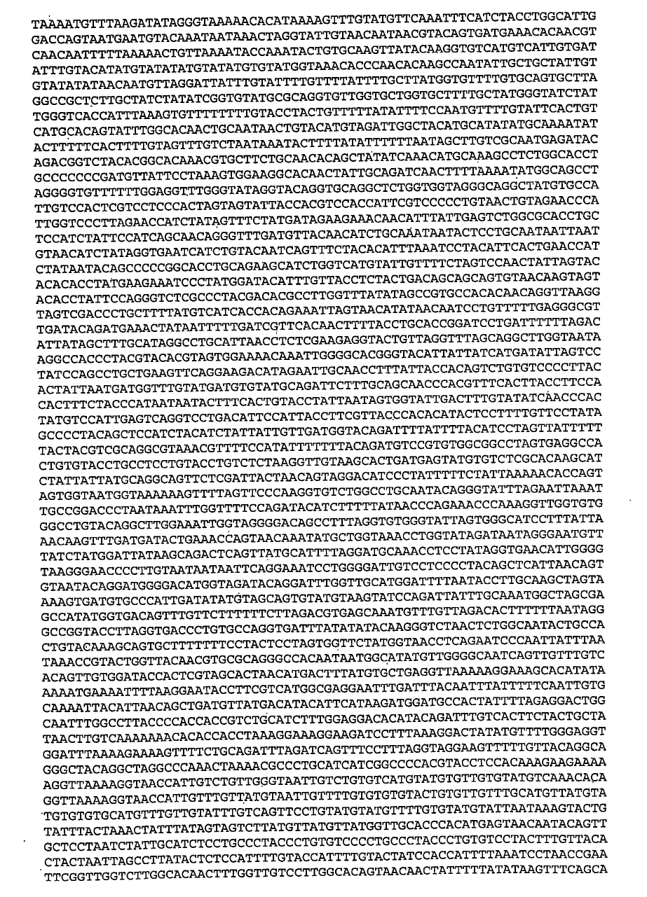

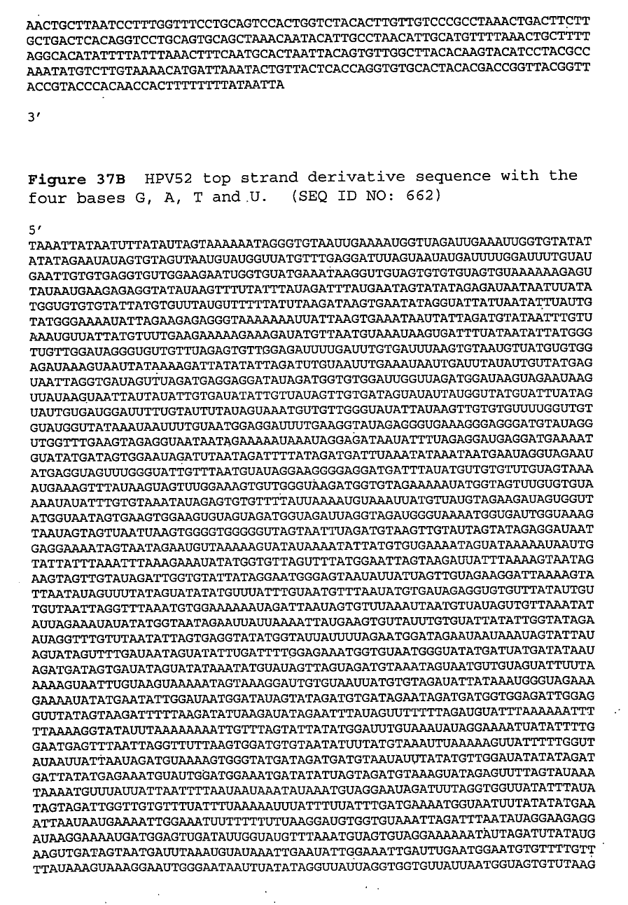

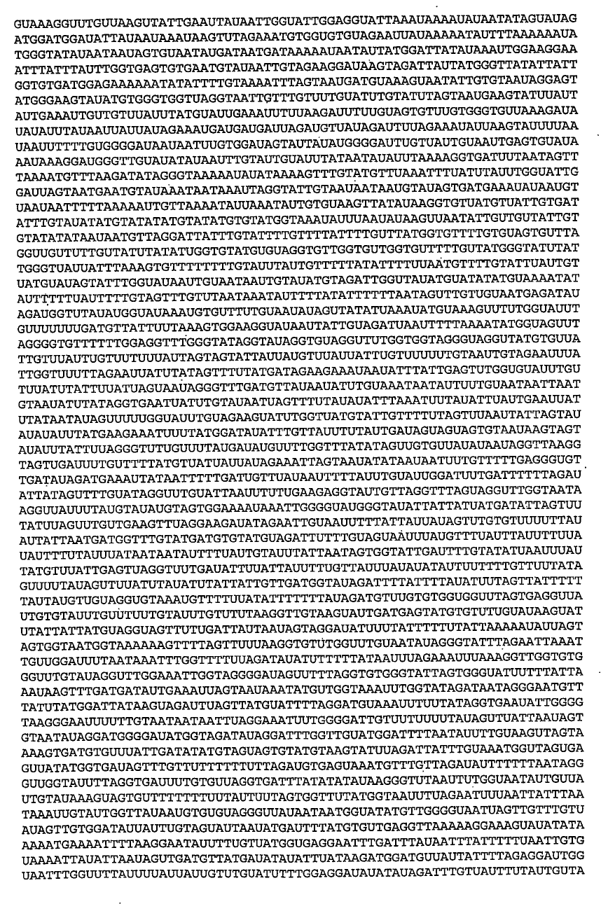

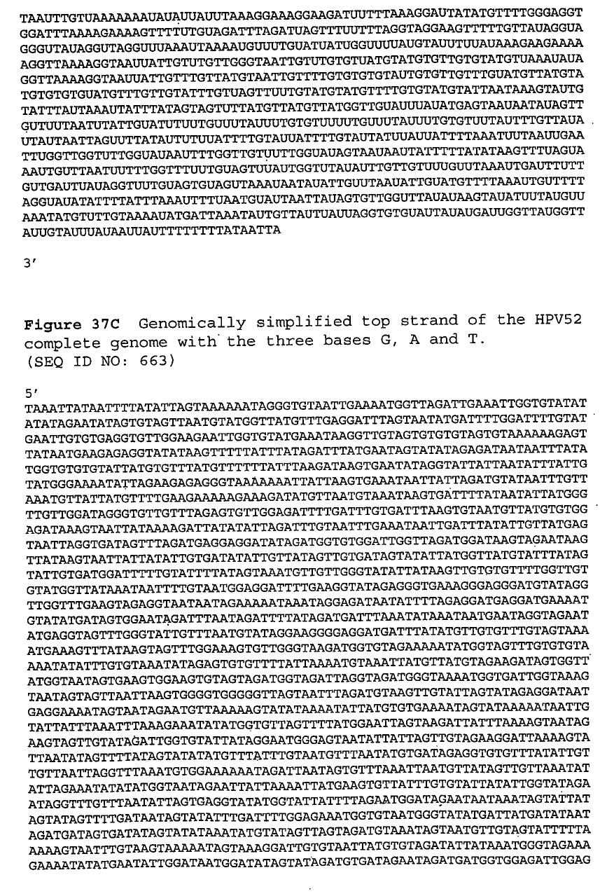

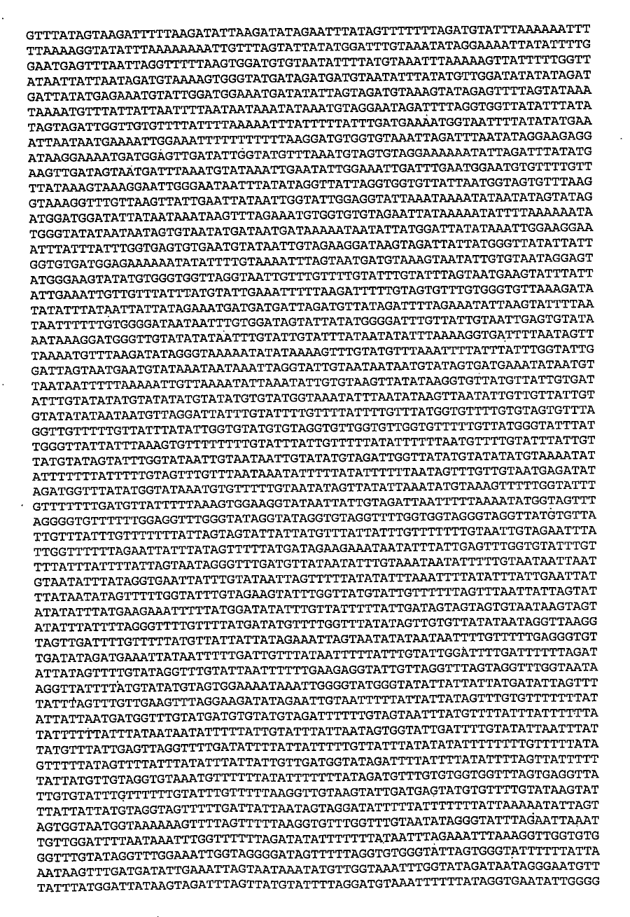

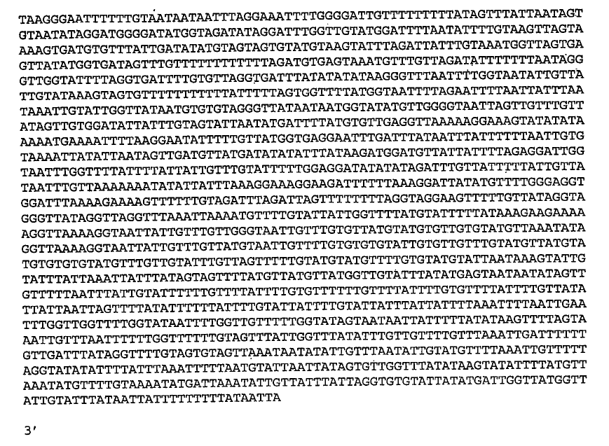

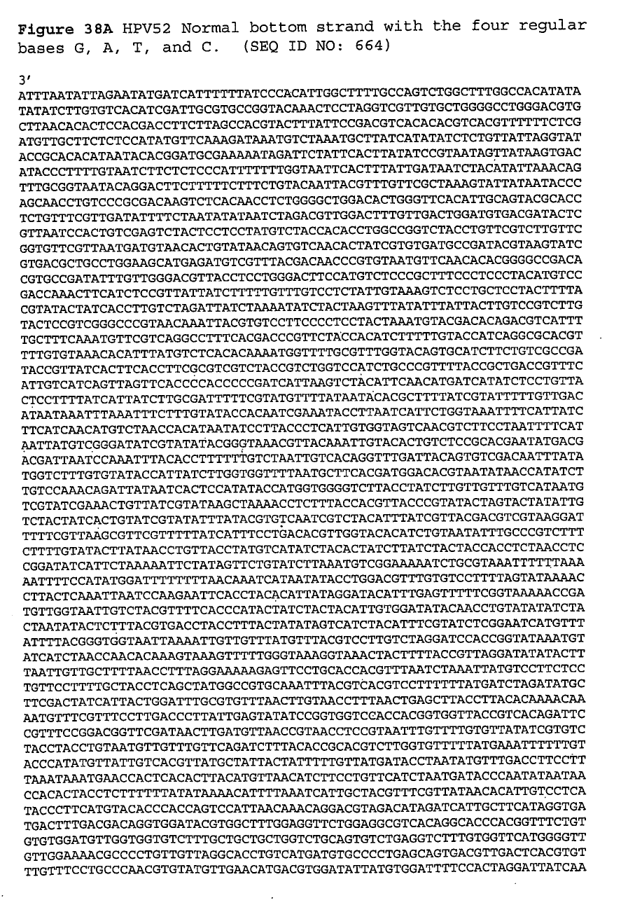

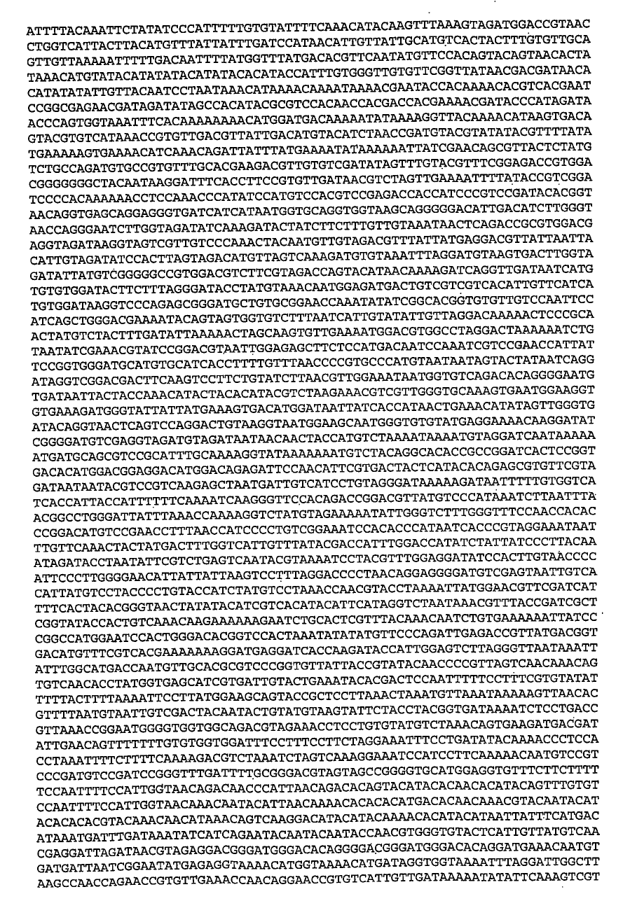

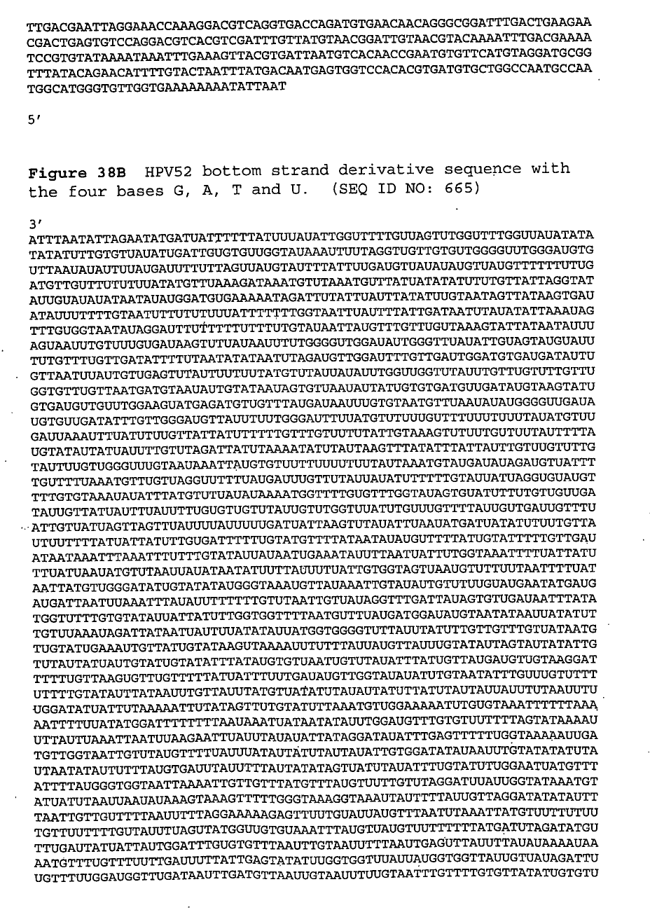

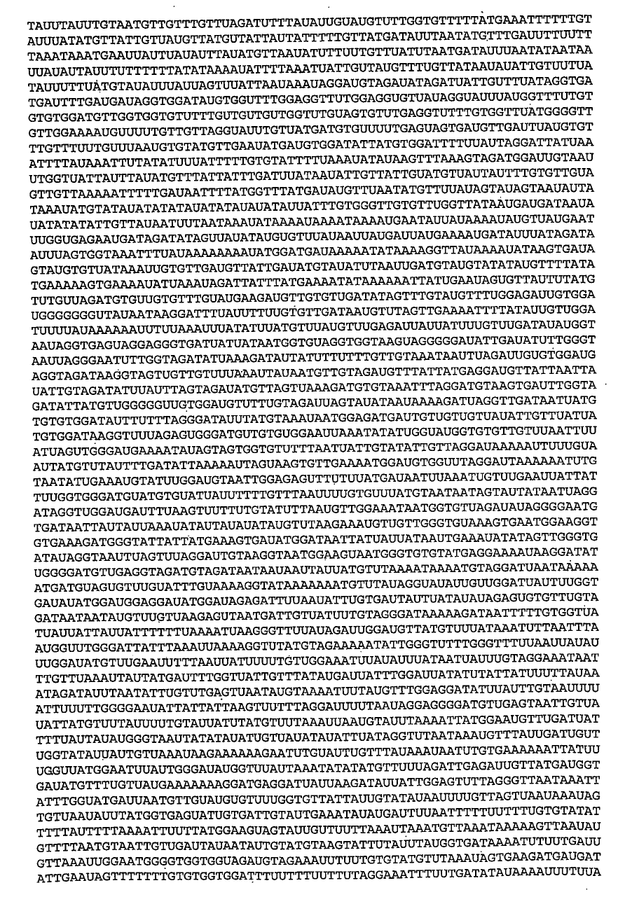

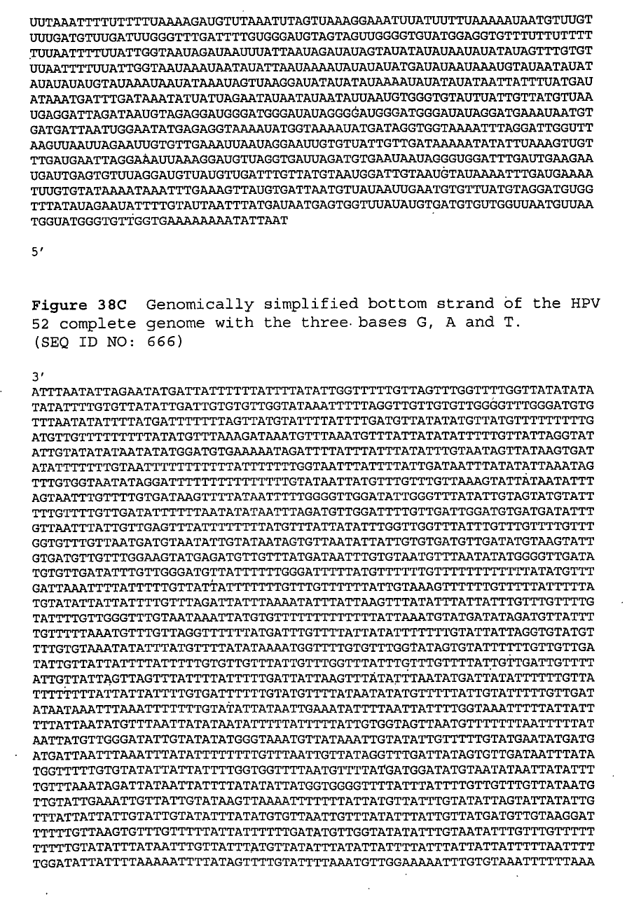

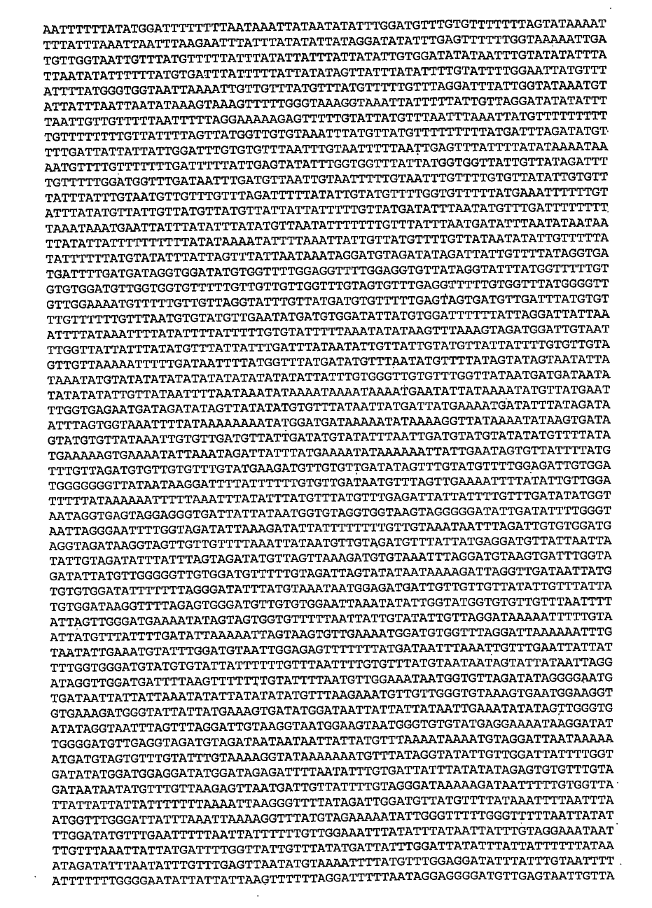

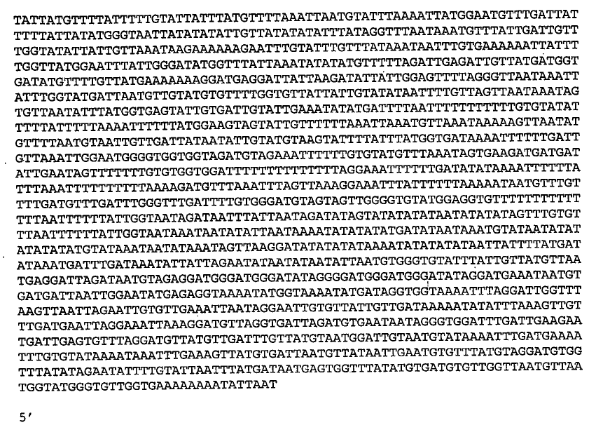

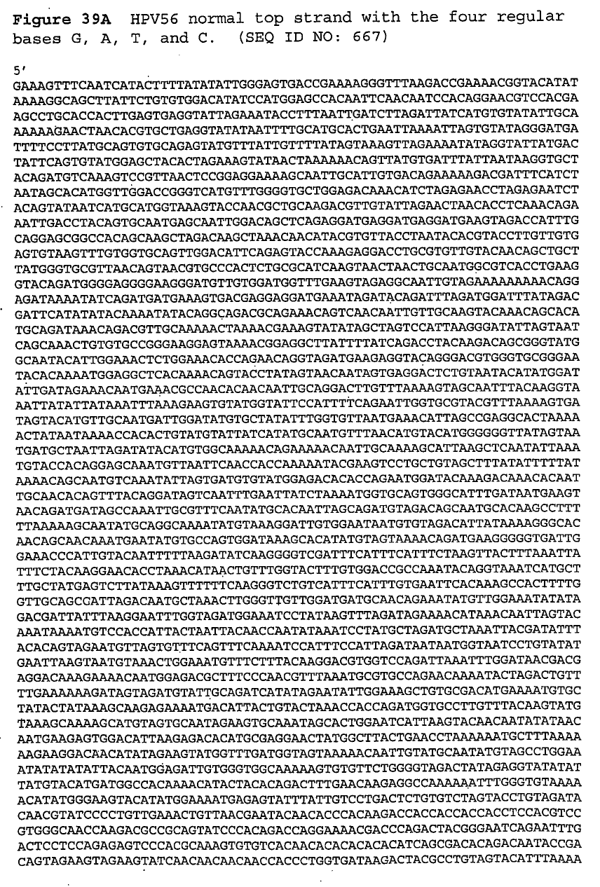

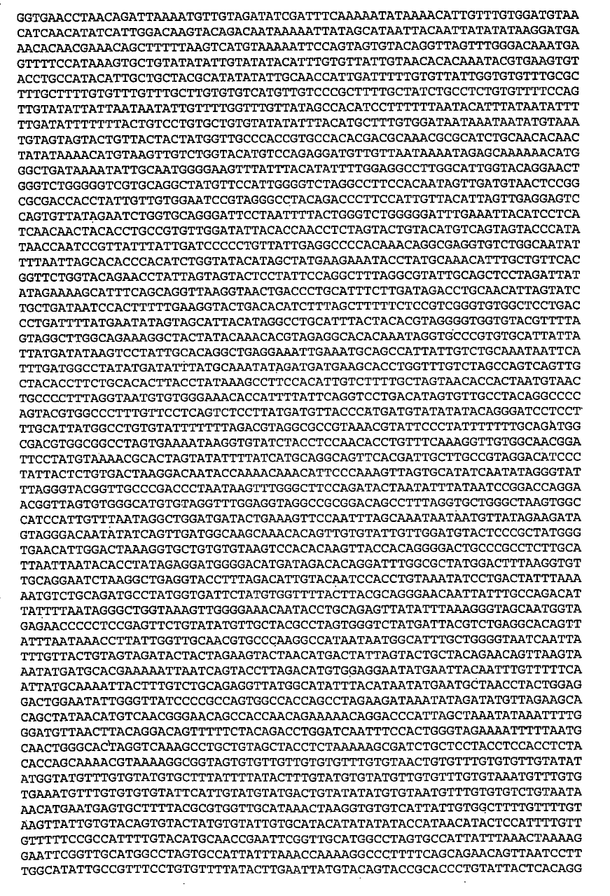

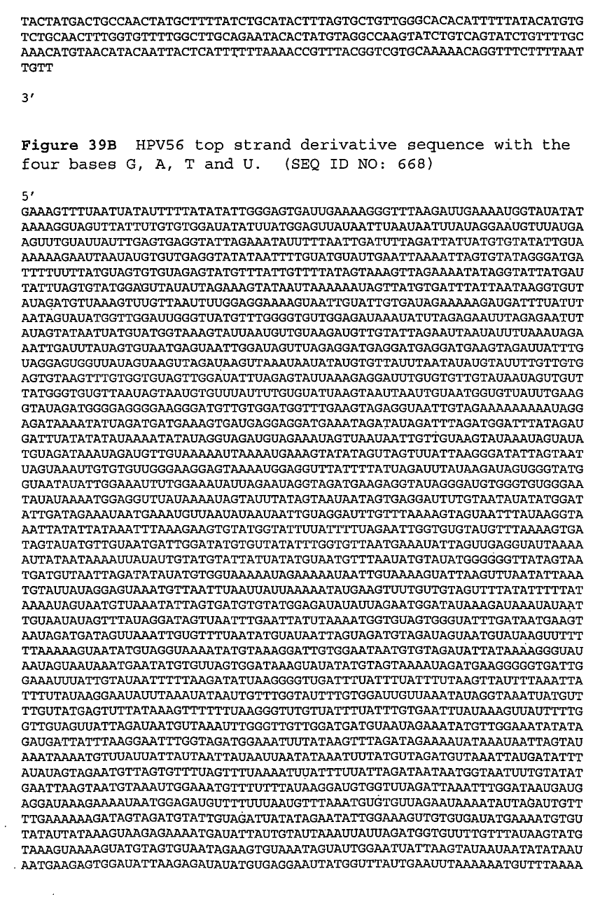

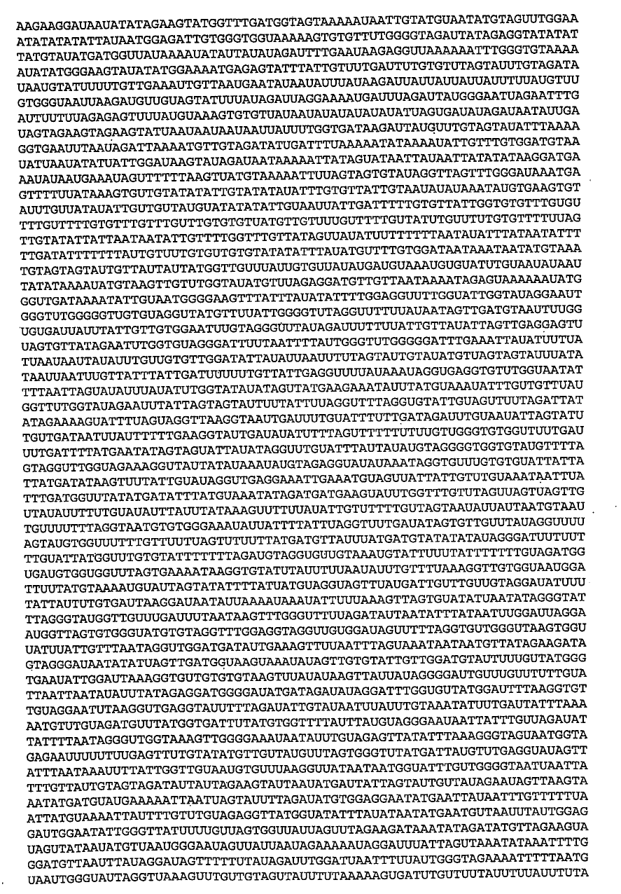

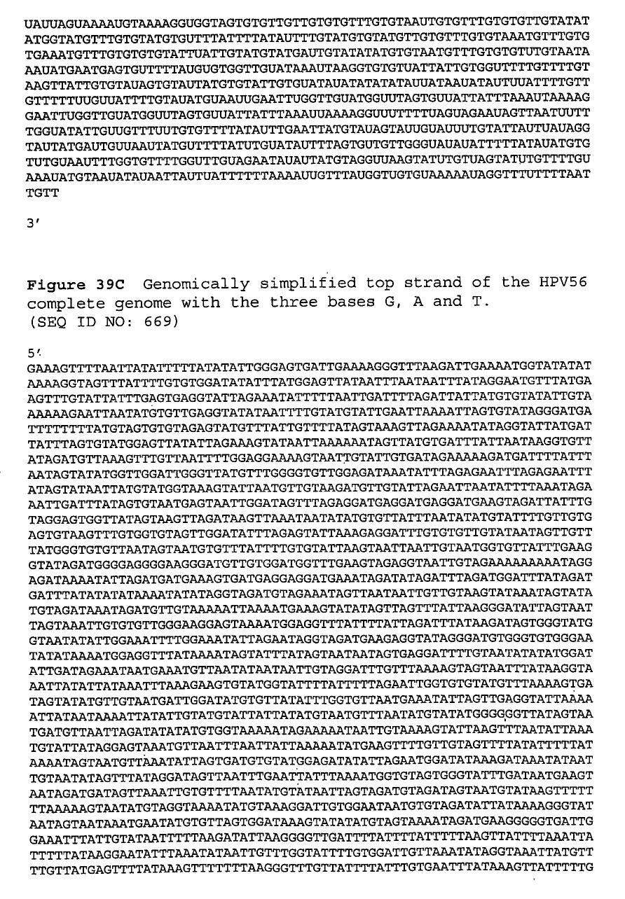

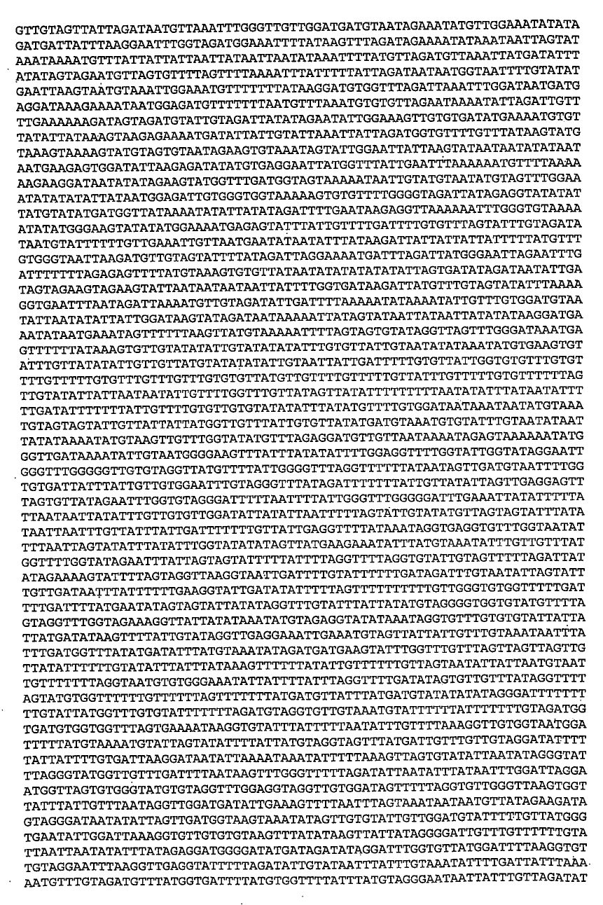

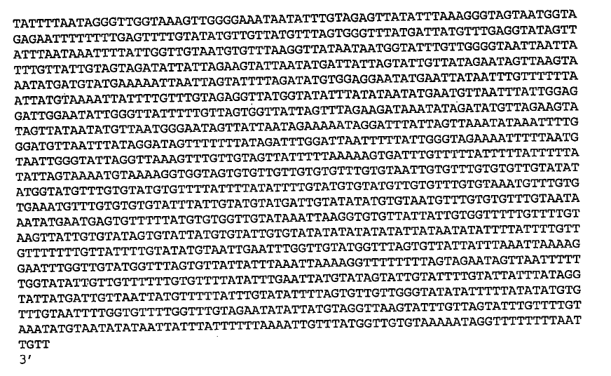

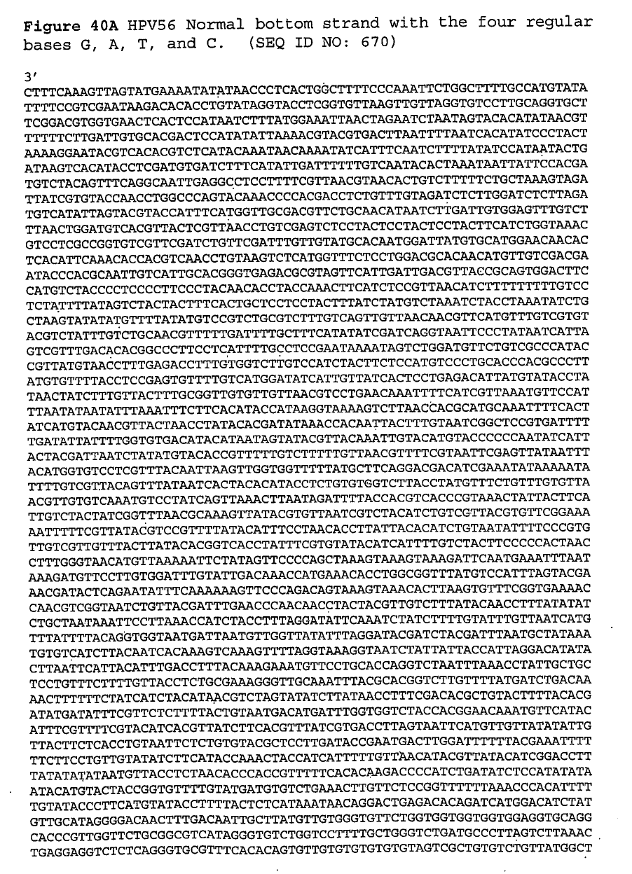

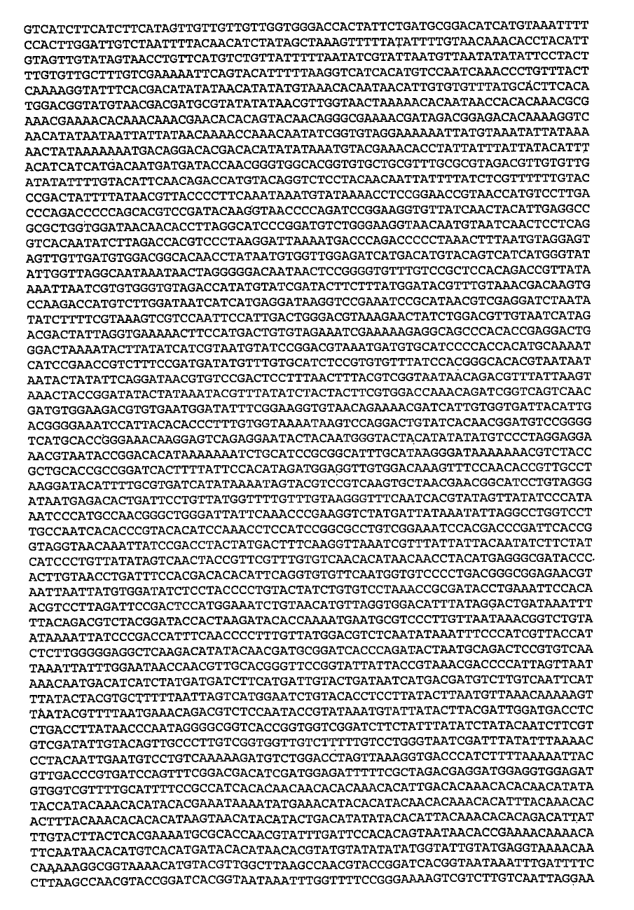

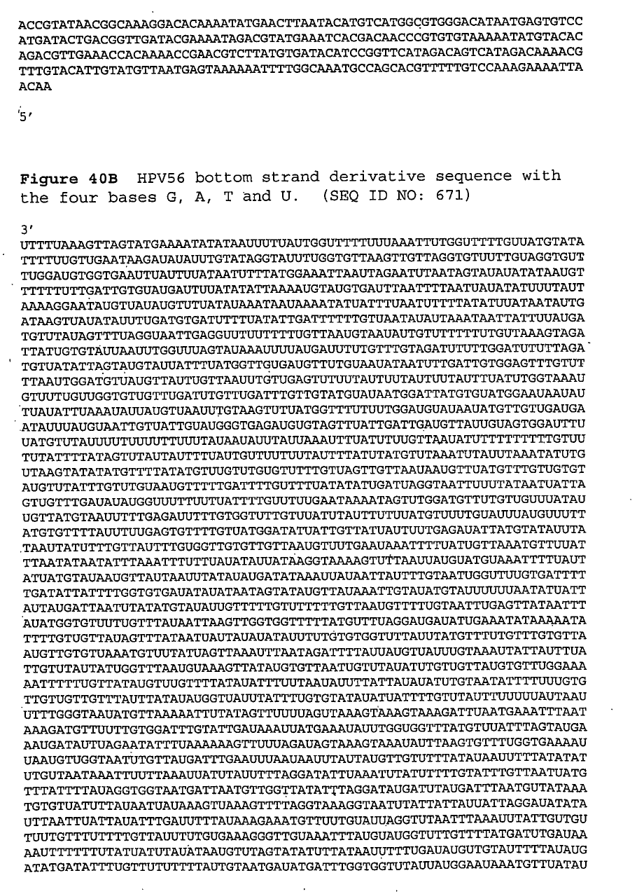

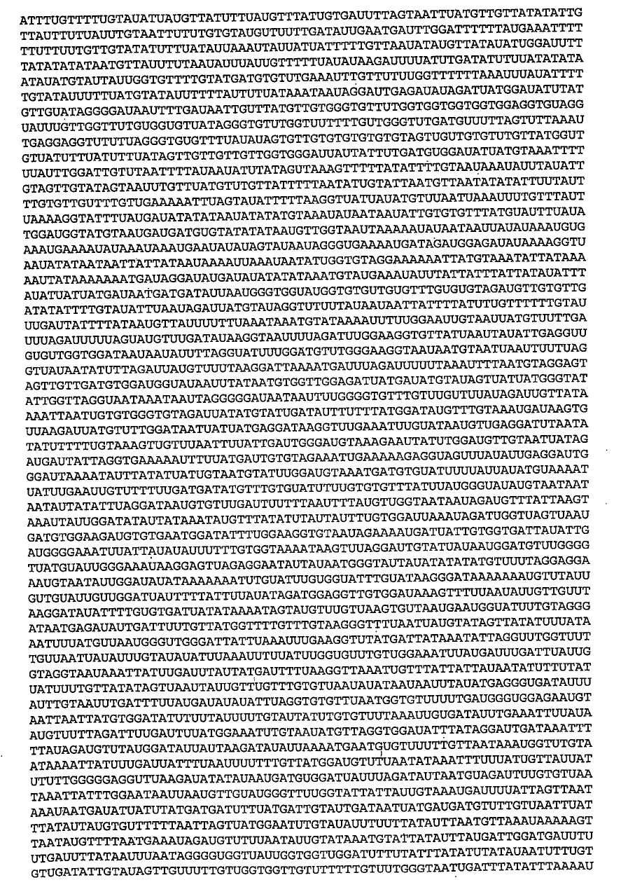

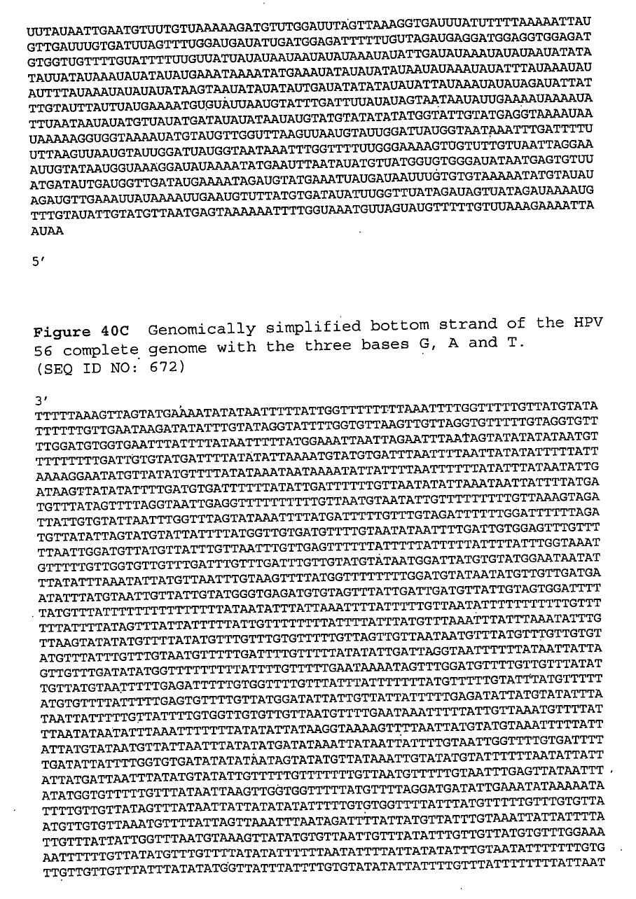

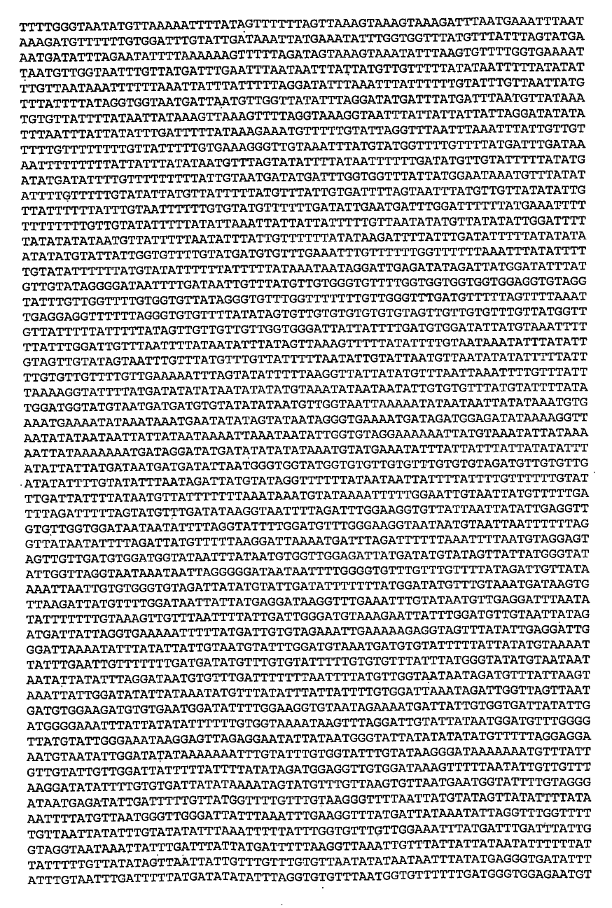

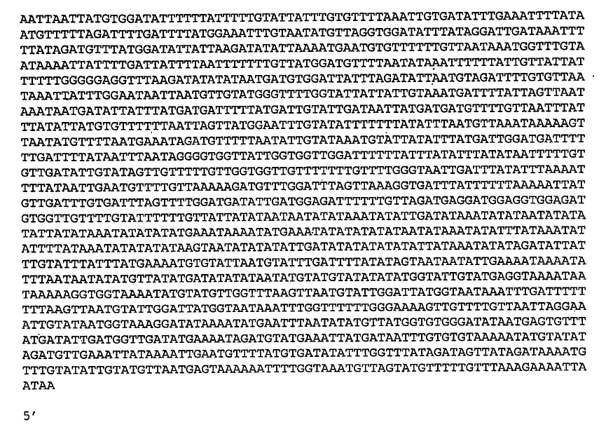

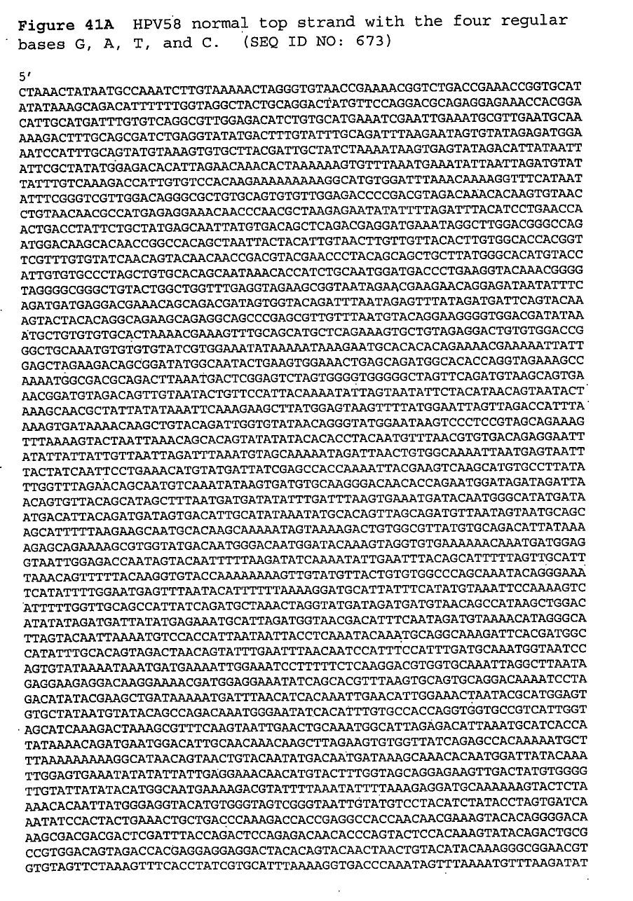

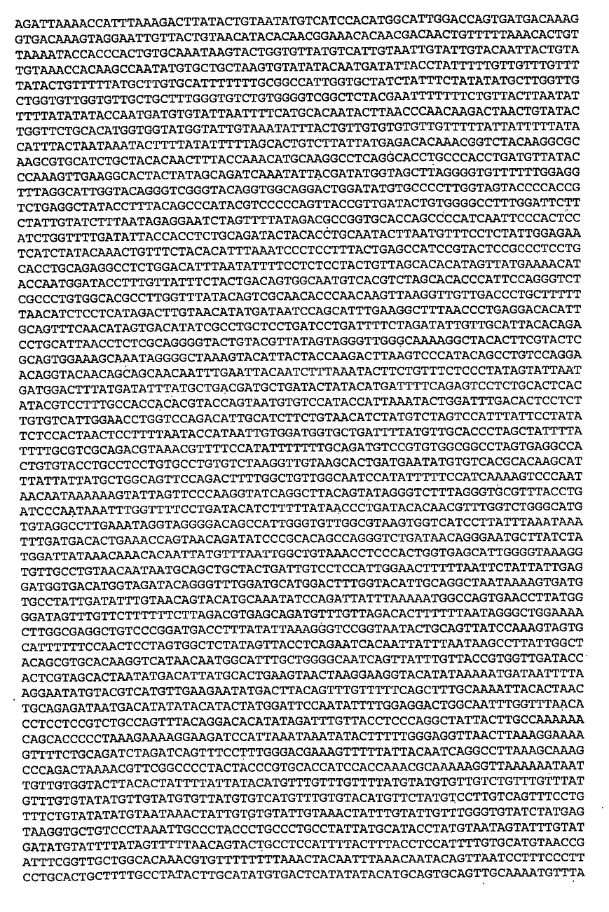

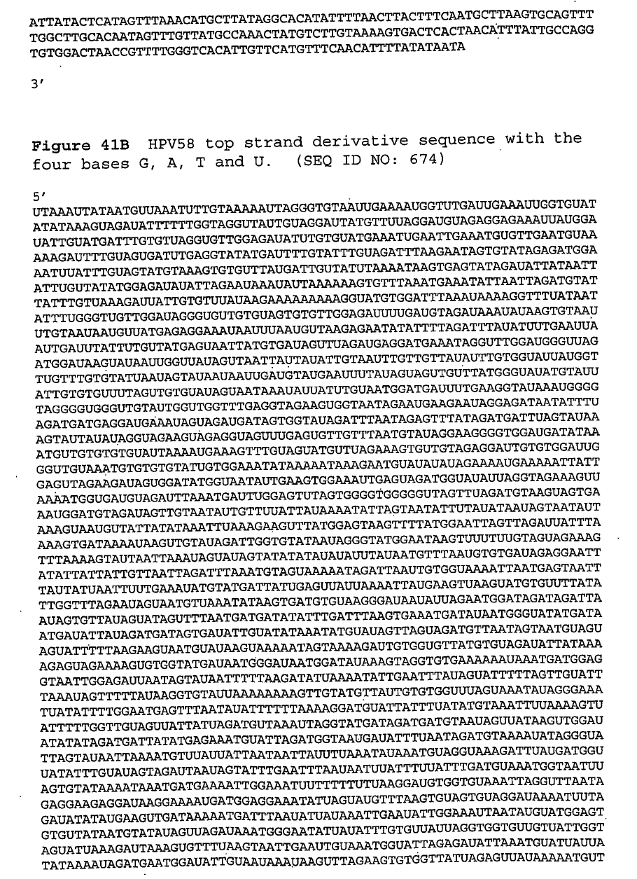

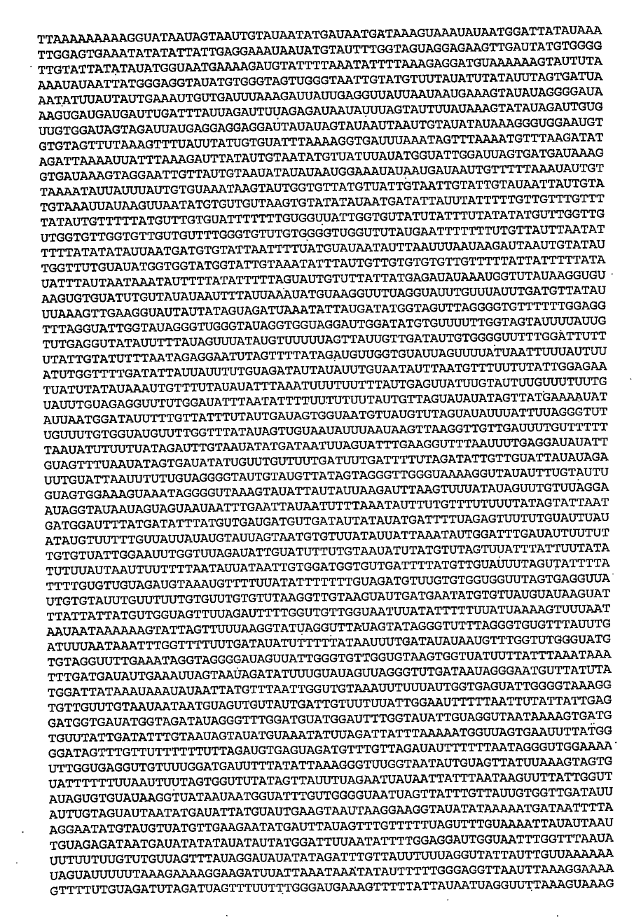

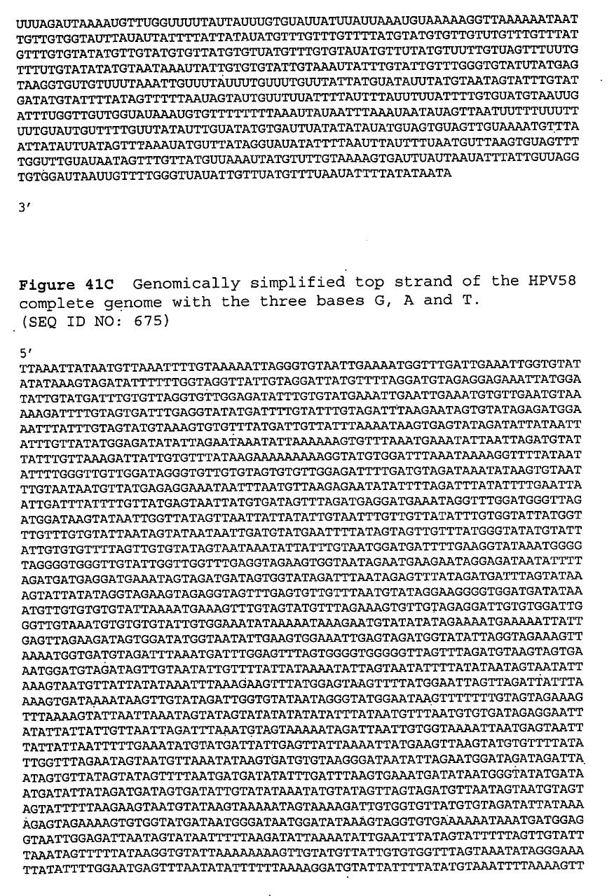

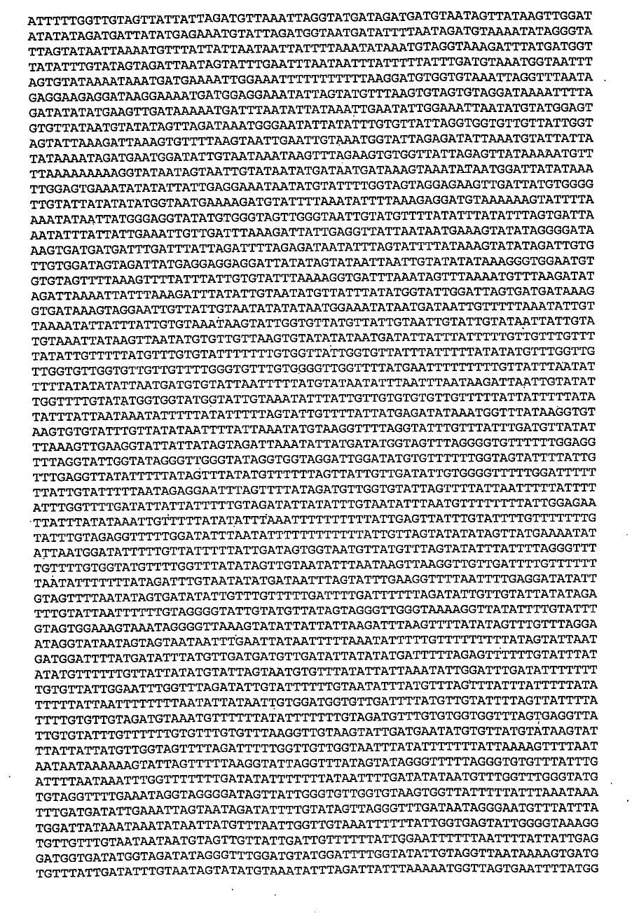

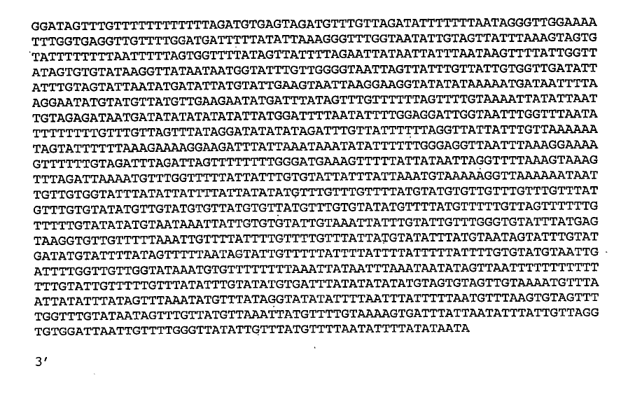

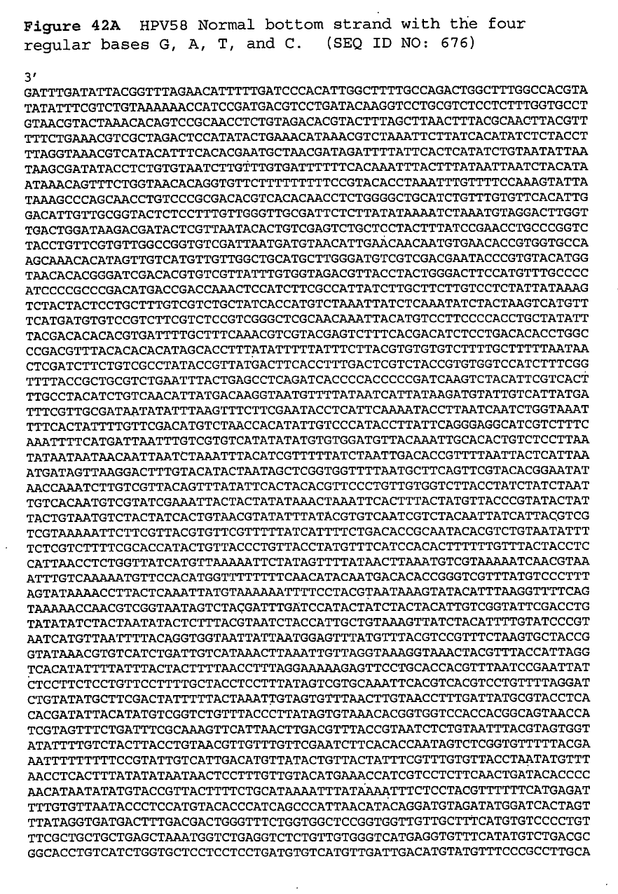

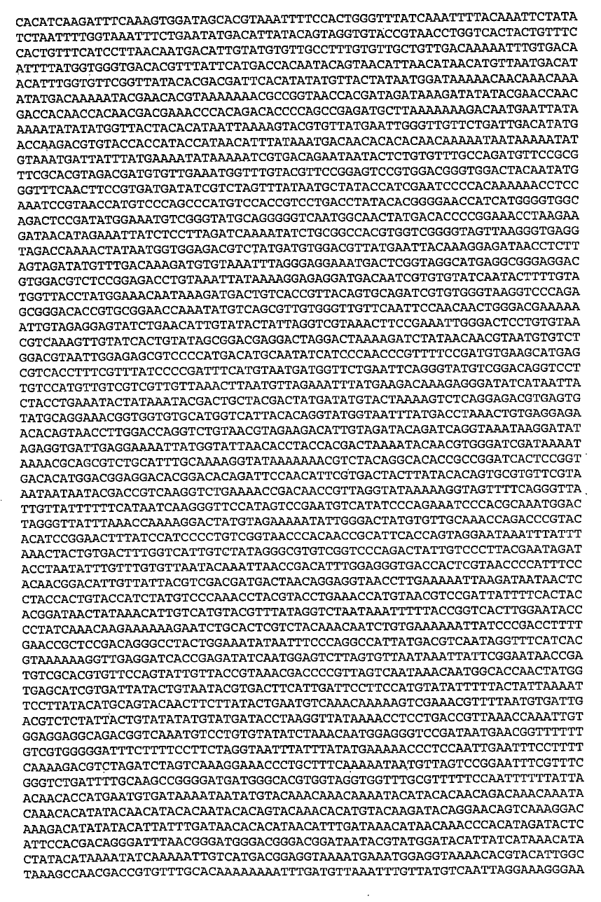

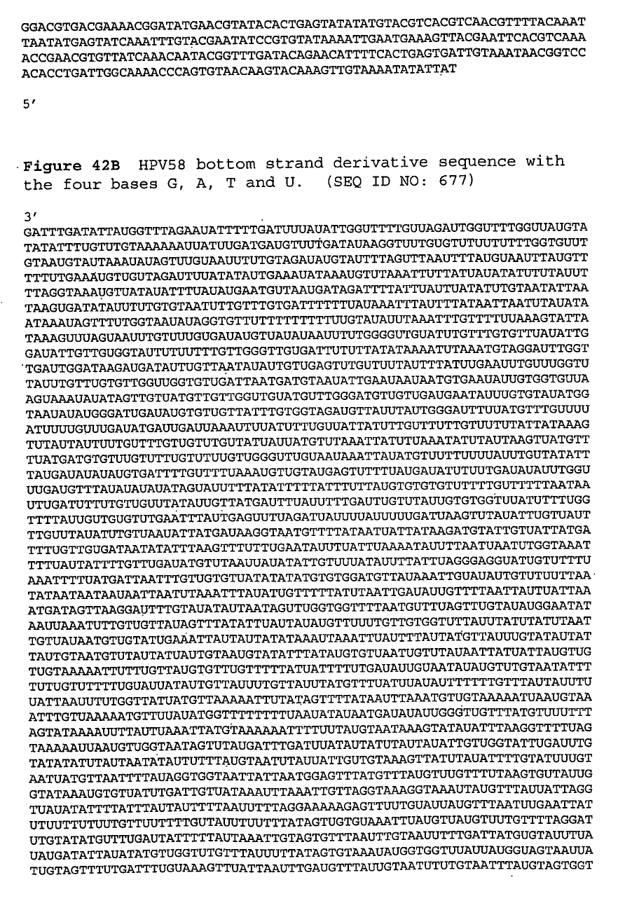

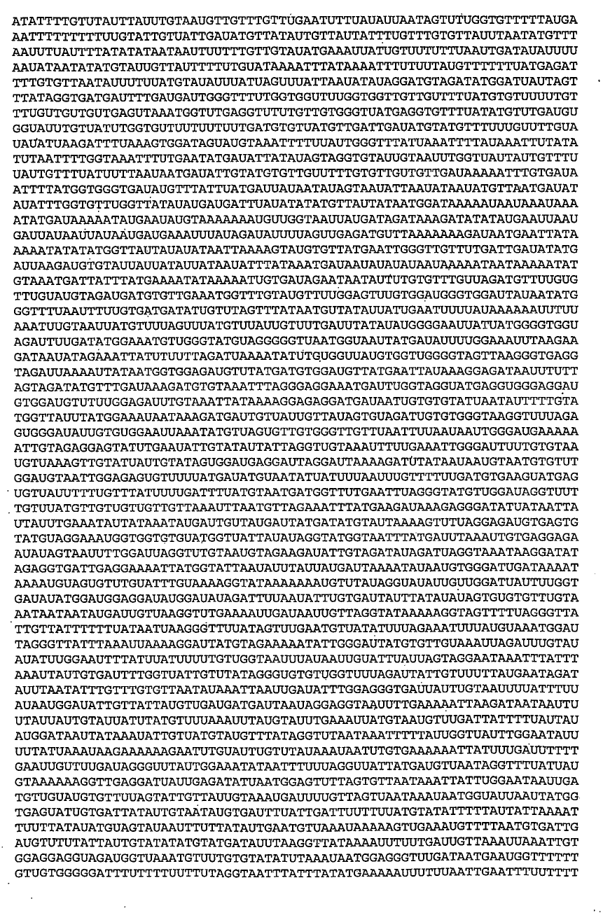

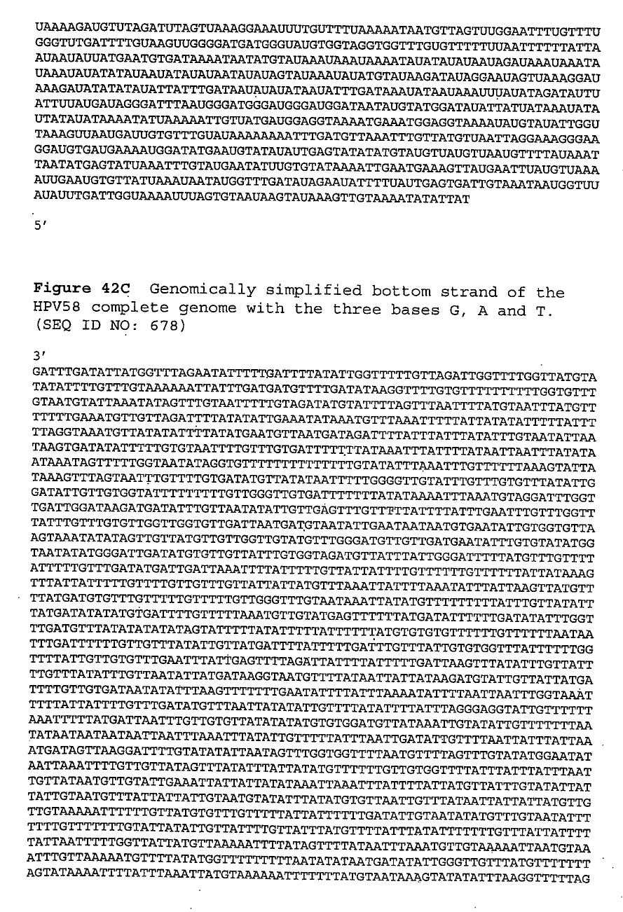

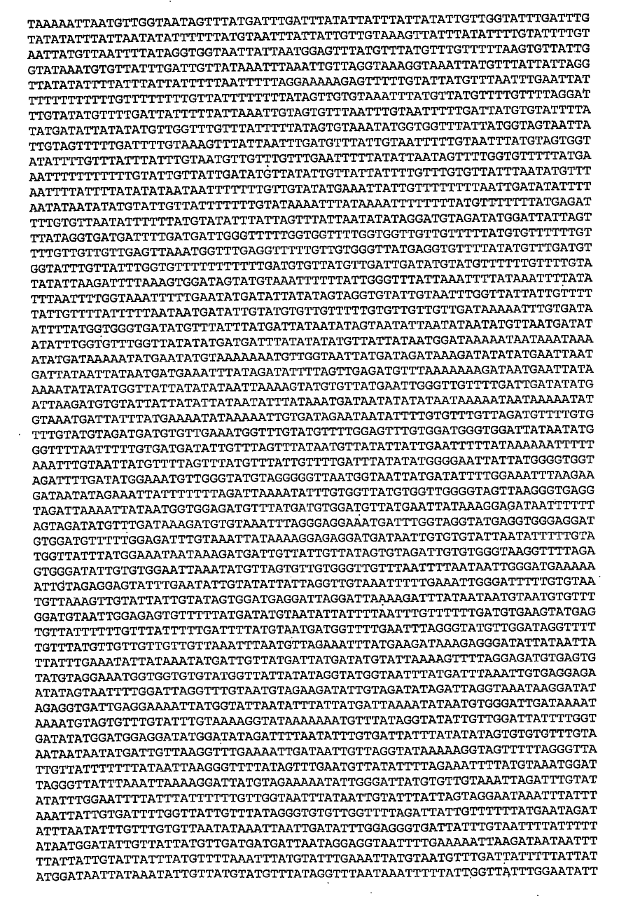

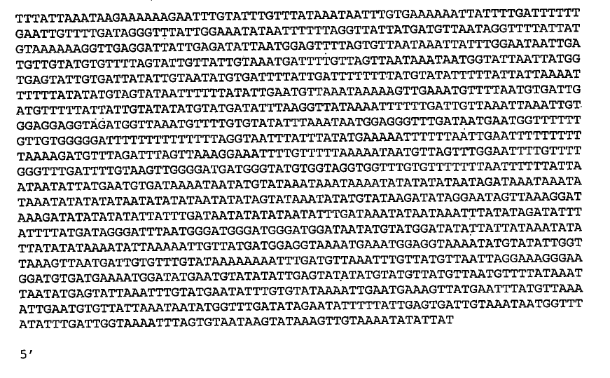

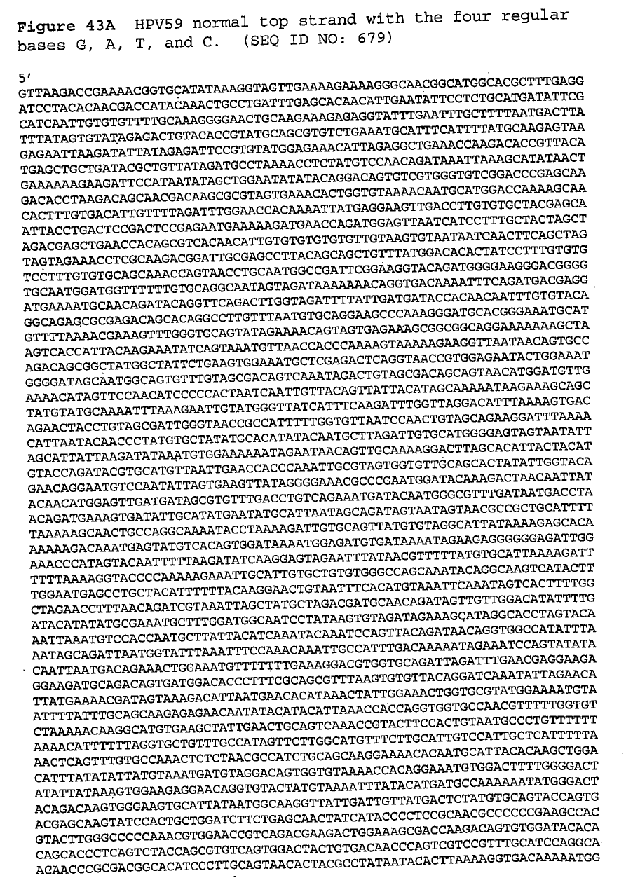

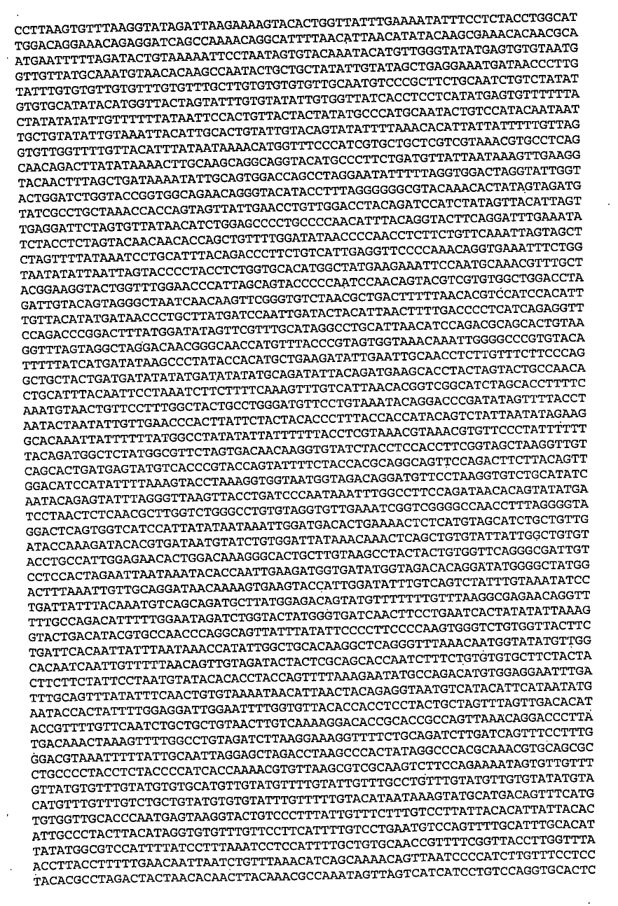

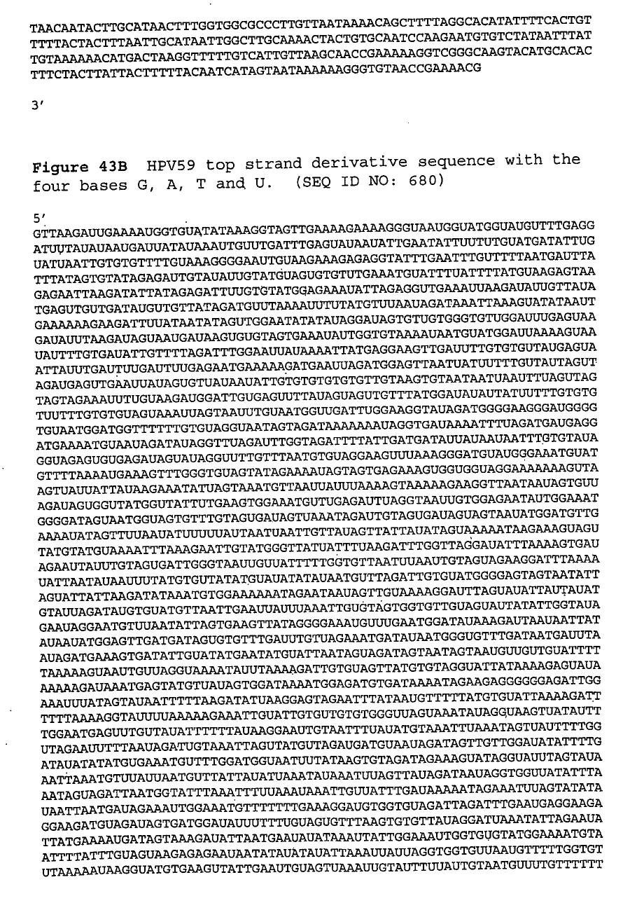

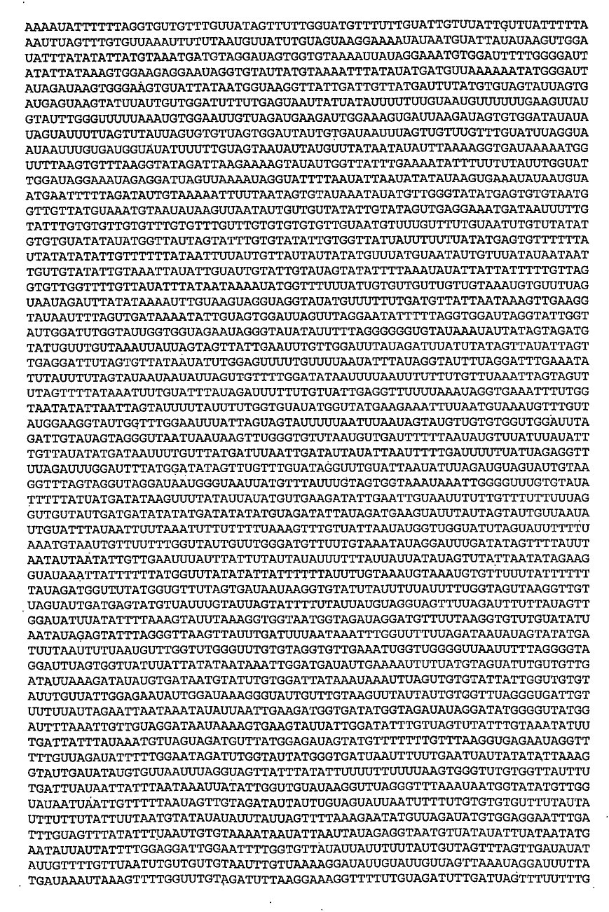

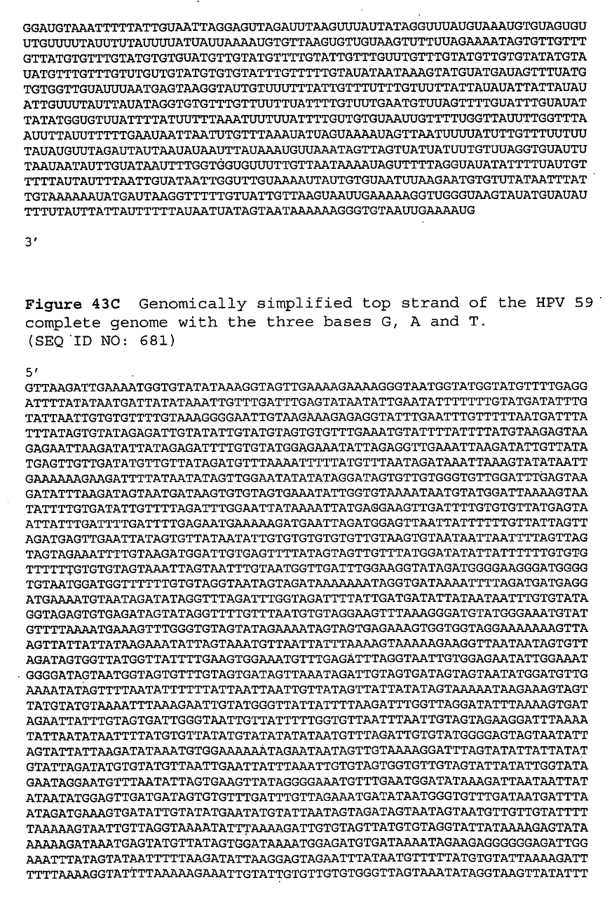

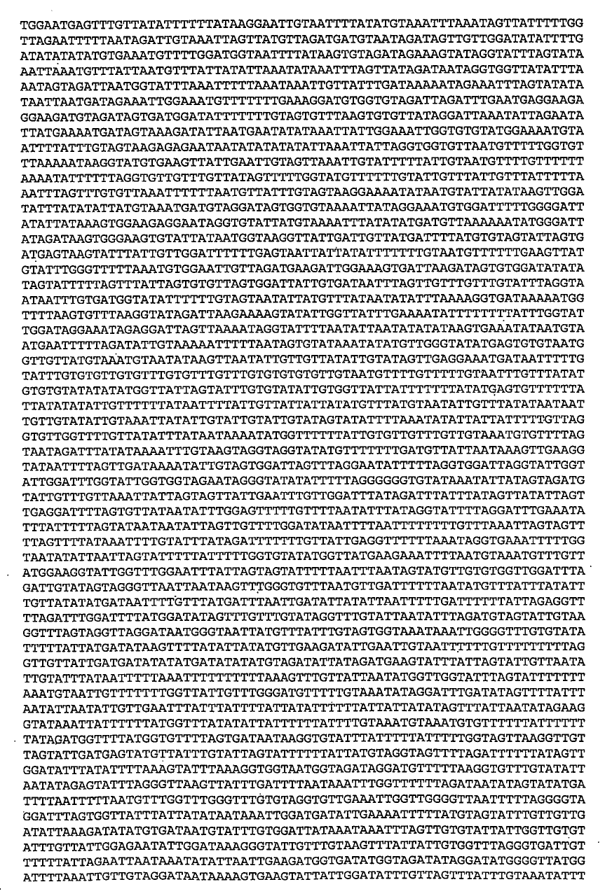

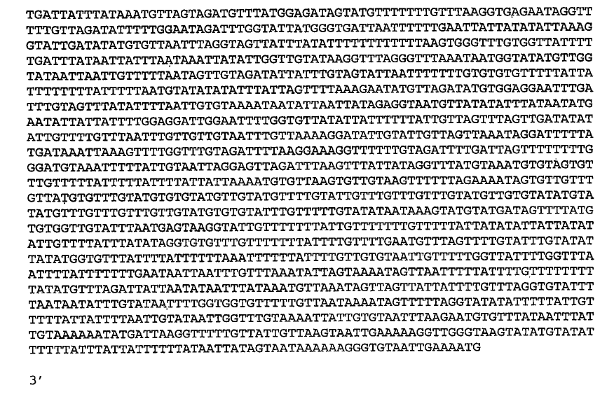

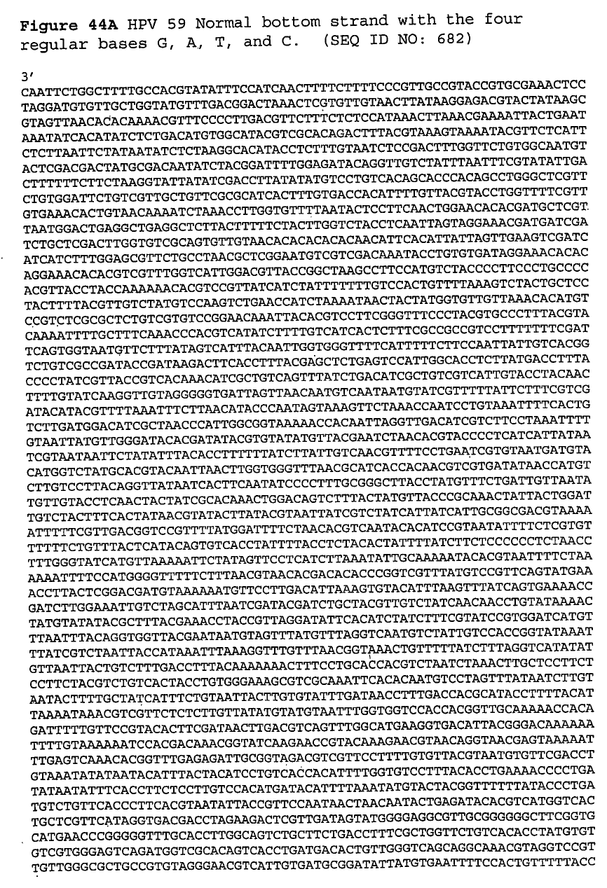

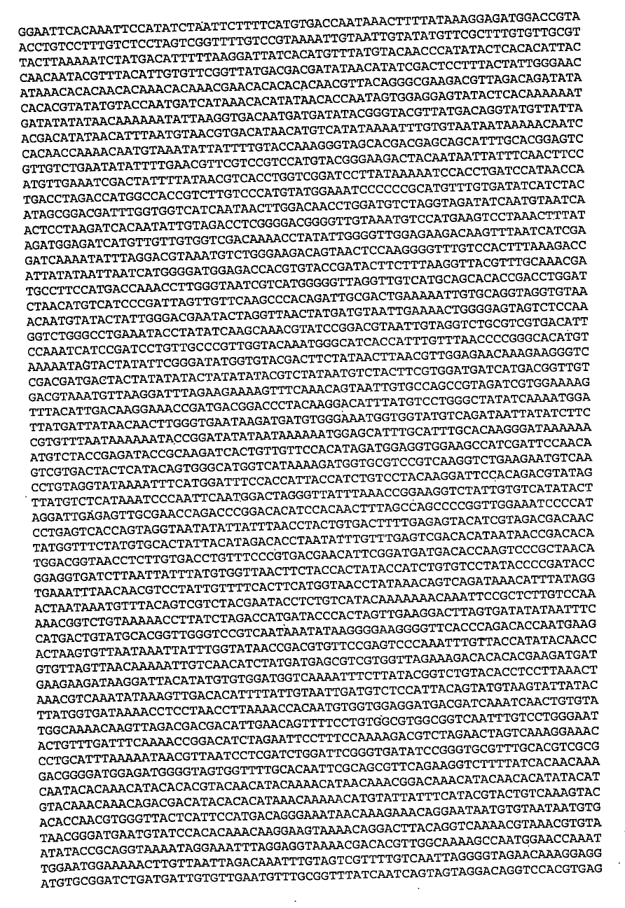

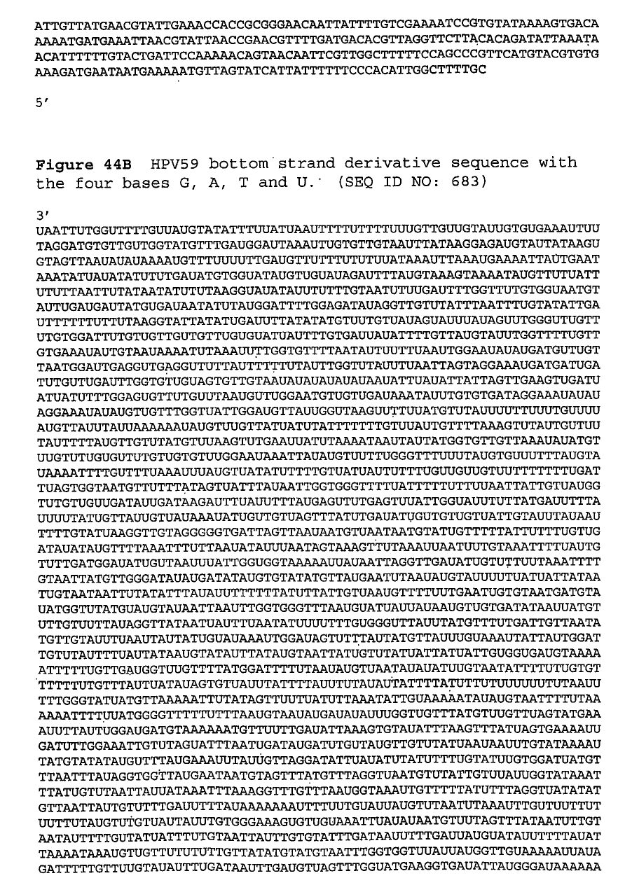

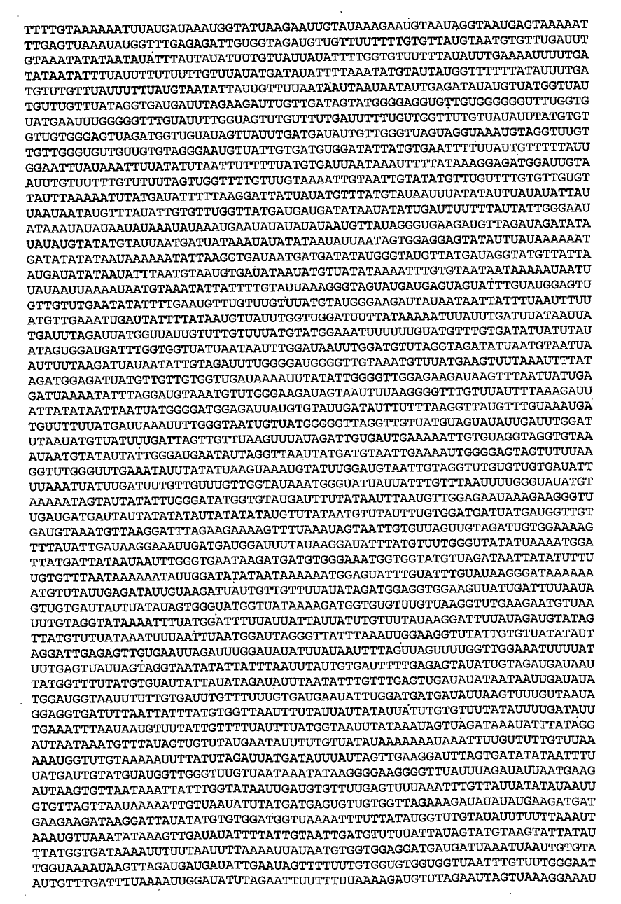

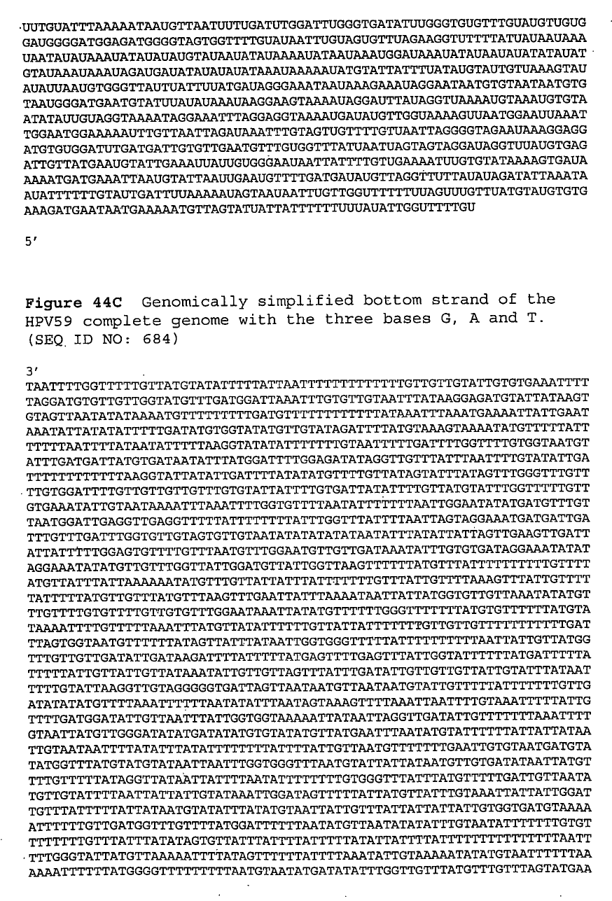

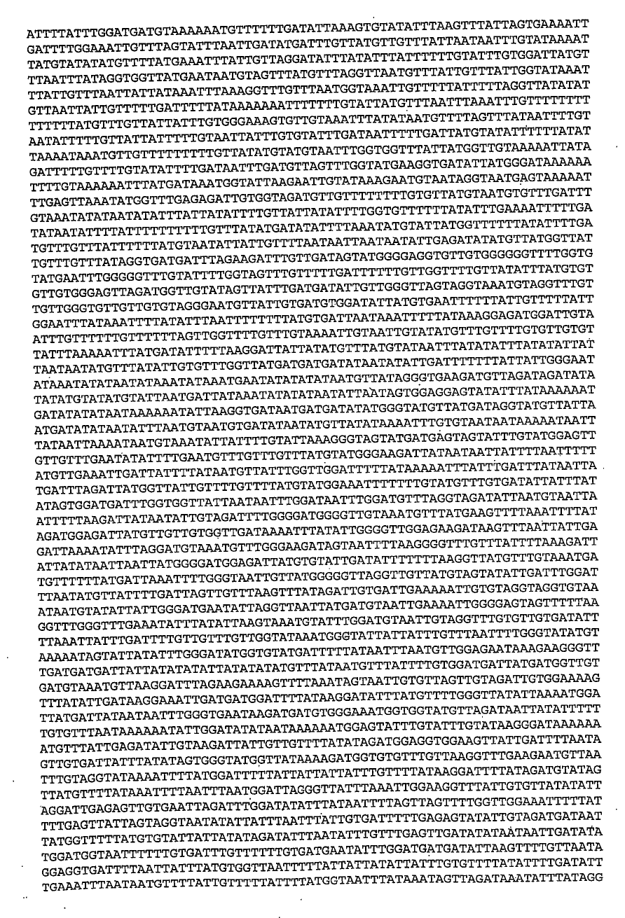

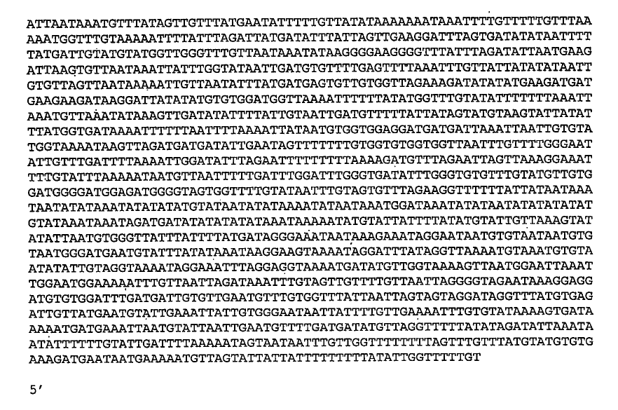

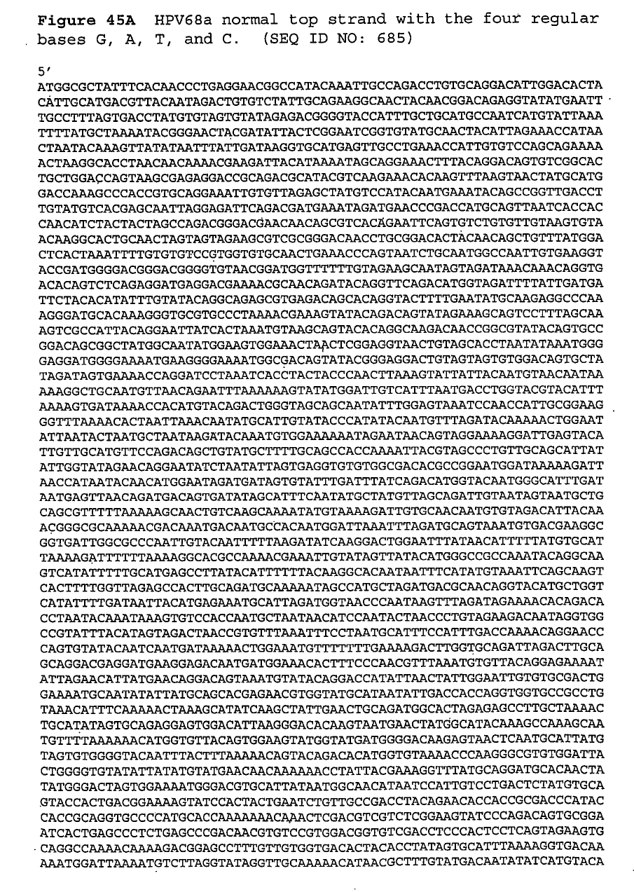

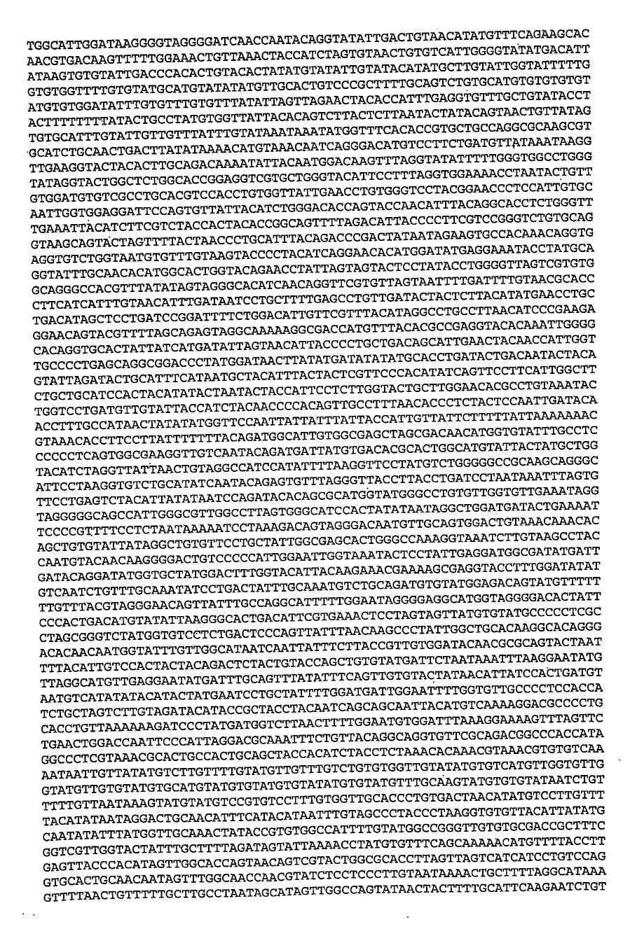

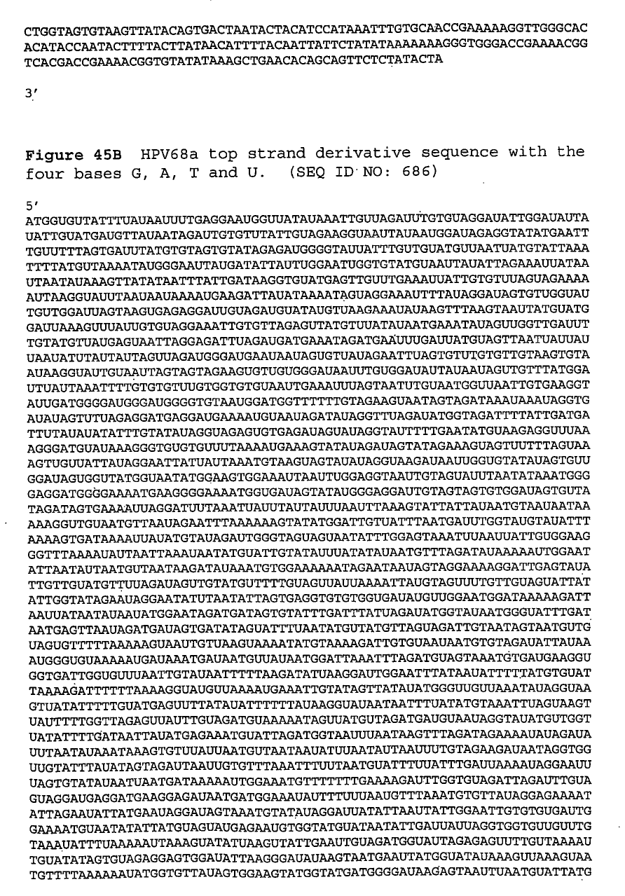

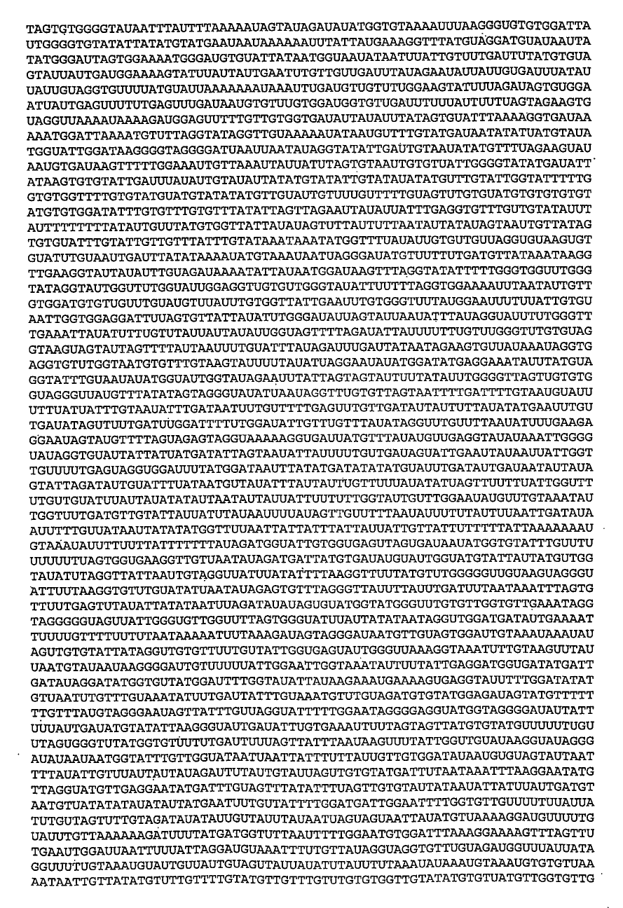

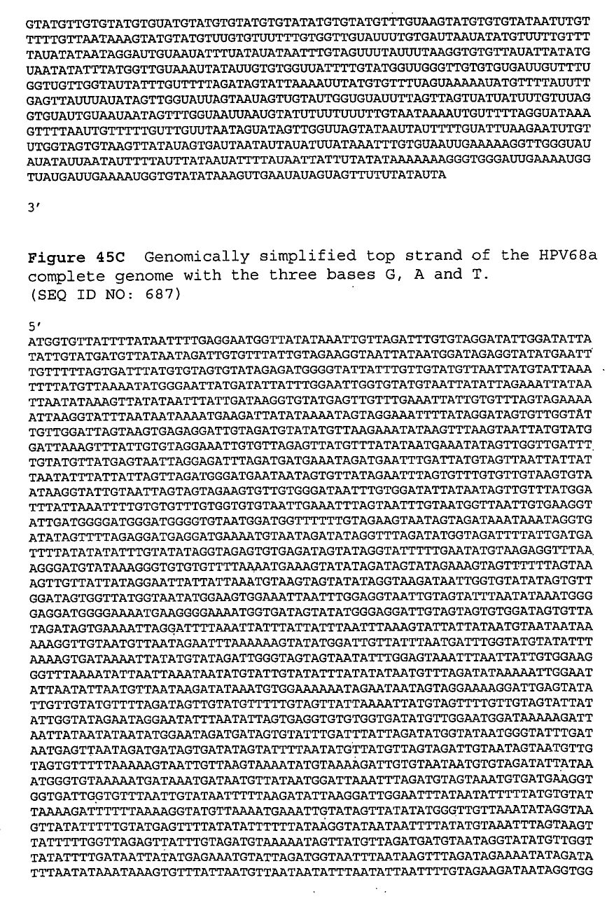

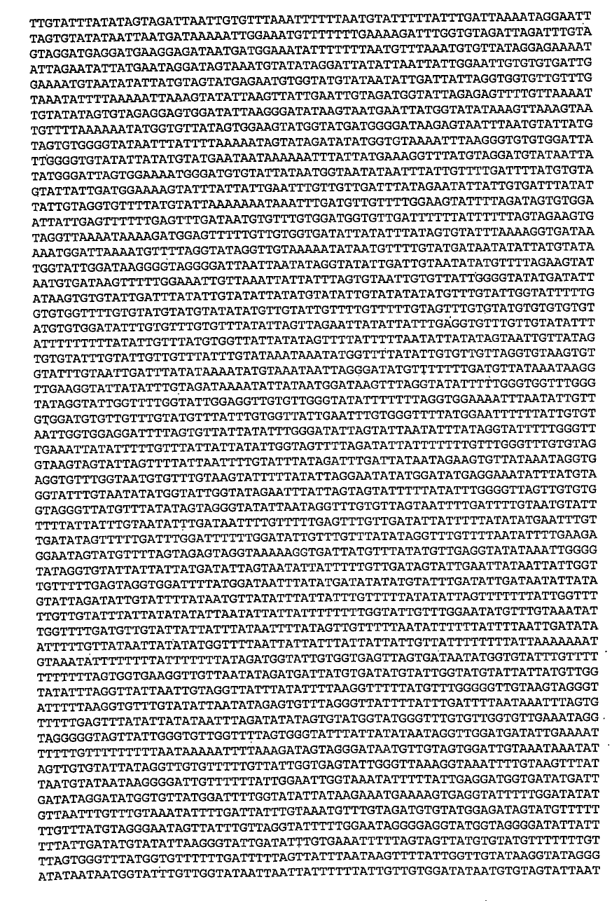

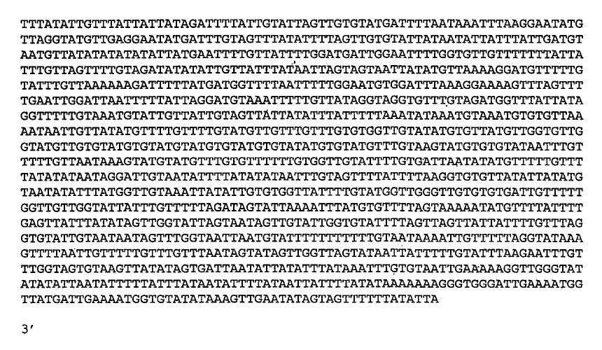

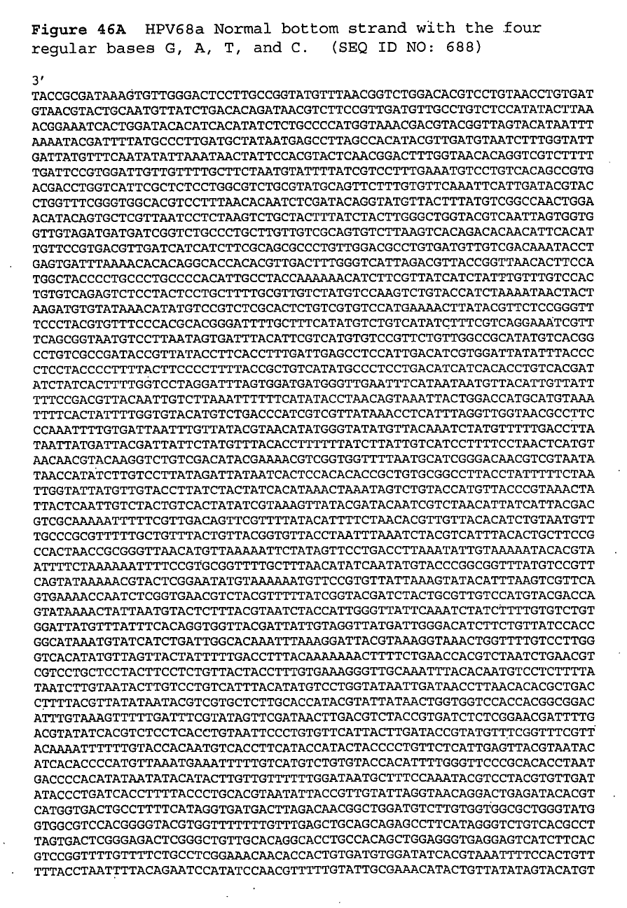

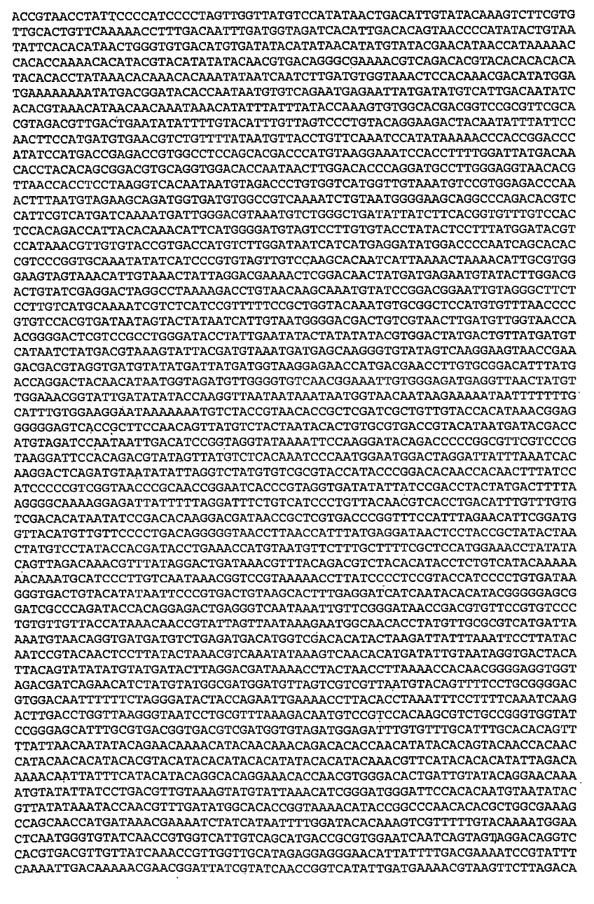

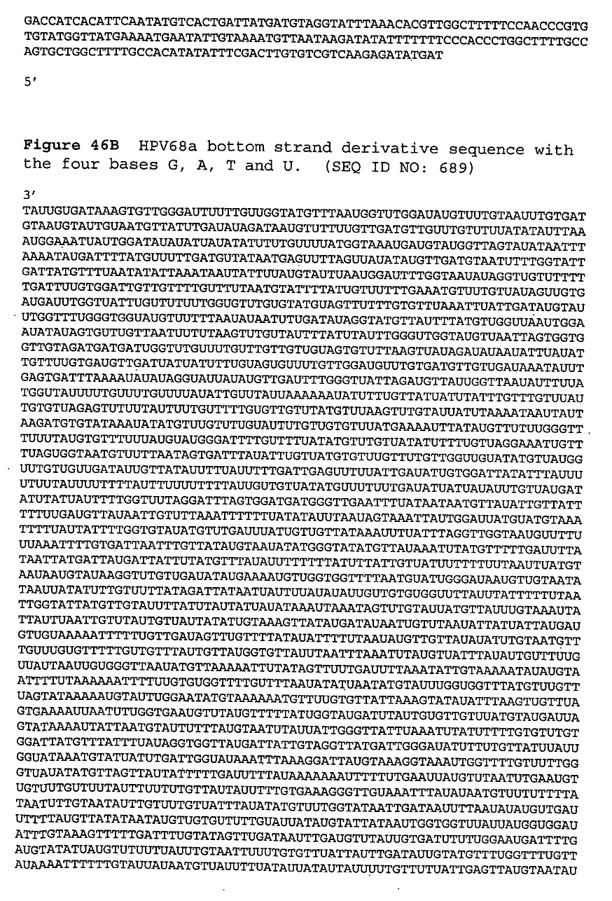

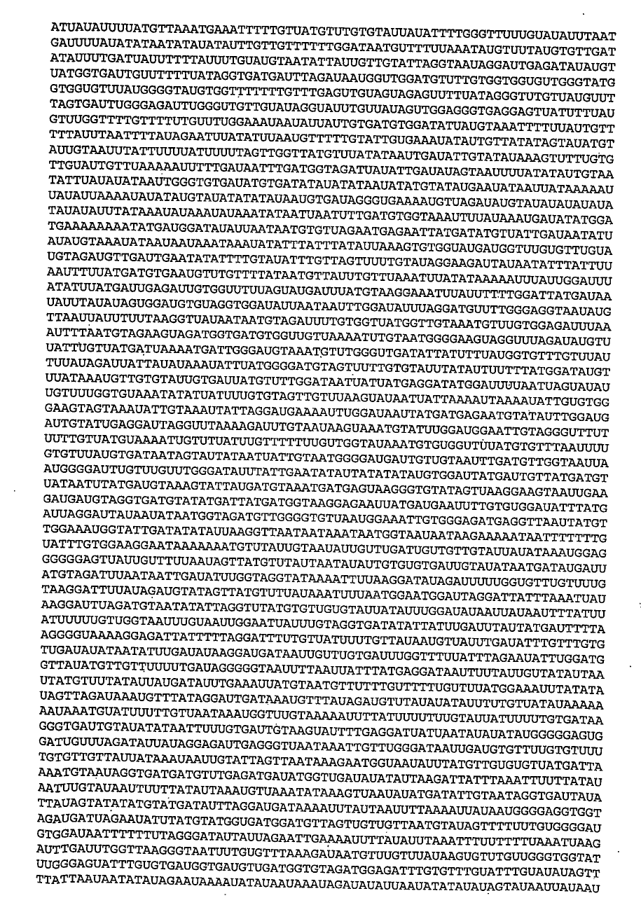

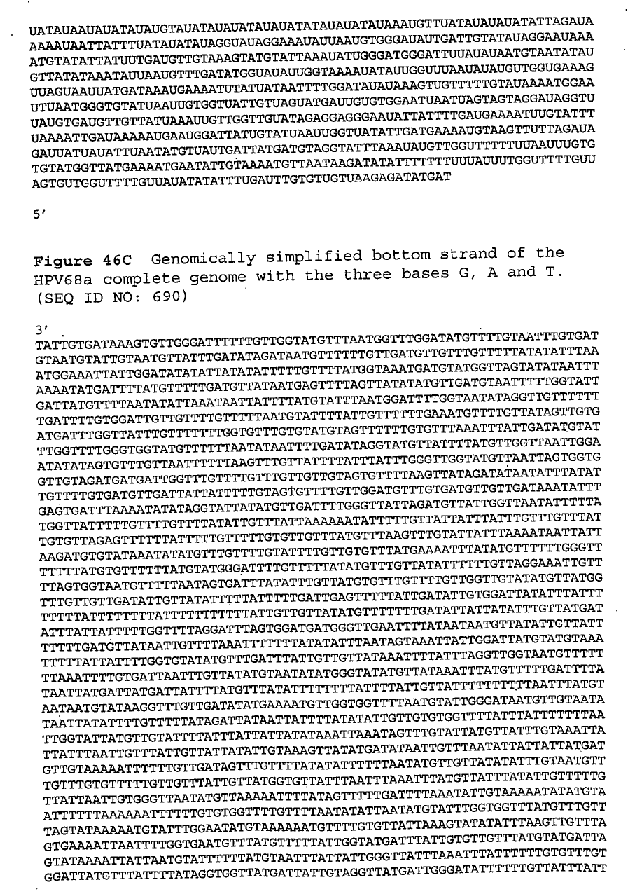

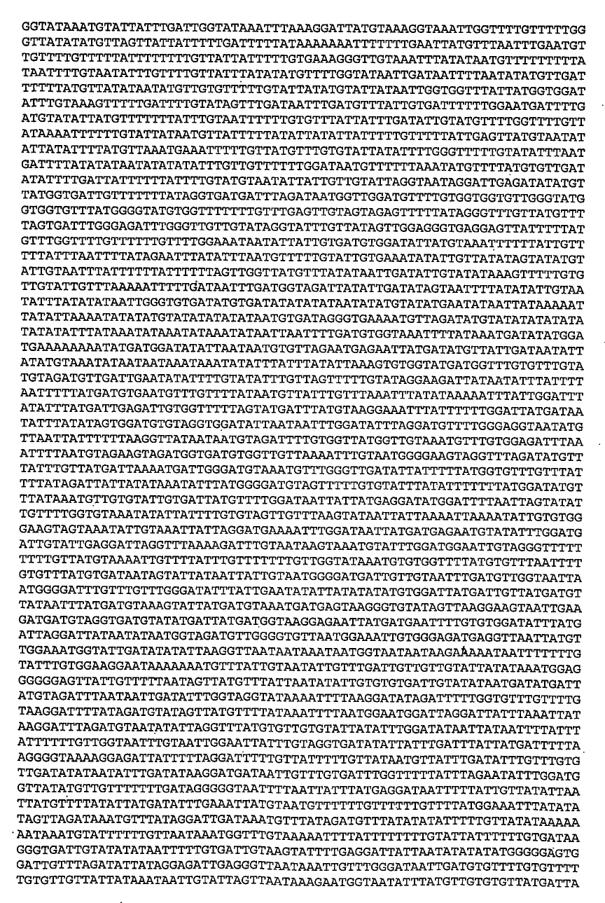

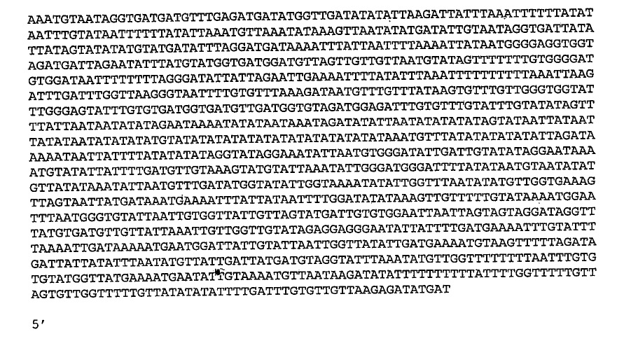

FIGS. 23 to 46 show natural (A), derivative (B) and simplified (C) HPV nucleic acid sequences for top and bottom strands of high-risk HPV18, 45 or 56 and medium risk HPV 31, 33, 35, 39, 51, 52, 58, 59 and 68. Invention relates to B and C sequences.

FIG. 1 shows the multiple DNA alignment of the same 8 base pair genomic region of individual viral types HPV 33, 35, 39, 52, 58, 16, 18, 45 and 56, before and after complexity-reduction using bisulphite treatment. The region under consideration is that within the L1 gene at positions 6600-6607, (anchored using the standard coordinates of HPV16). The different HPV types vary in their nucleotide sequence at positions 6590, 6593 and 6956, having either a C or a T at these positions, (bolded). However after chemical conversion of HPV DNA, all of these HPV types now have an identical DNA sequence between ‘top’ strand positions 6590 and 6597, (namely TATAATA) SEQ ID NO: 518), and hence a single primer or probe can be synthesized, (that together with a nearby appropriate primer), will amplify this region from a primer pair. The ability to employ unique primers instead of degenerate ones is the key to increases in accuracy and to the generation of specific amplification products, an issue of major importance when viral types are being used for diagnostic purposes in the clinic and for subsequent treatment regimens. The use of a second nearby sequence allows amplification of all the viral types given in this illustration, (namely HPV types, 33, 35, 39, 52, 58, 16, 18, 45 and 56), using one set of non degenerate primers.

It should be stressed that a major failing of the prior art in the HPV PCR area has been the inability to circumvent the use of degenerate primers, which by necessity, contain a mixture of bases at those positions in which a base is different between different viral types. Thus, to amplify the sequence of the non-bisulphite treated sample, the PCR primers in FIG. 1 would have to be of the sequence YCAYAAYA (SEQ ID NO: 701) (where Y=C or T at that position). In contrast, with a bisulphite treated HPV derivative, the primer TTATAATA (SEQ ID NO: 518) becomes an identical match for all viral types. The main problem with the degenerate primer approach is that in the conventional 4-base genome, the primers very quickly become so degenerate that either they do not produce an amplified product or produce multiple products or smears due to non-specific hybridisation to non-target DNA sequences.

FIG. 2 shows DNA alignment of a 17 base pair genomic region of individual HPV types 6, 11, 43, 44, 53, 55, 30, 31, 39, 51, 52, 16, 18 and 45, and the complexity-reduction following bisulphite treatment of the DNA sample. This region is also in the L1 gene but is at positions 6581-6597. The different HPV types vary in their nucleotide sequence at positions 6581, 6584, 6590, 6593 and 6596 (as defined by HPV16 positional numbering). The consensus primer before bisulphite treatment is NGCNCAGGGHCAHAAYA (SEQ ID NO: 702) (where in standard notation; N=G, A, T or C; and H=A, T or C; and D=G, A, or T; and Y=A or C). The consensus primer after bisulphite treatment is DGTDTAGGGYTATAATA (SEQ ID NO: 703). As can be seen, the primer derived from the bisulphite treated derivative is much less degenerate than the primer based on the non-converted genomic sequence. In the case of the non-converted consensus primer there are a total of 288 primer combinations, while in the converted derivative only 18 primer combinations are required. In addition, the primer from the non-converted sequence has up to 4 base degeneracy at each site, while the converted derivative only has a maximum of 3 base degeneracy at any one site.

Conventional PCR primers are generally 20 to 30 nucleotides in length on complementary strands and at either end of the region to be amplified. Primers less than this generally have a low melting temperature especially if the primers are degenerate, which make PCR amplification problematic. Using the bisulphite complexity-reduction technique described herein, it is possible to locate regions of almost 100% sequence similarity between individual HPV types that ensure reliable amplification without the need to include such a large number of mismatched bases in the PCR primer as is the case for conventional degenerate primer sets.

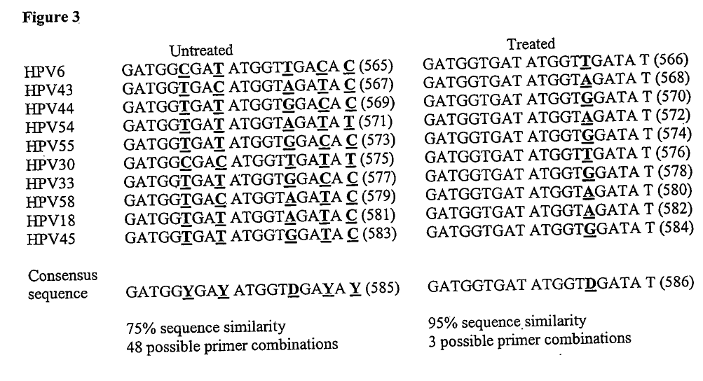

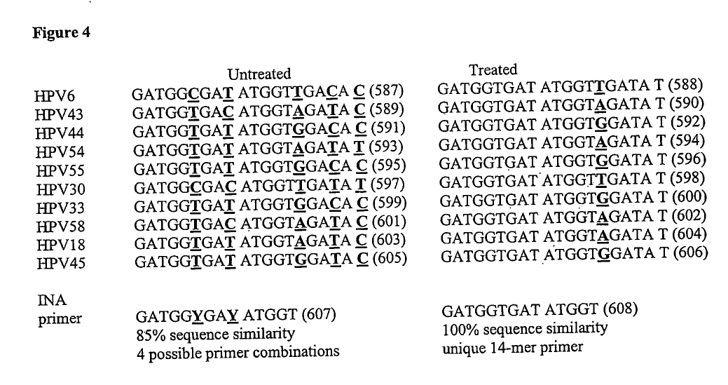

FIG. 3 shows DNA alignment of a 20 base pair region on the ‘top’ strand in the L1 region of HPV types (HPV 6, 43, 44, 54, 55, 30, 33, 58, 18 and 45) from positions 6225 to 6243. This region exhibits a sequence similarity of 75% before bisulphite treatment and over 90% sequence similarity after bisulphite treatment. The consensus primer of GATGGYGAYATGGTDGAYAY (SEQ ID NO: 704) has 48 possible primer combinations, but after complexity-reduction the HGS complexity-reduced consensus primer, GATGGTGATATGGTDGATAT (SEQ ID NO: 705), needs only 3 primer combinations. Even further improvements can be implemented by using Intercalating Nucleic Acids as primers and probes in hybridization reactions. These improvements are described in FIG. 4.

FIG. 4 shows the DNA alignment of the same 20 base pair region of individual HPV types as in FIG. 3 (6, 43, 44, 54, 55, 30, 33, 58, 18 and 45) from positions 6225 to 6243 as well as the sequence of high affinity INA primers and probes that can be used more effectively in hybridization reactions than standard oligonucleotides. Since INA primers can be far shorter in length than standard oligonucleotides, the first 14 bases of the above 20 base sequence can be constructed in INA form. Prior to bisulphite treatment, a 14 base INA with appropriately placed IPNs would have 85% sequence similarity over 14 bases, a figure which would rise to 100% sequence similarity over the same 14 base pair region after bisulphite treatment. This HGS complexity-reduced primer or probe, (GATGGTGATATGGT) (SEQ ID NO: 706), has no degeneracy whatsoever.

The significant advantages of INAs over the standard oligonucleotide primers and probes are that first, INAs can be made much shorter than conventional oligonucleotides due to the very high affinity of INA for complementary DNA. In fact, it has been shown that INAs as small as 12-14 bases can produce reliable signals in a PCR amplification reaction. Furthermore, any loss of specificity in the first round of amplification due to reduction in primer length is overcome in the second round. Second, INAs have a very high affinity for complementary DNA, with stabilisations of up to 10 degrees for internally placed intercalator pseudonucleotides (IPNs) and up to 11 degrees for end position IPNs. In addition, IPNs maximally stabilise DNA in AT-rich surroundings which make them especially advantageous when applied to bisulphite treated DNA. The IPNs are typically placed as bulge or end insertions in to the INA molecule. Thus by combining INAs with the bisulphite conversion methodology it is possible to reduce the size of the primer. This allows the creation of perfect matches for PCR amplification primers for the derivatives of individual HPV types, thus ensuring the reliable amplifications seen in FIG. 4.

We illustrate the general molecular detection methodology in step by step examples beginning with the use of ‘universal’ primers in the L1 region of different HPV types. The illustrations are for the ‘top’ strand only. It will be appreciated, however, that similar example can be obtained using the bottom strand.

### Is any HPV DNA of any Type Detectable in a Clinical Sample?

FIG. 5 shows PCR amplification products visualized after gel electrophoresis using HGS complexity-reduced primers for the L1 region of bisulphite-treated. HPV DNA extracted from liquid based cytology (LBC) specimens from sixteen female patients. The DNA amplification product is of the same size from all patients, and has been sequenced to verify that it is the correct amplified nucleic acid product from the region under scrutiny. The lengths of all the primers used in the generation of data in FIGS. 5 to 12 are shown in Table 2 and the sequences of the ‘universal’ complexity-reduced primers for FIG. 5 are also given in the Table 1.

TABLE 2
Expected fragment sizes in base pairs of amplified nucleic
acid products generated from different HPV derivatives
selected from the three major risk types.
HPV Risk Category PCR product band size (bp)

| High | Size | Medium | Size | Low | Size |
|---|---|---|---|---|---|
| HPV16 | 205 | HPV31 | 216 | HPV6 | 353 |
| HPV18 | 231 | HPV33 | 234 | HPV11 | 268 |
| HPV45 | 217 | HPV35 | 351 | HPV30 | 302 |
| HPV56 | 272 | HPV39 | 230 | HPV42 | 228 |
| | | HPV51 | 251 | HPV43 | 251 |
| | | HPV52 | 259 | HPV44 | 246 |
| | | HPV58 | 182 | HPV53 | 207 |
| | | | | HPV54 | 248 |
| | | | | HPV55 | 303 |
| | | | | HPV66 | 255 |

The data of FIG. 5 revealed that LBC samples from eleven of the 16 patients, (patient #1, #2, #3, #4, #6, #9, #11, #13, #14, #15, and #16) were positive for part of an HPV viral derivative. Given that these patient samples are HPV positive, what different types of viral genomes do these derivatives represent?

### Determining the Presence, or Absence, of a High-Risk Category of HPV Type—are There any High Risk Hpv Types Present in the Positive Patient Samples?

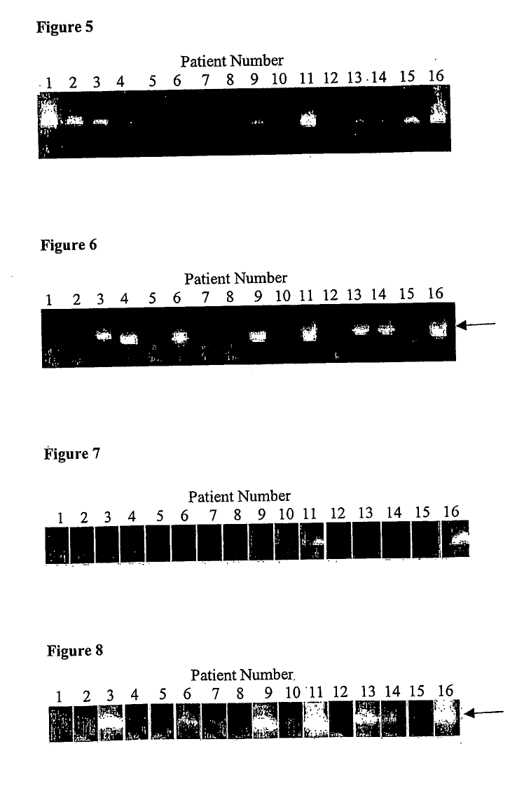

FIG. 6 shows multiplex PCR amplifications using HGS complexity-reduced primers for the E7 region where the primers are a mix made from the high risk HPV16, HPV18, HPV45 and HPV56 genomes. These primers will report on whether sequences from these four high-risk types are present, but not on which specific type it may be. The data reveal that positive amplifications are found in samples of patients #3, #4, #6, #9, #11, #13, #14 and #16. These eight patients thus harbour at least one high risk HPV type. Since the assay is a multiplex one, further PCR amplifications with primers specific for each high-risk HPV type are the next step. It should be noted that the negative cases provide an excellent control for the PCR reactions. The samples from patients #5, #7, #8, #10 and #12 should have yielded no amplified products (since they revealed no virus in the initial screen), and such is indeed the case.

Which of the Four High Risk Hpv Types does a Patient Harbour?

We first tested for the presence of the high-risk HPV16 type using HGS complexity-reduced PCR primers for the E7 region and analysis by gel electrophoresis. Only samples from patients #11 and #16 were positive, indicating that they carry at least part of the genome of the high risk HPV16 strain (FIG. 7).

In a similar manner, we tested for the presence of the high-risk HPV18 type using HGS complexity-reduced PCR primers for the E7 region and analysis by gel electrophoresis. Samples from patients #3, #6, #9, #11, #13 and #16 were positive, indicating that they carry this part of the genome of the high risk HPV18 strain (FIG. 8). Thus samples from patients #11 and #16 carried portions of the genome of both HPV16 and HPV18, indicating that they are infected with at least two high-risk HPV types.

The methods can be adapted to determine whether all the genomic regions of these high-risk HPV types are present in a sample (as would be the case if the entire virus was replicating as a full length episome or if it were fully integrated into the host genome), or has the viral genome undergone any deletions and is either replicating as a deleted entity, or is only part of the virus integrated into a human chromosome.

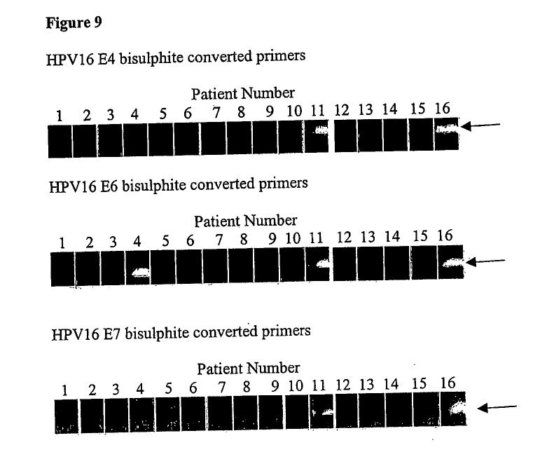

To determine whether additional regions of the high-risk HPV types, other than E7 were present in the various patient samples, a PCR amplification using HGS complexity-reduced primers for the E4, E6 and E7 regions of HPV16 were carried out and analysed by gel electrophoresis, (FIG. 9, top, middle and lower panels). Patients #11 and #16 carried all three tested regions, namely, E4, E6 and E7, whereas patient #4 only carried E6. Since samples from patients #11 and #16 were originally positive for L1, it is clear that these two patients carried the L1, E4, E6 and E7 regions, whereas patient #4 carried only the L1 and E4 regions.

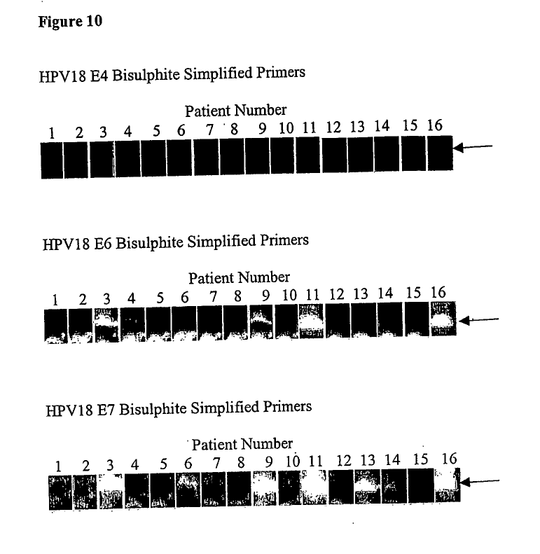

Similarly we determined if genomic regions E4, E6 and E7 were present in the high-risk HPV18 type. FIG. 10 reveals that patients #11 and #16 carried all three regions for HPV18, patients #3 and #9 carried E6 and E7, but not E4; patients #6 and #13 only carried fragment E7. Since samples from these patients were originally positive for L1, it can be seen that patients #11 and #16 carried the L1, E4, E6 and E7 regions; patients #3, #9 and #11 carried L1, E6′ and E7 regions; and patients #6 and #13 only carried L1 and E7.

Thus patients #11 and #16 were infected with two high risk HPV types, HPV16 and HPV18 and they carried all four genomic segments for which they had been tested.

Further analyses using additional patients revealed both the flow and the consistency of data production. Data for twenty patients, (denoted #A to #T are presented in FIG. 11 where the variation in viral risk type, in genomic fragment type and consistency of detection is evident.

First, patients #B, #C, #D, #E, #F, #G, #H, #I, #K, #N and #R were negative, denoted [neg], for PCR products based on the initial ‘universal’ complexity-reduced primer, and as expected, were subsequently negative for all further 28 PCR assays using high-, medium- and low-risk primers.

Patient #A was positive for HPV, denoted [pos] in column 1, but the sample did not contain any of the tested HPV high-, medium-, or low-risk types for each of the 28 different PCR amplifications. This patient was likely to carry one of the 80 or so HPV risk types which are not included in our test panel of 21 different HPV types.

Patients #J, #Q and #T were positive for high- and medium-risk HPV and subsequently were found to only carried genomic fragments from high- and medium-risk HPV types. Thus patient #J carried the E7 fragment of high-risk HPV16 and fragments from the medium-risk HPV31, 33 and 35 types.

Patients #L and #M were initially only positive for a medium-risk HPV and subsequent assays reveal only a medium-risk HPV33 type.

Patient #O was initially positive only for medium- and low-risk HPV types and subsequently was found to carry only sequences from the medium-risk HPV39 and the low-risk HPV 42 and 53 types.

Patients #P and #S were initially positive for all three risk categories and subsequently revealed all three risk category types when analysed in finer detail.

It will be appreciated that the examples described above are only illustrative of some of the range of testing possible. For example, in order to begin with an assay for any HPV type, instead of just the universal L1 fragment, we could have harnessed a multiplex complexity-reduced primer set that covered the entire HPV derivative. In this manner, there would be no ambiguity if the initial PCR amplification was negative.

In addition, one of the major problems that afflicts the prior art on sequence amplification is revealed in an analysis of the primer degeneracy problem (FIG. 12). PCR alpha is a PCR on samples from patients #s 21-42, for high- and medium-risk types, whereas PCR beta is for high-, medium-, and low-risk types on the same samples. FIG. 12 shows the effect of increasing primer degeneracy on PCR amplification efficiency. As can be seen, increasing the degeneracy of primer #1 in PCR reaction beta results in a complete failure to PCR amplify any sequences. The primer population has now become so degenerate that only a smear is produced. This is a result of the primer now binding to and extending off numerous less specific decoy loci in the derivative.

### The Details of the HPV Sequence Conversions and Properties of Primers

The results of the step by step conversion of an HPV sequence and the generation of appropriate primers is illustrated in FIGS. 13 and 14. Each HPV type has two complementary strands, denoted top and bottom, and each will be illustrated separately.

FIG. 13 shows the top strand of the HPV16 viral nucleic acid molecule in its three possible sequences; the normal viral sequence, the derivative sequence with uracils replacing cytosines, and the genomically simplified sequence where uracils have been replaced by thymines. The normal sequence containing all four regular bases begins as 5′ ACTACAATAATTCATG (SEQ ID NO: 706). When the cytosines are converted to uracils to form the derivative strand, the sequence still contains four bases, but one is now uracil, and it becomes 5′ AUTAUAATAATTUATG (SEQ ID NO: 707). When amplification takes place and the uracils are replaced by thymines, the sequence becomes 5′ ATTATAATAATTTATG (SEQ ID NO: 708) and is termed to be genomically simplified since it contains only three bases A, T and G. This formation of a derivative molecule followed by simplification is termed 4 to 3. It will be appreciated that if any part of the viral sequence becomes methylated on a cytosine, then that particular modified base, at that position, will not be converted to a uracil.

FIG. 14 shows the bottom strand of the HPV16 viral nucleic acid molecule in its three possible sequences; the normal viral sequence, the derivative sequence with uracils replacing thymines, and the genomically simplified sequence where uracils have been replaced by thymines. The bottom strand begins 5′ TGATGTTATTAAGTAC (SEQ ID NO: 709), becomes the derivative beginning 5′ TGATGTTATTAAGTAU (SEQ ID NO: 710), and finally a genomically simplified sequence beginning 5′ TGATGTTATTAAGTAT (SEQ ID NO: 711), etc.

Although the top and bottom strands were initially complementary, it can now be appreciated that in their genomically simplified forms they are quite different and non complementary. Hence primers used in amplifying regions of these two strands occur in different regions of the two genomically simplified landscapes. This is illustrated in the “helicopter” view of the different top and bottom strands.

FIG. 15 is a schematic of the genomic landscape of the top strand of HPV 16 from nucleotide position #1 to nucleotide position #7904 with the boxes indicating the positions of various nested primer sets used for amplification purposes. The positions of primers that are useful for amplifying DNA from a combinations of HPV types, such as high and medium risk, (denoted HM) and high, medium and low risk, (denoted HML); high, (denoted H) and high and medium, (denoted HM) are as indicated. Some regions of the top strand for example, have been found more useful for amplification purposes than other regions. It will be appreciated that using the present invention to simplify the genome of HPV, other regions of interest and use can be identified.

FIG. 16 is a schematic of the genomic landscape of the bottom strand of HPV 16 from nucleotide position #1 to nucleotide position #7904 with the boxes indicating the positions of various nested primer sets used for amplification purposes. The positions of primers that are useful for amplifying DNA from a combinations of HPV types are as indicated. The regions of the bottom strand that are useful for amplification purposes differ from those of the top strand. Some regions of the bottom strand for example, have been found more useful for amplification purposes than other regions. It will be appreciated that using the present invention to simplify the genome of HPV, other regions of interest and use can be identified.

### Clinical Samples and the Comparisons Between the FDA Approved Diagnostic Methodology Using Hybrid Capture 2 Versus the HGS “Derivative” and “Genomically Simplified” Amplification Technology

Currently the only FDA approved diagnostic test for the presence of various HPV types utilizes thirteen HPV types, as described earlier. We have found that the genomically simplified methodology according to the present invention is superior to that of the commercially available methods. FIGS. 17 through 21, Tables 1 through 4, and finally our description of a High Throughput High-Risk HPV DNA Detection and Typing Kit further teach the present invention.

In what follows, many of the clinical samples have been examined cytologically, and hence the cytological data can be correlated with the molecular data to determine the sensitivity and specificity of the competing technologies.

To begin cytologically, FIG. 17 shows a tissue section from a patient with cervical carcinoma. Arrow 1 reveals a darkened area of cancerous cells with large nuclei. Arrow 2 shows normal connective tissue. The cytological descriptions are termed normal if no abnormalities are visible cytologically; Low grade Squamous Intraepithelial Lesions (LSILs; CIN1); High grade Squamous Intraepithelial Lesions (HSILs); CIN2, CIN3 (Cervical Intraepithelial Neoplasia) as described in earlier classical descriptions by some pathologists, or as ASC-US, (Atypical Squamous Cells of Unknown Significance).

FIGS. 18 and 19 are illustrative of how to type for the presence of a high level HPV type, namely, is any one of thirteen HPV types present in a clinical sample, and if so, (as revealed by whether any sample is positive by visualization of an amplicon on a gel), to drill down and ask what specific HPV type was actually present. These steps were performed on 12 patients all of whom had a cytological examination and some of whom had surgical treatment for their medical condition.

FIG. 18 shows the results of PCR amplifications using the high-medium risk HGS complexity-reduced primers for the detection of thirteen HPV types, namely HPV 16, 18, 31, 33, 35, 39, 45, 51, 52, 56, 58, 59 and 68) for the ‘top’ strand of the E7 region of bisulphite-treated HPV DNA extracted from liquid-based cytology (LBC) specimens from twelve patient samples in which cytological analyses had been completed, (denoted #s 1 to 12). Positive results are seen from patients #2, 4, 7 and 11, three of whom were deemed to have high grade lesions as determined cytologically. None of the remaining individuals who had normal cytology, namely patients #1, 3, 5, 8, 10, 12 revealed any high-medium risk HPV, nor did the two patients who had received treatment for HSIL, #6 and 9.

To determine which HPV types were present in the four patients who tested positive for High-medium HPV types, further genotyping was performed.

FIG. 19 shows the results of a PCR amplification using material from clinical samples #2, #4, #7 and #11 from the patients that were positive for a high-medium risk HPV in FIG. 18 and a determination of exactly which of the HPV types (HPV 16, 18, 31, 33, 35, 39, 45, 51, 52, 56, 58, 59 and 68) were responsible for each of the amplicons visible in FIG. 18. As can be seen by visualization of amplicons in the four gels illustrated, patient #2 had HPV31, patients #4 and #7 had had HPV16 while patient #11 had HPV18 and HPV35.

While Liquid Based Cytology sampling is becoming the norm in HPV testing, many tests are still carried out on samples that have been taken from the urinogenital areas, fixed, sectioned and available on slides that, in general, have been archived. To determine how well the HGS genomically simplified method performs on such archival material, amplifications were performed on samples obtained from patients with High Grade Squamous Intraepithelial Lesions.

FIG. 20 shows the results of PCR amplification from archival paraffin sections from material from 16 patients with High grade Squamous Intraepithelial Lesions (HSILs) using high-medium risk primer sets (HPV 16, 18, 31, 33, 35, 39, 45, 51, 52, 56, 58, 59 and 68), made to the genomically simplified top strand of HPV. Fifteen of the 16 patients (94%) were positive by this methodology consistent with the literature on the presence of HPV in HSIL.

Finally, since most of the results described herein utilized the top strand of HPV for primer production, it was necessary to demonstrate the bottom strand would also be of equal use in HPV detection systems. This is illustrated in FIG. 21.

Primers used for the detection of the bottom strand HPV DNA sequences were designed for the detection of both high-medium risk types sets (HPV 16, 18, 31, 33, 35, 39, 45, 51, 52, 56, 58, 59 and 68) and low-risk types (HPV 6, 11, 42, 43, 44, 53, 54 and 55) resulting in a primer set that picks up ano-genital HPV types in a more universal fashion. Thus these primers detected the presence of HPV in samples A3 and A4 while the top strand high-medium primers did not. This indicates the presence of an HPV type not considered in the high-medium risk category.

FIG. 21 A shows the results of PCR amplification from Liquid Based Cytology samples using primers made to the bottom strand of bisulphite converted, genomically simplified DNA. The primers targeted the thirteen HPV types (high-medium risk HPV 16, 18, 31, 33, 35, 39, 45, 51, 52, 56, 58, 59, 68 and low-risk HPV 6, 11, 42, 43, 44, 53, 54 and 55). Amplicons are found in 40 of the 60 samples tested (67%) indicating the presence of an anogenital HPV infection.

FIG. 21 B shows the results of PCR amplification from Liquid Based Cytology samples using primers made to the top strand of bisulphite converted, genomically simplified DNA. The primers targeted the thirteen HPV types, (HPV 16, 18, 31, 33, 35, 39, 45, 51, 52, 56, 58, 59 and 68). Amplicons are visible in 28 of the 60 samples tested (47%) indicating the presence of a high-medium type HPV infection.

### Hybrid Capture 2 Tests for HPV Versus HGS Testing on the Same Samples

Results of the use of the present invention is shown in even greater detail in Tables 3 and 4 which show hundreds of clinical samples tested not only by competing methods, but which also have a cytological description of the material used for testing.

Tables 3A, B, C. Three different sets of Liquid Based Cytology clinical samples initially tested using the Digene methodology of Hybrid Capture 2, and then tested using the HGS amplification methodology for the presence of various HPV types.

TABLE 3A

| | | HM | HM-E7 | HC2 | | |
|---|---|---|---|---|---|---|
| ID# | Control | HPV | genotype | HR | RFU | Cytology |
| 1 | POS | NEG | | NEG | | Negative |
| 2 | POS | NEG | | NEG | | Negative |
| 3 | POS | NEG | | NEG | | Negative |
| 4 | POS | NEG | NEG | NEG | | Negative |
| 5 | POS | NEG | NEG | NEG | | Negative |
| 6 | POS | NEG | | NEG | | Negative |
| 7 | POS | NEG | | NEG | | Negative |
| 8 | POS | POS | | POS | | Low Grade |
| 9 | POS | POS | | POS | | High Grade |
| 10 | POS | NEG | | NEG | | Negative |
| 11 | POS | NEG | | NEG | | Negative |
| 12 | POS | NEG | | NEG | | Negative |
| 13 | POS | NEG | | NEG | | Negative |
| 14 | POS | NEG | | POS | 323 | Negative |
| 15 | POS | POS | | POS | 4103 | Low Grade |
| 16 | POS | POS | | POS | 428708 | Low Grade |
| 17 | POS | POS | 56 | NEG | | Not Done |
| 18 | POS | POS | 56 | POS | 377 | Not Done |
| 19 | POS | POS | | POS | 301 | Low Grade |
| 20 | POS | POS | | POS | 7562 | Low Grade |
| 21 | POS | NEG | | NEG | | Negative |
| 22 | POS | POS | | POS | 890 | Low Grade |
| 23 | POS | POS | 59 | NEG | | Negative |
| 24 | POS | NEG | | NEG | | Low Grade |
| 25 | POS | POS | | POS | 39404 | Low Grade |
| 26 | POS | POS | | POS | 67964 | Negative |
| 27 | POS | NEG | | NEG | | Negative |
| 28 | POS | NEG | | NEG | | Negative |
| 30 | POS | NEG | | NEG | | Negative |
| 31 | POS | NEG | | NEG | | Negative |
| 32 | POS | POS | | POS | 412424 | Low Grade |
| 33 | POS | POS | 16, 31 | NEG | | Negative |
| 34 | POS | NEG | | NEG | | Negative |
| 35 | POS | NEG | | | | |
| 36 | POS | POS | | POS | | Negative |
| *37 | POS | POS | 16, 33, 52 | NEG | | Negative |
| 38 | POS | POS | 16, 52 | NEG | | Negative |
| 39 | POS | POS | | POS | 510642 | Low Grade |
| 40 | POS | POS | | POS | 580914 | Low Grade |
| 41 | POS | NEG | | NEG | | Negative |
| 42 | POS | NEG | | NEG | | Negative |
| 43 | POS | POS | NEG | POS | 7939 | Low Grade |
| 44 | POS | NEG | | NEG | | Negative |
| 45 | POS | NEG | | NEG | | Negative |
| 46 | POS | POS | NEG | NEG | | Low Grade |
| | | | | | | CIN 1 |
| 47 | POS | NEG | | NEG | | Negative |
| 48 | POS | NEG | | POS | 341 | Negative |
| 49 | POS | NEG | | NEG | | Negative |
| 50 | POS | NEG | | NEG | | Low Grade |
| 51 | POS | NEG | | NEG | | Negative |
| 52 | POS | NEG | | NEG | | Negative |
| 53 | POS | NEG | | NEG | | Negative |
| 54 | POS | POS | 16, 31, 51 | POS | 211637 | Low Grade?? |
| 55 | POS | NEG | | NEG | | Negative |
| 56 | POS | NEG | | NEG | | Negative |
| 57 | POS | NEG | | POS | 783 | Negative Bx |
| 58 | POS | NEG | | NEG | | Negative |
| 59 | POS | NEG | | NEG | | Unsat Neg |
| 60 | POS | NEG | | POS | 1081 | Negative |
| 61 | POS | POS | 51 | POS | 3542 | High Grade |
| 62 | POS | NEG | | NEG | | Negative |
| 63 | POS | NEG | | NEG | | Negative |
| 64 | POS | POS | NEG | NEG | | Not Done |
| 65 | POS | POS | 56 | POS | 140824 | Low Grade |
| 66 | POS | POS | 16 | NEG | | Hx CIN 2 |
| 67 | POS | NEG | | NEG | | Negative |
| 68 | POS | NEG | | NEG | | Negative |
| 69 | POS | NEG | | NEG | | Negative |
| 70 | POS | POS | | POS | 12222 | CIN 1 HPV |
| 71 | POS | NEG | | POS | 1657 | Negative |
| 72 | POS | NEG | | NEG | | Negative |
| 73 | POS | NEG | | NEG | | Negative |
| 74 | POS | NEG | | NEG | | Negative |
| 75 | POS | POS | | POS | 79295 | Low Grade |

*Swab

TABLE 3B

| | | HM- | HM E7 | HC2 | HC |
|---|---|---|---|---|---|
| ID# | Control | HPV | Genotype | LR | HR |
| 76 | POS | POS | 31, 56 | − | + |
| 77 | POS | POS | 52 | − | + |
| 78 | POS | NEG | | − | − |
| 79 | POS | POS | 31 | − | − |
| 80 | POS | POS | 39, 59, 68 | + | + |
| 81 | POS | NEG | | − | − |
| 82 | POS | POS | 18 | − | + |
| 83 | POS | POS | 31 | + | − |
| 84 | POS | POS | 51 | − | + |
| 85 | POS | NEG | | − | − |
| 86 | NEG | POS | | − | − |
| 87 | POS | NEG | | − | − |
| 88 | POS | POS | 31 | − | − |
| 89 | POS | POS | 31 | − | − |
| 90 | POS | NEG | | − | − |
| 91 | POS | NEG | | − | − |
| 92 | POS | NEG | | − | − |
| 93 | POS | POS | 59 | − | + |
| 94 | POS | POS | 16, 51 | + | + |
| 95 | POS | POS | 39 | + | + |
| 96 | POS | NEG | | − | − |
| 97 | POS | POS | | − | − |
| 98 | POS | POS | | − | − |
| 99 | POS | NEG | | − | − |
| 100 | POS | NEG | | − | − |
| 101 | POS | NEG | | − | − |
| 102 | POS | POS | 18 | − | − |
| 103 | POS | NEG | | − | − |
| 104 | POS | POS | 33 | − | − |
| 105 | POS | NEG | | − | − |
| 106 | POS | POS | | − | − |
| 107 | POS | POS | 56 | − | + |
| 108 | POS | NEG | | − | − |
| 109 | POS | POS | 31 | − | − |
| 110 | POS | POS | 16, 31, 51, 52 | − | + |
| 111 | POS | POS | 52, 59 | − | + |
| 112 | POS | POS | 56 | − | + |
| 113 | POS | NEG | | − | − |
| 114 | POS | NEG | | − | − |
| 115 | POS | NEG | | − | − |
| 116 | POS | NEG | | − | − |
| 117 | POS | NEG | | − | − |
| 118 | POS | NEG | | − | − |
| 119 | POS | POS | 45 | − | − |
| 120 | POS | POS | 16, 45, 68 | − | + |
| 121 | POS | NEG | | − | − |
| 122 | POS | POS | 39, 68 | − | − |
| 123 | POS | POS | 39, 68 | − | + |
| 124 | POS | NEG | | + | − |
| 125 | POS | NEG | | − | − |
| 126 | POS | NEG | | − | − |
| 127 | POS | NEG | | − | − |
| 128 | POS | POS | 31 | − | − |
| 129 | POS | NEG | | − | − |
| 130 | POS | POS | 31 | + | − |
| 131 | POS | NEG | | − | − |
| 132 | POS | NEG | | + | − |
| 133 | POS | NEG | | − | − |
| 134 | POS | NEG | | − | − |
| 135 | POS | POS | 68 | − | − |
| 136 | POS | NEG | | − | − |
| 137 | POS | NEG | | − | − |
| 138 | POS | POS | 16 | − | + |
| 139 | POS | NEG | | − | − |
| 140 | POS | NEG | | − | − |
| 141 | POS | NEG | | − | − |
| 142 | POS | NEG | | − | − |
| 143 | POS | POS | 45 | − | − |
| 144 | POS | POS | 16, 45 | + | + |

TABLE 3C

| | | | HM-E7 | LR-E7 | | | | | |
|---|---|---|---|---|---|---|---|---|---|
| ID# | Control | HM-HPV | Genotype | Genotype | HC2-HR | RFU | HC2-LR | RFU | Cytology |
| 172 | POS | NEG | | | NEG | 55 | NEG | 65 | Normal |
| 173 | POS | NEG | | | NEG | 153 | NEG | 101 | Normal |
| 174 | NEG | POS | | | NEG | 292 | NEG | 105 | Normal |
| 175 | POS | POS | 59 | | POS | 768212 | NEG | 58 | LSIL |
| 176 | POS | NEG | | | NEG | 152 | NEG | 78 | Normal |
| 177 | POS | NEG | Not Detected | | POS | 734 | NEG | 46 | Normal |
| 178 | POS | NEG | | | NEG | 64 | NEG | 84 | LSIL |
| 179 | POS | POS | 45 | | POS | 204963 | NEG | 54 | LSIL |
| 180 | POS | NEG | 18 | | POS | 103404 | NEG | 42 | Normal |
| 181 | POS | NEG | | | NEG | 68 | NEG | 46 | Normal |
| 182 | POS | POS | 16 | | POS | 754 | POS | 29164 | LSIL |
| 183 | NEG | POS | NEG | 42 | POS | 568 | POS | 45548 | LSIL |
| 184 | POS | NEG | | | NEG | 168 | NEG | 58 | Normal |
| 185 | NEG | NEG | | | NEG | 62 | NEG | 40 | Normal |
| 186 | POS | POS | 18 | | POS | 3153 | NEG | 55 | HSIL |
| 187 | POS | NEG | | | NEG | 71 | NEG | 47 | Normal |
| 188 | POS | NEG | | | NEG | 66 | NEG | 36 | Normal |
| 189 | POS | NEG | | | NEG | 55 | NEG | 55 | Normal |
| 190 | POS | NEG | | | NEG | 34 | NEG | 34 | Normal |
| 191 | POS | NEG | | | NEG | 96 | NEG | 82 | ASCUS |
| 192 | POS | POS | 33 | | POS | 132844 | POS | 156190 | LSIL |
| 193 | POS | NEG | | | NEG | 115 | NEG | 63 | Normal |
| 194 | POS | POS | 51, 58, 68 | | POS | 153881 | POS | 52619 | LSIL |
| 195 | POS | NEG | | | NEG | 43 | NEG | 47 | Normal |
| 196 | POS | NEG | | | NEG | 112 | NEG | 60 | ASCUS |
| 197 | POS | NEG | | | NEG | 135 | NEG | 143 | Normal |
| 198 | NEG | NEG | | | NEG | 80 | NEG | 48 | Normal |
| 199 | POS | POS | 52 | | POS | 237578 | NEG | 103 | LSIL |
| 200 | NEG | POS | 33, 58 | | POS | 55052 | NEG | 80 | LSIL |
| 201 | POS | NEG | | | NEG | 57 | NEG | 69 | Normal |
| 202 | POS | POS | 51 | | POS | 105731 | NEG | 81 | Normal |
| 203 | POS | POS | 51, 56 | | POS | 712162 | NEG | 167 | LSIL |
| 204 | POS | NEG | | | NEG | 115 | NEG | 185 | Normal |
| 205 | POS | NEG | | | NEG | 92 | NEG | 36 | Normal |
| 206 | POS | NEG | | | NEG | 60 | NEG | 82 | Normal |
| 207 | POS | NEG | NEG | NEG | NEG | 105 | NEG | 55 | LSIL |
| 208 | NEG | POS | 51 | | POS | 336180 | NEG | 154 | LSIL |
| 209 | POS | POS | 16, 35 | | POS | 142232 | NEG | 150 | LSIL |
| 210 | NEG | NEG | | | NEG | 83 | NEG | 37 | Normal |
| 211 | POS | POS | 56 | | POS | 514728 | NEG | 49 | LSIL |
| 212 | POS | NEG | | | NEG | 56 | NEG | 52 | Normal |
| 213 | POS | NEG | | | NEG | 122 | NEG | 38 | Normal |
| 214 | POS | POS | 68 | | POS | 2536 | NEG | 48 | LSIL |
| 215 | POS | NEG | | | NEG | 42 | NEG | 60 | Normal |
| 216 | POS | POS | Not Detected | | POS | 2304 | POS | 31290 | LSIL |
| 217 | POS | POS | 56 | | POS | 13416 | NEG | 28 | LSIL |
| 218 | POS | POS | 16, 31 | | POS | 197601 | NEG | 99 | LSIL |
| 219 | NEG | NEG | | | NEG | 51 | NEG | 53 | Normal |
| 220 | POS | NEG | Not Detected | | POS | 1128 | NEG | 126 | Normal |
| 221 | POS | NEG | | | NEG | 116 | NEG | 100 | Normal |
| 222 | POS | POS | 56 | | POS | 456079 | NEG | 60 | LSIL |
| 223 | POS | NEG | | | NEG | 125 | POS | 114103 | ASCUS |
| 224 | NEG | NEG | | | NEG | 105 | NEG | 65 | ASCUS |
| 225 | POS | NEG | | | NEG | 65 | NEG | 73 | Normal |
| 226 | POS | NEG | | | NEG | 64 | NEG | 60 | Normal |
| 227 | POS | POS | 56 | | POS | 85613 | POS | 217685 | LSIL |
| 228 | POS | POS | 52 | | POS | 97012 | POS | 846 | Normal |
| 229 | POS | POS | 52 | | POS | 72556 | POS | 3114 | ASCUS |
| 230 | POS | NEG | | | NEG | 160 | NEG | 112 | Normal |
| 231 | POS | NEG | | | NEG | 154 | NEG | 52 | Normal |
| 232 | POS | POS | NEG | 53 | POS | 2628 | NEG | 86 | LSIL |
| 233 | POS | NEG | | | NEG | 86 | NEG | 58 | Normal |
| 234 | POS | POS | 16 | | POS | 96880 | NEG | 66 | ASCUS |
| 235 | POS | POS | 16, 51 | | POS | 568895 | NEG | 69 | LSIL |
| 236 | POS | POS | 56 | | POS | 95587 | NEG | 47 | Normal |
| 237 | POS | NEG | | | NEG | 117 | NEG | 113 | Normal |
| 238 | POS | POS | 16 | | POS | 27156 | NEG | 82 | LSIL |
| 239 | POS | NEG | Not Detected | | POS | 678 | NEG | 30 | Normal |
| 240 | POS | NEG | | | NEG | 90 | NEG | 82 | Normal |
| 241 | POS | NEG | Not Detected | | POS | 3130 | NEG | 88 | ASCUS |
| 242 | POS | POS | 59 | | POS | 95149 | NEG | 65 | LSIL |
| 243 | ND | ND | | | NEG | 56 | NEG | 184 | Normal |
| 244 | POS | POS | 56 | | POS | 22698 | POS | 81756 | LSIL |
| 245 | POS | POS | 18, 39 | | POS | 519145 | POS | 53404 | LSIL |
| 246 | POS | POS | 52, 68 | | POS | 454883 | POS | 4884 | LSIL |
| 247 | POS | NEG | | | NEG | 74 | NEG | 74 | ASCUS |
| 248 | POS | NEG | | | NEG | 98 | NEG | 36 | Normal |
| 249 | POS | POS | NEG | 30, 66 | POS | 6376 | NEG | 44 | LSIL |
| 250 | POS | NEG | | | NEG | 82 | NEG | 44 | Normal |
| 251 | POS | NEG | | | NEG | 43 | POS | 693 | Normal |
| 252 | POS | NEG | | | NEG | 150 | NEG | 60 | Normal |
| 253 | POS | NEG | | | NEG | 75 | NEG | 71 | Normal |
| 254 | POS | NEG | | | NEG | 127 | NEG | 51 | Normal |
| 255 | NEG | NEG | | | NEG | 64 | NEG | 68 | Normal |
| 256 | POS | POS | 33, 52, 58 | | POS | 356051 | NEG | 61 | LSIL |
| 257 | POS | NEG | | | NEG | 53 | NEG | 57 | Normal |
| 258 | POS | NEG | | | NEG | 267 | NEG | 111 | Normal |
| 259 | POS | NEG | | | NEG | 47 | POS | 1223 | Normal |
| 260 | POS | NEG | | | NEG | 110 | NEG | 32 | ASCUS |
| 261 | POS | NEG | | | NEG | 144 | NEG | 40 | Normal |
| 262 | POS | NEG | | | NEG | 55 | NEG | 67 | ASCUS |
| 263 | POS | NEG | | | NEG | 133 | NEG | 59 | Normal |
| 264 | POS | NEG | NEG | NEG | NEG | 94 | NEG | 52 | LSIL |
| 265 | POS | NEG | | | NEG | 93 | NEG | 63 | Normal |
| 266 | NEG | POS | 16, 31 | | POS | 1800 | NEG | 52 | HSIL |
| 267 | POS | NEG | | | NEG | 48 | NEG | 66 | Normal |
| 268 | ND | | | | NEG | 113 | NEG | 177 | Normal |
| 269 | ND | | | | POS | 1166 | NEG | 46 | Normal |
| 270 | ND | | | | NEG | 79 | NEG | 49 | Normal |
| 271 | POS | NEG | Not Detected | | POS | 1052 | NEG | 36 | Normal |

Table 3 has three parts, A, B and C, which reflect the different sources of discarded material used in the analyses. Three different sets of Liquid Based Cytology clinical samples which had initially been tested using the Digene methodology of Hybrid Capture 2, were then tested using the HGS amplification methodology for the presence of various HPV types.

Table 3A used discarded samples from patients tested in Australia. Column 1 gives the HGS identification number; column 2 is a control which determined whether any genomic DNA was present in a sample; column 3 describes whether the sample was positive for any high-medium risk HPV type (HPV 16, 18, 31, 33, 35, 39, 45, 51, 52, 56, 58, 59 and 68); column 4 provides the status of the type(s) of high-medium risk HPV found; column 5 shows the results obtained using the Hybrid Capture 2 test; column 6 provides the relative fluorescent units that are a characteristic of the Hybrid Capture 2 test, where relative fluorescent units are compared with internal standards to determine the cut-off for a positive or negative signal, and column 7 lists the cytological characteristics of the sample (if available). Finally *37 shows a swab sample rather than a LBC.

The comparison between the two methodologies is startling. Many Hybrid Capture 2 tests which are deemed to be negative, were in fact found positive by the HGS genomically simplified HGS test, and HGS test identified the type of HPV present. The Hybrid Capture 2 test therefore generated a high proportion of “false negatives”. These are individual patients who leave the clinic with a false sense of security after a test, believing that they are virus free, when in fact they are carriers. In addition, while the cytology may be negative for some individuals, the HGS test nevertheless unambiguously types the HPV which is present.

Furthermore, many Hybrid Capture 2 tests which were deemed to be positive on the basis of fluorescence were actually found negative by the HGS test. Since the HGS test is so sensitive, many patients found to be positive by the Hybrid Capture 2 test were in fact “false positives” determined by the HGS test. Patients in this category by the Hybrid Capture 2 test would leave the clinic with the anxiety of being potential cervical cancer victims, when in fact no virus is present.

Table 3B utilized discarded Liquid Based Cytology samples from patients in Hong Kong. Columns are similar except that the Hybrid Capture 2 tests have been carried out for both the low risk and high risk types. Again, the HGS genomically simplified test revealed many discordances between the two types of tests, even though the samples are from two quite different geographical locations and predominantly different ethnic groups.

Table 3C is also material from Hong Kong based samples and again the HGS test is discordant in a high proportion of cases with the Hybrid Capture 2 test. Column 1 and 2 represent the ID# and the positive control for the presence of human genomic DNA; column 3 indicates the presence or absence of high-medium risk HPV types; column 4 represents high-medium E7 genotype; column 5 shows the low-risk E7 genotype; column 6 represents the HC2 high risk call and column 7 the corresponding relative fluorescent units for that sample; column 8 and 9 show the HC2 low risk call and the corresponding relative fluorescent units for that sample; column 10 illustrates the cytology for that particular sample using standard descriptors.

TABLE 4
Genotyping of Liquid Based Cytology clinical
samples for various HPV types using primers
to the E7, E5 and E4 regions of HPV virus.

| | E7 | E6 | E5 | E4 |
|---|---|---|---|---|
| HR-HPV | Genotype | Genotype | Genotype | Genotype |
| POS | 31, 56 | | | 31 |
| POS | 52 | 52 | | |
| POS | (31) | | | |
| POS | 39, 59, 68 | 59 | | 59 |
| POS | 18 | | | |
| POS | (31) | | | |
| POS | 51 | 51 | | 51? |
| POS | (31) | | | |
| POS | (31) | | | |
| POS | 59 | | | |
| POS | 16, 51 | | | |
| POS | 39 | 39 | 39 | 39 |
| POS | 18 | | | |
| POS | 33 | | | |
| POS | 56 | 56 | | 56 |
| POS | 31 | | | |
| POS | 16, 31, 51, 52 | 31. 52 | | 31, 52 |
| POS | 52, 59 | 59 | | |
| POS | 56 | 56 | | 56 |
| POS | 45 | | | |
| POS | 16, 45, 68 | 16, 45 | 16 | 45? |
| POS | 39, 68 | | | 39 |
| POS | 39, 68 | | | |
| POS | (31) | | | |
| POS | (31) | | | |
| POS | 68 | | | |
| POS | 16 | 16 | 16 | |
| POS | 45 | | | |
| POS | 16, 45 | 45 | | 45? |
| POS | 33, 52, 58 | 33, 58 | | 33, 58 |
| POS | 52, 58 | 52 | | 52 |
| POS | 18, 33, 52, 56, 58 | 52, 56, 58 | | 52, 56 |
| POS | 18, 56, 58, 68 | 58 | | |
| POS | 56, 59, 68 | 56, 59 | | 56 |
| POS | 33, 51, 52, 58 | | | |
| POS | 33, 51, 52 58 | 51, 58 | | 51? |
| POS | 33, 52, 58 | 33 | | 33 |

Table 4 reveals that primers made to the E7 region of HPV are very useful primer sets (in preference to primers made to the E5, E6 and E4 regions of the virus).

Table 4 shows the results of genotyping Liquid Based Cytology clinical samples for various HPV types using primers to the E7, E6, E5 and E4 regions of HPV virus. The presence of HPV types in column 1 and the presence or absence of amplicons using the different primer sets to the E7, E6, E5 and E4 regions of the virus is shown in column 2-5. It is salient that E7 primers picked up the particular HPV types that are present, but in many cases E6, E5 and E4 fail to do so. E7 is therefore an excellent region to use. The reasons for this are that HPV often deletes portions of it's genome after infecting a cell, and E7 is a region that is retained with higher probability than others.

Amplified DNA is from HPV

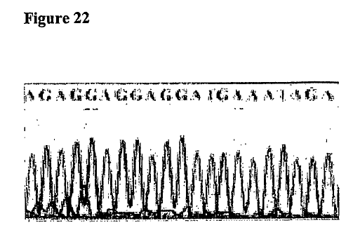

A clinical sample from the region of a human cervix, or from a Liquid Based Cytology sample, usually contains a heterogeneous population of human cells, together with a microorganism flora that can be extensive. Amplification of HPV sequences from such a heterogeneous source (in all cases we have tested), yield amplicons of the correct size as estimated from their migration on gels. However, the best indicator that the amplicons are indeed from HPV, and not from a source that serendipitously has the same molecular weight as the visible bands on a gel, is to excise a given band from a gel and subject the DNA within it to direct sequence analysis. We have carried this analysis and have confirmed that the DNA sequence does indeed correspond to that of HPV16. Results of such an analysis are shown in FIG. 22.

### High Throughput HPV Assay

The present invention can be used step by step in a high throughput manner using a 96 well plate in which many samples are simultaneously tested for HPV. This is illustrated by instructions for a potential commercial kit as follows.

TABLE 5
Contents of an HPV High Throughput
DNA Bisulphite Modification Kit

| Component Name | Contents | | Part Number |
|---|---|---|---|
| Lysis Buffer | 1 × 23 | ml | |
| Proteinase K | 2 × 1 | ml | |
| Reagent 1 | 1 × 20.8 | ml | |
| Reagent 2 | 1 × 8 | g | |
| Reagent 3 | 1 × 25 | ml | |
| Reagent 4 | 1 × 7 | ml | |
| Control Sample 1 | 1 × 40 | μl | |
| Control Sample 2 | 1 × 20 | μl | |
| Control Primers 3A & 3B | 2 × 40 | μl | |
| Plate 1: Incubation plate | 1 × 96 | well | |
| Plate 2: Conversion plate | 1 × 96 | well | |
| Plate 3: Purification plate | 1 × 96 | well | |
| Plate 4: Wash plate | 1 × 96 | well | |
| Plate 5: Elution plate | 1 × 96 | well | |
| Sealing caps | 36 × 8 | cap strips | |
| Plate 6: High Risk HPV plate | 2 × 96 | well | |
| Plate 7: HPV Typing Plate | 8 × 96 | well | |
| Plate 8: Control Plate | 2 × 96 | well | |

NB. Individual High-Risk Typing primers sets are available from Human Genetic Signatures (enquire at <hpv@geneticsignatures.com>)
Note:
Control Samples/Primers 1, 2, 3A and 3B should be stored at −20° C. upon receipt.

### Materials and Equipment Required (not Supplied)

Either a vacuum manifold or a centrifuge is required as follows:

A vacuum manifold for 96 well plates with a pump to apply at least −10 in Hg (4.9 psi) pressure. (In-house testing was carried out using the Biorad Aurum Manifold but other manifolds may be adapted for use.) or

A centrifuge with a rotor compatible with a high clearance 96 well format plate. (In-house testing was carried out using an Eppendorf 5810).
Heated lid PCR Thermal Cycler compatible for 96 well format 0.2 ml low profile plates
Heated lid PCR Thermal Cycler compatible for 384 well format (for HPV typing)
80% isopropanol (molecular biology grade)
Water (molecular biology grade)
NaOH pellets (Analytical Grade)
2×PCR master-mix (Promega Cat#M7505 1000r×n)
E-Gel System Mother E-Base™ device (Invitrogen EB-M03)
E-gels 96 High-Throughput 2% Agarose (Invitrogen Cat#G7008-02)
E-gel Low range marker (Invitrogen Cat#12373031)
Reagent reservoirs ×5

### Standard Laboratory Equipment (not Supplied)

Multi-channel pipette, up to 1 ml volume (200 μl-1000 μl)
Multi-channel pipette, up to 200 μL volume (20 μl-200 μl)
Multi-channel pipette, up to 10 μL volume (1 μl-10 μl)
Lint-free tissue
Timer
Aerosol barrier tips (10 μl-1000 μl)
Transilluminator
Gel Documentation system
Glison P1000
Gilson P200
Gilson P20

### Methods

If using HPV High Throughput DNA Bisulphite Modification Kit for the first time, it is highly recommended that the detailed methodology in the User Guide be read before carrying out the bisulphite conversion method.

Using the HPV High Throughput DNA Bisulphite Modification Kit eliminates the need for pre-digestion of genomic DNA prior to conversion.

Do not reduce the volume of the bisulphite reagent added to the DNA sample. In-house tests have shown that reduction of the bisulphite reagent is detrimental to the reaction.

This kit is optimized for starting DNA concentrations from 1 ng up to 4 μg of genomic DNA.

### Sample Preparation

Shake the Liquid Based Sample (PreservCyt®) vial vigorously by hand to resuspend any sedimented cells and ensure the solution is homogeneous.
Transfer 4 ml of the resuspended cells to a 15 ml Costar centrifuge tube. If there is less than 4 ml of media transfer all the material to a 15 ml Costar centrifuge tube and make the volume to 4 ml with sterile distilled water. A minimum volume of 1 ml sample is required for accurate testing.
Centrifuge the tubes in a swing-out bucket rotor at 3000×g/15 minutes.
Carefully decant and discard the supernatant without disturbing the pelleted cellular material.
Resuspend the pelleted cells in 200 μl of lysis buffer and mix well until the solution is homogeneous.
Add 20 μl of Proteinase K and incubate to each well of the incubation plate.
Transfer 80 μl of the sample to the Incubation plate (Plate 1) cover with sealing caps and incubate at 55° C./1 hour.

### Protocol Preparation

Combine the total volume of Reagent 1 to the Reagent 2 bottle and mix by gentle inversion. Note: Once mixed Reagents 1 and 2 are stable for up to 1 month at 4° C. in the dark. Reagents 1, 2, 3 and 4 are stable at room temperature for 1 year from the date of manufacture.
Make a fresh NaOH solution each time (eg. 1 g NaOH in 8.3 ml water) and add 5 μl to each well of the Conversion plate (Plate 2).
Add 5 μl of Control Sample 1 to 15 μl of water (molecular biology grade) and treat in parallel with the test samples.
Transfer 20 μl of the cell lysate to the Conversion plate (Plate 2) and mix gently.
Seal the Conversion plate (Plate 2) with the sealing film provided and incubate in an oven at 37° C./15 minutes. After incubation, centrifuge the plate briefly before removing the film to precipitate any condensation on the film.
Seal the Incubation plate (Plate 1) with sealing caps provided and store at −20° C.
Ensure that Reagent 3 has not formed a solid precipitate. If so, warm the solution (not higher than 80° C.) and mix.

### Centrifugation Protocol

Add 220 μl of the combined Reagent 1 and Reagent 2 into each well of the Conversion plate (Plate 2), using a multi-channel pipette then mix by gentle pipetting and seal the plate with the 8 strip sealing caps provided.
Incubate the Conversion plate (Plate 2) in an oven at 55° C./3 hours.

Bisulphite treatment can be carried out in as little as one hour, however, reducing incubation time can result in regional non-conversion within the amplicon. Incubation times of less than 3 hours are therefore not recommended.
Following incubation add 240 μl of Reagent 3 (Refer to Important Protocol Preparation) to each well of the Conversion plate (Plate 2).
Place the Purification plate (Plate 3) on top of the Wash plate (Plate 4).
Transfer the samples from the Conversion plate (Plate 2) to the corresponding wells of the Purification plate (Plate 3) and cover with the sealing film provided.
Place the Purification plate (Plate 3)/Wash plate (Plate 4) combination into the centrifuge and spin at 1,000 rcf at room temperature/4-5 minutes.
Discard the flow-through from the Wash plate (Plate 4) then replace it under the Purification plate (Plate 3). Add 0.8 ml of 80% isopropanol (molecular biology grade) to each well of the Purification plate (Plate 3).
Centrifuge at 1,000 rcf at room temperature/1 minute.
Remove the Wash plate (Plate 4), discard the flow-through then replace and centrifuge at 1,000 rcf/2 minutes at room temperature.
Place the Purification plate (Plate 3) on top of the Elution plate (Plate 5) ensuring the tips of the Purification plate (Plate 3) are positioned within the appropriate wells of the Elution plate (Plate 5).
Add 50 μl of Reagent 4 to each sample well of the Purification plate (Plate 3) using a multi-channel pipette, placing the pipette tip close to the membrane surface without touching it.
Incubate at room temperature 1-2 minute.
Centrifuge the Purification plate (Plate 3)/Elution plate (Plate 5) combination at 1,000 rcf at room temperature/1 minute.
Remove the Elution plate (Plate 5) and seal with the sealing caps provided.
Incubate the plate in a heated lid PCR machine at 95° C./30 minutes

The DNA samples are now converted and ready for PCR amplification. After incubation centrifuge the plate briefly to remove any condensation from the sealing caps.

### Internal Control PCR Reaction

Genomic DNA and control PCR primers have been provided to allow for easy troubleshooting. Control Samples 1 (purple) and 2 (green) are provided as process controls. Control Sample 1 is untreated DNA with sufficient material provided for 8 conversion reactions. Control Sample 2 is bisulphite treated DNA with sufficient material provided for 20 PCR amplifications. Control Primers 3A (yellow) and 3B (red) are PCR primers and may be used to check the integrity of the recovered DNA (sufficient for 20 PCR amplifications provided).

‘Nested’ PCR primers are used to further improve the sensitivity of the detection that is achieved with HPV High Throughput DNA Bisulphite Modification Kit. The control primers are conventional bisulphite PCR primers and have been optimised for two rounds of PCR amplification. The use of these PCR primers for single round PCR is not recommended as in most cases no visible amplicon band will be seen following agarose gel electrophoresis.

Note: This protocol is based on the use of a heated-lid thermal cycler. If a heated-lid thermal cycler is unavailable, overlay reactions with mineral oil.

### Control Reactions:

Control Sample 1 (purple) contains untreated genomic DNA (50 ng/μl)
Control Sample 2 (green) contains bisulphite treated human DNA (20 ng/μl)
Control Primers 3A (yellow) contains First round PCR primers
Control Primers 3B (red) contains Second round PCR primers

### Control PCR

Control Primers 3A (First round PCR primers) and Control Primers 3B (Second round PCR primers) are validated ‘nested’ primers with sufficient volume supplied for up to 20 control PCR reactions. These primer samples have been supplied to facilitate the trouble-shooting process if required, and may also be used to assess the quality of your modified DNA.

Note: The Second round PCR Reactions may be prepared in parallel with the First round PCR Reactions and frozen until required.

### High-Risk PCR Amplification

### First Round Amplification

For each reaction, add 12.5 μl of PCR Master Mix (for example, Promega Master Mix) and 9.5 μl water (molecular biology grade) in the High-Risk PCR plate provided. If you are setting up 96 samples combine 1.25 ml Master mix, 850 μl of water and 200 μl of primer mix in an appropriate tube and mix well. Then using a multi channel pipette add 23 μl of the reaction mix to each well in the High-Risk HPV plate (Plate 6) provided.
Add 2 μl of Control Primers 3A to the appropriate well to control well H10 and H11.
Add 2 μl of the required modified DNA from the Elution plate (Plate 5) to the High-Risk HPV plate (Plate 6) provided and 2 μl of Control Sample 2 to well H11 then store the remainder at −20° C. for subsequent HPV typing (see below for High-Risk plate lay-out).
Run the following PCR program.

| | |
|---|---|
| 95° C./3 min | 1 cycle |
| 95° C./1 min | 30 cycles |
| 42° C./2 min | |
| 60° C./2 min | |
| 60° C./10 min | 1 cycle |

### Second Round Amplification

Add 2 μl of the first round amplified DNA to second round mixes, prepared exactly the same as for the first round amplifications.
Run the following PCR program

| | |
|---|---|
| 95° C./3 min | 1 cycle |
| 95° C./1 min | 30 cycles |
| 42° C./2 min | |
| 60° C./2 min | |
| 60° C./10 min | 1 cycle |

### Electrophoresis

Remove the 96 well 2% E-gel from the foil wrapper and remove the red 96 well comb.
Add 10 μl of sterile water to each well of the gel using a multi-channel pipette.
Add 10 μl of DNA marker to the marker wells.
Transfer 10 μl of amplified product to each well of the E-gel using a multichannel pipette.
Set the E-base for 5-7 minutes and press pwr/prg.
Record the results using an UV transilluminator and gel documentation software.

### HPV Typing

### First Round Amplification

The High-Risk Typing plate (Plate 8) contains strain specific primers directed against the following high-risk HPV types: 16, 18, 31, 33, 35, 39, 45, 51, 52, 56, 58, 59 and 68. There is sufficient DNA remaining in the Elution plate (Plate 5) to type each sample for all high-risk strains.
Remove the Elution plate (Plate 5) from the −20° C. freezer.
Any samples positive by the high-risk universal amplification can now be typed using the strain specific primers (see below for typing plate set-up)
For each reaction, add 12.5 μl of PCR Master Mix (for example, Promega Master Mix) and 8.5 μl water into each well of the PCR plate provided. If you have 6 samples to type add 1187.5 μl of Master Mix and 807.5 μl of water into an appropriate tube, mix well then add 21 μl to each well of the HPV Typing plate (Plate 7) as indicated below.
Add 2 μl of the appropriate primer set to each well as indicated below.
If the typing is being carried out in 384 well format and 24 samples are available for typing add 4.5 ml of Master Mix and 3.42 ml of water into an appropriate tube, mix well then add 21 μl to each well of the 384 well plate as indicated below. Then add 2 μl of the appropriate primer set to each well as indicated below.
Add 2 μl of High-Risk positive sample (from Elution plate, Plate 5) to the appropriate wells of the typing plate.
Set up sufficient tubes for each of your samples and a ‘no template’ (negative) control.
Run the following PCR program.

| | |
|---|---|
| 95° C./3 min | 1 cycle |
| 95° C./1 min | 30 cycles |
| 45° C./2 min | |
| 65° C./2 min | |
| 65° C./10 min | 1 cycle |

### Second Round Amplification

Add 2 μl of the First round amplified DNA to Second round mixes, prepared exactly the same as for the First round amplifications.
Run the following PCR program

| | |
|---|---|
| 95° C./3 min | 1 cycle |
| 95° C./1 min | 30 cycles |
| 45° C./2 min | |
| 65° C./2 min | |
| 65° C./10 min | 1 cycle |

### Electrophoresis

Remove the 96 well 2% E-gel from the foil wrapper and remove the red 96 well comb.
Add 10 μl of sterile water to each well of the gel using a multi-channel pipette.
Add 10 μl of DNA marker to the marker wells.
Transfer 10 μl of amplified product to each well of the E-gel using a multichannel pipette.
Set the E-base for 5-7 minutes and press run.
Record the results using an UV transilluminator and gel documentation software.
The sample has now been typed.

Troubleshooting

| PROBLEMS | POSSIBLE SOLUTIONS |
|---|---|
| No PCR product was | PCR has failed - make sure all the |
| found for any sample | components were added to the tube and |
| | that the PCR cycle was correct. |
| | Confirm that the polymerase is within its |
| | storage date and that it retains its activity. |
| No PCR product was | Modification has failed - check that the |
| found for any sample | NaOH solution was fresh and that |
| except for Control | combined Reagent # 1 and Reagent 2 was |
| Sample 2 | no older than 4 weeks. |
| | Make sure that all the steps in the |
| | modification and clean up protocols were |
| | followed. |
| | DNA was degraded during modification - |
| | check that all reagents and tubes used |
| | during the procedure were of molecular |
| | biology quality (ie DNase free). |
| | Modification was incomplete. Return the |
| | samples to 95° C. for a further 15 minutes. |
| | Sample DNA was degraded before |
| | modification- check that the DNA has been |
| | stored/handled correctly. |
| PCR products were | Check that the DNA concentration is not |
| present only in the | too dilute. |
| control reactions | Check that the PCR-grade water and not |
| | the template was added to the negative |
| | control. |
| PCR products were | Make sure that the PCR is being set up in |
| present in all the | a separate area with dedicated reagents |
| lanes including the | and equipment to prevent cross |
| ‘no-template’ | contamination. |
| (negative) control | |

Bisulfite-treated HPV DNA from sources, when amplified using genomically simplified primers, be they oligonucleotides or modified nucleic acids such as INAs provide an unsurpassed detection system for finding HPV of any type within a sample, be that sample from human clinical material or at another extreme from an environmental source. The present invention has been developed for a clinically relevant virus (HPV) believed to be causative for a human cancer.

The practical implications of the detection assay according to the present invention can be varied. While the principles described in detail above have been demonstrated using PCR for amplification, readouts can be engaged via any methodology known in the art. With the current emphasis on microarray detection systems, one would be able to detect a great diversity of HPV using genomically simplified DNA since the bisulfite treatment reduces the genomic complexity and hence allows for more types of HPV to be tested on microarrays with a smaller number of detectors (features).

In summary, the HGS genomically simplified primer methodology yields consistent data sets that has been correlated with the clinical phenotypes of a number of patients.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An assay for detecting human papilloma virus (HPV) comprising:
treating the viral nucleic acid with an agent that modifies cytosine to form derivative viral nucleic acid;
amplifying at least a part of the derivative viral nucleic acid to form an HPV-specific nucleic acid molecule; and
looking for the presence of an HPV-specific nucleic acid molecule, wherein detection of the HPV-specific nucleic acid molecule is indicative of HPV.

2. The assay according to claim 1 further comprising:
providing HPV primers capable of allowing amplification of an HPV-specific nucleic acid molecule.

3. The assay according to claim 1 or 2 wherein the virus is in a sample selected from the group consisting of swab, biopsy, smear, Pap smear, blood, plasma, serum, blood product, surface scrape, spatula, liquid suspension, frozen material, paraffin blocks, glass slides, forensic collection systems and archival material.

4. The assay according to claim 3 wherein the sample is smear, Pap smear or liquid suspension of cells.

5. The assay according to any one of claims 1 to 4 wherein the agent modifies cytosine to form uracil in the derivative nucleic acid.

6. The assay according to claim 5 wherein the agent is selected from bisulfite, acetate or citrate.

7. The assay according to claim 6 wherein the agent is sodium bisulfite.

8. The assay according to any one of claims 1 to 7 wherein the agent modifies an cytosine to a uracil in each strand of complementary double stranded viral nucleic acid forming two derivative but non-complementary viral nucleic acid molecules.

9. The assay according to any one of claims 1 to 8 wherein the derivative viral nucleic acid has a reduced total number of cytosines compared with the corresponding untreated viral nucleic acid.

10. The assay according to any one of claims 1 to 9 wherein the amplification is carried out by polymerase chain reaction (PCR), ligase chain reaction (LCR), isothermal amplification, signal amplification or combination thereof.

11. The assay according to claim 10 wherein the amplification is carried out by PCR.

12. The assay according to any one of claims 1 to 11 wherein amplification forms an HPV-specific nucleic acid molecule that does not form part of a natural HPV genome.

13. The assay according to any one of claims 1 to 12 wherein the HPV-specific nucleic acid molecule is specific for an HPV species, a type of HPV or sub-type of HPV.

14. The assay according to claim 13 wherein the HPV type can confer a high, medium or low level oncogenic status on a given tissue in a particular human ethnic lineage.

15. The assay according to claim 14 wherein high risk HPV types are HPV16, 18, 45 and 56, medium risk HPV types are HPV31, 33, 35, 39, 51, 52, 56, 58, 59 and 68, and low risk types are HPV6, 11, 26, 30, 40, 42, 43, 44, 53, 54, 55, 66, 73, 82, 83 and 84.

16. The assay according to claim 15 wherein HPV16, 18, 31, 33, 35, 39, 45, 51, 52, 56, 58, 59 and 68 are detected.

17. The assay according to any one of claims 1 to 16 wherein the HPV-specific nucleic acid is detected by gel electrophoresis, hybridisation with labelled probes, use of tagged primers that allow subsequent identification, an enzyme linked assay, or use of fluorescently-tagged primers that give rise to a signal upon hybridisation with the target DNA.

18. An HPV primer or probe comprising one or more of SEQ ID NO: 1 to SEQ ID NO: 516.

19. The HPV primer or probe according to claim 18 for detecting high-risk HPV strains comprising one or more of SEQ ID NO: 333 to SEQ ID NO: 350.

20. The HPV primer or probe according to claim 18 for detecting HPV comprising SEQ ID NO: 462, SEQ ID NO: 479, SEQ ID. NO: 463, SEQ ID NO: 478, SEQ ID NO: 470, SEQ ID NO: 485, and SEQ ID NO: 486.

21. A kit for the detection of HPV comprising two or more HPV primers or probes according to any one of claims 18 to 20 together with suitable reagent or diluent.

22. A derivative HPV nucleic acid comprising at least 15 nucleotides as herein before defined.

23. The derivative HPV nucleic acid according to claim 22 comprising high-risk HPV16, 18, 45 or 56.

24. The derivative HPV nucleic acid according to claim 22 comprising medium risk HPV 31, 33, 35, 39, 51, 52, 58, 59 and 68.

25. The derivative HPV nucleic add according to claim 23 or 24 comprising SEQ ID NO: 614, SEQ ID NO: 617, SEQ ID NO: 620, SEQ ID NO: 623, SEQ ID NO: 626, SEQ ID NO: 629, SEQ ID NO: 632, SEQ ID NO: 635, SEQ ID NO: 638, SEQ ID NO: 641, SEQ ID NO: 644, SEQ ID NO: 647, SEQ ID NO: 650, SEQ ID NO: 653, SEQ ID NO: 656, SEQ ID NO: 659, SEQ ID NO: 662, SEQ ID NO: 665, SEQ ID NO: 668, SEQ ID NO: 671, SEQ ID NO: 674, SEQ ID NO: 677, SEQ ID NO: 680, SEQ ID NO: 683, SEQ ID NO: 686, or SEQ ID NO: 689, parts thereof comprising at least 15 nucleotides, and nucleic acid molecules capable of hybridizing under stringent conditions to SEQ ID NO: 614, SEQ ID NO: 617, SEQ ID NO: 620, SEQ ID NO: 623, SEQ ID NO: 626, SEQ ID NO: 629, SEQ ID NO: 632, SEQ ID NO: 635, SEQ ID NO: 638, SEQ ID NO: 641, SEQ ID NO: 644, SEQ ID NO: 647, SEQ ID NO: 650, SEQ ID NO: 653, SEQ ID NO: 656, SEQ ID NO: 659, SEQ ID NO: 662, SEQ ID NO: 665, SEQ ID NO: 668, SEQ ID NO: 671, SEQ ID NO: 674, SEQ ID NO: 677, SEQ ID NO: 680, SEQ ID NO: 683, SEQ ID NO: 686, or SEQ ID NO: 689.

26. A simplified HPV nucleic acid comprising at least 15 nucleotides as herein before defined.

27. The simplified HPV nucleic acid according to claim 26 comprising high-risk HPV16, 18, 45 or 56.

28. The simplified HPV nucleic acid according to claim 26 being medium risk HPV 31, 33, 35, 39, 51, 52, 58, 59 and 68.

29. The simplified HPV nucleic acid according to claim 27 or 28 comprising SEQ ID NO: 615, SEQ ID NO: 618, SEQ ID NO: 621, SEQ ID NO: 624, SEQ ID NO: 627, SEQ ID NO: 630, SEQ ID NO: 633, SEQ ID NO: 636, SEQ ID NO: 639, SEQ ID NO: 642, SEQ ID NO: 645, SEQ ID NO: 648, SEQ ID NO: 651, SEQ ID NO: 654, SEQ ID NO: 657, SEQ ID NO: 660, SEQ ID NO: 663, SEQ ID NO: 666, SEQ ID NO: 669, SEQ ID NO: 672, SEQ ID NO: 675, SEQ ID NO: 678, SEQ ID NO: 681, SEQ ID NO: 684, SEQ ID NO: 687, or SEQ ID NO: 690; parts thereof comprising at least 15 nucleotides, and nucleic acid molecules capable of hybridizing under stringent conditions to SEQ ID NO: 615, SEQ ID NO: 618, SEQ ID NO: 621, SEQ ID NO: 624, SEQ ID NO: 627, SEQ ID NO: 630, SEQ ID NO: 633, SEQ ID NO: 636, SEQ ID NO: 639, SEQ ID NO: 642, SEQ ID NO: 645, SEQ ID NO: 648, SEQ ID NO: 651, SEQ ID NO: 654, SEQ ID NO: 657, SEQ ID NO: 660, SEQ ID NO: 663, SEQ ID NO: 666, SEQ ID NO: 669, SEQ ID NO: 672, SEQ ID NO: 675, SEQ ID NO: 678, SEQ ID NO: 681, SEQ ID NO: 684, SEQ ID NO: 687, or SEQ ID NO: 690.

30. Use of the derivative or simplified HPV nucleic acid according to any one of claims 22 to 29 to obtain probes, primers or nucleic acid sequences for HPV detection.

31. An assay for detecting the presence of HPV in a sample comprising:
obtaining viral nucleic acid from a sample;
treating the viral nucleic acid with bisulphite under conditions that cause cytosines in the viral nucleic acid to be converted to uracil to form derivative viral nucleic acid;
providing primers capable of binding to regions of derivative viral nucleic acid, the primers being capable of allowing amplification of a desired HPV-specific nucleic acid molecule to the derivative viral nucleic acid;
carrying out an amplification reaction on the derivative viral nucleic acid; and
looking for the presence of a desired amplified nucleic acid product, wherein detection of the amplified product is indicative of the presence of HPV in the sample.

32. The assay according to claim 31 further comprising:
treating a sample having HPV present with an additional test which can determine the type, subtype, variant or genotype of HPV in the sample.
